(19) United States
(12) Patent Application Publication
(10) Pub. No.: US 2019/0192517 A1
(43) Pub. Date: Jun. 27, 2019

(54) TREATMENT OF SQUAMOUS CELL CARCINOMAS WITH INHIBITORS OF ERK

(71) Applicant: Kura Oncology, Inc., San Diego, CA (US)

(72) Inventors: Francis Burrows, Solana Beach, CA (US); Dana Hu-Lowe, Encinitas, CA (US); Linda Kessler, Poway, CA (US)

(21) Appl. No.: 16/222,398

(22) Filed: Dec. 17, 2018

Related U.S. Application Data
(63) Continuation-in-part of application No. PCT/US2017/038084, filed on Jun. 19, 2017.
(60) Provisional application No. 62/352,533, filed on Jun. 20, 2016.
(60) Provisional application No. 62/428,379, filed on Nov. 30, 2016.
(60) Provisional application No. 62/502,996, filed on May 8, 2017.

(51) Int. Cl. A61K 31/517; C12Q 1/6886; C12Q 1/6827; C12Q 1/6851; C12Q 1/686; A61P 35/00

(52) U.S. Cl. CPC A61K 31/517; C12Q 1/6886; C12Q 1/6827; A61K 45/06; C12Q 1/686; A61P 35/00; C12Q 1/6851

(57) ABSTRACT

The present disclosure provides methods and systems for identifying and/or treating subjects having cancer, such as squamous cell carcinoma, who are more likely to respond to treatment with an ERK inhibitor.

### CROSS-REFERENCE

This application is a continuation in part of International Patent Application PCT/US2017/038084, filed Jun. 19, 2017, which claims the benefit of U.S. Provisional Application No. 62/352,533, filed Jun. 20, 2016; U.S. Provisional Application No. 62/428,379, filed Nov. 30, 2016; and U.S. Provisional Application No. 62/502,996, filed May 8, 2017, each incorporated herein by reference in its entirety.

### BACKGROUND OF THE INVENTION

ERK kinases are serine/threonine kinases that mediate intracellular signal transduction pathways involved in tumor growth, progression and metastasis. ERK is involved in the RAS/RAF/MEK/ERK pathway, which plays a central role in regulating cellular processes by relaying extracellular signals from ligand-bound cell surface receptor tyrosine kinases (RTKs) such as ErbB (e.g. EGFR, Her-2, etc), VEGF, PDGF, and FGF receptor tyrosine kinases. Activation of an RTK triggers a series of phosphorylation events, beginning with the activation of RAS, followed by recruitment and activation of RAF. Activated RAF then phosphorylates MAP kinase kinase (MEK) 1/2, which then phosphorylates ERK 1/2. ERK phosphorylation by MEK occurs on Y204 and T202 for ERK1 and Y185 and T183 for ERK2 (Ahn et al., Methods in Enzymology 2001, 332, 417-431). Phosphorylated ERK dimerizes and translocates to and accumulates in the nucleus (Khokhlatchev et al., Cell 1998, 93, 605-615). In the nucleus, ERK is involved in several important cellular functions, including but not limited to nuclear transport, signal transduction, DNA repair, nucleosome assembly and translocation, and mRNA processing and translation (Ahn et al., Molecular Cell 2000, 6, 1343-1354). ERK2 phosphorylates a multitude of regulatory proteins, including the protein kinases RSK90 and MAPKAP2 ((Bjorbaek et al., 1995, J. Biol. Chem. 270, 18848; Rouse et al., 1994, Cell 78, 1027), and transcription factors such as ATF2, ELK-1, c-FOS, and c-MYC (Raingeaud et al., 1996, Mol . Cell Biol. 16, 1247; Chen et al., 1993, Proc. Natl. Acad. Sci. U.S.A. 90, 10952; Oliver et al., 1995, Proc. Soc. Exp. Biol. Med. 210, 162). Overall, treatment of cells with growth factors leads to the activation of ERK1 and ERK2 which results in proliferation and, in some cases, differentiation (Lewis et al., Adv. Cancer Res. 1998, 74, 49-139).

A wealth of studies have shown that genetic mutations and/or overexpression of protein kinases in the RAS/RAF/MEK/ERK pathway lead to uncontrolled cell proliferation and tumor formation in proliferative diseases such as cancer. For example, some cancers contain mutations which result in the continuous activation of this pathway due to continuous production of growth factors. Other mutations can lead to defects in the deactivation of the activated GTP-bound RAS complex, again resulting in activation of the MAP kinase pathway. Mutated, oncogenic forms of RAS are found in 50% of colon and >90% pancreatic cancers as well as many others types of cancers (Kohl et al., Science 1993, 260, 1834-1837). Recently, bRAF mutations have been identified in more than malignant melanomas (60%), thyroid cancers (greater than 40%) and colorectal cancers. These mutations in bRAF result in a constitutively active RAS/RAF/MEK/ERK kinase cascade. Studies of primary tumor samples and cell lines have also shown constitutive or overactivation of the RAS/RAF/MEK/ERK kinase pathway in cancers of pancreas, colon, lung, ovary and kidney (Hoshino, R. et al., Oncogene 1999, 18, 813-822). Further, ERK2 has been shown to play a role in the negative growth control of breast cancer cells (Frey and Mulder, 1997, Cancer Res. 57, 628) and hyperexpression of ERK2 in human breast cancer has been reported (Sivaraman et al., 1997, J Clin. Invest. 99, 1478). Activated ERK2 has also been implicated in the proliferation of endothelin-stimulated airway smooth muscle cells, suggesting a role for this kinase in asthma (Whelchel et al., 1997, Am. J. Respir. Cell Mol. Biol. 16, 589). In view of the multitude of upstream (e.g. RAS, RAF) and downstream (e.g. ATF2, c-FOS, c-MYC) signaling proteins in the RAF/RAS/MEK/ERK pathway that have been implicated in a wide range of disorders, including but not limited to cancer, ERK has emerged as a prime target for drug development.

Cancer is the second leading cause of human death. Worldwide, millions of people die from cancer every year. In the United States alone, cancer causes the death of well over a half-million people annually, with some 1.7 million new cases diagnosed per year (excluding basal cell and squamous cell skin cancers). Squamous-cell carcinoma (SCC) is a histologically distinct form of cancer that arises from uncontrolled growth of abnormal squamous cells. An estimated one million cases of SCC are diagnosed per year in the United States. Certain therapies are known to be more effective in some patient populations than others. Understanding these drug-responsive subtypes is of significant interest to patients and health care professionals so as to avoid a trial and error approach of treatment.

### SUMMARY OF THE INVENTION

As such, there is a pressing need for a method of stratifying patients into populations based on the predicted sensitivity or resistance of a patient population to a particular treatment, including treatment with an ERK inhibitor. The present disclosure addresses this need in the art through the assessment of biomarkers that are indicative of patient populations that would be responsive to treatment with an ERK inhibitor. This allows for more timely and aggressive treatment as opposed to a trial and error approach. The compositions and methods herein may be useful for treating diseases dependent on the activity of ERK, such as cancer. Preferably, the cancer is a squamous cell carcinoma, such as a squamous cell carcinoma of the lung, esophagus, head and neck or cervix.

In certain aspects, the present disclosure provides a method of treating squamous cell carcinoma in a subject in need thereof comprising administering an effective dose of an inhibitor of an extracellular signal-regulated kinase (ERK) to the subject. The subject may comprise a genome that exhibits (1) a first total expression level of at least two mitogen-activated protein kinase (MAPK) pathway genes that is greater than a first reference level, (2) a second total expression level of at least two RAS-ERK feedback regulators that is greater than a second reference level and/or (3) a third total expression level of at least one MAPK pathway gene and at least one RAS-ERK feedback regulator that is greater than a third reference level, wherein the first reference level, the second reference level and the third reference level are each indicative of low sensitivity to the ERK inhibitor.

In certain aspects, the present disclosure provides a method of treating a subject having squamous cell carcinoma, comprising (a) screening the subject for the presence or absence of a gene signature indicative of sensitivity to an ERK inhibitor; and (b) administering the ERK inhibitor to the subject if the gene signature is determined to be present. The method may further comprise applying an alternative therapy, such as chemotherapy, immunotherapy, radiotherapy or surgery, to the subject if the gene signature is determined to be absent. In some embodiments, the gene signature comprises a first total expression level of at least two MAPK pathway genes that is greater than a first reference level. In some embodiments, the gene signature comprises a second total expression level of at least two RAS-ERK feedback regulators that is greater than a second reference level. In some embodiments, the gene signature comprises a third total expression level of at least one MAPK pathway gene and at least one RAS-ERK feedback regulator that is greater than a third reference level. In some embodiments, the gene signature comprises copy number amplification of at least one MAPK pathway gene. In some embodiments, the screening comprises performing nucleic acid analysis of a nucleic acid isolated from the subject. The nucleic acid may be from a squamous cell carcinoma cell.

In certain aspects, the present disclosure provides a method of downregulating MAPK signaling output in a plurality of squamous cell carcinoma cells with an ERK inhibitor. The method may comprise (a) assessing, in a biological sample comprising a nucleic acid from the subject, (1) a first total expression level of at least two MAPK pathway genes, (2) a second total expression level of at least two RAS-ERK feedback regulators and/or (3) a third total expression level of at least one MAPK pathway gene and at least one RAS-ERK feedback regulator. Optionally, the method further comprises administering an effective dose of the ERK inhibitor to the plurality of cells if the first total expression level is greater than a first reference level, the second total expression level is greater than a second reference level and/or the third total expression level is greater than a third reference level, wherein the first reference level, the second reference level and the third reference level are each indicative of low sensitivity to the ERK inhibitor.

In certain aspects, the present disclosure provides a method of categorizing a squamous cell carcinoma status of a subject, comprising (a) obtaining a biological sample from the subject, the sample comprising genomic and/or transcriptomic material from a squamous cell carcinoma cell of the subject; (b) assessing (1) a first total expression level of at least two MAPK pathway genes in the sample, (2) a second total expression level of at least two RAS-ERK feedback regulators in the sample, and/or (3) a third total expression level of at least one MAPK pathway gene and at least one RAS-ERK feedback regulator in the sample; (c) generating an expression profile based on (1) a comparison between the first total expression level and a first reference level, (2) a comparison between the second total expression level and a second reference level, and/or (3) a comparison between the third total expression level and a third reference level, wherein the first reference level, the second reference level and the third reference level are derivable from a reference sample from a different subject having a known squamous cell carcinoma status; and (d) categorizing the squamous cell carcinoma status of the subject of (a) based on the expression profile. The squamous cell carcinoma status may be categorized as likely sensitive to treatment with an ERK inhibitor if the first total expression level is greater than the first reference level, wherein the first reference level is indicative of low sensitivity to the ERK inhibitor. The squamous cell carcinoma status may be categorized as likely sensitive to treatment with an ERK inhibitor if the second total expression level is greater than a second reference level, wherein the second reference level is indicative of low sensitivity to the ERK inhibitor. The squamous cell carcinoma status may be categorized as likely sensitive to treatment with an ERK inhibitor if the third total expression level is greater than a third reference level, wherein the third reference level is indicative of low sensitivity to the ERK inhibitor. In some embodiments, the known squamous cell carcinoma status of the different subject is categorized as resistant to an ERK inhibitor or sensitive to an ERK inhibitor. In some embodiments, the categorizing step includes calculating, using a computer system, a likelihood of response of the subject to treatment with an ERK inhibitor based on the expression profile, wherein the likelihood is adjusted upward for each fold increase in the first total expression level relative to the first reference level, for each fold increase in the second total expression level relative to the second reference level, and for each fold increase in the third total expression level relative to the third reference level, wherein the first reference level, the second reference level and the third reference level are each indicative of low sensitivity to the ERK inhibitor. The method may further comprise preparing a report comprising a prediction of the likelihood of response of the subject to treatment with the ERK inhibitor.

In certain aspects, the present disclosure provides a method of assessing a likelihood of a subject having squamous cell carcinoma exhibiting a clinically beneficial response to treatment with an ERK inhibitor, the method comprising: (a) assessing, in a biological sample comprising genomic and/or transcriptomic material from a squamous cell carcinoma cell, (1) a first total expression level of at least two MAPK pathway genes, (2) a second total expression level of at least two RAS-ERK feedback regulators, and/or (3) a third total expression level of at least one MAPK pathway gene and at least one RAS-ERK feedback regulator; and (b) calculating, using a computer system, a weighted probability of ERK inhibitor responsiveness based on (1) a comparison between the first total expression level and a first reference level, (2) a comparison between the second total expression level and a second reference level, and/or (3) a comparison between the third total expression level and a third reference level, wherein the first reference level, the second reference level and the third reference level are derivable from one or more reference samples. The method may further comprise designating the subject as having a high probability of exhibiting a clinically beneficial response to treatment with the ERK inhibitor if the weighted probability corresponds to at least 1.5 times a baseline probability, wherein the baseline probability represents a likelihood that the subject will exhibit a clinically beneficial response to treatment with the ERK inhibitor before obtaining the weighted probability of (b). Optionally, the method further comprises transmitting information concerning the likelihood to a receiver. Optionally, the method further comprises providing a recommendation based on the weighted probability. The recommendation may comprise treating the subject with the ERK inhibitor. Alternatively, the recommendation may comprise discontinuing therapy, chemotherapy, immunotherapy, radiotherapy or surgery. A method described herein may further comprise selecting a treatment based on the weighted probability. In some embodiments, the method further comprises administering the ERK inhibitor based on the weighted probability.

In practicing any of the subject methods, the first total expression level, the second total expression level and/or the third total expression level may be assessed by detecting a level of mRNA transcribed from: the at least two MAPK pathway genes; the at least two RAS-ERK feedback regulators; and/or the at least one MAPK pathway gene and the at least one RAS-ERK feedback regulator. In some embodiments, the first total expression level, the second total expression level and/or the third total expression level is assessed by detecting a level of cDNA produced from reverse transcription of mRNA transcribed from: the at least two MAPK pathway genes; the at least two RAS-ERK feedback regulators; and/or the at least one MAPK pathway gene and the at least one RAS-ERK feedback regulator. In some embodiments, the first total expression level, the second total expression level and/or the third total expression level is assessed by detecting a level of polypeptide encoded by: the at least two MAPK pathway genes; the at least two RAS-ERK feedback regulators; and/or the at least one MAPK pathway gene and the at least one RAS-ERK feedback regulator. Detecting a level of polypeptide may comprise at least one technique selected from the group consisting of immunohistochemistry (IHC), mass spectrometry, Western blotting, enzyme-linked immunosorbent assay (ELISA), immunocytochemistry, immunofluorescence and flow cytometry. In some embodiments, the first total expression level, the second total expression level and/or the third total expression level is assessed by a nucleic acid amplification assay, a hybridization assay, sequencing, or a combination thereof. The nucleic acid amplification assay, the hybridization assay, or the sequencing may be performed using a nucleic acid sample from the subject. A nucleic acid sample may comprise a nucleic acid selected from the group consisting of genomic DNA, cDNA, ctDNA, cell-free DNA, RNA and mRNA, optionally from a squamous cell carcinoma cell. In some embodiments, the first total expression level, the second total expression level and/or the third total expression level is assessed using an nCounter® analysis system.

In practicing any of the subject methods, the first reference level, the second reference level and/or the third reference level may be obtained by assessing, in a biological sample from a subject having a squamous cell carcinoma exhibiting low sensitivity to treatment with the ERK inhibitor, expression of: the at least two MAPK pathway genes; the at least two RAS-ERK feedback regulators; and/or the at least one MAPK pathway gene and the at least one RAS-ERK feedback regulator. In some embodiments, the first reference level represents the average total expression level of the at least two MAPK pathway genes in a plurality of squamous cell carcinoma samples. In some embodiments, the second reference level represents the average total expression level of the at least two RAS-ERK feedback regulators in a plurality of squamous cell carcinoma samples. In some embodiments, the third reference level represents the average total expression level of the at least one MAPK pathway gene and the at least one RAS-ERK feedback regulator in a plurality of squamous cell carcinoma samples.

In practicing any of the subject methods, the at least two MAPK pathway genes may consist of four MAPK pathway genes, six MAPK pathway genes or eight MAPK pathway genes. In some embodiments, the at least two MAPK pathway genes are selected from CDK4, CDK6, EGFR, ERK1, CCND1, KRAS, ERK2, and HRAS. In some embodiments, the at least two MAPK pathway genes are selected from EGFR, ERK1, CCND1, KRAS, ERK2, and HRAS. In some embodiments, the at least two MAPK pathway genes are selected from EGFR, ERK1, CCND1 and KRAS. In some embodiments, the at least two MAPK pathway genes are selected from EGFR, ERK1 and CCND1. In some embodiments, the at least two MAPK pathway genes are selected from EGFR, ERK 1 and KRAS. In some embodiments, the at least two MAPK pathway genes are selected from ERK1 and CCND1 . In some embodiments, the at least two MAPK pathway genes are selected from ERK1 and EGFR. In some embodiments, the at least two MAPK pathway genes are selected from EGFR and CCND1 .

In practicing any of the subject methods, the at least two RAS-ERK feedback regulators may consist of four RAS-ERK feedback regulators or five RAS-ERK feedback regulators. In some embodiments, the at least two RAS-ERK feedback regulators are selected from DUSP5 , DUSP6, SPRY2 , SPRY4 and SPRED1. In some embodiments, the at least two RAS-ERK feedback regulators are selected from DUSP5 , DUSP6, DUSP2 and DUSP4 . In some embodiments, the at least two RAS-ERK feedback regulators are selected from DUSP5 and DUSP6 .

In practicing any of the subject methods, the at least one MAPK pathway gene and the at least one RAS-ERK feedback regulator may be selected from EGFR, ERK 1 , CCND1, KRAS, ERK2, HRAS, DUSP5 , DUSP6, DUSP2 , DUSP4, SPRY2, SPRY4 , SPRED1, and CRAF . In some embodiments, the at least one MAPK pathway gene and the at least one RAS-ERK feedback regulator are selected from CCND1, CRAF , DUSP5 , EGFR, ERK1, and KRAS.

In certain aspects, the present disclosure provides a method of treating head and neck squamous cell carcinoma in a subject in need thereof, comprising administering an effective dose of an inhibitor of an extracellular signal-regulated kinase (ERK) to the subject. In some embodiments, the subject comprises a genome that exhibits (1) a fourth total expression level of AREG, CDH3 , COL17A1, EGFR, HIF1A, ITGB1, KRT1 , KRT9 , NRG1 , SLC16A1 , SLC22A1 and VEGFA that is greater than a fourth reference level; (2) a fifth total expression level of DCUN1D1, PIK3CA, PRKCI, SOX2 and TP63 that is less than a fifth reference level; (3) a ratio of the fourth total expression level to the fifth total expression level that is greater than 0.1, such as greater than 1; and/or (4) a ratio of HIF1A to TP63 expression levels that is greater than 0.1, such as greater than 1, wherein the fourth reference level and the fifth reference level are each indicative of low sensitivity to the ERK inhibitor.

In certain aspects, the present disclosure provides a method of treating a subject having head and neck squamous cell carcinoma, comprising (a) screening the subject for the presence or absence of a gene signature indicative of sensitivity to an ERK inhibitor; and (b) administering the ERK inhibitor to the subject if the gene signature is determined to be present. In some embodiments, the method further comprises applying an alternative therapy, such as chemotherapy, immunotherapy, radiotherapy or surgery, to the subject if the gene signature is determined to be absent. In some embodiments, the gene signature comprises a fourth total expression level of AREG, CDH3, COL17A1 , EGFR, HIF1A, ITGB1, KRT1, KRT9 ,NRG1, SLC16A1 , SLC22A1 and VEGFA that is greater than a fourth reference level. In some embodiments, the gene signature comprises a fifth total expression level of DCUN1D1, PIK3CA, PRKCI, SOX2 and TP63 that is less than a fifth reference level. In some embodiments, the gene signature comprises a ratio of a fourth total expression level of AREG, CDH3, COL17A1, EGFR, HIF1A, ITGB1 , KRT1 , KRT9, NRG1 , SLC16A1 , SLC22A1 and VEGFA to a fifth total expression level of DCUN1D1, PIK3CA, PRKCI, SOX2 and TP63. In some embodiments, the gene signature comprises a ratio of HIF1A to TP63 expression levels. In some embodiments, the gene signature comprises a ratio of HIF1A to TP63 protein levels. The screening may comprise performing nucleic acid analysis of a nucleic acid isolated from the subject, optionally from a head and neck squamous cell carcinoma cell.

In certain aspects, the present disclosure provides a method of downregulating MAPK signaling output in a plurality of head and neck squamous cell carcinoma cells with an ERK inhibitor, comprising (a) assessing, in a biological sample comprising a nucleic acid from the subject, (1) a fourth total expression level of AREG, CDH3, COL17A1 , EGFR, HIF1A, ITGB1, KRT1, KRT9, NRG1 , SLC16A1, SLC22A1 and VEGFA; (2) a fifth total expression level of DCUN1D1, PIK3CA, PRKCI, SOX2 and TP63; (3) a ratio of the fourth total expression level to the fifth total expression level; and/or (4) a ratio of HIF1A to TP63 expression levels; and (b) administering an effective dose of the ERK inhibitor to the plurality of cells if (1) the fourth total expression level is greater than a fourth reference level, (2) the fifth total expression level is less than a fifth reference level, (3) the ratio of the fourth total expression level to the fifth total expression level is greater than 1, and/or (4) the ratio of HIF1A to TP63 is greater than 1, wherein the fourth reference level and the fifth reference level are each indicative of low sensitivity to the ERK inhibitor.

In certain aspects, the present disclosure provides a method of categorizing a head and neck squamous cell carcinoma status of a subject, comprising (a) obtaining a biological sample from the subject, the sample comprising genomic and/or transcriptomic material from a squamous cell carcinoma cell of the subject; (b) assessing, in the sample, (1) a fourth total expression level of AREG, CDH3, COL17A1 , EGFR, HIF1A, ITGB1, KRT1 , KRT9, NRG1 , SLC16A1 , SLC22A1 and VEGFA; (2) a fifth total expression level of DCUN1D1, PIK3CA, PRKCI, SOX2 and TP63; and/or (3) expression levels of HIF1A and TP63; (c) generating an expression profile based on (1) a comparison between the fourth total expression level and a fourth reference level, (2) a comparison between the fifth total expression level and a fifth reference level, (3) a comparison between the fourth total expression level to the fifth total expression level, and/or (4) a comparison between expression levels of HIF1A and TP63, wherein the fourth reference level and the fifth reference level are derivable from a reference sample from a different subject having a known squamous cell carcinoma status; and (d) categorizing the squamous cell carcinoma status of the subject of (a) based on the expression profile. The squamous cell carcinoma status may be categorized as likely sensitive to treatment with an ERK inhibitor if the fourth total expression level is greater than the fourth reference level, wherein the fourth reference level is indicative of low sensitivity to the ERK inhibitor. The squamous cell carcinoma status may be categorized as likely sensitive to treatment with an ERK inhibitor if the fifth total expression level is less than a fifth reference level, wherein the fifth reference level is indicative of low sensitivity to the ERK inhibitor. The squamous cell carcinoma status may be categorized as likely sensitive to treatment with an ERK inhibitor if a ratio of the fourth total expression level to the fifth total expression level is greater than 1. The squamous cell carcinoma status may be categorized as likely sensitive to treatment with an ERK inhibitor if a ratio of HIF1A to TP63 expression levels is greater than 1. Optionally, the categorizing step includes calculating, using a computer system, a likelihood of response of the subject to treatment with an ERK inhibitor based on the expression profile, wherein the likelihood is adjusted upward for each fold increase in the fourth total expression level relative to the fourth reference level and downward for each fold increase in the fifth total expression level relative to the fifth reference level, wherein the fourth reference level and the fifth reference level are each indicative of low sensitivity to the ERK inhibitor. In some embodiments, the method further comprises preparing a report comprising a prediction of the likelihood of response of the subject to treatment with the ERK inhibitor.

In certain aspects, the present disclosure provides a method of assessing a likelihood of a subject having head and neck squamous cell carcinoma exhibiting a clinically beneficial response to treatment with an ERK inhibitor, the method comprising: (a) assessing, in a biological sample comprising genomic and/or transcriptomic material from a squamous cell carcinoma cell, (1) a fourth total expression level of AREG, CDH3, COL17A1 , EGFR, HIF1A, ITGB1, KRT1, KRT9, NRG1 , SLC16A1 , SLC22A1 and VEGFA; (2) a fifth total expression level of DCUN1D1, PIK3CA, PRKCI, SOX2 and TP63; and/or (3) expression levels of HIF1A and TP63; and (b) calculating, using a computer system, a weighted probability of ERK inhibitor responsiveness based on (1) a comparison between the fourth total expression level and a fourth reference level, (2) a comparison between the fifth total expression level and a fifth reference level, (3) a comparison between the fourth total expression level to the fifth total expression level, and/or (4) a comparison between expression levels of HIF1A and TP63, wherein the fourth reference level and the fifth reference level are derivable from one or more reference samples. In some embodiments, the method further comprises designating the subject as having a high probability of exhibiting a clinically beneficial response to treatment with the ERK inhibitor if the weighted probability corresponds to at least 1.5 times a baseline probability, wherein the baseline probability represents a likelihood that the subject will exhibit a clinically beneficial response to treatment with the ERK inhibitor before obtaining the weighted probability of (b). In some embodiments, the method further comprises transmitting information concerning the likelihood to a receiver. In some embodiments, the method further comprises providing a recommendation based on the weighted probability. The recommendation may comprise treating the subject with the ERK inhibitor. In some embodiments, the method further comprises selecting a treatment based on the weighted probability. In some embodiments, the method further comprises administering the ERK inhibitor based on the weighted probability.

In practicing any of the subject methods, expression levels may be assessed by detecting a level of mRNA. In some embodiments, expression levels are assessed by detecting a level of cDNA produced from reverse transcription of mRNA. In some embodiments, expression levels are assessed by detecting a level of polypeptide. Detecting a level of polypeptide may comprise at least one technique selected from the group consisting of immunohistochemistry (IHC), mass spectrometry, Western blotting, enzyme-linked immunosorbent assay (ELISA), immunocytochemistry, immunofluorescence and flow cytometry. In some embodiments, expression levels are assessed by a nucleic acid amplification assay, a hybridization assay, sequencing, or a combination thereof. The nucleic acid amplification assay, the hybridization assay, or the sequencing may be performed using a nucleic acid sample from the subject. In some embodiments, the nucleic acid sample comprises a nucleic acid selected from the group consisting of genomic DNA, cDNA, ctDNA, cell-free DNA, RNA and mRNA, optionally from a head and neck squamous cell carcinoma cell. In some embodiments, the expression levels are assessed using an nCounter® analysis system.

In practicing any of the subject methods, the fourth reference level and/or the fifth reference level may be obtained by assessing expression of (1) AREG, CDH3 , COL17A1 , EGFR, HIF1A, ITGB1, KRT1, KRT9 , NRG1, SLC16A1, SLC22A1 and VEGFA; and/or (2) DCUN1D1, PIK3CA, PRKCI, SOX2 and TP 63 , respectively, in a biological sample from a subject having a squamous cell carcinoma exhibiting low sensitivity to treatment with the ERK inhibitor. In some embodiments, the fourth reference level represents the average total expression level of AREG, CDH3, COL17A1 , EGFR, HIF1A, ITGB1 , KRT1 , KRT9 , NRG1 , SLC16A1 , SLC22A1 and VEGFA in a plurality of squamous cell carcinoma samples. In some embodiments, the fifth reference level represents the average total expression level of DCUN1D1 , PIK3CA, PRKCI, SOX2 and TP63 in a plurality of squamous cell carcinoma samples.

In certain aspects, the present disclosure provides a method of treating squamous cell carcinoma in a subject in need thereof, comprising administering an effective dose of an inhibitor of an extracellular signal-regulated kinase (ERK) to the subject, said subject comprising a genome having a copy number profile that comprises copy number amplification of at least one mitogen-activated protein kinase (MAPK) pathway gene.

In certain aspects, the present disclosure provides a method of downregulating MAPK signaling output in a plurality of squamous cell carcinoma cells with an ERK inhibitor, comprising (a) assessing, in a biological sample comprising a nucleic acid from the subject, a copy number profile of at least one MAPK pathway gene; and (b) administering an effective dose of the ERK inhibitor to the plurality of cells if the copy number profile comprises an average copy number of the at least one MAPK pathway gene of greater than 2.

In certain aspects, the present disclosure provides a method of categorizing a squamous cell carcinoma status of a subject, comprising (a) obtaining a biological sample from the subject, the sample comprising genomic and/or transcriptomic material from a squamous cell carcinoma cell of the subject; (b) assessing a copy number profile of at least one MAPK pathway gene in the sample; and (c) categorizing the squamous cell carcinoma status of the subject based on the copy number profile. The squamous cell carcinoma status may be categorized as likely sensitive to treatment with an ERK inhibitor if the copy number profile comprises an average copy number of the at least one MAPK pathway gene of greater than 2. In some embodiments, the categorizing step includes calculating, using a computer system, a likelihood of response of the subject to treatment with an ERK inhibitor based on the copy number profile, wherein the likelihood is adjusted upward for each additional copy number of the at least one MAPK pathway gene in excess of 2. Optionally, the method further comprises preparing a report comprising a prediction of the likelihood of response of the subject to treatment with the ERK inhibitor.

In certain aspects, the present disclosure provides a method of assessing a likelihood of a subject having squamous cell carcinoma exhibiting a clinically beneficial response to treatment with an ERK inhibitor, the method comprising (a) assessing a copy number profile of at least one MAPK pathway gene in a biological sample comprising genomic and/or transcriptomic material from a squamous cell carcinoma cell; and (b) calculating, using a computer system, a weighted probability of ERK inhibitor responsiveness based on the copy number profile. In some embodiments, the method further comprises designating the subject as having a high probability of exhibiting a clinically beneficial response to treatment with the ERK inhibitor if the weighted probability corresponds to at least 1.5 times a baseline probability, wherein the baseline probability represents a likelihood that the subject will exhibit a clinically beneficial response to treatment with the ERK inhibitor before obtaining the weighted probability of (b). Optionally, the method further comprises transmitting information concerning the likelihood to a receiver. In some embodiments, the method further comprises providing a recommendation based on the weighted probability. The recommendation may comprise treating the subject with the ERK inhibitor. The recommendation may comprise discontinuing therapy, chemotherapy, immunotherapy, radiotherapy or surgery. In some embodiments, the method further comprises selecting a treatment based on the weighted probability. In some embodiments, the method further comprises administering the ERK inhibitor based on the weighted probability.

In practicing any of the subject methods, the copy number profile of the at least one MAPK pathway gene may be assessed by a method selected from the group consisting of in situ hybridization, Southern blot, immunohistochemistry (IHC), polymerase chain reaction (PCR), quantitative PCR (qPCR), quantitative real-time PCR (qRT-PCR), comparative genomic hybridization, microarray-based comparative genomic hybridization, and ligase chain reaction (LCR). In some embodiments, the copy number profile of the at least one MAPK pathway gene is assessed by a method selected from the group consisting of fluorescent in situ hybridization, chromogenic in situ hybridization, and silver in situ hybridization. In some embodiments, the copy number profile is assessed using a nucleic acid sample from the subject, optionally wherein the nucleic acid sample comprises a nucleic acid selected from the group consisting of genomic DNA, cDNA, ctDNA, cell-free DNA, RNA and mRNA. In some embodiments, the nucleic acid is from a squamous cell carcinoma cell.

In practicing any of the subject methods, the at least one MAPK pathway gene may be selected from CDK4 , CDK6, EGFR, ERK1, CCND1 , KRAS, ERK2 , and HRAS, such as EGFR. In some embodiments, the squamous cell carcinoma is esophageal squamous cell carcinoma.

In practicing any of the subject methods, the biological sample may be a tissue sample, optionally wherein the tissue sample is fixed, paraffin-embedded, fresh or frozen. The tissue sample may be derived from fine needle, core or other types of biopsy. In some embodiments, the biological sample is a whole blood or plasma sample.

In practicing any of the subject methods, the squamous cell carcinoma may be selected from lung, esophagus, cervical and head and neck squamous cell carcinomas. In some embodiments, the ERK inhibitor is administered as a monotherapy. In some embodiments, the ERK inhibitor is administered with at least one other anti-cancer therapy.

In certain aspects, the present disclosure provides a method of treating cancer in a subject in need thereof, comprising administering an effective dose of an inhibitor of an extracellular signal-regulated kinase (ERK) to the subject, wherein the subject exhibits resistance to a treatment with a Ras, Raf or MEK inhibitor.

In certain aspects, the present disclosure provides a method of treating a subject having cancer, comprising (a) screening the subject for resistance to a treatment with a Ras, Raf or MEK inhibitor; and (b) administering an ERK inhibitor to the subject if the subject is determined to be resistant to a treatment with the Ras, Raf or MEK inhibitor.

Optionally, the subject exhibits resistance to a treatment with a B-Raf inhibitor. The B-Raf inhibitor may be selected from vemurafenib, GDC-0879, PLX-4720, PLX-3603, PLX-4032, RAF265, XL281, AZ628, sorafenib, dabrafenib and LGX818, such as vemurafenib. Optionally, the subject exhibits resistance to a treatment with an MEK inhibitor. The MEK inhibitor may be selected from trametinib, cobimetinib, binimetinib, selumetinib, PD-325901, CI-1040, PD-035901, TAK-733, PD98059, PD184352, U0126, RDEA119, AZD8330, RO4987655, RO4927350, R05068760, AS703026 and E6201, such as trametinib. In some embodiments, the cancer comprises a B-Raf or N-Ras mutation. Optionally, the cancer is selected from breast cancer, pancreatic cancer, lung cancer, thyroid cancer, seminomas, melanoma, bladder cancer, liver cancer, kidney cancer, myelodysplastic syndrome, acute myelogenous leukemia and colorectal cancer. Optionally, the cancer is selected from pancreatic cancer, lung cancer, melanoma and colorectal cancer, such as melanoma.

In certain aspects, the present disclosure provides a method of inhibiting growth of a cancer cell, the method comprising administering to the cell an ERK inhibitor, wherein the cell exhibits resistance to a treatment with a Ras, Raf or MEK inhibitor. Optionally, the cell exhibits resistance to a treatment with a B-Raf inhibitor, such as vemurafenib, GDC-0879, PLX-4720, PLX-3603, PLX-4032, RAF265, XL281, AZ628, sorafenib, dabrafenib and LGX818. Optionally, the B-Raf inhibitor is vemurafenib. Optionally, the cell exhibits resistance to a treatment with an MEK inhibitor, such as trametinib, cobimetinib, binimetinib, selumetinib, PD-325901, CI-1040, PD-035901, TAK-733, PD98059, PD184352, U0126, RDEA119, AZD8330, RO4987655, RO4927350, R05068760, AS703026 and E6201. Optionally, the MEK inhibitor is trametinib. In some embodiments, the cell comprises a B-Raf or N-Ras mutation. Optionally, the cell is selected from a pancreatic cancer cell, a lung cancer cell, a melanoma cell and a colorectal cancer cell, such as a melanoma cell.

In certain aspects, the present disclosure provides a method of treating cancer in a subject in need thereof, comprising administering an effective dose of an inhibitor of an extracellular signal-regulated kinase (ERK) to the subject. The subject may comprise a genome that exhibits amplification and/or overexpression of at least one gene located at chromosome 11q13.3-13.4. In some examples, the method further comprises (a) screening the subject for amplification and/or overexpression of the at least one gene located at chromosome 11q13.3-13.4; and (b) administering the ERK inhibitor to the subject if the amplification and/or overexpression is determined to be present.

In certain aspects, the present disclosure provides a method of treating a subject having cancer, comprising (a) screening the subject for amplification and/or overexpression of at least one gene located at chromosome 11q13.3-13.4 or a gene that co-amplifies with a gene located at chromosome 11q13.3-13.4; and (b) administering an ERK inhibitor to the subject if the amplification and/or overexpression is determined to be present.

A method of the present disclosure may further comprise applying an alternative therapy, such as chemotherapy, immunotherapy, radiotherapy or surgery, to the subject if the amplification and/or overexpression is absent. In some embodiments, the screening comprises performing nucleic acid analysis of a nucleic acid isolated from the subject. The nucleic acid may be from a cancer cell. In some embodiments, the method further comprises administering the ERK inhibitor to the subject if both amplification and overexpression of the at least one gene are determined to be present. The method may comprise administering the ERK inhibitor to the subject if the subject exhibits amplification and/or overexpression of CCND1 or ANO1 . The method may comprise administering the ERK inhibitor to the subject if the subject exhibits amplification or overexpression of CCND1 and ANO1 . The method may comprise administering the ERK inhibitor to the subject if the subject exhibits amplification and overexpression of CCND1 and ANO1 .

In certain aspects, the present disclosure provides a method of downregulating MAPK signaling output in a plurality of cancer cells with an ERK inhibitor. The method may comprise (a) assessing, in a biological sample comprising a nucleic acid from the plurality of cells, a copy number profile and/or expression profile of at least one gene located at chromosome 11q13.3-13.4; and (b) administering an effective dose of the ERK inhibitor to the plurality of cells if the copy number profile comprises an average copy number of the at least one gene of >2 and/or if the expression profile is greater than a reference level, wherein the reference level is indicative of low sensitivity to the ERK inhibitor.

In certain aspects, the present disclosure provides a method of categorizing a cancer status of a subject, comprising (a) obtaining a biological sample from the subject, the sample comprising genomic and/or transcriptomic material from a cancer cell of the subject; (b) assessing a copy number profile and/or expression profile of at least one gene located at chromosome 11q13.3-13.4 in the sample; and (c) categorizing the cancer status of the subject of (a) based on the copy number profile and/or the expression profile. The cancer status may be categorized as likely sensitive to treatment with an ERK inhibitor if the copy number profile comprises an average copy number of the at least one gene of >2. In some embodiments, the cancer status is categorized as likely sensitive to treatment with an ERK inhibitor if the expression profile is greater than a reference level, wherein the reference level is indicative of low sensitivity to the ERK inhibitor. The categorizing step may include calculating, using a computer system, a likelihood of response of the subject to treatment with an ERK inhibitor based on the copy number profile and/or the expression profile, wherein the likelihood is adjusted upward for each additional copy number of the at least one gene in excess of 2 and for each fold increase in the expression profile relative to a reference level, wherein the reference level is indicative of low sensitivity to the ERK inhibitor. In some embodiments, the method further comprises preparing a report comprising a prediction of the likelihood of response of the subject to treatment with the ERK inhibitor.

In certain aspects, the present disclosure provides a method of assessing a likelihood of a subject having cancer exhibiting a clinically beneficial response to treatment with an ERK inhibitor, the method comprising (a) assessing a copy number profile and/or expression profile of at least one gene located at chromosome 11q13.3-13.4 in a biological sample comprising genomic and/or transcriptomic material from a cancer cell; and (b) calculating, using a computer system, a weighted probability of ERK inhibitor responsiveness based on the copy number profile and/or the expression profile. The method may further comprise designating the subject as having a high probability of exhibiting a clinically beneficial response to treatment with the ERK inhibitor if the weighted probability corresponds to at least 1.5 times a baseline probability, wherein the baseline probability represents a likelihood that the subject will exhibit a clinically beneficial response to treatment with the ERK inhibitor before obtaining the weighted probability of (b). Optionally, the method further comprises transmitting information concerning the likelihood to a receiver. Optionally, the method further comprises providing a recommendation based on the weighted probability. The recommendation may comprise treating the subject with the ERK inhibitor. Alternatively, the recommendation may comprise discontinuing therapy, chemotherapy, immunotherapy, radiotherapy or surgery. A method described herein may further comprise selecting a treatment based on the weighted probability. In some embodiments, the method further comprises administering the ERK inhibitor based on the weighted probability.

In practicing any of the subject methods, the expression may be assessed by detecting a level of mRNA transcribed from the at least one gene. In some embodiments, the expression is assessed by detecting a level of cDNA produced from reverse transcription of mRNA transcribed from the at least one gene. In some embodiments, the expression is assessed by detecting a level of polypeptide encoded by the at least one gene. The detecting a level of polypeptide may comprise at least one technique selected from the group consisting of immunohistochemistry (IHC), mass spectrometry, Western blotting, enzyme-linked immunosorbent assay (ELISA), immunocytochemistry, immunofluorescence and flow cytometry. In some embodiments, the expression is assessed by a nucleic acid amplification assay, a hybridization assay, sequencing, or a combination thereof. The nucleic acid amplification assay, the hybridization assay, or the sequencing may be performed using a nucleic acid sample from the subject. The nucleic acid sample may comprise a nucleic acid selected from the group consisting of genomic DNA, cDNA, ctDNA, cell-free DNA, RNA and mRNA. In some embodiments, the nucleic acid is from a cancer cell. In some embodiments, the expression is assessed using an nCounter® analysis system.

In practicing any of the subject methods, the reference level may be obtained by assessing, in a biological sample from a subject having a cancer exhibiting low sensitivity to treatment with the ERK inhibitor, expression of the at least one gene. In some embodiments, the reference level represents the average total expression level of the at least one gene in a plurality of cancer samples.

In practicing any of the subject methods, the copy number profile of the at least one gene may be assessed by a method selected from the group consisting of in situ hybridization, Southern blot, immunohistochemistry polymerase chain reaction (PCR), quantitative PCR (qPCR), quantitative real-time PCR (qRT-PCR), comparative genomic hybridization, microarray-based comparative genomic hybridization, and ligase chain reaction (LCR). Optionally, the copy number profile of the at least one gene is assessed by a method selected from the group consisting of fluorescent in situ hybridization, chromogenic in situ hybridization, and silver in situ hybridization. In some embodiments, the copy number profile is assessed using a nucleic acid sample from the subject. The nucleic acid sample may comprise a nucleic acid selected from the group consisting of genomic DNA, cDNA, ctDNA, cell-free DNA, RNA and mRNA. In some embodiments, the nucleic acid is from a cancer cell.

In practicing any of the subject methods, the at least one gene may be selected from CCND1, CTTN, FADD, ORAOV1, ANO1, PPFIA1 and SHANK2 . In some embodiments, the at least one gene is CCND1 or ANO1 . In some embodiments, the at least one gene is CCND1 and ANO1 .

In practicing any of the subject methods, the biological sample may be a tissue sample. The tissue sample may be fixed, paraffin-embedded, fresh or frozen. In some embodiments, the tissue sample is derived from fine needle, core or other types of biopsy. In some embodiments, the biological sample is a whole blood or plasma sample.

In practicing any of the subject methods, the cancer may be selected from the group consisting of squamous cell carcinoma and adenocarcinoma, such as a squamous cell carcinoma selected from the group consisting of lung, esophageal, cervical, head and neck, bladder and gastric squamous cell carcinomas. In some embodiments, the squamous cell carcinoma is esophageal squamous cell carcinoma. In some embodiments, the cancer is an adenocarcinoma selected from the group consisting of esophageal and pancreatic adenocarcinomas. In some embodiments, the cancer is selected from the group consisting of lung, esophageal, cervical, head and neck, bladder, gastric and pancreatic cancer. In some embodiments, the cancer is selected from breast cancer, pancreatic cancer, lung cancer, thyroid cancer, seminomas, melanoma, bladder cancer, liver cancer, kidney cancer, myelodysplastic syndrome, acute myelogenous leukemia and colorectal cancer. In some embodiments, the ERK inhibitor is administered as a monotherapy. In some embodiments, the ERK inhibitor is administered with at least one other anti-cancer therapy.

Optionally, the ERK inhibitor is a compound of Formula I:

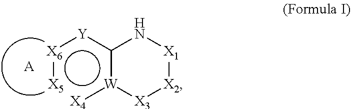

wherein:

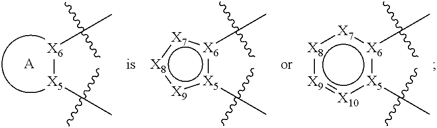

X₁ is C═O, C═S, SO, SO₂, or PO₂⁻; Y is CR₅; W is N or C;

X₂ is NR₁ or CR₁R₁′ and X₃ is null, CR₃R₃′ or C═O; or X₂-X₃ is R₁C═CR₃ or R₁C═N or N═CR₃ or NR₁₂-CR₁₁═CR₃;

X₄ is N or CR₄; X₅ is N or C; X₆ is N or C; X₇ is O, N, NR₇₂ or CR₇₁; X₈ is O, N, NR₈₂ or CR₈₁; X₉ is O, N, NR₂₂ or CR₂₁; X₁₀ is O, N, NR₉₂ or CR₉₁;

R₁ is —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —C₁₋₁₀alkyl-C₃₋₁₀aryl, —C₁₋₁₀alkyl-C₁₋₁₀hetaryl, —C₁₋₁₀alkyl-C₃₋₁₀cycloalkyl, —C₁₋₁₀alkyl-C₁₋₁₀heterocyclyl, —C₂₋₁₀alkenyl-C₃₋₁₀aryl, —C₂₋₁₀alkenyl-C₁₋₁₀hetaryl, —C₂₋₁₀alkenyl-C₃₋₁₀cycloalkyl, —C₂₋₁₀alkenyl-C₁₋₁₀heterocyclyl, —C₂₋₁₀alkynyl-C₃₋₁₀aryl, —C₂₋₁₀alkynyl-C₁₋₁₀hetaryl, —C₂₋₁₀alkynyl-C₃₋₁₀cycloalkyl, —C₂₋₁₀alkynyl-C₁₋₁₀heterocyclyl, —C₁₋₁₀heteroalkyl-C₃₋₁₀aryl, —C₁₋₁₀heteroalkyl-C₁₋₁₀hetaryl, —C₁₋₁₀heteroalkyl-C₃₋₁₀cycloalkyl, —C₁₋₁₀heteroalkyl-C₁₋₁₀heterocyclyl, —C₁₋₁₀alkoxy-C₃₋₁₀aryl, —C₁₋₁₀alkoxy-C₁₋₁₀hetaryl, —C₁₋₁₀alkoxy-C₃₋₁₀cycloalkyl, —C₁₋₁₀alkoxy-C₁₋₁₀heterocyclyl, —C₃₋₁₀aryl-C₁₋₁₀alkyl, —C₃₋₁₀aryl-C₂₋₁₀alkenyl, —C₃₋₁₀aryl-C₂₋₁₀alkynyl, —C₃₋₁₀aryl-C₃₋₁₀hetaryl, —C₃₋₁₀aryl-C₃₋₁₀cycloalkyl, —C₃₋₁₀aryl-C₁₋₁₀heterocyclyl, —C₁₋₁₀hetaryl-C₁₋₁₀alkyl, —C₁₋₁₀hetaryl-C₂₋₁₀alkynyl, —C₃₋₁₀hetaryl-C₃₋₁₀aryl, C₁₋₁₀hetaryl-C₃₋₁₀cycloalkyl, —C₁₋₁₀hetaryl-C₁₋₁₀heterocyclyl, —C₃₋₁₀cycloalkyl-C₁₋₁₀alkyl, —C₃₋₁₀cycloalkyl-C₂₋₁₀alkenyl, —C₃₋₁₀cycloalkyl-C₂₋₁₀alkynyl, —C₃₋₁₀cycloalkyl-C₃₋₁₀aryl, —C₃₋₁₀cycloalkyl-C₁₋₁₀hetaryl, —C₃₋₁₀cycloalkyl-C₁₋₁₀heterocyclyl, C₁₋₁₀heterocyclyl-C₁₋₁₀alkyl, —C₁₋₁₀heterocyclyl-C₂₋₁₀alkenyl, —C₁₋₁₀heterocyclyl-C₂₋₁₀alkynyl, —C₁₋₁₀heterocyclyl-C₃₋₁₀aryl, —C₁₋₁₀heterocyclyl-C₁₋₁₀hetaryl, or —C₁₋₁₀heterocyclyl-C₃₋₁₀cycloalkyl, each of which is unsubstituted or substituted by one or more independent R₁₀ or R₁₁ sub stituents;

R₁′ is hydrogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —C₁₋₁₀alkyl-C₃₋₁₀aryl, —C₁₋₁₀alkyl-C₁₋₁₀hetaryl, —C₁₋₁₀alkyl-C₃₋₁₀cycloalkyl, —C₁₋₁₀alkyl-C₁₋₁₀heterocyclyl, —C₂₋₁₀alkenyl-C₃₋₁₀aryl, —C₂₋₁₀alkenyl-C₁₋₁₀hetaryl, —C₂₋₁₀alkenyl-C₃₋₁₀cycloalkyl, —C₂₋₁₀alkenyl-C₁₋₁₀heterocyclyl, —C₂₋₁₀alkynyl-C₃₋₁₀aryl, —C₂₋₁₀alkynyl-C₁₋₁₀hetaryl, C₂₋₁₀alkynyl-C₃₋₁₀cycloalkyl, —C₂₋₁₀alkynyl-C₁₋₁₀heterocyclyl, —C₁₋₁₀heteroalkyl-C₃₋₁₀aryl, —C₁₋₁₀heteroalkyl-C₁₋₁₀hetaryl, —C₁₋₁₀heteroalkyl-C₃₋₁₀cycloalkyl, c₁₋₁₀heteroalkyl-C₁₋₁₀heterocyclyl, —C₁₋₁₀alkoxy-C₃₋₁₀aryl, —C₁₋₁₀alkoxy-C₁₋₁₀hetaryl, —C₁₋₁₀alkoxy-C₃₋₁₀cycloalkyl, —C₁₋₁₀alkoxy-C₁₋₁₀heterocyclyl, —C₃₋₁₀aryl-C₁₋₁₀alkyl, —C₃₋₁₀aryl-C₂₋₁₀alkenyl, —C₃₋₁₀aryl-C₂₋₁₀alkynyl, —C₃₋₁₀aryl-C₃₋₁₀hetaryl, —C₃₋₁₀aryl-C₃₋₁₀cycloalkyl, —C₃₋₁₀aryl-C₁₋₁₀heterocyclyl, —C₁₋₁₀hetaryl-C₁₋₁₀alkyl, —C₁₋₁₀hetaryl-C₂₋₁₀alkenyl, —C₁₋₁₀hetaryl-C₂₋₁₀alkynyl, —C₃₋₁₀hetaryl-C₃₋₁₀aryl, —C₁₋₁₀hetaryl-C₃₋₁₀cycloalkyl, —C₁₋₁₀hetaryl-C₁₋₁₀heterocyclyl, —C₃₋₁₀cycloalkyl-C₁₋₁₀alkyl, —C₃₋₁₀cycloalkyl-C₂₋₁₀alkenyl, —C₃₋₁₀cycloalkyl-C₂₋₁₀alkynyl, —C₃₋₁₀cycloalkyl-C₃₋₁₀aryl, —C₃₋₁₀cycloalkyl-C₁₋₁₀hetaryl, —C₃₋₁₀cycloalkyl-C₁₋₁₀heterocyclyl, —C₁₋₁₀heterocyclyl-C₁₋₁₀alkyl, —C₁₋₁₀heterocyclyl-C₂₋₁₀alkenyl, —C₁₋₁₀heterocyclyl-C₂₋₁₀alkynyl, —C₁₋₁₀heterocyclyl-C₃₋₁₀aryl, —C₁₋₁₀heterocyclyl-C₁₋₁₀hetaryl, or —C₁₋₁₀heterocyclyl-C₃₋₁₀cycloalkyl, each of which is unsubstituted or substituted by one or more independent R₁₀ or R₁₁ sub stituents;

R₂₁ is hydrogen, halogen, —OH, —CF₃, —OCF₃, —OR³¹, —NR³¹R³², —C(O)R³¹, —CO₂R³¹, —C(═O)NR³¹, —NO₂, —CN, —S(O)₀₋₂R³¹, —SO₂NR³¹R³², —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³²R³³, —NR³¹C(═NR³²)OR³³, —NR³¹C(═NR³²)SR³³, —OC(═O)OR³³, —OC(═O)NR³¹R³², —OC(═O)SR³¹, —SC(═O)SR³¹, —P(O)OR⁻OR³², —SC(═O)NR³¹R³², -L-C₁₋₁₀alkyl, -L-C₂₋₁₀alkenyl, -L-C₂₋₁₀alkynyl, -L-C₁₋₁₀heteroalkyl, -L-C₃₋₁₀aryl, -L-C₁₋₁₀hetaryl, -L-C₃₋₁₀cycloalkyl, -L-C₁₋₁₀heterocyclyl, -L-C₁₋₁₀alkyl-C₃₋₁₀aryl, -L-C₁₋₁₀alkyl-C₁₋₁₀hetaryl, -L-C₁₋₁₀alkyl-C₃₋₁₀cycloalkyl, -L-C₁₋₁₀alkyl-C₁₋₁₀heterocyclyl, -L-C₂₋₁₀alkenyl-C₃₋₁₀aryl, -L-C₂₋₁₀alkenyl-C₁₋₁₀hetaryl, -L-C₂₋₁₀alkenyl-C₃₋₁₀cycloalkyl, -L-C₂₋₁₀alkenyl-C₁₋₁₀heterocyclyl, -L-C₂₋₁₀alkynyl-C₃₋₁₀aryl, -L-C₂₋₁₀alkynyl-C₁₋₁₀hetaryl, -L-C₂₋₁₀alkynyl-C₃₋₁₀cycloalkyl, -L-C₂₋₁₀alkynyl-C₁₋₁₀heterocyclyl, -L-C₁₋₁₀heteroalkyl-C₃₋₁₀aryl, -L-C₁₋₁₀heteroalkyl-C₁₋₁₀hetaryl, -L-C₁₋₁₀heteroalkyl-C₃₋₁₀cycloalkyl, -L-C₁₋₁₀heteroalkyl-C₁₋₁₀heterocyclyl, -L-C₁₋₁₀alkoxy-C₃₋₁₀aryl, -L-C₁₋₁₀alkoxy-C₁₋₁₀hetaryl, -L-C₁₋₁₀alkoxy-C₃₋₁₀cycloalkyl, -L-C₁₋₁₀alkoxy-C₁₋₁₀heterocyclyl, -L-C₃₋₁₀aryl-C₁₋₁₀alkyl, -L-C₃₋₁₀aryl-C₂₋₁₀alkenyl, -L-C₃₋₁₀aryl-C₂₋₁₀alkynyl, -L-C₃₋₁₀aryl-C₁₋₁₀hetaryl, -L-C₃₋₁₀aryl-C₃₋₁₀cycloalkyl, -L-C₃₋₁₀aryl-C₁₋₁₀heterocyclyl, -L-C₁₋₁₀hetaryl-C₁₋₁₀alkyl, -L-C₁₋₁₀hetaryl-C₂₋₁₀alkenyl, -L-C₁₋₁₀hetaryl-C₂₋₁₀alkynyl, -L-C₁₋₁₀hetaryl-C₃₋₁₀aryl, -L-C₁₋₁₀hetaryl-C₃₋₁₀cycloalkyl, -L-C₁₋₁₀hetaryl-C₁₋₁₀heterocyclyl, -L-C₃₋₁₀cycloalkyl-C₁₋₁₀alkyl, -L-C₃₋₁₀cycloalkyl-C₂₋₁₀alkenyl, -L-C₃₋₁₀cycloalkyl-C₂₋₁₀alkynyl, -L-C₃₋₁₀cycloalkyl-C₃₋₁₀aryl, -L-C₃₋₁₀cycloalkyl-C₁₋₁₀hetaryl, -L-C₃₋₁₀cycloalkyl-C₁₋₁₀heterocyclyl, -L-C₁₋₁₀heterocyclyl-C₁₋₁₀alkyl, -L-C₁₋₁₀heterocyclyl-C₂₋₁₀alkenyl, -L-C₁₋₁₀heterocyclyl-C₂₋₁₀alkynyl, -L-C₁₋₁₀heterocyclyl-C₃₋₁₀aryl, -L-C₁₋₁₀heterocyclyl-C₁₋₁₀hetaryl, or -L-C₁₋₁₀heterocyclyl-C₃₋₁₀cycloalkyl, each of which is unsubstituted or substituted by one or more independent R₁₂ substituents;

R₂₂ is hydrogen, —OH, —CF₃, —C(O)R³¹, —CO₂R³¹, —C(═O)NR³¹, —S(O)₀₋₂R³¹, —C(═S)OR³¹, —C(═O)SR³¹, -L-C₁₋₁₀alkyl, -L-C₂₋₁₀alkenyl, -L-C₂₋₁₀alkynyl, -L-C₁₋₁₀heteroalkyl, -L-C₃₋₁₀aryl, -L-C₁₋₁₀hetaryl, -L-C₃₋₁₀cycloalkyl, -L-C₁₋₁₀heterocyclyl, -L-C₁₋₁₀alkyl-C₃₋₁₀aryl, -L-C₁₋₁₀alkyl-C₁₋₁₀hetaryl, -L-C₁₋₁₀alkyl-C₃₋₁₀cycloalkyl, -L-C₁₋₁₀alkyl-C₁₋₁₀heterocyclyl, -L-C₂₋₁₀alkenyl-C₃₋₁₀aryl, -L-C₂₋₁₀alkenyl-C₁₋₁₀hetaryl, -L-C₂₋₁₀alkenyl-C₃₋₁₀cycloalkyl, -L-C₂₋₁₀alkenyl-C₁₋₁₀heterocyclyl, -L-C₂₋₁₀alkynyl-C₃₋₁₀aryl, -L-C₂₋₁₀alkynyl-C₁₋₁₀hetaryl, -L-C₂₋₁₀alkynyl-C₃₋₁₀cycloalkyl, -L-C₂₋₁₀alkynyl-C₁₋₁₀heterocyclyl, -L-C₁₋₁₀heteroalkyl-C₃₋₁₀aryl, -L-C₁₋₁₀heteroalkyl-C₁₋₁₀hetaryl, -L-C₁₋₁₀heteroalkyl-C₃₋₁₀cycloalkyl, -L-C₁₋₁₀heteroalkyl-C₁₋₁₀heterocyclyl, -L-C₁₋₁₀alkoxy-C₃₋₁₀aryl, -L-C₁₋₁₀alkoxy-C₁₋₁₀hetaryl, -L-C₁₋₁₀alkoxy-C₃₋₁₀cycloalkyl, -L-C₁₋₁₀alkoxy-C₁₋₁₀heterocyclyl, -L-C₃₋₁₀aryl-C₁₋₁₀alkyl, -L-C₃₋₁₀aryl-C₂₋₁₀alkenyl, -L-C₃₋₁₀aryl-C₂₋₁₀alkynyl, -L-C₃₋₁₀aryl-C₁₋₁₀hetaryl, -L-C₃₋₁₀aryl-C₃₋₁₀cycloalkyl, -L-C₃₋₁₀aryl-C₁₋₁₀heterocyclyl, -L-C₁₋₁₀hetaryl-C₁₋₁₀alkyl, -L-C₁₋₁₀hetaryl-C₂₋₁₀alkenyl, -L-C₁₋₁₀hetaryl-C₂₋₁₀alkynyl, -L-C₁₋₁₀hetaryl-C₃₋₁₀aryl, -L-C₁₋₁₀hetaryl-C₃₋₁₀cycloalkyl, -L-C₁₋₁₀hetaryl-C₁₋₁₀heterocyclyl, -L-C₃₋₁₀cycloalkyl-C₁₋₁₀alkyl, -L-C₃₋₁₀cycloalkyl-C₂₋₁₀alkenyl, -L-C₃₋₁₀cycloalkyl-C₂₋₁₀alkynyl, -L-C₃₋₁₀cycloalkyl-C₃₋₁₀aryl, -L-C₃₋₁₀cycloalkyl-C₁₋₁₀hetaryl, -L-C₃₋₁₀cycloalkyl-C₁₋₁₀heterocyclyl, -L-C₁₋₁₀heterocyclyl-C₁₋₁₀alkyl, -L-C₁₋₁₀heterocyclyl-C₂₋₁₀alkenyl, -L-C₁₋₁₀heterocyclyl-C₂₋₁₀alkynyl, -L-C₁₋₁₀heterocyclyl-C₃₋₁₀aryl, -L-C₁₋₁₀heterocyclyl-C₁₋₁₀hetaryl, or -L-C₁₋₁₀heterocyclyl-C₃₋₁₀cycloalkyl, each of which is unsubstituted or substituted by one or more independent R₁₂ substituents;

L is a bond, —O—, —N(R³¹)—, —S(O)₀₋₂—, —C(═O)—, —C(═O)O—, —OC(═O)—, —C(═O)N(R³¹)—, —N(R³¹)C(═O)—, —NR³¹C(═O)O—, —NR³¹C(═O)NR³²—, —NR³¹S(O)₀₋₂—, —S(O)₀₋₂N(R³¹)—, —C(═S)O—, —C(═O)S—, —NR³¹C(═NR³²)NR³²—, —NR³¹C(═NR³²)0-, —NR³¹C(═NR³²)S—, —OC(═O)O—, —OC(═O)NR³¹—, —OC(═O)S—, —SC(═O)S—, —P(O)OR⁻O—, —SC(═O)NR³¹—;

each of R₃, R₃′ and R₄ is independently hydrogen, halogen, —OH, —CF₃, —OCF₃, —OR³¹, —NR³¹R³², —C(O)R³¹, —CO₂R⁻, —C(═O)NR³¹, —NO₂, —CN, —S(O)₀₋₂R³¹, —SO₂NR³¹R³², —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³²R³³, —NR³¹C(═NR³²)OR³³, —NR³¹C(═NR³²)SR³³, —OC(═O)OR³³, —OC(═O)NR³¹R³², —OC(═O)SR³¹, —SC(═O)SR³¹, —P(O)OR⁻OR³², —SC(═O)NR³¹R³², —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —C₁₋₁₀alkyl-C₃₋₁₀aryl, —C₁₋₁₀alkyl-C₁₋₁₀hetaryl, —C₁₋₁₀alkyl-C₃₋₁₀cycloalkyl, —C₁₋₁₀alkyl-C₁₋₁₀heterocyclyl, —C₂₋₁₀alkenyl-C₃₋₁₀aryl, —C₂₋₁₀alkenyl-C₁₋₁₀hetaryl, —C₂₋₁₀alkenyl-C₃₋₁₀cycloalkyl, —C₂₋₁₀alkenyl-C₁₋₁₀heterocyclyl, —C₂₋₁₀alkynyl-C₃₋₁₀aryl, —C₂₋₁₀alkynyl-C₁₋₁₀hetaryl, —C₂₋₁₀alkynyl-C₃₋₁₀cycloalkyl, —C₂₋₁₀alkynyl-C₁₋₁₀heterocyclyl, —C₁₋₁₀heteroalkyl-C₃₋₁₀aryl, —C₁₋₁₀heteroalkyl-C₁₋₁₀hetaryl, —C₁₋₁₀heteroalkyl-C₃₋₁₀cycloalkyl, —C₁₋₁₀heteroalkyl-C₁₋₁₀heterocyclyl, —C₁₋₁₀alkoxy-C₃₋₁₀aryl, —C₁₋₁₀alkoxy-C₁₋₁₀hetaryl, —C₁₋₁₀alkoxy-C₃₋₁₀cycloalkyl, —C₁₋₁₀alkoxy-C₁₋₁₀heterocyclyl, —C₃₋₁₀aryl-C₁₋₁₀alkyl, —C₃₋₁₀aryl-C₂₋₁₀alkenyl, —C₃₋₁₀aryl-C₂₋₁₀alkynyl, —C₃₋₁₀aryl-C₃₋₁₀hetaryl, —C₃₋₁₀aryl-C₃₋₁₀cycloalkyl, —C₃₋₁₀aryl-C₁₋₁₀heterocyclyl, —C₁₋₁₀hetaryl-C₁₋₁₀alkyl, —C₁₋₁₀hetaryl-C₂₋₁₀alkenyl, —C₁₋₁₀hetaryl-C₂₋₁₀alkynyl, —C₃₋₁₀hetaryl-C₃₋₁₀aryl, —C ₁₋₁₀hetaryl-C₃₋₁₀cycloalkyl, —C₁₋₁₀hetaryl-C₁₋₁₀heterocyclyl, —C₃₋₁₀cycloalkyl-C₁₋₁₀alkyl, —C₃₋₁₀cycloalkyl-C₂₋₁₀alkenyl, —C₃₋₁₀cycloalkyl-C₂₋₁₀alkynyl, —C₃₋₁₀cycloalkyl-C₃₋₁₀aryl, —C₃₋₁₀cycloalkyl-C₁₋₁₀hetaryl, —C₃₋₁₀cycloalkyl-C₁₋₁₀heterocyclyl, —C₁₋₁₀heterocyclyl-C₁₋₁₀alkyl, —C ₁₋₁₀heterocyclyl-C₂₋₁₀alkenyl, —C₁₋₁₀heterocyclyl-C₂₋₁₀alkynyl, —C₁₋₁₀heterocyclyl-C₃₋₁₀aryl, —C₁₋₁₀heterocyclyl-C₁₋₁₀hetaryl, or —C₁₋₁₀heterocyclyl-C₃₋₁₀cycloalkyl, each of which is unsubstituted or substituted by one or more independent R₁₃ substituents; or R₃′ is —OR⁶, —NR⁶R³⁴, —S(O)₀₋₂R⁶, —C(═O)R⁶, —C(═O)OR⁶, —OC(═O)R⁶, —C(═O)N(R³⁴)R⁶, or —N(R³⁴)C(═O)R⁶, wherein R⁶ together with R³⁴ can optionally form a heterocyclic ring; or R₃^{′} is —OR⁶, —NR⁶R³⁴, —S(O)₀₋₂R⁶, —C(═O)R⁶, —C(═O)OR⁶, —OC(═O)R⁶, —C(═O)N(R³⁴)R⁶, or —N(R³⁴)C(═O)R⁶, wherein R⁶ together with R³⁴ can optionally form a heterocyclic ring;

each of R₅, R₇₁, R₈₁ and R₉₁ is independently hydrogen, halogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —OH, —CF₃, —OCF₃, —OR³¹, —NR³¹R³², —C(O)R³¹, —CO₂R³¹, —C(═O)NR³¹, —NO₂, —CN, —S(O)₀₋₂R³¹, —SO₂NR³¹R³², —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³²R³³, —NR³¹C(═NR³²)OR³³, —NR³¹C(═NR³²)SR³³, —OC(═O)OR³³, —OC(═O)NR³¹R³², —OC(═O)SR³¹, —SC(═O)SR³¹, —P(O)OR⁻OR³², or —SC(═O)NR³¹NR³²;

R₆ is hydrogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —C₁₋₁₀alkyl-C₃₋₁₀aryl, —C₁₋₁₀alkyl-C₁₋₁₀hetaryl, —C₁₋₁₀alkyl-C₃₋₁₀cycloalkyl, —C₁₋₁₀alkyl-C₁₋₁₀heterocyclyl, —C₂₋₁₀alkenyl-C₃₋₁₀aryl, —C₂₋₁₀alkenyl-C₁₋₁₀hetaryl, —C₂₋₁₀alkenyl-C₃₋₁₀cycloalkyl, —C₂₋₁₀alkenyl-C₁₋₁₀heterocyclyl, —C₂₋₁₀alkynyl-C₃₋₁₀aryl, —C₂₋₁₀alkynyl-C₁₋₁₀hetaryl, —C₂₋₁₀alkynyl-C₃₋₁₀cycloalkyl, —C₂₋₁₀alkynyl-C₁₋₁₀heterocyclyl, —C₁₋₁₀heteroalkyl-C₃₋₁₀aryl, —C₁₋₁₀heteroalkyl-C₁₋₁₀hetaryl, —C₁₋₁₀heteroalkyl-C₃₋₁₀cycloalkyl, —C₁₋₁₀heteroalkyl-C₁₋₁₀heterocyclyl, —C₁₋₁₀alkoxy-C₃₋₁₀aryl, —C₁₋₁₀alkoxy-C₁₋₁₀hetaryl, —C₁₋₁₀alkoxy-C₃₋₁₀cycloalkyl, —C₁₋₁₀alkoxy-C₁₋₁₀heterocyclyl, —C₃₋₁₀aryl-C₁₋₁₀alkyl, —C₃₋₁₀aryl-C₂₋₁₀alkenyl, —C₃₋₁₀aryl-C₂₋₁₀alkynyl, —C₃₋₁₀aryl-C₃₋₁₀hetaryl, —C₃₋₁₀aryl-C₃₋₁₀cycloalkyl, —C₃₋₁₀aryl-C₁₋₁₀heterocyclyl, —C₁₋₁₀hetaryl-C₁₋₁₀alkyl, —C₁₋₁₀hetaryl-C₂₋₁₀alkenyl, —C₁₋₁₀hetaryl-C₂₋₁₀alkynyl, —C₃₋₁₀hetaryl-C₃₋₁₀aryl, —C₁₋₁₀hetaryl-C₃₋₁₀cycloalkyl, —C₁₋₁₀hetaryl-C₁₋₁₀heterocyclyl, —C₃₋₁₀cycloalkyl-C₁₋₁₀alkyl, —C₃₋₁₀cycloalkyl-C₂₋₁₀alkenyl, —C₃₋₁₀cycloalkyl-C₂₋₁₀alkynyl, —C₃₋₁₀cycloalkyl-C₃₋₁₀aryl, —C₃₋₁₀cycloalkyl-C₁₋₁₀hetaryl, —C₃₋₁₀cycloalkyl-C₁₋₁₀heterocyclyl, —C₁₋₁₀heterocyclyl-C₁₋₁₀alkyl, —C₁₋₁₀heterocyclyl-C₂₋₁₀alkenyl, —C₁₋₁₀heterocyclyl-C₂₋₁₀alkynyl, —C₁₋₁₀heterocyclyl-C₃₋₁₀aryl, —C₁₋₁₀heterocyclyl-C₁₋₁₀hetaryl, or —C₁₋₁₀heterocyclyl-C₃₋₁₀cycloalkyl, each of which is unsubstituted or substituted by one or more independent R₁₄ or R₁₅ substituents;

each of R₇₂, R₈₂ and R₉₂ is independently hydrogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —OH, —CF₃, —C(O)R³¹, —CO₂R³¹, —C(═O)NR³¹, —S(O)₀₋₂R³¹, —C(═S)OR³¹, —C(═O)SR³¹;

each of R₁₀and R₁₄ is independently —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, optionally substituted by one or more independent R₁₁ substituents;

each of R₁₁, R_{12,} R₁₃ and R₁₅ is independently hydrogen, halogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —OH, —CF₃, —OCF₃, —OR³¹, —NR³¹R³², —C(O)R³¹, —CO₂R³¹, —C(═O)NR³¹, —NO₂, —CN, —S(O)₀₋₂R³¹, —SO₂NR³¹R³², —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³²R³³, —NR³¹C(═NR³²)OR³³, —NR³¹C(═NR³²)SR³³, —OC(═O)OR³³, —OC(═O)NR³¹R³², —OC(═O)SR³¹, —SC(═O)SR³¹, —P(O)OR⁻OR³², or —SC(═O)NR³¹NR³²;

each of R³¹, R³², R³³ and R³⁴ is independently hydrogen, halogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, or wherein R³¹ together with R³² form a heterocyclic ring;

wherein ring A comprises one or more heteroatoms selected from N, O, or S; and

wherein if X₇ is O or X₂-X₃ is R₁C═CR₃, ring A comprises at least two heteroatoms selected from N, O, or S; and

wherein if X₂-X₃ is R₁C═N, at least one of X₇ or X₉ is not N.

In some embodiments, the ERK inhibitor is a compound of Formula I-A:

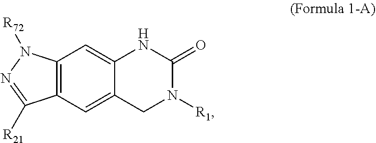

or a pharmaceutically acceptable salt thereof.

In some embodiments, for a compound of Formula I or I-A:

R₁ is —C₁₋₁₀alkyl, —C₁₋₁₀alkyl-C₃₋₁₀aryl, or —C₁₋₁₀heterocyclyl-C₁₋₁₀alkyl, each of which is unsubstituted or substituted by one or more independent R₁₀ or R₁₁ substituents;

R₂₁ is -L-C₃₋₁₀aryl or -L-C₁₋₁₀hetaryl, each of which is unsubstituted or substituted by one or more independent R₁₂ substituents;

L is a bond or —N(R³¹)—;

R₇₂ is hydrogen;

each of R₁₀ is independently —C₃₋₁₀aryl, —C₁₋₁₀hetaryl, or —C₁₋₁₀heterocyclyl, optionally substituted by one or more independent R₁₁ substituents;

each of R₁₁ and R₁₂ is independently halogen, —C₁₋₁₀alkyl, —OH, —CF₃ or —OR³¹; and each of R³¹ is independently hydrogen or —C₁₋₁₀alkyl.

In practicing any of the subject methods, the ERK inhibitor may be selected from the group consisting of:

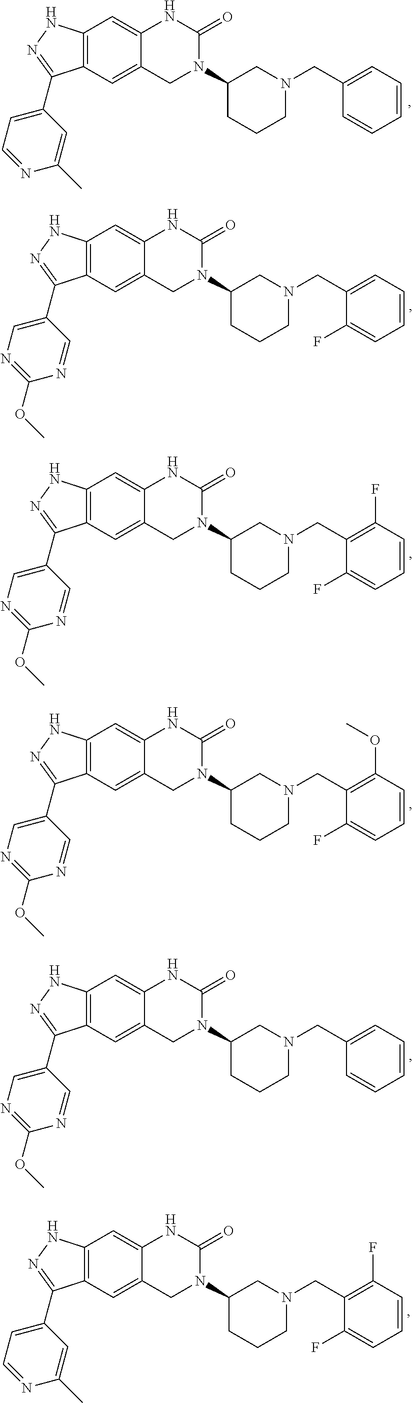

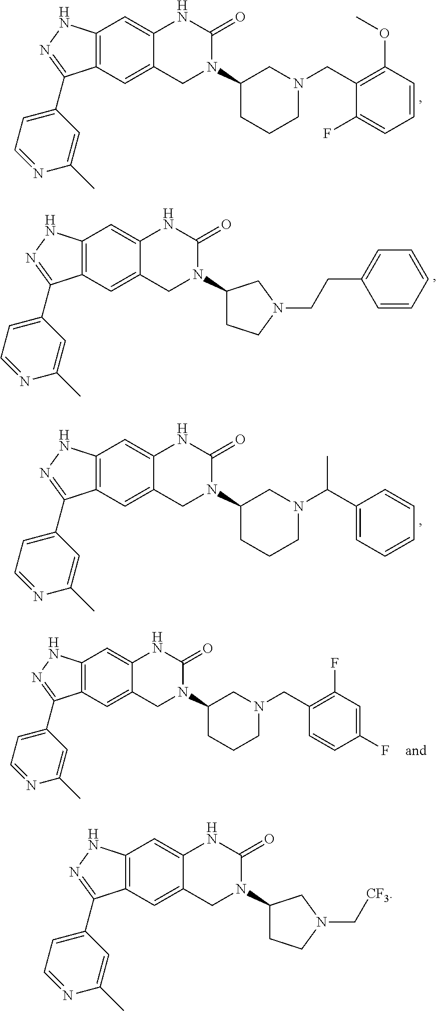

In some embodiments, the ERK inhibitor is selected from the group consisting of ulixertinib, BVD-523, RG7842, GDC-0094, GDC-0994, CC-90003, LTT-462, ASN-007, AMO-01, KO-947, AEZS-134, AEZS-131, AEZS-140, AEZS-136, AEZS-132, D-87503, KIN-2118, RB-1, RB-3, SCH-722984, SCH-772984, MK-8353, SCH-900353, FR-180204, IDN-5491, hyperforin trimethoxybenzoate, ERK1-2067, ERK1-23211, and ERK1-624. In some embodiments, the ERK inhibitor is selected from the group consisting of:

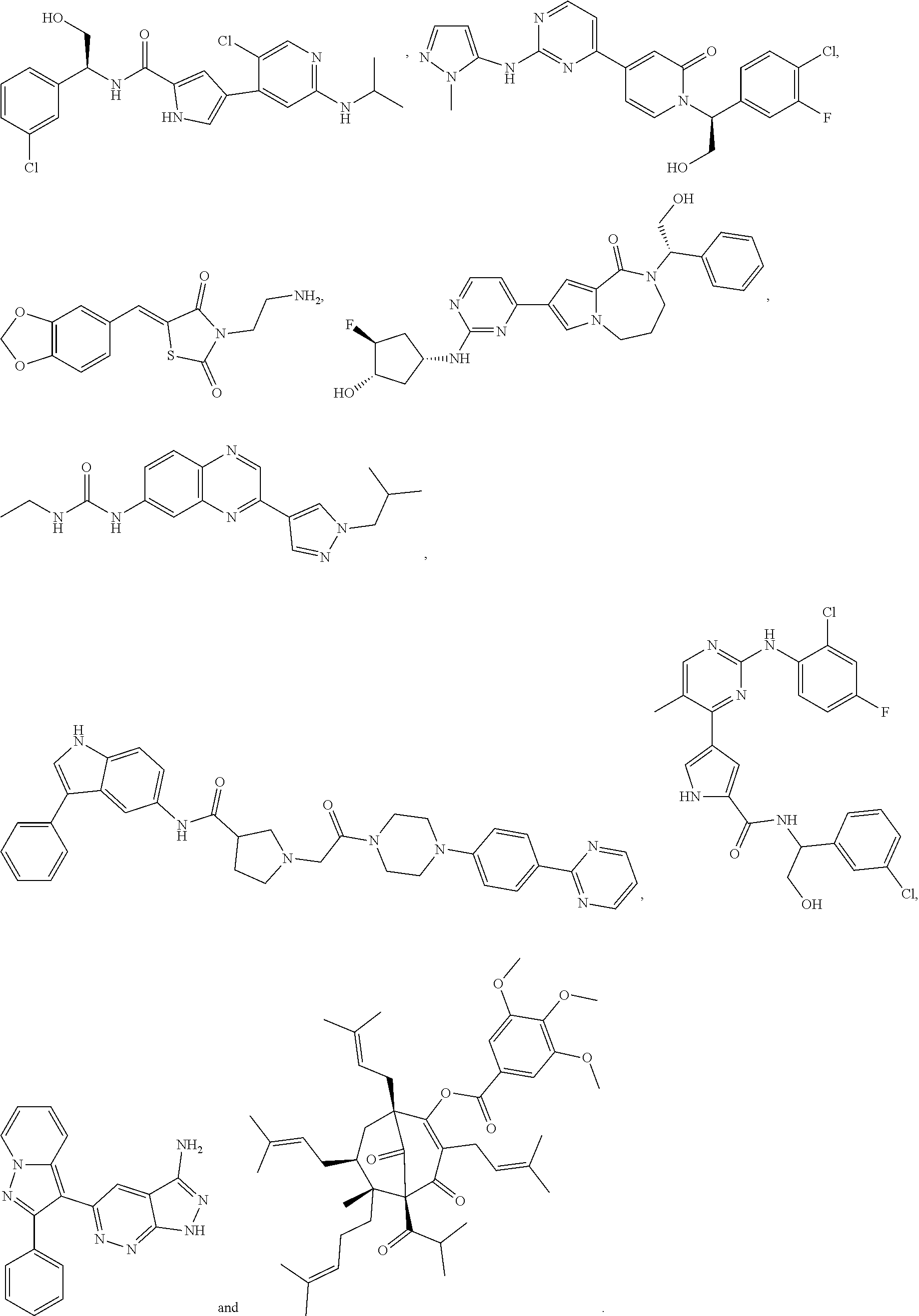

A method described herein may further comprise administering a second therapeutic agent to the subject. In certain aspects, the present disclosure provides a method of treating squamous cell carcinoma in a subject in need thereof, comprising administering to said subject an ERK inhibitor and a second therapeutic agent. In some embodiments, the second therapeutic agent is a chemotherapeutic agent. In some embodiments, the second therapeutic agent is selected from gemcitabine, cisplatin, an EGFR inhibitor and a CDK inhibitor. In some embodiments, the second therapeutic agent is selected from gemcitabine, cisplatin, palbociclib, osimertinib, olmutinib, icotinib hydrochloride, afatinib, necitumumab, lapatinib, pertuzumab, vandetanib, nimotuzumab, panitumumab, erlotinib, gefitinib and cetuximab. In some embodiments, the second therapeutic agent is selected from gemcitabine, cisplatin, cetuximab, erlotinib and palbociclib. A method described herein may further comprise administering chemotherapy, immunotherapy or radiotherapy to the subject.

In certain embodiments, the present disclosure provides a system for assessing a likelihood of a subject having squamous cell carcinoma exhibiting a clinically beneficial response to treatment with an ERK inhibitor. In some embodiments, the system comprises (a) a memory unit configured to store information concerning: (i) a first total expression level of at least two genes selected from the group consisting of EGFR, ERK1, CCND1 , KRAS, ERK2, and HRAS; (ii) a second total expression level of at least two genes selected from the group consisting of DUSP5 , DUSP6, DUSP2 , DUSP4 , SPRY2 , SPRY4, and SPRED1; (iii) a third total expression level of at least two genes selected from the group consisting of CCND1 , CRAF, DUSP5 , EGFR, ERK1, and KRAS; (iv) a copy number profile of at least one MAPK pathway gene; (v) a fourth total expression level of AREG, CDH3, COL17A1 , EGFR, HIF1A, ITGB1, KRT1, KRT9 , NRG1 , SLC16A1 , SLC22A _I and VEGFA; (vi) a fifth total expression level of DCUN1D1, PIK3CA, PRKCI, SOX2 and TP63; and/or (vii) expression levels of HIF1A and TP63; in a biological sample comprising genomic and/or transcriptomic material from a squamous cell carcinoma cell; (b) one or more processors alone or in combination programmed to (1) determine a weighted probability of ERK inhibitor responsiveness based on the first total expression level, the second total expression level, the copy number profile, the third total expression level, the fourth total expression level, the fifth total expression level, and/or the expression levels of HIF1A and TP63; and (2) designate the subject as having a high probability of exhibiting a clinically beneficial response to treatment with the ERK inhibitor if the weighted probability corresponds to at least 1.5 times a baseline probability, wherein the baseline probability represents a likelihood that the subject will exhibit a clinically beneficial response to treatment with the ERK inhibitor before obtaining the weighted probability of (b)(1). In some embodiments, the first total expression level, the second total expression level, the third total expression level, the fourth total expression level, the fifth total expression level, and/or the expression levels of HIF1A and TP63 are assessed by (a) detecting a level of mRNA; (b) detecting a level of cDNA produced from reverse transcription of mRNA; (c) detecting a level of polypeptide; (d) detecting a level of cell-free DNA; or (e) a nucleic acid amplification assay, a hybridization assay, sequencing, or a combination thereof In some embodiments, the copy number profile of the at least one MAPK pathway gene is assessed by a method selected from the group consisting of in situ hybridization, Southern blot, immunohistochemistry (IHC), polymerase chain reaction (PCR), quantitative PCR (qPCR), quantitative real-time PCR (qRT-PCR), comparative genomic hybridization, microarray-based comparative genomic hybridization, and ligase chain reaction (LCR). In some embodiments, the at least one MAPK pathway gene is selected from EGFR, ERK1 , CCND1, KRAS, ERK2 and HRAS, such as EGFR. In some embodiments, the squamous cell carcinoma is selected from lung, esophagus, cervical and head and neck squamous cell carcinomas, such as head and neck squamous cell carcinoma.

In certain aspects, the present disclosure provides a system for assessing a likelihood of a subject having cancer exhibiting a clinically beneficial response to treatment with an ERK inhibitor. In some embodiments, the system comprises (a) a memory unit configured to store information concerning a copy number profile and/or expression level of at least one gene located at chromosome 11q13.3-13.4 in a biological sample comprising genomic and/or transcriptomic material from a cancer cell; and (b) one or more processors alone or in combination programmed to (1) determine a weighted probability of ERK inhibitor responsiveness based on the copy number profile and/or the expression level; and (2) designate the subject as having a high probability of exhibiting a clinically beneficial response to treatment with the ERK inhibitor if the weighted probability corresponds to at least 1.5 times a baseline probability, wherein the baseline probability represents a likelihood that the subject will exhibit a clinically beneficial response to treatment with the ERK inhibitor before obtaining the weighted probability of (b)(1). In some embodiments, the expression level is assessed by (a) detecting a level of mRNA; (b) detecting a level of cDNA produced from reverse transcription of mRNA; (c) detecting a level of polypeptide; (d) detecting a level of cell-free DNA; or (e) a nucleic acid amplification assay, a hybridization assay, sequencing, or a combination thereof In some embodiments, the copy number profile of the at least one gene is assessed by a method selected from the group consisting of in situ hybridization, Southern blot, immunohistochemistry (IHC), polymerase chain reaction (PCR), quantitative PCR (qPCR), quantitative real-time PCR (qRT-PCR), comparative genomic hybridization, microarray-based comparative genomic hybridization, and ligase chain reaction (LCR). In some embodiments, the at least one gene is selected from CCND1, CTTN, FADD, ORAOV1, ANO1, PPFIA1 and SHANK2. In some embodiments, the at least one gene is CCND1 or ANO1 . In some embodiments, the at least one gene is CCND1 and ANO1 . In some embodiments, the cancer is selected from the group consisting of squamous cell carcinoma and adenocarcinoma. In some embodiments, the cancer is a squamous cell carcinoma selected from the group consisting of lung, esophageal, cervical, head and neck, bladder and gastric squamous cell carcinomas, such as esophageal squamous cell carcinoma. In some embodiments, the cancer is an adenocarcinoma selected from the group consisting of esophageal and pancreatic adenocarcinomas. In some embodiments, the cancer is selected from the group consisting of lung, esophageal, cervical, head and neck, bladder, gastric and pancreatic cancer. In some embodiments, the cancer is selected from breast cancer, pancreatic cancer, lung cancer, thyroid cancer, seminomas, melanoma, bladder cancer, liver cancer, kidney cancer, myelodysplastic syndrome, acute myelogenous leukemia and colorectal cancer.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts tumor volumes of six groups of non-small cell lung squamous cell carcinoma models over 2- and 4-week treatment schedules with vehicle or an ERK inhibitor.

FIG. 2 presents gene copy numbers for eleven non-small cell lung squamous cell carcinoma models and percent tumor growth inhibition after treatment with an ERK inhibitor.

FIG. 3 depicts tumor volumes of five groups of esophageal squamous cell carcinoma models over 6- and 3-week treatment schedules with vehicle or an ERK inhibitor.

FIG. 4 presents gene copy numbers for nine esophageal squamous cell carcinoma models and percent tumor growth inhibition after treatment with an ERK inhibitor.

FIG. 5 depicts tumor volumes of five groups of head and neck squamous cell carcinoma models over 3- and 4-week treatment schedules with vehicle or an ERK inhibitor.

FIG. 6 presents gene copy numbers for nine head and neck squamous cell carcinoma models and percent tumor growth inhibition after treatment with an ERK inhibitor.

FIG. 7 illustrates the correlation between percent tumor growth inhibition and EGFR gene copy number.

FIG. 8 shows the mean tumor volumes of three groups of head and neck squamous cell carcinoma models after treatment with vehicle or an ERK inhibitor.

FIG. 9 depicts the tumor regression observed in mice bearing subcutaneous head and neck squamous cell carcinomas following treatment with an ERK inhibitor.

FIG. 10 illustrates that 6- and 4-gene signatures comprising MAPK pathway genes predict response to ERK inhibition in squamous cell carcinoma models.

FIG. 11 illustrates that two 3-gene signatures comprising MAPK pathway genes predict response to ERK inhibition in squamous cell carcinoma models.

FIG. 12 illustrates that three 2-gene signatures comprising MAPK pathway genes predict response to ERK inhibition in squamous cell carcinoma models.

FIG. 13 illustrates that both a 6-gene signature comprising NRAS, ARAF, BRAF, CRAF, MEK1 and MEK2 and a 1-gene signature comprising EGFR fail to predict response to ERK inhibition in squamous cell carcinoma models.

FIG. 14 illustrates that 6- and 8-gene signatures comprising MAPK pathway genes and RAS-ERK feedback regulators predict response to ERK inhibition in squamous cell carcinoma models.

FIG. 15 illustrates that 5-, 4- and 2-gene signatures comprising RAS-ERK feedback regulators predict response to ERK inhibition in squamous cell carcinoma models.

FIG. 16 illustrates that a 12-gene signature associated with the ‘basal’ subtype of squamous cell carcinomas of the head and neck (HNSCC) predicts for good response to ERK inhibition, whereas a 5-gene signature derived from genes located in a region of chromosome 3 that is commonly amplified (Ch3A) in HNSCC predicts for poor response to ERK inhibition. This figure also shows that a ratio of the 12- to the 5-gene signature, and even a ratio of HIF1A to TP63, predicts for good response to ERK inhibition.

FIG. 17 illustrates the activity of an ERK inhibitor in models of clinical B-Raf and MEK inhibitor resistance.

FIG. 18 depicts tumor volumes of eleven esophageal squamous cell carcinoma models treated with either vehicle (black squares) or an ERK inhibitor (open circles). Treatment response is categorized as complete response (CR, >90% regression), partial response (PR, >30% regression), stable disease (SD, <30% regression) or progressive disease (PD, >20% tumor growth).

FIG. 19 illustrates the relationship between CCND1 amplification and cyclin D1 overexpression.

FIG. 20 illustrates the dependence of adenocarcinomas and squamous cell carcinomas on the MAP kinase pathway.

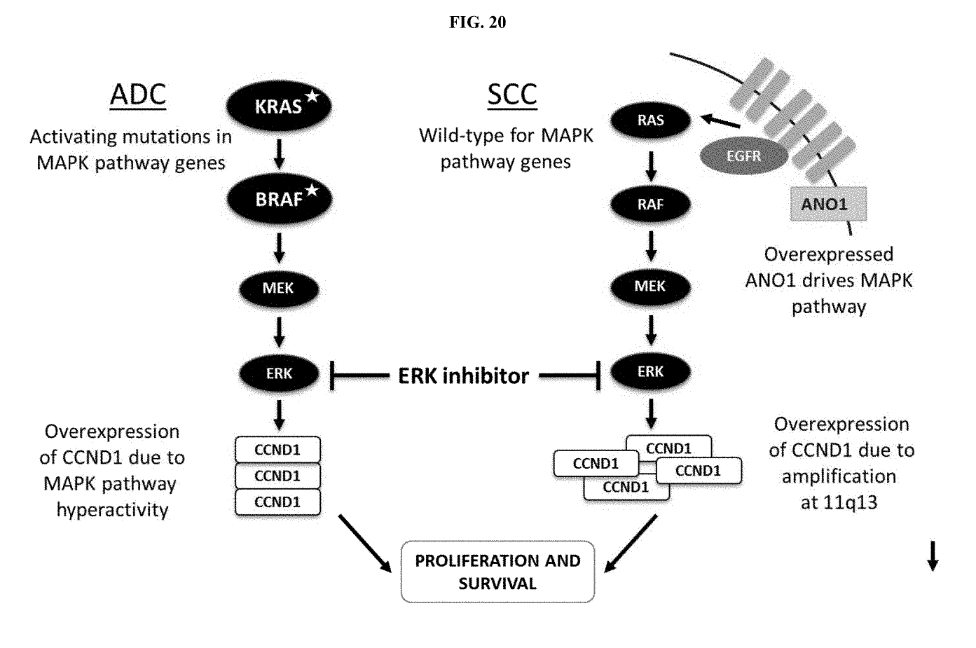

FIG. 21 presents copy numbers of CCND1 and six additional genes located at chromosome 11q13.3-13.4 that are co-amplified with CCND1 in 22 esophageal squamous cell carcinoma models.

FIG. 22 illustrates expression levels of six additional genes located in the 11q13 amplicon in amplified responding (“AMP”) and unamplified, non-responding (“WT”) esophageal squamous cell carcinoma models.

FIG. 23 illustrates the correlation between CCND1 and ANO1 amplification in numerous cancer subtypes.

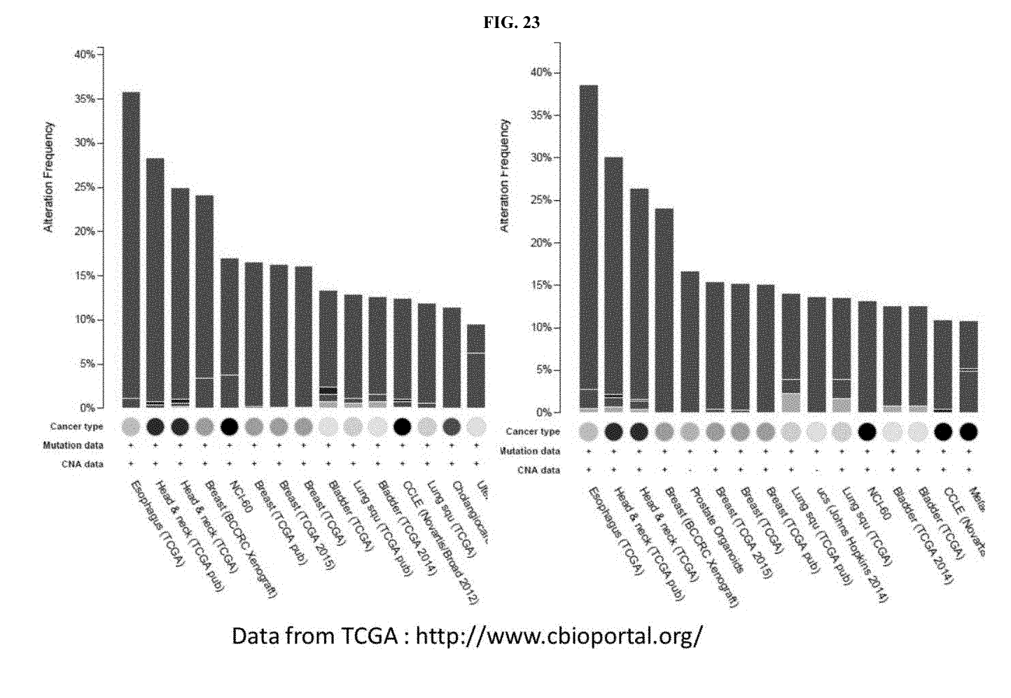

FIG. 24 illustrates the relationship between CCND1 amplification, ANO1 amplification, and response to treatment with an ERK inhibitor in esophageal squamous cell carcinoma models.

FIG. 25 compares the CCND1 amplification status to response to treatment with an ERK inhibitor in lung squamous cell carcinoma models.

FIG. 26 compares the CCND1 amplification status to response to treatment with an ERK inhibitor in head and neck squamous cell carcinoma models.

FIG. 27 depicts tumor volumes of four KRAS-mutant pancreatic cancer models treated with either vehicle (black squares) or an ERK inhibitor (open circles).

FIG. 28 depicts tumor volumes of bladder and gastric cancer models treated with either vehicle (diamonds), 120 mg/kg EOD ERK inhibitor (squares) or 300 mg/kg QW ERK inhibitor (triangles).

FIG. 29 illustrates percent tumor growth for esophageal squamous cell carcinoma models treated with an ERK inhibitor.

FIG. 30 illustrates percent tumor growth for esophageal squamous cell carcinoma models treated with an ERK inhibitor. 11q13-amplified and 11q13 wild-type models are distinguished as white and black bars, respectively.

FIG. 31 illustrates percent tumor growth for esophageal squamous cell carcinoma models treated with an ERK inhibitor. 11q13-amplified/AN01⁺and 11q13 wild-type models are distinguished as white and black bars, respectively.

FIG. 32 illustrates the correlation between CCND1 and ANO1 expression in esophageal adenocarcinoma models.

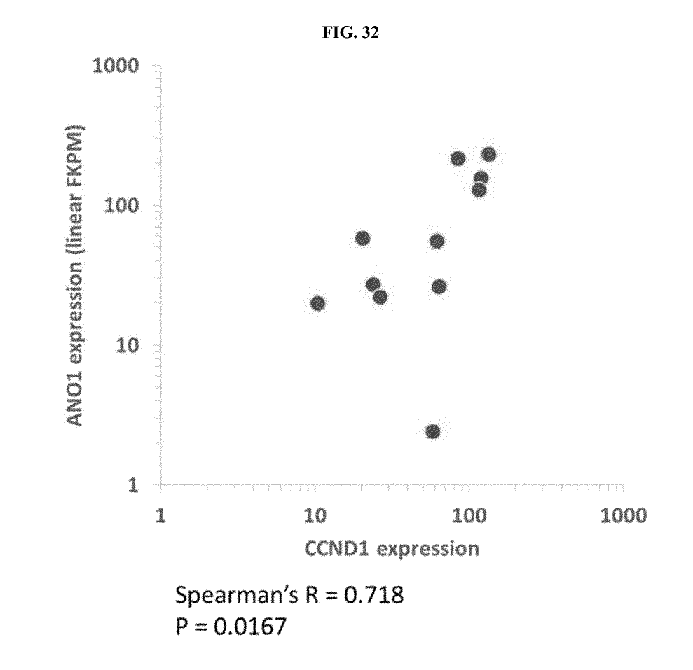

FIG. 33 depicts tumor volumes of HNSCC models treated with either vehicle (black squares), an ERK inhibitor (black circles), a CDK4/6 inhibitor (open triangles) or the ERK inhibitor and the CDK4/6 inhibitor (open diamonds).

FIG. 34 depicts tumor volumes of HNSCC and ESCC models treated with either vehicle (black squares), an ERK inhibitor (black circles), a CDK4/6 inhibitor (open triangles) or the ERK inhibitor and the CDK4/6 inhibitor (open diamonds).

FIG. 35 depicts body weights of HNSCC and ESCC murine models treated with either vehicle (black squares), an ERK inhibitor (black circles), a CDK4/6 inhibitor (open triangles) or the ERK inhibitor and the CDK4/6 inhibitor (open diamonds) over the study duration.

### DETAILED DESCRIPTION OF THE INVENTION

Unless defined otherwise, all technical and scientific terms used herein have the same meaning as is commonly understood by one of skill in the art to which this invention belongs.

“About” as used herein when referring to a measurable value such as an amount, a duration, and the like, is meant to encompass variations of ±10% of a stated number or value.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, primers, cell-free DNA (cfDNA), and circulating tumor DNA (ctDNA). A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

A “nucleotide probe” or “probe” refers to a polynucleotide used for detecting or identifying its corresponding target polynucleotide in a hybridization reaction.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR, or the enzymatic cleavage of a polynucleotide by a ribozyme.

As used herein, “expression” refers to the process by which a polynucleotide is transcribed into mRNA and/or the process by which the transcribed mRNA (also referred to as a “transcript”) is subsequently translated into peptides, polypeptides, or proteins. The transcripts and the encoded polypeptides are collectedly referred to as “gene product.” If the polynucleotide is derived from genomic DNA, expression may include splicing of the mRNA in a eukaryotic cell. The level of expression (or alternatively, the “expression level”) of an EGFR gene can be determined, for example, by determining the level of EGFR polynucleotides, polypeptides, and/or gene products.

“Differentially expressed” or “differential expression” as applied to a nucleotide sequence (e.g., a gene) or polypeptide sequence in a subject, refers to the differential production of the mRNA transcribed and/or translated from the nucleotide sequence or the protein product encoded by the nucleotide sequence. A differentially expressed sequence may be overexpressed or underexpressed as compared to the expression level of a reference sample (i.e., a reference level). As used herein, overexpression is an increase in expression and generally is at least 1.25 fold, or alternatively, at least 1.5 fold, or alternatively, at least 2 fold, or alternatively, at least 3 fold, or alternatively, at least 4 fold, or alternatively, at least 10 fold expression over that detected in a reference sample. As used herein, underexpression is a reduction in expression and generally is at least 1.25 fold, or alternatively, at least 1.5 fold, or alternatively, at least 2 fold, or alternatively, at least 3 fold, or alternatively, at least 4 fold, or alternatively, at least 10 fold expression under that detected in a reference sample. Underexpression also encompasses absence of expression of a particular sequence as evidenced by the absence of detectable expression in a test subject when compared to a reference sample.

“Signal transduction” is a process during which stimulatory or inhibitory signals are transmitted into and within a cell to elicit an intracellular response. A molecule can mediate its signaling effect via direct or indirect interaction with downstream molecules of the same pathway or related pathway(s). For instance, MAPK signaling can involve a host of downstream molecules including but not limited to one or more of the following proteins: EGFR, ERK1, CCND1, KRAS, ERK2 and HRAS.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The terms “biomarker” and “marker” are used interchangeably herein to refer to a molecule which is differentially present in a sample taken from a subject of one phenotypic status (e.g., having a squamous cell carcinoma that is sensitive to an ERK inhibitor) as compared with another phenotypic status (e.g., having a squamous cell carcinoma that has low sensitivity to an ERK inhibitor). A biomarker is differentially present between different phenotypic statuses if the mean or median expression level of the biomarker in the different groups is calculated to be statistically significant. Common tests for statistical significance include, for example, t-test, ANOVA, Kruskal-Wallis, Wilcoxon, Mann-Whitney and odds ratio. Biomarkers, alone or in combination, can provide measures of relative risk that a subject belongs to one phenotypic status or another. Therefore, they are useful as markers for disease (diagnostics), therapeutic effectiveness of a drug (theranostics) and drug toxicity. The polynucleotides and polypeptides described herein can be used as biomarkers for certain cancers described herein.

A “reference sample” is an alternative sample or subject used in an experiment for comparison purpose.

The term “reference level” refers to a control level used to evaluate a test level. In some examples, a reference level may be a control. For example, a biomarker may be considered to be underexpressed when the expression level of that biomarker is lower than a reference level. The reference level can be determined by a plurality of methods, provided that the resulting reference level accurately provides a level of a biomarker above which exists a first group of subjects having a different probability of exhibiting a clinically beneficial response to treatment with an ERK inhibitor than that of a second group of patients having levels of the biomarker below the reference level. The reference level may be determined, for example, by measuring the level of expression of a biomarker in tumorous or non-tumorous cancer cells from the same tissue as the tissue of the cancer cells to be tested. In some examples, the reference level may be a level of a biomarker determined in vitro. A reference level may be determined by comparison of the level of a biomarker in populations of subjects having the same cancer. Two or more separate groups of subjects may be determined by identification of subsets of populations of the cohort that have the same or similar levels of a biomarker. Determination of a reference level can then be made based on a level that distinguishes these separate groups. A reference level may be a single number, equally applicable to every subject, or a reference level can vary according to specific subpopulations of subjects. For example, older men may have a different reference level than younger men for the same cancer, and women may have a different reference level than men for the same cancer. Furthermore, the reference level may be some level determined for each subject individually. For example, the reference level may be a ratio of a biomarker level in a cancer cell of a subject relative to the biomarker level in a normal cell within the same subject. In some embodiments, a reference level is a numerical range of gene expression that is obtained from a statistical sampling from a population of individuals having cancer. The sensitivity of the individuals having cancer to treatment with an ERK inhibitor may be known. In certain embodiments, the reference level is derived by comparing gene expression to a control gene that is expressed in the same cellular environment at relatively stable levels (e.g. a housekeeping gene such as an actin). Comparison to a reference level may be a qualitative assessment or a quantitative determination.

The terms “determining,” “measuring,” “evaluating,” “assessing,” “assaying,” “testing,” and “analyzing” are used interchangeably herein to refer to any form of measurement, and include determining if an analyte is present or not (e.g., detection). These terms can include both quantitative and/or qualitative determinations. Assessing may be relative or absolute. A relative amount could be, for example, high, medium or low. An absolute amount could reflect the measured strength of a signal or the translation of this signal strength into another quantitative format, such as micrograms/mL. “Detecting the presence of” can include determining the amount of something present, as well as determining whether it is present or absent.

As used herein, “agent” or “biologically active agent” refers to a biological, pharmaceutical, or chemical compound or other moiety. Non-limiting examples include a simple or complex organic or inorganic molecule, a peptide, a protein, an oligonucleotide, an antibody, an antibody derivative, antibody fragment, a vitamin derivative, a carbohydrate, a toxin, or a chemotherapeutic compound. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like. A skilled artisan can readily recognize that there is no limit as to the structural nature of the agents of the present disclosure.

The terms “antagonist” and “inhibitor” are used interchangeably, and they refer to a compound having the ability to inhibit a biological function of a target protein (e.g., ERK), whether by inhibiting the activity or expression of the target protein. Accordingly, the terms “antagonist” and “inhibitors” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition. A preferred biological activity inhibited by an antagonist is associated with the development, growth, or spread of a squamous cell carcinoma.

The term “cell proliferation” refers to a phenomenon by which the cell number has changed as a result of division. This term also encompasses cell growth by which the cell morphology has changed (e.g., increased in size) consistent with a proliferative signal.

The terms “co-administration,” “administered in combination with,” and their grammatical equivalents, encompass administration of two or more agents to a subject so that both agents and/or their metabolites are present in the subject at the same time. Co-administration includes simultaneous administration in separate compositions, administration at different times in separate compositions, or administration in a composition in which both agents are present.

The term “effective amount” or “therapeutically effective amount” refers to that amount of a compound described herein that is sufficient to effect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

As used herein, the terms “treatment”, “treating”, “palliating” and “ameliorating” are used interchangeably. These terms refer to an approach for obtaining beneficial or desired results including, but are not limited to, therapeutic benefit and/or a prophylactic benefit. By therapeutic benefit is meant eradication or amelioration of the underlying disorder being treated (e.g., squamous cell carcinoma). Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, the pharmaceutical compositions may be administered to a patient at risk of developing a particular disease, or to a patient reporting one or more of the physiological symptoms of a disease, even though a diagnosis of this disease may not have been made.

A “therapeutic effect,” as used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof

The term “selective inhibition” or “selectively inhibit” as applied to a biologically active agent refers to the agent's ability to selectively reduce the target signaling activity as compared to off-target signaling activity, via direct or indirect interaction with the target.

The term “subject” includes, but is not limited to, humans of any age group, e.g., a pediatric subject (e.g., infant, child or adolescent) or adult subject (e.g., young adult, middle-aged adult or senior adult)) and/or other primates (e.g., cynomolgus monkeys or rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. The methods described herein can be useful in both human therapeutics and veterinary applications. In some embodiments, the patient is a mammal, and in some embodiments, the patient is human.

“Radiation therapy” or “radiation treatment” means exposing a patient, using routine methods and compositions known to the practitioner, to radiation emitters such as alpha-particle emitting radionucleotides (e.g., actinium and thorium radionuclides), low linear energy transfer (LET) radiation emitters (e.g., beta emitters), conversion electron emitters (e.g., strontium-89 and samarium-153-EDTMP), or high-energy radiation, including without limitation x-rays, gamma rays, and neutrons.

The term “in vivo” refers to an event that takes place in a subject's body.

The term “in vitro” refers to an event that takes place outside of a subject's body. For example, an in vitro assay encompasses any assay run outside of a subject's body. In vitro assays encompass cell-based assays in which cells alive or dead are employed. In vitro assays also encompass a cell-free assay in which no intact cells are employed.

“ERK1 and/or ERK2 activity” as applied to a biologically active agent refers to the agent's ability to modulate signal transduction mediated by ERK1 and/or ERK2. For example, modulation of ERK1 and/or ERK2 activity is evidenced by alteration in signaling output from the Ras/Raf/MEK/ERK pathway.

The term “inhibiting ERK activity”, as used herein, refers to slowing, reducing, altering, as well as completely eliminating and/or preventing ERK activity.

The present inventors have discovered certain genes that are amplified and/or differentially expressed in squamous cell carcinoma cells that are sensitive to therapy with an ERK inhibitor, such as a compound described herein. More specifically, the disclosure relates to the use of an inhibitor of extracellular signal-regulated kinases 1 and 2 (ERK1 and ERK2) to treat squamous cell carcinomas, such as squamous cell carcinomas of the lung (LSCC), esophagus (ESCC), head and neck (HNSCC) and cervix. Methods of using information about the amplification or expression status of the genes and/or the gene expression products to identify squamous cell carcinoma cells that will likely respond to therapy with an ERK inhibitor as well as methods of identifying subjects having squamous cell carcinoma that are predicted to exhibit a clinically beneficial response to treatment with an ERK inhibitor are described herein. In particular, copy number amplification of one or more of the genes may be indicative of sensitivity to therapy with an ERK inhibitor. Use of certain DNA- and RNA-based biomarkers to identify LSCC, ESCC and HNSCC tumors more likely to display a robust therapeutic response to ERK inhibition are described.

In certain embodiments, the present disclosure provides a method of treating squamous cell carcinoma in a subject in need thereof In some embodiments, the method comprises administering an effective dose of an inhibitor of an extracellular signal-regulated kinase (ERK) to the subject, said subject comprising a genome that exhibits (1) a first total expression level of at least two mitogen-activated protein kinase (MAPK) pathway genes that is greater than a first reference level and/or (2) a second total expression level of at least two RAS-ERK feedback regulators that is greater than a second reference level, wherein the first reference level and the second reference level are each indicative of low sensitivity to the ERK inhibitor.

In certain embodiments, the present disclosure provides a method of treating head and neck squamous cell carcinoma in a subject in need thereof, comprising administering an effective dose of an inhibitor of an extracellular signal-regulated kinase (ERK) to the subject, said subject comprising a genome that exhibits (1) a fourth total expression level of AREG, CDH3, COL17A1 , EGFR, HIF1A, ITGB1 , KRT1 , KRT9 , NRG1 , SLC16A1 , SLC22A _I and VEGFA that is greater than a fourth reference level; (2) a fifth total expression level of DCUN1D1, PIK3CA, PRKCI, SOX2 and TP63 that is less than a fifth reference level; (3) a ratio of the fourth total expression level to the fifth total expression level that is greater than 1; and/or (4) a ratio of HIF1A to TP63 expression levels that is greater than 1, wherein the fourth reference level and the fifth reference level are each indicative of low sensitivity to the ERK inhibitor.

In certain embodiments, the present disclosure provides a method of treating squamous cell carcinoma in a subject in need thereof, comprising administering an effective dose of an inhibitor of an extracellular signal-regulated kinase (ERK) to the subject, said subject comprising a genome having a copy number profile that comprises copy number amplification of at least one mitogen-activated protein kinase (MAPK) pathway gene. In certain embodiments, the present disclosure provides a method of treating subjects with malignancies of squamous histology without evidence of EGFR gene amplification. In certain embodiments, the present disclosure provides a method of treating subjects with malignancies of squamous histology with evidence of EGFR gene amplification.

In certain embodiments, the present disclosure provides a method of treating a subject having squamous cell carcinoma, comprising (a) screening the subject for the presence or absence of a gene signature indicative of sensitivity to an ERK inhibitor; and (b) administering the ERK inhibitor to the subject if the gene signature is determined to be present. An alternative therapy, such as chemotherapy, immunotherapy, radiotherapy or surgery, may be applied to the subject if the gene signature is determined to be absent. In some embodiments, the gene signature comprises a first total expression level of at least two MAPK pathway genes that is greater than a first reference level. In some embodiments, the gene signature comprises a second total expression level of at least two RAS-ERK feedback regulators that is greater than a second reference level. In some embodiments, the gene signature comprises a fourth total expression level of AREG, CDH3, COL17A1 , EGFR, HIF1A, ITGB1 , KRT1, KRT9 , NRG1 , SLC16A1 , SLC22A _I and VEGFA that is greater than a fourth reference level. In some embodiments, the gene signature comprises a fifth total expression level of DCUN1D1, PIK3CA, PRKCI, SOX2 and TP63 that is less than a fifth reference level. In some embodiments, the gene signature comprises a ratio of a fourth total expression level of AREG, CDH3, COL17A1 , EGFR, HIF1A, ITGB1, KRT1, KRT9 ,NRG1, SLC16A1 , SLC22A1 and VEGFA to a fifth total expression level of DCUN1D1, PIK3CA, PRKCI, SOX2 and TP63 that is greater than a reference level. In some embodiments, the gene signature comprises a ratio of HIF1A to TP63 expression levels that is greater than a reference level. In some embodiments, the gene signature comprises copy number amplification of at least one MAPK pathway gene. Exemplary MAPK pathway genes and RAS-ERK feedback regulators are described herein. The gene signature may comprise only one of an elevated first total expression level, an elevated second total expression level, an elevated fourth total expression level, a depressed fifth total expression level, or copy number amplification, or the gene signature may comprise any combination thereof, such as an elevated first total expression level and copy number amplification. In some embodiments, screening the subject for the presence or absence of a gene signature comprises performing nucleic acid analysis of a nucleic acid isolated from the subject. The nucleic acid may be from a squamous cell carcinoma cell.

In certain embodiments, the present disclosure provides a method of downregulating MAPK signaling output in a plurality of squamous cell carcinoma cells with an ERK inhibitor. In some embodiments, the method comprises (a) assessing, in a biological sample comprising a nucleic acid from the subject, (1) a first total expression level of at least two MAPK pathway genes and/or (2) a second total expression level of at least two RAS-ERK feedback regulators; and (b) administering an effective dose of the ERK inhibitor to the plurality of cells if the first total expression level is greater than a first reference level, and/or if the second total expression level is greater than a second reference level, wherein the first reference level and the second reference level are each indicative of low sensitivity to the ERK inhibitor.

In certain embodiments, the present disclosure provides a method of downregulating MAPK signaling output in a plurality of head and neck squamous cell carcinoma cells with an ERK inhibitor, comprising: (a) assessing, in a biological sample comprising a nucleic acid from the subject, (1) a fourth total expression level of AREG , CDH3 , COL17A1 , EGFR, HIF1A, ITGB1, KRT1, KRT9 , NRG1, SLC16A1, SLC22A1 and VEGFA; (2) a fifth total expression level of DCUN1D1, PIK3CA, PRKCI, SOX2 and TP63; (3) a ratio of the fourth total expression level to the fifth total expression level; and/or (4) a ratio of HIF1A to TP63 expression levels; and (b) administering an effective dose of the ERK inhibitor to the plurality of cells if (1) the fourth total expression level is greater than a fourth reference level, (2) the fifth total expression level is less than a fifth reference level, (3) the ratio of the fourth total expression level to the fifth total expression level is greater than 1, and/or (4) the ratio of HIF1A to TP63 is greater than 1, wherein the fourth reference level and the fifth reference level are each indicative of low sensitivity to the ERK inhibitor.

In certain embodiments, the present disclosure provides a method of downregulating MAPK signaling output in a plurality of squamous cell carcinoma cells with an ERK inhibitor, comprising (a) assessing, in a biological sample comprising a nucleic acid from the subject, a copy number profile of at least one MAPK pathway gene; and (b) administering an effective dose of the ERK inhibitor to the plurality of cells if the copy number profile comprises an average copy number of the at least one MAPK pathway gene of greater than 2.

In certain embodiments, the present disclosure provides a method of categorizing a squamous cell carcinoma status of a subject. In some embodiments, the method comprises (a) obtaining a biological sample from the subject, the sample comprising genomic and/or transcriptomic material from a squamous cell carcinoma cell of the subject; (b) assessing (1) a first total expression level of at least two MAPK pathway genes in the sample and/or (2) a second total expression level of at least two RAS-ERK feedback regulators in the sample; (c) generating an expression profile based on (1) a comparison between the first total expression level and a first reference level, and/or (2) a comparison between the second total expression level and a second reference level, wherein the first reference level and the second reference level are derived from a reference sample from a different subject having a known squamous cell carcinoma status; and (d) categorizing the squamous cell carcinoma status of the subject of (a) based on the expression profile. The squamous cell carcinoma status may be categorized as likely sensitive to treatment with an ERK inhibitor if the first total expression level is greater than the first reference level, wherein the first reference level is indicative of low sensitivity to the ERK inhibitor. Similarly, the squamous cell carcinoma status may be categorized as likely sensitive to treatment with an ERK inhibitor if the second total expression level is greater than a second reference level, wherein the second reference level is indicative of low sensitivity to the ERK inhibitor. In some embodiments, the known squamous cell carcinoma status of the different subject is categorized as resistant to an ERK inhibitor or sensitive to an ERK inhibitor. In some embodiments, the categorizing step includes calculating, using a computer system, a likelihood of response of the subject to treatment with an ERK inhibitor based on the expression profile, wherein the likelihood is adjusted upward for each fold increase in the first total expression level relative to the first reference level and for each fold increase in the second total expression level relative to the second reference level, wherein the first reference level and the second reference level are each indicative of low sensitivity to the ERK inhibitor. Optionally, the method further comprises preparing a report comprising a prediction of the likelihood of response of the subject to treatment with the ERK inhibitor.

In certain embodiments, the present disclosure provides a method of categorizing a head and neck squamous cell carcinoma status of a subject, comprising (a) obtaining a biological sample from the subject, the sample comprising genomic and/or transcriptomic material from a squamous cell carcinoma cell of the subject; (b) assessing, in the sample, (1) a fourth total expression level of AREG, CDH3, COL17A1 , EGFR, HIF1A, ITGB1 , KRT1 , KRT9 , NRG1 , SLC16A1 , SLC22A1 and VEGFA; (2) a fifth total expression level of DCUN1D1 , PIK3CA, PRKCI, SOX2 and TP63; and/or (3) expression levels of HIF1A and TP63; (c) generating an expression profile based on (1) a comparison between the fourth total expression level and a fourth reference level, (2) a comparison between the fifth total expression level and a fifth reference level, (3) a comparison between the fourth total expression level to the fifth total expression level, and/or (4) a comparison between expression levels of HIF1A and TP63, wherein the fourth reference level and the fifth reference level are derived from a reference sample from a different subject having a known squamous cell carcinoma status; and (d) categorizing the squamous cell carcinoma status of the subject of (a) based on the expression profile. The squamous cell carcinoma status may be categorized as likely sensitive to treatment with an ERK inhibitor if the fourth total expression level is greater than the fourth reference level, wherein the fourth reference level is indicative of low sensitivity to the ERK inhibitor. In some embodiments, the squamous cell carcinoma status is categorized as likely sensitive to treatment with an ERK inhibitor if the fifth total expression level is less than a fifth reference level, wherein the fifth reference level is indicative of low sensitivity to the ERK inhibitor. In some embodiments, the squamous cell carcinoma status is categorized as likely sensitive to treatment with an ERK inhibitor if a ratio of the fourth total expression level to the fifth total expression level is greater than 1. In some embodiments, the squamous cell carcinoma status is categorized as likely sensitive to treatment with an ERK inhibitor if a ratio of HIF1A to TP63 expression levels is greater than 1. In some embodiments, the categorizing step includes calculating, using a computer system, a likelihood of response of the subject to treatment with an ERK inhibitor based on the expression profile, wherein the likelihood is adjusted upward for each fold increase in the fourth total expression level relative to the fourth reference level and downward for each fold increase in the fifth total expression level relative to the fifth reference level, wherein the fourth reference level and the fifth reference level are each indicative of low sensitivity to the ERK inhibitor.

In certain embodiments, the present disclosure provides a method of categorizing a squamous cell carcinoma status of a subject, comprising (a) obtaining a biological sample from the subject, the sample comprising genomic and/or transcriptomic material from a squamous cell carcinoma cell of the subject; (b) assessing a copy number profile of at least one MAPK pathway gene in the sample; and (c) categorizing the squamous cell carcinoma status of the subject based on the copy number profile. The squamous cell carcinoma status may be categorized as likely sensitive to treatment with an ERK inhibitor if the copy number profile comprises an average copy number of the at least one MAPK pathway gene of greater than 2. In some embodiments, the categorizing step includes calculating, using a computer system, a likelihood of response of the subject to treatment with an ERK inhibitor based on the copy number profile, wherein the likelihood is adjusted upward for each additional copy number of the at least one MAPK pathway gene in excess of 2. Optionally, the method further comprises preparing a report comprising a prediction of the likelihood of response of the subject to treatment with the ERK inhibitor.

In certain embodiments, the present disclosure provides a method of assessing a likelihood of a subject having head and neck squamous cell carcinoma exhibiting a clinically beneficial response to treatment with an ERK inhibitor, the method comprising: (a) assessing, in a biological sample comprising genomic and/or transcriptomic material from a squamous cell carcinoma cell, (1) a fourth total expression level of AREG, CDH3, COL17A1 , EGFR, HIF1A, ITGB1, KRT1, KRT9, NRG1, SLC16A1, SLC22A1 and VEGFA; (2) a fifth total expression level of DCUN1D1, PIK3CA, PRKCI, SOX2 and TP63; and/or (3) expression levels of HIF1A and TP63; (b) calculating, using a computer system, a weighted probability of ERK inhibitor responsiveness based on (1) a comparison between the fourth total expression level and a fourth reference level, (2) a comparison between the fifth total expression level and a fifth reference level, (3) a comparison between the fourth total expression level to the fifth total expression level, and/or (4) a comparison between expression levels of HIF1A and TP63, wherein the fourth reference level and the fifth reference level are derived from one or more reference samples.

In certain embodiments, the present disclosure provides a method of assessing a likelihood of a subject having squamous cell carcinoma exhibiting a clinically beneficial response to treatment with an ERK inhibitor. In some embodiments, the method comprises (a) assessing (1) a first total expression level of at least two MAPK pathway genes and/or (2) a second total expression level of at least two RAS-ERK feedback regulators in a biological sample comprising genomic and/or transcriptomic material from a squamous cell carcinoma cell; (b) calculating, using a computer system, a weighted probability of ERK inhibitor responsiveness based on (1) a comparison between the first total expression level and a first reference level, and/or (2) a comparison between the second total expression level and a second reference level, wherein the first reference level and the second reference level are derived from one or more reference samples. In some embodiments, the method further comprises designating the subject as having a high probability of exhibiting a clinically beneficial response to treatment with the ERK inhibitor if the weighted probability corresponds to at least 1.5 times a baseline probability, wherein the baseline probability represents a likelihood that the subject will exhibit a clinically beneficial response to treatment with the ERK inhibitor before obtaining the weighted probability of (b). In some embodiments, the method further comprises transmitting information concerning the likelihood to a receiver. In some embodiments, the method further comprises providing a recommendation based on the weighted probability. The recommendation may comprise treating the subject with the ERK inhibitor, or, alternatively, discontinuing therapy, or administering one or more of chemotherapy, immunotherapy, radiotherapy or surgery. In some embodiments, the method further comprises selecting a treatment based on the weighted probability. In some embodiments, the method further comprises administering the ERK inhibitor to the subject based on the weighted probability.

In certain embodiments, the present disclosure provides a method of assessing a likelihood of a subject having squamous cell carcinoma exhibiting a clinically beneficial response to treatment with an ERK inhibitor, the method comprising (a) assessing a copy number profile of at least one MAPK pathway gene in a biological sample comprising genomic and/or transcriptomic material from a squamous cell carcinoma cell; (b) calculating, using a computer system, a weighted probability of ERK inhibitor responsiveness based on the copy number profile. In some embodiments, the method further comprises designating the subject as having a high probability of exhibiting a clinically beneficial response to treatment with the ERK inhibitor if the weighted probability corresponds to at least 1.5 times a baseline probability, wherein the baseline probability represents a likelihood that the subject will exhibit a clinically beneficial response to treatment with the ERK inhibitor before obtaining the weighted probability of (b). Information concerning the likelihood may be transmitted to a receiver. In some embodiments, the method further comprises providing a recommendation based on the weighted probability. The recommendation may comprise treating the subject with the ERK inhibitor, or, alternatively, discontinuing therapy, or administering one or more of chemotherapy, immunotherapy, radiotherapy or surgery. A treatment may be selected based on the weighted probability. In some embodiments, the method further comprises administering the ERK inhibitor based on the weighted probability.

In some embodiments, the copy number profile of the at least one MAPK pathway gene is assessed by a method selected from the group consisting of in situ hybridization (ISH), Southern blot, immunohistochemistry (IHC), polymerase chain reaction (PCR), quantitative PCR (qPCR), quantitative real-time PCR (qRT-PCR), comparative genomic hybridization (CGH), microarray-based comparative genomic hybridization, and ligase chain reaction (LCR). In some embodiments, the in situ hybridization is selected from fluorescence in situ hybridization (FISH), chromogenic in situ hybridization (CISH) and silver in situ hybridization (SISH). In some embodiments, the copy number profile is assessed using a nucleic acid sample from the subject, such as genomic DNA, cDNA, ctDNA, cell-free DNA, RNA or mRNA. In some embodiments, the nucleic acid is from a squamous cell carcinoma cell. In some embodiments, the at least one MAPK pathway gene is selected from CDK4, CDK6, EGFR, ERK1, CCND1 , KRAS, ERK2, and HRAS. In some embodiments, the at least one MAPK pathway gene is EGFR. In some embodiments, the squamous cell carcinoma is esophageal squamous cell carcinoma

In practicing any of the subject methods, individual expression levels of each of the at least two MAPK pathway genes may be added together to provide the first total expression level. The at least two MAPK pathway genes may comprise at least 2, at least 3, at least 4, at least 5, at least 6, at least 7, or at least 8 MAPK pathway genes, such as 2, 3, 4, 5, 6, 7 or 8 MAPK pathway genes. In some embodiments, as few as two MAPK pathway genes, such as ERK1 and CCND1 , ERK1 and EGFR, or EGFR and CCND1 , may be predictive of sensitivity of a squamous cell carcinoma to an ERK inhibitor. In some embodiments, three MAPK pathway genes, such as EGFR, ERK1 and CCND1 or EGFR, ERK1 and KRAS, may be predictive of sensitivity of a squamous cell carcinoma to an ERK inhibitor. In some embodiments, four MAPK pathway genes, such as EGFR, ERK1, CCND1 and KRAS, may be predictive of sensitivity of a squamous cell carcinoma to an ERK inhibitor. In some embodiments, six MAPK pathway genes, such as EGFR, ERK1, CCND1 , KRAS, ERK2 and HRAS, may be predictive of sensitivity of a squamous cell carcinoma to an ERK inhibitor. In some embodiments, eight MAPK pathway genes, such as CDK4, CDK6, EGFR, ERK1, CCND1 , KRAS, ERK2 and HRAS, may be predictive of sensitivity of a squamous cell carcinoma to an ERK inhibitor.

A squamous cell carcinoma having a first total expression level that is greater than a first reference level may be more likely to respond to treatment with an ERK inhibitor than a squamous cell carcinoma having a first total expression level that is less than a first reference level. The predictive power of the at least two MAPK pathway genes may increase as the absolute difference between the first total expression level and the first reference level increases.

The first reference level may be obtained by assessing a total expression level of the at least two MAPK pathway genes in a biological sample from one or more subjects having a squamous cell carcinoma exhibiting low sensitivity to treatment with the ERK inhibitor. In some examples, the first reference level is the average total expression level of the at least two MAPK pathway genes in a plurality of squamous cell carcinoma samples. The plurality may comprise at least 5, 10, 20, 30, 40 or at least 50 samples.

In practicing any of the subject methods, individual expression levels of each of the at least two RAS-ERK feedback regulators may be added together to provide the second total expression level. The at least two RAS-ERK feedback regulators may comprise at least 2, at least 3, at least 4, at least 5, at least 6, at least 7, or at least 8 RAS-ERK feedback regulators, such as 2, 3, 4, 5, 6, 7 or 8 RAS-ERK feedback regulators. In some embodiments, as few as two RAS-ERK feedback regulators, such as DUSP5 and DUSP6, may be predictive of sensitivity of a squamous cell carcinoma to an ERK inhibitor. In some embodiments, four RAS-ERK feedback regulators, such as DUSP5, DUSP6, DUSP2 and DUSP4, may be predictive of sensitivity of a squamous cell carcinoma to an ERK inhibitor. In some embodiments, five RAS-ERK feedback regulators, such as DUSP5, DUSP6, SPRY2, SPRY4 and SPRED1, may be predictive of sensitivity of a squamous cell carcinoma to an ERK inhibitor.

A squamous cell carcinoma having a second total expression level that is greater than a second reference level may be more likely to respond to treatment with an ERK inhibitor than a squamous cell carcinoma having a second total expression level that is less than a second reference level. The predictive power of the at least two RAS-ERK feedback regulators may increase as the absolute difference between the second total expression level and the second reference level increases.

The second reference level may be obtained by assessing a total expression level of the at least two RAS-ERK feedback regulators in a biological sample from one or more subjects having a squamous cell carcinoma exhibiting low sensitivity to treatment with the ERK inhibitor. In some examples, the second reference level is the average total expression level of the at least two RAS-ERK feedback regulators in a plurality of squamous cell carcinoma samples. The plurality may comprise at least 5, 10, 20, 30, 40 or at least 50 samples.

Any of the methods and systems described herein may utilize combinations of MAPK pathway genes and RAS-ERK feedback regulators in selecting a squamous cell carcinoma suitable for treatment with an ERK inhibitor. Therefore, when a method described herein recites a selection of a first total expression level of at least two MAPK pathway genes and/or a second total expression level of at least two RAS-ERK feedback regulators, it is recognized that the expression of at least one MAPK pathway gene and at least one RAS-ERK feedback regulator could be added together to give a total expression level that could be substituted for any method described herein. For example, a total expression level of CCND1, CRAF, DUSP5 , EGFR, ERK1 and KRAS could be compared to a corresponding reference level, wherein a total expression level greater than the reference level indicates that treatment of the subject with an ERK inhibitor is likely to produce a clinically beneficial response. The total expression level of the at least one MAPK pathway gene and at the least one RAS-ERK feedback regulator could be compared to a corresponding reference level. The reference level may be indicative of low sensitivity to the ERK inhibitor. In some embodiments, the reference level is obtained by assessing a total expression level of the at least one MAPK pathway gene and the at least one RAS-ERK feedback regulator in a biological sample from one or more subjects having a squamous cell carcinoma exhibiting low sensitivity to treatment with the ERK inhibitor. In some examples, the reference level is the average total expression level of the at least one MAPK pathway gene and the at least one RAS-ERK feedback regulator in a plurality of squamous cell carcinoma samples. The plurality may comprise at least 5, 10, 20, 30, 40 or at least 50 samples. When a method described herein recites a selection of a first total expression level of at least two MAPK pathway genes and/or a second total expression level of at least two RAS-ERK feedback regulators, also contemplated is a third total expression level of at least one MAPK pathway gene and at least one RAS-ERK feedback regulator. The third total expression level may be compared to a third reference level. The at least one MAPK pathway gene and at least one RAS-ERK feedback regulator of the third total expression level may be selected from the group consisting of EGFR, ERK1 , CCND1 , KRAS, ERK2, HRAS, DUSP5 , DUSP6, DUSP2, DUSP4 , SPRY2 , SPRY4 , SPRED1 , and CRAF, such as CCND1, CRAF, DUSP5 , EGFR, ERK1 , and KRAS, such as CCND1, CRAF, DUSP5 , EGFR, ERK1 and KRAS.

In practicing any of the subject methods, individual expression levels of each of AREG, CDH3, COL17A1, EGFR, HIF1A, ITGB1, KRT1 , KRT9, NRG1, SLC16A1, SLC22A1 and VEGFA may be added together to provide the fourth total expression level. A squamous cell carcinoma, such as a head and neck squamous cell carcinoma, having a fourth total expression level that is greater than a fourth reference level may be more likely to respond to treatment with an ERK inhibitor than a squamous cell carcinoma having a fourth total expression level that is less than a fourth reference level. The predictive power may increase as the absolute difference between the fourth total expression level and the fourth reference level increases. The fourth reference level may be obtained by assessing a total expression level of AREG, CDH3, COL17A1 , EGFR, HIF1A, ITGB1, KRT1 , KRT9 , NRG1 , SLC16A1 , SLC22A _I and VEGFA in a biological sample from one or more subjects having a squamous cell carcinoma exhibiting low sensitivity to treatment with the ERK inhibitor.

In practicing any of the subject methods, individual expression levels of each of DCUN1D1, PIK3CA, PRKCI, SOX2 and TP63 may be added together to provide the fifth total expression level. A squamous cell carcinoma, such as a head and neck squamous cell carcinoma, having a fifth total expression level that is less than a fifth reference level may be more likely to respond to treatment with an ERK inhibitor than a squamous cell carcinoma having a fifth total expression level that is greater than a fifth reference level. The predictive power may increase as the absolute difference between the fifth total expression level and the fifth reference level increases. The fifth reference level may be obtained by assessing a total expression level of DCUN1D1 , PIK3CA, PRKCI, SOX2 and TP63 in a biological sample from one or more subjects having a squamous cell carcinoma exhibiting low sensitivity to treatment with the ERK inhibitor.

In some embodiments, the fourth total expression level and the fifth total expression level are compared directly without the need for a determination of corresponding reference levels. For example, a squamous cell carcinoma, such as a head and neck squamous cell carcinoma, having a ratio of the fourth total expression level to the fifth total expression level that is greater than 0.4, greater than 0.5, greater than 0.6, greater than 0.7, greater than 0.8, greater than 0.9, greater than 1, greater than 1.1, greater than 1.2, greater than 1.3, greater than 1.4, greater than 1.5, greater than 2, greater than 2.5, greater than 3, greater than 4, greater than 5, greater than 6, greater than 7, greater than 8, greater than 9, or greater than 10, such as greater than 1, may be more likely to respond to treatment with an ERK inhibitor than a squamous cell carcinoma having a ratio that is less than 0.4. The predictive power may increase as the ratio increases. In some preferred embodiments, a ratio of the fourth total expression level to the fifth total expression level that is greater than 1 may be more likely to respond to treatment with an ERK inhibitor than a squamous cell carcinoma having a ratio that is less than 1.

In some embodiments, expression levels of HIF1A to TP63 are compared directly without the need for a determination of corresponding reference levels. For example, a squamous cell carcinoma, such as a head and neck squamous cell carcinoma, having a ratio of HIF1A to TP63 that is greater than 0.4, greater than 0.5, greater than 0.6, greater than 0.7, greater than 0.8, greater than 0.9, greater than 1, greater than 1.1, greater than 1.2, greater than 1.3, greater than 1.4, greater than 1.5, greater than 2, greater than 2.5, greater than 3, greater than 4, greater than 5, greater than 6, greater than 7, greater than 8, greater than 9, or greater than 10, such as greater than 1, may be more likely to respond to treatment with an ERK inhibitor than a squamous cell carcinoma having a ratio that is less than 0.4. The predictive power may increase as the ratio increases. In some preferred embodiments, a ratio of HIF1A to TP63 that is greater than 1 may be more likely to respond to treatment with an ERK inhibitor than a squamous cell carcinoma having a ratio that is less than 1.

In practicing any of the subject methods, the average copy number of at least one MAPK pathway gene may be assessed. The at least one MAPK pathway gene may comprise at least 1, at least 2, at least 3, at least 4, at least 5, at least 6, at least 7, or at least 8 MAPK pathway genes, such as 1, 2, 3, 4, 5, 6, 7 or 8 MAPK pathway genes. In some embodiments, one MAPK pathway gene, such as EGFR, may be predictive of sensitivity of a squamous cell carcinoma to an ERK inhibitor. The at least one MAPK pathway gene may be selected from CDK4, CDK6, EGFR, ERK 1 , CCND1, KRAS, ERK2, and HRAS, such as EGFR.

A squamous cell carcinoma having copy number amplification of at least one MAPK pathway gene may be more likely to respond to treatment with an ERK inhibitor. For example, a squamous cell carcinoma having an average copy number of the at least one MAPK pathway gene that is greater than 2 may be more likely to respond to treatment with an ERK inhibitor than a squamous cell carcinoma having an average copy number of the at least one MAPK pathway gene that is less than 2. The predictive power of the at least one MAPK pathway gene may increase as the average copy number increases. For example, an average copy number greater than 3, greater than 4, greater than 5, greater than 6, greater than 7, greater than 8, greater than 9, or greater than 10 of the at least one MAPK pathway gene may be predictive of sensitivity of a squamous cell carcinoma to an ERK inhibitor. In some embodiments, the predictive power of the at least one MAPK pathway gene increases if more than one MAPK pathway gene exhibits copy number amplification,

The first total expression level may be compared to the first reference level to calculate a weighted probability of ERK inhibitor responsiveness. In some embodiments, the second total expression level is compared to the second reference level to calculate a weighted probability of ERK inhibitor responsiveness. In some embodiments, the third total expression level is compared to the third reference level to calculate a weighted probability of ERK inhibitor responsiveness. In some embodiments, the fourth total expression level is compared to the fourth reference level to calculate a weighted probability of ERK inhibitor responsiveness. In some embodiments, the fifth total expression level is compared to the fifth reference level to calculate a weighted probability of ERK inhibitor responsiveness. In some embodiments, the copy number status of at least one MAPK pathway gene is used to calculate a weighted probability of ERK inhibitor responsiveness. Optionally, calculation of a weighted probability of ERK inhibitor responsiveness comprises assessment of one or more of the first total expression level, the second total expression level, the third total expression level, the fourth total expression level, the fifth total expression level, or the copy number status of the at least one MAPK pathway gene. Optionally, calculation of a weighted probability of ERK inhibitor responsiveness comprises assessment of one or more of the first reference level, the second reference level, the third reference level, the fourth reference level, the fifth reference level, or the copy number status of the at least one MAPK pathway gene. Optionally, the calculation is performed by a computer system. Any method of the present disclosure may further comprise designating a subject having squamous cell carcinoma as having a high probability of exhibiting a clinically beneficial response to treatment with an ERK inhibitor if the weighted probability corresponds to at least 1.5, at least 2, at least 3, at least 4, at least 5, at least 6, at least 7, at least 8, at least 9, at least 10, at least 15, or at least 20, such as at least 2 times a baseline probability, wherein the baseline probability represents a likelihood that the subject will exhibit a clinically beneficial response to treatment with the ERK inhibitor before obtaining the weighted probability.

In some embodiments, a method of the disclosure comprises a group of biomarkers that is differentially expressed in cancer cells, such as squamous cell carcinoma cells. The relative expression of these biomarkers may be used to identify cells that are more likely to respond to treatment with an ERK inhibitor. In some embodiments, a method of the disclosure comprises a biomarker that is a predictor of ERK inhibitor sensitivity. In some embodiments, the biomarker is a gene or gene product associated with a cellular pathway, including, for example, the MAP kinase (MAPK) pathway or the RAS-ERK feedback regulatory pathway. In some embodiments, a MAPK pathway gene is selected from the group consisting of CDK4, CDK6, CRAF , EGFR, ERK1 , CCND1, KRAS, ERK2, and HRAS . In some embodiments, a RAS-ERK feedback regulator is selected from the group consisting of DUSP2, DUSP4 , DUSP5, DUSP6, SPRY2 , SPRY4 and SPRED1 . As used herein, the term biomarker may refer to one or more of a MAPK pathway gene and/or a RAS-ERK feedback regulator. Further biomarkers may include AREG, CDH3, COL17A1 , EGFR, HIF1A, ITGB1 , KRT1 , KRT9, NRG1 , SLC16A1, SLC22A1 and VEGFA, overexpression of which is associated with sensitivity to treatment with an ERK inhibitor. Further biomarkers may include DCUN1D1, PIK3CA, PRKCI, SOX2 and TP63, overexpression of which is associated with resistance to treatment with an ERK inhibitor.

In some embodiments, a method of the disclosure may comprise the identification of cells that are more likely to respond to treatment with an ERK inhibitor by assessing the relative copy number of one or more MAPK pathway genes. In some embodiments, the MAPK pathway gene is selected from the group consisting of CDK4, CDK6, CRAF , EGFR, ERK1, CCNDJ, KRAS, ERK2, and HRAS.

The methods described herein for qualifying or quantifying the expression of polypeptides and/or polynucleotides provide information which can be correlated with pathological conditions, predisposition to disease, therapeutic monitoring, risk stratification, among others. In some embodiments, a method of the disclosure is particularly useful for diagnosing conditions, evaluating whether an ERK inhibitor will have a desired effect, i.e., predicting responsiveness to an ERK inhibitor, and determining prognoses. The present methods may be used for the optimization of treatment protocols. In this context, evaluation of the expression profile of the biomarkers disclosed herein can be used to gain information on the treatment potential of a tissue sample with an ERK inhibitor.

In some embodiments, the disclosure provides methods for measuring a likelihood that a subject having cancer, especially squamous cell carcinoma, will exhibit a clinically beneficial response to treatment with an ERK inhibitor based on an expression profile of at least two genes or gene products. An “expression profile” refers to a pattern of expression of at least one biomarker, such as at least two biomarkers, that recurs in multiple samples and reflects a property shared by those samples, such as tissue type, response to treatment with an ERK inhibitor, or activation of a particular biological process or pathway in the cells. Furthermore, an expression profile differentiates between samples that share that common property and those that do not with better accuracy than would likely be achieved by assigning the samples to the two groups at random. An expression profile may be used to predict whether samples of unknown status share that common property or not. Some variation between the levels of at least one biomarker and the typical profile is to be expected, but the overall similarity of the expression levels to the typical profile is such that it is statistically unlikely that the similarity would be observed by chance in samples not sharing the common property that the expression profile reflects.

An expression profile may be generated based on a comparison between a total expression level of at least two biomarkers in a sample from a test subject and a corresponding reference level. The at least two biomarkers may comprise a MAPK pathway gene and/or a RAS-ERK feedback regulator that is a predictor of ERK inhibitor sensitivity. In some embodiments, an expression profile is generated based on the expression of 2 or more, 3 or more, 4 or more, 5 or more, 6 or more, 7 or more, or 8 or more biomarkers. In some embodiments, an expression profile is generated based on the expression of 2, 3, 4, 5, 6, 7, or 8 biomarkers.

In some embodiments, the expression profile is used in a method of the disclosure to assess a likelihood of response to treatment with an ERK inhibitor. The likelihood of response may be adjusted upward for each biomarker that is a predictor of ERK inhibitor sensitivity that is overexpressed. In some embodiments, the likelihood of response may be adjusted downward for each biomarker that is a predictor of ERK inhibitor sensitivity that is underexpressed. The magnitude of under- or over-expression may be used to weight the amount of adjustment to the likelihood of response. Preferably, the individual expression levels of at least two biomarkers that are predictors of ERK inhibitor sensitivity are summed to give a total expression level.

In some embodiments, a method of the disclosure provides a reference level above which a biomarker must be expressed to be considered in assessing the likelihood of response to treatment with an ERK inhibitor. The biomarker may be differentially expressed at least 1.1 fold, at least 1.2 fold, at least 1.3 fold, at least 1.4 fold, at least 1.5 fold, at least 2.0 fold, at least 2.25 fold, at least 2.5 fold, at least 2.75 fold, at least 3.0 fold, at least 3.5 fold, at least 4.0 fold, at least 5.0, or even at least 10 fold higher or lower relative to a reference level to be considered in adjusting the likelihood of response. In some embodiments, the reference level is a numerical range of biomarker expression that is obtained from a statistical sampling from a population of individuals having cancer that has low sensitivity to treatment with an ERK inhibitor. In some embodiments, the reference level is a numerical range of biomarker expression that is obtained from a statistical sampling from a population of individuals having cancer that is resistant to treatment with an ERK inhibitor. The reference level may be a numerical range of biomarker expression that is obtained from a statistical sampling from a population of individuals having cancer, e.g., the same cancer as the test subject. In some embodiments, the reference level is derived by comparison of sensitive and resistant populations.

The present inventors have discovered certain genes that are amplified and/or differentially expressed in squamous cell carcinoma cells or adenocarcinoma cells that are sensitive to therapy with an ERK inhibitor, such as a compound described herein. More specifically, the disclosure relates to the use of an inhibitor of extracellular signal-regulated kinases 1 and 2 (ERK1 and ERK2) to treat cancer, such as pancreatic cancer, bladder cancer, gastric cancer, and squamous cell carcinomas of the lung (LSCC), esophagus (ESCC), head and neck (HNSCC) and cervix. Methods of using information about the amplification and/or expression status of the genes and/or the gene expression products to identify carcinoma cells that will likely respond to therapy with an ERK inhibitor, as well as methods of identifying subjects having a carcinoma predicted to exhibit a clinically beneficial response to treatment with an ERK inhibitor are described herein. In particular, amplification and/or overexpression of at least one gene located at chromosome 11q13.3-13.4 may be indicative of sensitivity to therapy with an ERK inhibitor. Use of certain DNA- and RNA-based biomarkers to identify tumors, such as ESCC tumors, more likely to display a robust therapeutic response to ERK inhibition is described.

In certain embodiments, the present disclosure provides a method of treating cancer in a subject in need thereof. In some embodiments, the method comprises administering an effective dose of an inhibitor of an extracellular signal-regulated kinase (ERK) to the subject, said subject comprising a genome that exhibits amplification and/or overexpression of at least one gene located at chromosome 11q13.3-13.4. In some embodiments, the method further comprises (a) screening the subject for amplification and/or overexpression of the at least one gene located at chromosome 11q13.3-13.4; and (b) administering the ERK inhibitor to the subject if the amplification and/or overexpression is determined to be present. An alternative therapy, such as chemotherapy, immunotherapy, radiotherapy or surgery, may be applied to the subject if the amplification and/or overexpression are determined to be absent. In some embodiments, the screening comprises performing nucleic acid analysis of a nucleic acid isolated from the subject, such as from a cancer cell isolated from the subject. In some embodiments, the method comprises administering the ERK inhibitor to the subject if both amplification and overexpression of the at least one gene are determined to be present. In some embodiments, the at least one gene is selected from CCND1, CTTN, FADD, ORAOV1, ANO1, PPFIA1 and SHANK2. In some embodiments, the at least one gene is selected from CCND1, CTTN, FADD, ORAOV1, ANO1, PPFIA1 , SHANK2, FGF3, FGF4 and FGF 19 .

In certain embodiments, the present disclosure provides a method of treating a subject having cancer, comprising (a) screening the subject for amplification and/or overexpression of at least one gene located at chromosome 11q13.3-13.4 or a gene that co-amplifies with a gene located at chromosome 11q13.3-13.4; and (b) administering an ERK inhibitor to the subject if the amplification and/or overexpression is determined to be present. An alternative therapy, such as chemotherapy, immunotherapy, radiotherapy or surgery, may be applied to the subject if the amplification and/or overexpression are determined to be absent. In some embodiments, the screening comprises performing nucleic acid analysis of a nucleic acid isolated from the subject, such as from a cancer cell isolated from the subject. In some embodiments, the method comprises administering the ERK inhibitor to the subject if both amplification and overexpression of the at least one gene are determined to be present. In some embodiments, the at least one gene is selected from CCND1, CTTN, FADD, ORAOV1, ANO1, PPFIA1 and SHANK2. In some embodiments, the at least one gene is selected from CCND1, CTTN, FADD, ORAOV1, ANO1, PPFIA1, SHANK2, FGF3, FGF4 and FGF 19 .

In practicing any of the subject methods, the ERK inhibitor may be administered to the subject if the subject exhibits amplification and/or overexpression of CCND1 or ANO1. The ERK inhibitor may be administered to the subject if the subject exhibits amplification or overexpression of CCND1 and ANO1. The ERK inhibitor may be administered to the subject if the subject exhibits amplification and overexpression of CCND1 and ANO1 . In some embodiments, the ERK inhibitor is administered to the subject if amplification and/or overexpression of at least one gene selected from CCND1, CTTN, FADD, ORAOV1, ANO1, PPFIA1 and SHANK2 is detected. The ERK inhibitor may be administered to the subject if amplification, overexpression, or a combination thereof of one or more of CCND1, CTTN, FADD, ORAOV1, ANO1, PPFIA1 and SHANK2, or a combination thereof, is detected. In some embodiments, a total amplification and/or expression level of one or more genes located at chromosome 11q13.3-13.4 is assessed. In some embodiments, the ERK inhibitor is administered to the subject if amplification and/or overexpression of at least one gene selected from CCND1, CTTN, FADD, ORAOV1, ANO1, PPFIA1, SHANK2, FGF3, FGF4 and FGF 19 is detected. The ERK inhibitor may be administered to the subject if amplification, overexpression, or a combination thereof of one or more of CCND1, CTTN, FADD, ORAOV1, ANO1, PPFIA1, SHANK2, FGF3, FGF4 and FGF 19, or a combination thereof, is detected.

In certain embodiments, the present disclosure provides a method of downregulating MAPK signaling output in a plurality of cancer cells with an ERK inhibitor. In some embodiments, the method comprises (a) assessing, in a biological sample comprising a nucleic acid from the plurality of cells, a copy number profile and/or expression profile of at least one gene located at chromosome 11q13.3-13.4; and (b) administering an effective dose of the ERK inhibitor to the plurality of cells if the copy number profile comprises an average copy number of the at least one gene of >2 and/or if the expression profile is greater than a reference level, wherein the reference level is indicative of low sensitivity to the ERK inhibitor.

In certain embodiments, the present disclosure provides a method of categorizing a cancer status of a subject. In some embodiments, the method comprises (a) obtaining a biological sample from the subject, the sample comprising genomic and/or transcriptomic material from a cancer cell of the subject; (b) assessing a copy number profile and/or expression profile of at least one gene located at chromosome 11q13.3-13.4 in the sample; and (c) categorizing the cancer status of the subject of (a) based on the copy number profile and/or the expression profile. The cancer status may be categorized as likely sensitive to treatment with an ERK inhibitor if the copy number profile comprises an average copy number of the at least one gene of >2. Similarly, the cancer status may be categorized as likely sensitive to treatment with an ERK inhibitor if the expression profile is greater than a reference level, wherein the reference level is indicative of low sensitivity to the ERK inhibitor. In some embodiments, the categorizing step includes calculating, using a computer system, a likelihood of response of the subject to treatment with an ERK inhibitor based on the copy number profile and/or the expression profile, wherein the likelihood is adjusted upward for each additional copy number of the at least one gene in excess of 2 and for each fold increase in the expression profile relative to a reference level, wherein the reference level is indicative of low sensitivity to the ERK inhibitor. Optionally, the method further comprises preparing a report comprising a prediction of the likelihood of response of the subject to treatment with the ERK inhibitor.

In certain embodiments, the present disclosure provides a method of assessing a likelihood of a subject having cancer exhibiting a clinically beneficial response to treatment with an ERK inhibitor, the method comprising: (a) assessing a copy number profile and/or expression profile of at least one gene located at chromosome 11q13.3-13.4 in a biological sample comprising genomic and/or transcriptomic material from a cancer cell; and (b) calculating, using a computer system, a weighted probability of ERK inhibitor responsiveness based on the copy number profile and/or the expression profile. In some embodiments, the method further comprises designating the subject as having a high probability of exhibiting a clinically beneficial response to treatment with the ERK inhibitor if the weighted probability corresponds to at least 1.5 times a baseline probability, wherein the baseline probability represents a likelihood that the subject will exhibit a clinically beneficial response to treatment with the ERK inhibitor before obtaining the weighted probability of (b). In some embodiments, the method further comprises transmitting information concerning the likelihood to a receiver. In some embodiments, the method further comprises providing a recommendation based on the weighted probability. The recommendation may comprise treating the subject with the ERK inhibitor, or, alternatively, discontinuing therapy, chemotherapy, immunotherapy, radiotherapy or surgery. In some embodiments, the method further comprises selecting a treatment based on the weighted probability. In some embodiments, the method further comprises administering the ERK inhibitor based on the weighted probability.

In some embodiments, the copy number profile of the at least one gene located at chromosome 11q13.3-13.4 is assessed by a method selected from the group consisting of in situ hybridization (ISH), Southern blot, immunohistochemistry (IHC), polymerase chain reaction (PCR), quantitative PCR (qPCR), quantitative real-time PCR (qRT-PCR), comparative genomic hybridization (CGH), microarray-based comparative genomic hybridization, and ligase chain reaction (LCR). In some embodiments, the in situ hybridization is selected from fluorescence in situ hybridization (FISH), chromogenic in situ hybridization (CISH) and silver in situ hybridization (SISH). In some embodiments, the copy number profile is assessed using a nucleic acid sample from the subject, such as genomic DNA, cDNA, ctDNA, cell-free DNA, RNA or mRNA. In some embodiments, the copy number profile is assessed using a cell-free DNA sample from the subject. In some embodiments, the nucleic acid is from a cancer cell. In some embodiments, the at least one gene located at chromosome 11q13.3-13.4 is selected from CCND1, CTTN, FADD, ORAOV 1 , ANO1, PPFIA1 and SHANK2. In some embodiments, the at least one gene located at chromosome 11q13.3-13.4 is selected from CCND1, CTTN, FADD, ORAOV1, ANO1 , PPFIA 1 , SHANK2 , FGF 3, FGF 4 and FGF 19 . In some embodiments, the at least one gene located at chromosome 11q13.3-13.4 is CCND1 and ANO1 . In some embodiments, the at least one gene located at chromosome 11q13.3-13.4 is CCND1 or ANO1 . In some embodiments, the cancer is a squamous cell carcinoma, such as esophageal squamous cell carcinoma, lung squamous cell carcinoma, or head and neck squamous cell carcinoma. In some embodiments, the cancer is esophageal squamous cell carcinoma.

In practicing any of the subject methods, individual expression levels each of the at least one gene located at chromosome 11q13.3-13.4 may be added together to provide a total expression level. The at least one gene located at chromosome 11q13.3-13.4 may comprise at least 2, at least 3, at least 4, at least 5, at least 6 or at least 7 genes, such as 2, 3, 4, 5, 6 or 7 genes.

A cancer having a total expression level of the at least one gene located at chromosome 11q13.3-13.4 that is greater than a reference level may be more likely to respond to treatment with an ERK inhibitor than a cancer having a total expression level of the at least one gene located at chromosome 11q13.3-13.4 that is less than the reference level. The predictive power of the at least one gene located at chromosome 11q13.3-13.4 may increase as the absolute difference between the total expression level and the reference level increases.

The reference level may be obtained by assessing a total expression level of the at least one gene located at chromosome 11q13.3-13.4 in a biological sample from one or more subjects having a cancer exhibiting low sensitivity to treatment with the ERK inhibitor. In some examples, the reference level is the average total expression level of the at least one gene located at chromosome 11q13.3-13.4 in a plurality of cancer samples. The plurality may comprise at least 5, 10, 20, 30, 40 or at least 50 samples.

Any of the methods and systems described herein may utilize combinations of MAPK pathway genes, RAS-ERK feedback regulators, and genes located at chromosome 11q13.3-13.4 in selecting a cancer suitable for treatment with an ERK inhibitor.

In practicing any of the subject methods, the average copy number of the at least one gene located at chromosome 11q13.3-13.4 may be assessed. The at least one gene located at chromosome 11q13.3-13.4 may comprise at least 1, at least 2, at least 3, at least 4, at least 5, at least 6 or at least 7 genes, such as 1, 2, 3, 4, 5, 6 or 7 genes. In some embodiments, one gene located at chromosome 11q13.3-13.4, such as CCND1 , may be predictive of sensitivity of a cancer to an ERK inhibitor. The at least one gene located at chromosome 11q13.3-13.4 may be selected from CCND1, CTTN, FADD, ORAOV1, ANO1, PPFIA1 and SHANK2, such as CCND1 and ANO1 . The at least one gene located at chromosome 11q13.3-13.4 may be selected from CCND1 , CTTN, FADD, ORAOV1 , ANO1 , PPFIA1, SHANK2, FGF3, FGF4 and FGF19 .

A cancer having copy number amplification of at least one gene located at chromosome 11q13.3-13.4 may be more likely to respond to treatment with an ERK inhibitor. For example, a cancer having an average copy number of the at least one gene located at chromosome 11q13.3-13.4 that is greater than 2 may be more likely to respond to treatment with an ERK inhibitor than a cancer having an average copy number of the at least one gene located at chromosome 11q13.3-13.4 that is less than 2. The predictive power of the at least one gene located at chromosome 11q13.3-13.4 may increase as the average copy number increases. For example, an average copy number greater than 3, greater than 4, greater than 5, greater than 6, greater than 7, greater than 8, greater than 9, or greater than 10 of the at least one gene located at chromosome 11q13.3-13.4 may be predictive of sensitivity of a cancer to an ERK inhibitor. In some embodiments, the predictive power of the at least one gene located at chromosome 11q13.3-13.4 increases if more than one gene located at chromosome 11q13.3-13.4 exhibits copy number amplification.

The total expression level of at least one gene located at chromosome 11q13.3-13.4 may be compared to the reference level to calculate a weighted probability of ERK inhibitor responsiveness. In some embodiments, the copy number status of at least one gene located at chromosome 11q13.3-13.4 is used to calculate a weighted probability of ERK inhibitor responsiveness. Optionally, calculation of a weighted probability of ERK inhibitor responsiveness comprises assessment of one or more of the total expression level and the copy number status of the at least one gene located at chromosome 11q13.3-13.4. Optionally, the calculation is performed by a computer system. Any method of the present disclosure may further comprise designating a subject having cancer as having a high probability of exhibiting a clinically beneficial response to treatment with an ERK inhibitor if the weighted probability corresponds to at least 1.5, at least 2, at least 3, at least 4, at least 5, at least 6, at least 7, at least 8, at least 9, at least 10, at least 15, or at least 20, such as at least 2 times a baseline probability, wherein the baseline probability represents a likelihood that the subject will exhibit a clinically beneficial response to treatment with the ERK inhibitor before obtaining the weighted probability.

In some embodiments, a method of the disclosure comprises a group of biomarkers that is differentially expressed in cancer cells, such as cancer cells. The relative expression of these biomarkers may be used to identify cells that are more likely to respond to treatment with an ERK inhibitor. In some embodiments, a method of the disclosure comprises a biomarker that is a predictor of ERK inhibitor sensitivity. In some embodiments, the biomarker is a gene or product of a gene located at chromosome 11q13.3-13.4. In some embodiments, the chromosome 11q13.3-13.4 gene is selected from the group consisting of CCND1, CTTN, FADD, ORAOV1, ANO1 , PPFIA _I and SHANK2. In some embodiments, the chromosome 11q13.3-13.4 gene is selected from the group consisting of CCND1, CTTN, FADD, ORAOV1, ANO1,PPFIA1, SHANK2, FGF3, FGF4 and FGF 19 .

In some embodiments, a method of the disclosure may comprise the identification of cells that are more likely to respond to treatment with an ERK inhibitor by assessing the relative copy number of at least one gene located at chromosome 11q13.3-13.4. In some embodiments, the at least one gene located at chromosome 11q13.3-13.4 is selected from the group consisting of CCND1, CTTN, FADD, ORAOV1, ANO1,PPFIA1 and SHANK2. In some embodiments, the at least one gene located at chromosome 11q13.3-13.4 is selected from the group consisting of CCND1, CTTN, FADD, ORAOV1, ANO1,PPFIA1, SHANK2, FGF 3 , FGF4 and FGF 19 .

The methods described herein for qualifying or quantifying the expression of polypeptides and/or polynucleotides provide information which can be correlated with pathological conditions, predisposition to disease, therapeutic monitoring and risk stratification, among others. In some embodiments, a method of the disclosure is particularly useful for diagnosing conditions, evaluating whether an ERK inhibitor will have a desired effect, i.e., predicting responsiveness to an ERK inhibitor, and determining prognoses. The present methods may be used for the optimization of treatment protocols. In this context, evaluation of the expression profile of the biomarkers disclosed herein can be used to gain information on the treatment potential of a tissue sample with an ERK inhibitor.

In some embodiments, the disclosure provides methods for measuring a likelihood that a subject having cancer, especially squamous cell carcinoma, will exhibit a clinically beneficial response to treatment with an ERK inhibitor based on an expression profile and/or a copy number profile of at least one gene or gene products. An “expression profile” refers to a pattern of expression of at least one biomarker, such as at least two biomarkers, that recurs in multiple samples and reflects a property shared by those samples, such as tissue type, response to treatment with an ERK inhibitor, or activation of a particular biological process or pathway in the cells. Furthermore, an expression profile differentiates between samples that share that common property and those that do not with better accuracy than would likely be achieved by assigning the samples to the two groups at random. An expression profile may be used to predict whether samples of unknown status share that common property or not. Some variation between the levels of at least one biomarker and the typical profile is to be expected, but the overall similarity of the expression levels to the typical profile is such that it is statistically unlikely that the similarity would be observed by chance in samples not sharing the common property that the expression profile reflects.

An expression profile may be generated based on a comparison between a total expression level of at least one biomarker in a sample from a test subject and a corresponding reference level. The at least one biomarker may comprise a gene located at chromosome 11q13.3-13.4 that is a predictor of ERK inhibitor sensitivity. In some embodiments, an expression profile is generated based on the expression of 1 or more, 2 or more, 3 or more, 4 or more, 5 or more, 6 or more, or 7 or more biomarkers. In some embodiments, an expression profile is generated based on the expression of 1, 2, 3, 4, 5, 6, or 7 biomarkers.

In some embodiments, the expression profile is used in a method of the disclosure to assess a likelihood of response to treatment with an ERK inhibitor. The likelihood of response may be adjusted upward for each biomarker that is a predictor of ERK inhibitor sensitivity that is overexpressed. In some embodiments, the likelihood of response may be adjusted downward for each biomarker that is a predictor of ERK inhibitor sensitivity that is underexpressed. The magnitude of under- or over-expression may be used to weight the amount of adjustment to the likelihood of response. Preferably, individual expression levels of one or more biomarkers that are predictors of ERK inhibitor sensitivity are summed to give a total expression level.

In some embodiments, a method of the disclosure provides a reference level above which a biomarker must be expressed to be considered in assessing the likelihood of response to treatment with an ERK inhibitor. The biomarker may be differentially expressed at least 1.1 fold, at least 1.2 fold, at least 1.3 fold, at least 1.4 fold, at least 1.5 fold, at least 2.0 fold, at least 2.25 fold, at least 2.5 fold, at least 2.75 fold, at least 3.0 fold, at least 3.5 fold, at least 4.0 fold, at least 5.0, or even at least 10 fold higher or lower relative to a reference level to be considered in adjusting the likelihood of response. In some embodiments, the reference level is a numerical range of biomarker expression that is obtained from a statistical sampling from a population of individuals having cancer that has low sensitivity to treatment with an ERK inhibitor. In some embodiments, the reference level is a numerical range of biomarker expression that is obtained from a statistical sampling from a population of individuals having cancer that is resistant to treatment with an ERK inhibitor. The reference level may be a numerical range of biomarker expression that is obtained from a statistical sampling from a population of individuals having cancer, e.g., the same cancer as the test subject. In some embodiments, the reference level is derived by comparison of sensitive and resistant populations.

In practicing any of the subject methods, the cancer may be selected from squamous cell carcinoma and adenocarcinoma. In some embodiments, the cancer is selected from lung, esophageal, cervical, head and neck, bladder and gastric squamous cell carcinomas. In some embodiments, the cancer is esophageal squamous cell carcinoma. In some embodiments, the cancer is an adenocarcinoma selected from esophageal and pancreatic adenocarcinomas. In some embodiments, the cancer is selected from lung, esophageal, cervical, head and neck, bladder, gastric and pancreatic cancer. In some embodiments, the cancer is selected from breast cancer, pancreatic cancer, lung cancer, thyroid cancer, seminomas, melanoma, bladder cancer, liver cancer, kidney cancer, myelodysplastic syndrome, acute myelogenous leukemia and colorectal cancer.

Certain embodiments contemplate a human subject such as a subject that has been diagnosed as having or being at risk for developing or acquiring cancer, such as squamous cell carcinoma. Certain other embodiments contemplate a non-human subject, for example a non-human primate such as a macaque, chimpanzee, gorilla, vervet, orangutan, baboon or other non-human primate, including such non-human subjects that can be known to the art as preclinical models. Certain other embodiments contemplate a non-human subject that is a mammal, for example, a mouse, rat, rabbit, pig, sheep, horse, bovine, goat, gerbil, hamster, guinea pig or other mammal. There are also contemplated other embodiments in which the subject or biological source can be a non-mammalian vertebrate, for example, another higher vertebrate, or an avian, amphibian or reptilian species, or another subject or biological source. In certain embodiments of the present disclosure, a transgenic animal is utilized. A transgenic animal is a non-human animal in which one or more of the cells of the animal includes a nucleic acid that is non-endogenous (i.e., heterologous) and is present as an extrachromosomal element in a portion of its cell or stably integrated into its germ line DNA (i.e., in the genomic sequence of most or all of its cells).

Any cancer may be analyzed and/or treated according to the methods of the disclosure. The methods described herein are particularly effective in analyzing and/or treating squamous cell carcinoma. Exemplary squamous cell carcinomas include squamous cell carcinomas of the skin, head and neck, thyroid, esophagus, lung, penis, prostate, vagina, cervix, and bladder. Preferably, the squamous cell carcinoma is selected from lung, esophagus, and head and neck squamous cell carcinomas. In some embodiments, the squamous cell carcinoma is squamous cell carcinoma of the lung. In some embodiments, the squamous cell carcinoma is squamous cell carcinoma of the esophagus. In some embodiments, the squamous cell carcinoma is squamous cell carcinoma of the head and neck. In some embodiments, the squamous cell carcinoma is squamous cell carcinoma of the cervix.

Typically, a sample of a subject (e.g. a biological sample) comprises cancerous or pre-cancerous cells. The biological sample may be a tissue sample. The sample may be a solid biological sample, for example, a tumor biopsy. A biopsy may be fixed, paraffin-embedded, fresh, or frozen. Samples may be obtained by any suitable means, including but not limited to needle aspiration, fine needle aspiration, core needle biopsy, vacuum assisted biopsy, large core biopsy, incisional biopsy, excisional biopsy, punch biopsy, shave biopsy, skin biopsy, and venipuncture. A sample may be derived from fine needle, core, or other types of biopsy, or may comprise circulating tumor cells. In some examples, a sample comprises cell-free DNA (cfDNA). A biological sample may be a whole blood or plasma sample. A sample may be analyzed directly for its contents, or may be processed to purify one or more of its contents for analysis. Methods of direct analysis of samples are known in the art and include, without limitation, mass spectrometry and histological staining procedures. In some embodiments, one or more components are purified from the sample for the detection of a biomarker for ERK inhibitor response. In some embodiments, the purified component of the sample is protein (e.g. total protein, cytoplasmic protein, or membrane protein). In some embodiments, the purified component of the sample is a nucleic acid, such as DNA (e.g. genomic DNA, cDNA, ctDNA, or cfDNA) or RNA (e.g. total RNA or mRNA). In some embodiments, the nucleic acid is from a cancer cell, such as a squamous cell carcinoma cell.

Methods for the extraction, purification, and amplification of nucleic acids are known in the art. For example, nucleic acids can be purified by organic extraction with phenol, phenol/chloroform/isoamyl alcohol, or similar formulations, including TRIzol and TriReagent. Other non-limiting examples of extraction techniques include: organic extraction followed by ethanol precipitation, e.g., using a phenol/chloroform organic reagent (Ausubel et al., 1993), with or without the use of an automated nucleic acid extractor, e.g., the Model 341 DNA Extractor available from Applied Biosystems (Foster City, Calif); stationary phase adsorption methods (U.S. Pat. No. 5,234,809; Walsh et al., 1991); and salt-induced nucleic acid precipitation methods (Miller et al., (1988), such precipitation methods being typically referred to as “salting-out” methods. Another example of nucleic acid isolation and/or purification includes the use of magnetic particles to which nucleic acids can specifically or non-specifically bind, followed by isolation of the beads using a magnet, and washing and eluting the nucleic acids from the beads (see e.g. U.S. Pat. No. 5,705,628). In some embodiments, the above isolation methods may be preceded by an enzyme digestion step to help eliminate unwanted protein from the sample, e.g., digestion with proteinase K, or other like proteases. See, e.g., U .S. Pat. No. 7,001,724. If desired, RNase inhibitors may be added to the lysis buffer. For certain cell or sample types, it may be desirable to add a protein denaturation/digestion step to the protocol. Purification methods may be directed to isolate DNA, RNA, or both. When both DNA and RNA are isolated together during or subsequent to an extraction procedure, further steps may be employed to purify one or both separately from the other. Sub-fractions of extracted nucleic acids can also be generated, for example, purification by size, sequence, or other physical or chemical characteristics. In addition to an initial nucleic acid isolation step, purification of nucleic acids can be performed after any step in the methods of the disclosure, such as to remove excess or unwanted reagents, reactants, or products.

In some embodiments, sample polynucleotides are fragmented into a population of fragmented DNA molecules of one or more specific size range(s). In some embodiments, fragments are generated from about or at least about 1, 10, 100, 1000, 10000, 100000, 300000, 500000, or more genome-equivalents of starting DNA. Fragmentation may be accomplished by methods known in the art, including chemical, enzymatic, and mechanical fragmentation. In some embodiments, the fragments have an average length from about 10 to about 10,000 nucleotides. In some embodiments, the fragments have an average length from about 50 to about 2,000 nucleotides. In some embodiments, the fragments have an average or median length from about 10-2,500, 10-1,000, 10-800, 10-500, 50-500, 50-250, 50-150, or 100-2,500 nucleotides. In some embodiments, the fragmentation is accomplished mechanically by subjecting sample polynucleotides to acoustic sonication. In some embodiments, the fragmentation comprises treating the sample polynucleotides with one or more enzymes under conditions suitable for the one or more enzymes to generate double-stranded nucleic acid breaks. Examples of enzymes useful in the generation of polynucleotide fragments include sequence specific and non-sequence specific nucleases. Non-limiting examples of nucleases include DNase I, Fragmentase, restriction endonucleases, variants thereof, and combinations thereof. For example, digestion with DNase I can induce random double-stranded breaks in DNA in the absence of Mg⁺⁺ and in the presence of Mn⁻⁺. In some embodiments, fragmentation comprises treating the sample polynucleotides with one or more restriction endonucleases. Fragmentation can produce fragments having 5′ overhangs, 3′ overhangs, blunt ends, or a combination thereof In some embodiments, such as when fragmentation comprises the use of one or more restriction endonucleases, cleavage of sample polynucleotides leaves overhangs having a predictable sequence. In some embodiments, the method includes the step of size selecting the fragments via standard methods such as column purification or isolation from an agarose gel.

In some embodiments, one or more polynucleotides from a sample of a subject are amplified. In general, amplification comprises generating one or more copies of all or a portion of polynucleotides in a template-dependent manner. Amplification may be primer-dependent, or primer-independent. When primer-dependent, amplification may be directed to one or more specific polynucleotides in a sample or portions thereof, such as one or more regions (e.g. about or more than about 1, 5, 10, 15, 20, 25, 30, 35, 40, 45, 50, 75, 100, 200, 500, or more regions), each region comprising one or more sequences of interest, and having a length of about, less than about, or more than about 1, 5, 10, 25, 50, 100, 150, 200, 250, 350, 500, 1000, 2000, or more nucleotides. Amplification may be linear or non-linear (e.g. exponential). Amplification may comprise directed changes in temperature, or may be isothermal. Methods for primer-directed amplification of target polynucleotides are known in the art, and include without limitation, methods based on the polymerase chain reaction (PCR). Conditions favorable to the amplification of target sequences by PCR are known in the art, can be optimized at a variety of steps in the process, and depend on characteristics of elements in the reaction, such as target type, target concentration, sequence length to be amplified, sequence of the target and/or one or more primers, primer length, primer concentration, polymerase used, reaction volume, ratio of one or more elements to one or more other elements, some or all of which can be altered. In general, PCR involves the steps of denaturation of the target to be amplified (if double stranded), hybridization of one or more primers to the target, and extension of the primers by a DNA polymerase, with the steps repeated (or “cycled”) in order to amplify the target sequence. Steps in this process can be optimized for various outcomes, such as to enhance yield, decrease the formation of spurious products, and/or increase or decrease specificity of primer annealing. Methods of optimization are well known in the art and include adjustments to the type or amount of elements in the amplification reaction and/or to the conditions of a given step in the process, such as temperature at a particular step, duration of a particular step, and/or number of cycles. In some embodiments, an amplification reaction comprises at least 5, 10, 15, 20, 25, 30, 35, 50, or more cycles. In some embodiments, an amplification reaction comprises no more than 5, 10, 15, 20, 25, 35, 50, or more cycles. Cycles can contain any number of steps, such as 1, 2, 3, 4, 5, 6, 7, 8, 9, 10 or more steps. Steps can comprise any temperature or gradient of temperatures, suitable for achieving the purpose of the given step, including but not limited to, primer annealing, primer extension, and strand denaturation. Steps can be of any duration, including but not limited to about, less than about, or more than about 1, 5, 10, 15, 20, 25, 30, 35, 40, 45, 50, 55, 60, 70, 80, 90, 100, 120, 180, 240, 300, 360, 420, 480, 540, 600, or more seconds, including indefinitely until manually interrupted. Cycles of any number comprising different steps can be combined in any order. In some embodiments, different cycles comprising different steps are combined such that the total number of cycles in the combination is about, less that about, or more than about 5, 10, 15, 20, 25, 30, 35, 50, or more cycles.

A total expression level of a biomarker, such as a MAPK pathway gene or a RAS-ERK feedback regulator, may be assessed by any appropriate method. The expression level of a biomarker may be assessed by detecting a level of mRNA transcribed from the biomarker, by detecting a level of cDNA produced from reverse transcription of mRNA transcribed from the biomarker, by detecting a level of polypeptide encoded by the biomarker, or by a nucleic acid amplification assay, a hybridization assay, sequencing, or a combination thereof. Regulation of a target gene or gene transcript can also be determined indirectly, such as by measuring the effect on a phenotypic indicator of the gene or gene transcript activity, such as by cellular assay. Methods of detecting gene expression products are known in the art, examples of which are described herein. These methods can be performed on a sample by sample basis or modified for high throughput analysis, for example, using Affymetrix™ U133 microarray chips.

Optionally, assessment of a total expression level of a gene, such as a MAPK pathway gene or a RAS-ERK feedback regulator, comprises forming a plurality of complexes, each complex comprising an association between an expression product of the gene and a nucleic acid probe that hybridizes to the expression product of the gene. The nucleic acid probe may comprise a first nucleic acid complex, wherein the complex comprises (i) a first target-specific sequence capable of binding to a target nucleic acid, (ii) a first label attachment region, which is non-overlapping with the first target-specific sequence, comprising a first DNA sequence hybridized to a first nucleic acid molecule that is attached to one or more detectable labels that emit light which constitutes a first signal, (iii) a second label attachment region, which is non-overlapping with the first target-specific sequence and the first label attachment region, comprising a second DNA sequence hybridized to a second nucleic acid molecule that is attached to one or more detectable labels that emit light which constitutes a second signal, and (iv) a first moiety that is capable of selectively binding to the substrate. Optionally, the nucleic acid probe further comprises a second nucleic acid complex, the second complex comprising (i) a second target-specific sequence capable of binding to the target nucleic acid, wherein the first target-specific sequence and the second target-specific sequence bind to different regions of the target nucleic acid, and (ii) a second moiety that is capable of selectively binding to the substrate. In some embodiments, the first nucleic acid molecule comprises at least one additional attachment region which is non-overlapping with other label attachment regions. The at least one additional label attachment region may comprise a DNA sequence hybridized to a nucleic acid molecule that is attached to at least one detectable label that emits light. The at least one additional label attachment region may comprise a DNA sequence hybridized to a nucleic acid molecule that is not attached to a detectable label that emits light. In some embodiments, the first and second nucleic acid molecules each comprise four or more aminoallyl-modified UTP nucleotides, wherein one or more fluorophore labels is attached to each aminoallyl-modified UTP nucleotide. The first moiety and/or the second moiety may each be independently selected from biotin, digoxigenin, FITC, avidin, streptavidin, antidigoxigenin and anti-FITC.

In a preferred embodiment, the nCounter® Analysis system is used to detect gene expression. The basis of the nCounter® Analysis system is the unique code assigned to each nucleic acid target to be assayed (see, e.g., WO2008/0124847, U.S. Pat. No. 8,415,102 and Geiss et al. Nature Biotechnology 2008 26(3): 317-325, the contents of which are each incorporated herein by reference in their entireties). The code is composed of an ordered series of colored fluorescent spots which create a unique barcode for each target to be assayed. A pair of nucleic acid probes is designed for each DNA or RNA target described herein, a capture probe and a reporter probe carrying the fluorescent barcode. This system is also referred to herein as the nanoreporter code system. See also WO2016/085841, WO2016/081740, WO2016/022559, and U.S. Pub. Nos. 2013/0017971, 2013/0230851 and 2014/0154681, each incorporated herein by reference.

Detection of nucleic acids may involve the use of a hybridization reaction, such as between a target nucleic acid and an oligonucleotide probe or primer (e.g., a nucleic acid hybridization assay). In some embodiments, the oligonucleotide probe is immobilized on a substrate. Substrates include, but are not limited to, arrays, microarrays, wells of a multi-well plate, and beads (e.g. non-magnetic, magnetic, paramagnetic, hydrophobic, and hydrophilic beads). Examples of materials useful as substrates include but are not limited to nitrocellulose, glass, silicon, and a variety of gene arrays. A preferred hybridization assay is conducted on high-density gene chips as described in U.S. Pat. No. 5,445,934.

The expression level of a gene may be determined through exposure of a nucleic acid sample to the probe-modified chip. Extracted nucleic acid is labeled, for example, with a fluorescent tag, preferably during an amplification step. Hybridization of the labeled sample is performed at an appropriate stringency level. The degree of probe-nucleic acid hybridization may be quantitatively measured using a detection device. See U.S. Pat. Nos. 5,578,832 and 5,631,734.

Alternatively any one of gene copy number, transcription, or translation can be determined using known techniques. For example, an amplification method such as PCR may be useful. General procedures for PCR are taught in MacPherson et al., PCR: A Practical Approach, (IRL Press at Oxford University Press (1991)). PCR conditions used for each application reaction are empirically determined. A number of parameters influence the success of a reaction. Among them are annealing temperature and time, extension time, Mg²⁺ and/or ATP concentration, pH, and the relative concentration of primers, templates, and deoxyribonucleotides. After amplification, the resulting DNA fragments can be detected by agarose gel electrophoresis followed by visualization with ethidium bromide staining and ultraviolet illumination.

The hybridized nucleic acids may be detected by detecting one or more labels attached to the sample nucleic acids. The labels can be incorporated by any of a number of means well known to those of skill in the art. However, in one embodiment, the label is simultaneously incorporated during the amplification step in the preparation of the sample nucleic acid. Thus, for example, polymerase chain reaction (PCR) with labeled primers or labeled nucleotides will provide a labeled amplification product. In a separate embodiment, transcription amplification, as described above, using a labeled nucleotide (e.g. fluorescein-labeled UTP and/or CTP) incorporates a label in to the transcribed nucleic acids.

Alternatively, a label may be added directly to the original nucleic acid sample (e.g., mRNA, polyA, cDNA, etc.) or to the amplification product after the amplification is completed. Means of attaching labels to nucleic acids are well known to those of skill in the art and include, for example nick translation or end-labeling (e.g. with a labeled RNA) by kinasing of the nucleic acid and subsequent attachment (ligation) of a nucleic acid linker joining the sample nucleic acid to a label (e.g., a fluorophore).

Suitable detectable labels may include any composition detectable by spectroscopic, photochemical, biochemical, immunochemical, electrical, optical or chemical means. Useful labels include, for example, biotin for staining with labeled streptavidin conjugate, magnetic beads (e.g., Dynabeads™), fluorescent dyes (e.g., fluorescein, Texas red, rhodamine, green fluorescent protein, and the like), radiolabels (e.g., 3H, 125I, 35S, 14C, or 32P) enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Detection of labels is well known to those of skill in the art. Thus, for example, radiolabels may be detected using photographic film or scintillation counters. Fluorescent markers may be detected using a photodetector to detect emitted light. Enzymatic labels are typically detected by providing the enzyme with a substrate and detecting the reaction product produced by the action of the enzyme on the substrate. calorimetric labels may be detected by simply visualizing the colored label.

A biomarker (e.g., a MAPK pathway gene or a RAS-ERK feedback regulator) may be detected in a biological sample using a microarray. Differential gene expression can also be identified, or confirmed using the microarray technique. Thus, the expression profile can be measured in either fresh or fixed tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. The source of mRNA typically is total RNA isolated from a biological sample, and corresponding normal tissues or cell lines may be used to determine differential expression.

In a specific embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the microarray chip is scanned by a device, such as confocal laser microscopy, or by another detection method, such as a CCD camera. Quantitation of hybridization of each arrayed element allows for assessment of corresponding mRNA abundance. With dual color fluorescence, separately labeled cDNA probes generated from two sources of RNA are hybridized pair-wise to the array. The relative abundance of the transcripts from the two sources corresponding to each specified gene is thus determined simultaneously. Microarray analysis can be performed by commercially available equipment, following manufacturer's protocols.

The biomarker may be detected in a biological sample using qRT-PCR, which can be used to compare mRNA levels in different sample populations, in normal and tumor tissues, with or without drug treatment, to characterize patterns of gene expression, to discriminate between closely related mRNAs, and to analyze RNA structure. The first step in gene expression profiling by RT-PCR is extracting RNA from a biological sample followed by the reverse transcription of the RNA template into cDNA and amplification by a PCR reaction. The reverse transcription reaction step is generally primed using specific primers, random hexamers, or oligo-dT primers, depending on the goal of expression profiling. The two commonly used reverse transcriptases are avilo myeloblastosis virus reverse transcriptase (AMV-RT) and Moloney murine leukemia virus reverse transcriptase (MLV-RT).

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqManTm PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect the nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

Differential expression of a biomarker (e.g., a MAPK pathway gene or a RAS-ERK feedback regulator) can also be determined by examining protein expression or the protein product of the biomarker, for example, using a suitable protein assay. Determining the protein level involves measuring the amount of any immunospecific binding that occurs between an antibody that selectively recognizes and binds to the polypeptide of the biomarker in a test sample and comparing this to the amount of immunospecific binding of at least one biomarker in a reference sample. The amount of protein expression of the biomarker may be increased or reduced when compared with a reference expression level. Optionally, all of the biomarkers disclosed herein may be assayed for as a single set.

A variety of techniques are available in the art for protein analysis. They include but are not limited to radioimmunoassays, ELISA (enzyme linked immunosorbent assays), “sandwich” immunoassays, immunoradiometric assays, in situ immunoassays (using e.g., colloidal gold, enzyme or radioisotope labels), western blot analysis, immunoprecipitation assays, immunofluorescent assays, flow cytometry, immunohistochemistry, confocal microscopy, enzymatic assays, surface plasmon resonance and PAGE-SDS.

The present disclosure provides methods for detecting biomarkers, such as a MAPK pathway gene or a RAS-ERK feedback regulator, in a biological sample. Useful analyte capture agents that can be used with the present disclosure include but are not limited to antibodies, such as crude serum containing antibodies, purified antibodies, monoclonal antibodies, polyclonal antibodies, synthetic antibodies, antibody fragments (for example, Fab fragments); antibody interacting agents, such as protein A, carbohydrate binding proteins, and other interactants; protein interactants (for example avidin and its derivatives); peptides; and small chemical entities, such as enzyme substrates, cofactors, metal ions/chelates, and haptens. Antibodies may be modified or chemically treated to optimize binding to targets or solid surfaces (e.g. biochips and columns).

In some embodiments, the biomarker can be detected in a biological sample using an immunoassay. Immunoassays are assays that use an antibody that specifically binds to or recognizes an antigen (e.g. site on a protein or peptide, biomarker target). The method includes the steps of contacting the biological sample with the antibody and allowing the antibody to form a complex with the antigen in the sample, washing the sample and detecting the antibody-antigen complex with a detection reagent. In one embodiment, antibodies that recognize the biomarkers may be commercially available. In another embodiment, an antibody that recognizes the biomarkers may be generated by known methods of antibody production.

Alternatively, the biomarker in the sample can be detected using an indirect assay, wherein, for example, a second, labeled antibody is used to detect bound biomarker-specific antibody. Exemplary detectable labels include magnetic beads (e.g., DYNABEADSTM), fluorescent dyes, radiolabels, enzymes (e.g., horse radish peroxide, alkaline phosphatase and others commonly used), and calorimetric labels such as colloidal gold or colored glass or plastic beads. The biomarker in the sample can be detected using and/or in a competition or inhibition assay wherein, for example, a monoclonal antibody which binds to a distinct epitope of the marker is incubated simultaneously with the mixture.

The conditions to detect an antigen using an immunoassay will be dependent on the particular antibody used. Also, the incubation time will depend upon the assay format, biomarker, volume of solution, concentrations and the like. In general, the immunoassays will be carried out at room temperature, although they can be conducted over a range of temperatures, such as 10 to 40 ° C., depending on the antibody used.

There are various types of immunoassays known in the art that as a starting basis can be used to tailor the assay for the detection of the biomarkers (e.g., MAPK pathway genes or RAS-ERK feedback regulators) of the present disclosure. Useful assays can include, for example, an enzyme immune assay (EIA) such as enzyme-linked immunosorbent assay (ELISA). There are many variants of these approaches, but those are based on a similar idea. For example, if an antigen can be bound to a solid support or surface, it can be detected by reacting it with a specific antibody, and the antibody can be quantitated by reacting it with either a secondary antibody or by incorporating a label directly into the primary antibody. Alternatively, an antibody can be bound to a solid surface and the antigen added. A second antibody that recognizes a distinct epitope on the antigen can then be added and detected. This is frequently called a ‘sandwich assay’ and can frequently be used to avoid problems of high background or non-specific reactions. These types of assays are sensitive and reproducible enough to measure low concentrations of antigens in a biological sample.

Proximity ligation assay (PLA) is another type of immunoassay known in the art useful for the detection of the biomarkers of the present disclosure. The term “proximity ligation assay” or “PLA” as used herein refers to an immunoassay utilizing so-called PLA probes affinity reagents such as antibodies modified with DNA oligonucleotides for detecting and reporting the presence of proteins either in solution or in situ. When two PLA probes bind the same or two interacting target molecules, the attached oligonucleotides are brought in close proximity. A proximity ligation assay may be tailored to detect the biomarkers disclosed herein.

Immunoassays can be used to determine presence or absence of a biomarker in a sample as well as the quantity of a biomarker in a sample. Methods for measuring the amount of, or presence of, an antibody-biomarker complex include but are not limited to, fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). In general these regents are used with optical detection methods, such as various forms of microscopy, imaging methods and non-imaging methods. Electrochemical methods include voltametry and amperometry methods. Radio frequency methods include multipolar resonance spectroscopy.

Biochips can be designed with immobilized nucleic acid molecules, full-length proteins, antibodies, affibodies (small molecules engineered to mimic monoclonal antibodies), aptamers (nucleic acid-based ligands) or chemical compounds. A chip could be designed to detect multiple macromolecule types on one chip. For example, a chip could be designed to detect nucleic acid molecules, proteins and metabolites on one chip. The biochip is used to and designed to simultaneously analyze a panel biomarker in a single sample, producing a subject's profile for these biomarkers. The use of the biochip allows for the multiple analyses to be performed reducing the overall processing time and the amount of sample required.

Protein microarrays are a particular type of biochip which can be used with the present disclosure. The chip consists of a support surface such as a glass slide, nitrocellulose membrane, bead, or microtitre plate, to which an array of capture proteins are bound in an arrayed format onto a solid surface. Protein array detection methods must give a high signal and a low background. Detection probe molecules, typically labeled with a fluorescent dye, are added to the array. Any reaction between the probe and the immobilized protein emits a fluorescent signal that is read by a laser scanner. Such protein microarrays are rapid, automated, and offer high sensitivity of protein biomarker read-outs for diagnostic tests. However, it would be immediately appreciated to those skilled in the art that there are a variety of detection methods that can be used with this technology.

The present disclosure provides for the detection of biomarkers using mass spectrometry. Mass spectrometry (MS) is an analytical technique that measures the mass-to-charge ratio of charged particles. It is primarily used for determining the elemental composition of a sample or molecule, and for elucidating the chemical structures of molecules, such as peptides and other chemical compounds. MS works by ionizing chemical compounds to generate charged molecules or molecule fragments and measuring their mass-to-charge ratios. MS instruments typically consist of three modules (1) an ion source, which can convert gas phase sample molecules into ions (or, in the case of electrospray ionization, move ions that exist in solution into the gas phase) (2) a mass analyzer, which sorts the ions by their masses by applying electromagnetic fields and (3) a detector, which measures the value of an indicator quantity and thus provides data for calculating the abundances of each ion present.

Suitable mass spectrometry methods to be used with the present disclosure include but are not limited to, one or more of electrospray ionization mass spectrometry (ESI-MS), ESI-MS/MS, ESI-MS/(MS)ₙ, matrix-assisted laser desorption ionization time-of-flight mass spectrometry (MALDI-TOF-MS), surface-enhanced laser desorption/ionization time-of-flight mass spectrometry (SELDI-TOF-MS), tandem liquid chromatography-mass spectrometry (LC-MS/MS) mass spectrometry, desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS), atmospheric pressure photoionization mass spectrometry (APPI-MS), APPI-MS/MS, and APPI-(MS)ₙ, quadrupole mass spectrometry, Fourier transform mass spectrometry (FTMS), and ion trap mass spectrometry, where n is an integer greater than zero.

To gain insight into the underlying proteomics of a sample, LC-MS is commonly used to resolve the components of a complex mixture. LC-MS method generally involves protease digestion and denaturation (usually involving a protease, such as trypsin, a denaturant (e.g., urea) to denature tertiary structure, and iodoacetamide to cap cysteine residues) followed by LC-MS with peptide mass fingerprinting or LC-MS/MS (tandem MS) to derive sequence of individual peptides. LC-MS/MS is most commonly used for proteomic analysis of complex samples where peptide masses may overlap even with a high-resolution mass spectrometer. Samples of complex biological fluids like human serum may be first separated on an SDS-PAGE gel or HPLC—SCX and then run in LC-MS/MS allowing for the identification of over 1000 proteins.

In some applications, HPLC and UHPLC can be coupled to a mass spectrometer. A number of other peptide and protein separation techniques can be performed prior to mass spectrometric analysis. Some exemplary separation techniques which can be used for separation of the desired analyte (e.g., peptide or protein) from the matrix background include but are not limited to Reverse Phase Liquid Chromatography (RP-LC) of proteins or peptides, offline Liquid Chromatography (LC), 1-dimensional gel separation, 2-dimensional gel separation, Strong Cation Exchange (SCX) chromatography, Strong Anion Exchange (SAX) chromatography, Weak Cation Exchange (WCX), and Weak Anion Exchange (WAX). One or more of the above techniques can be used prior to mass spectrometric analysis.

Methods for determining whether a MAPK pathway gene, such as EGFR, is amplified are widely known in the state of the art. Said methods include, without limitation, in situ hybridization (ISH) (such as fluorescence in situ hybridization (FISH), chromogenic in situ hybridization (CISH) or silver in situ hybridization (SISH)), genomic comparative hybridization or polymerase chain reaction (such as real time quantitative PCR). For any ISH method, the amplification or the copy number can be determined by counting the number of fluorescent points, colored points or points with silver in the chromosomes or in the nucleus.

The fluorescence in situ hybridization (FISH) is a cytogenetic technique which is used for detecting and locating the presence or absence of specific DNA sequences in chromosomes. FISH uses fluorescence probes which only bind to some parts of the chromosome with which they show a high degree of sequence similarity. In a typical FISH method, the DNA probe is labeled with a fluorescent molecule or a hapten, typically in the form of fluor-dUTP, digoxigenin-dUTP, biotin-dUTP or hapten-dUTP which is incorporated in the DNA using enzymatic reactions, such as nick translation or PCR. The sample containing the genetic material (the chromosomes) is placed on glass slides and is denatured by a formamide treatment. The labeled probe is then hybridized with the sample containing the genetic material under suitable conditions which will be determined by the person skilled in the art. After the hybridization, the sample is viewed either directly (in the case of a probe labeled with fluorine) or indirectly (using fluorescently labeled antibodies to detect the hapten). In the case of CISH, the probe is labeled with digoxigenin, biotin or fluorescein and is hybridized with the sample containing the genetic material in suitable conditions.

Copy number abnormalities can be detected using methods such as comparative genomic hybridization (CGH), microsatellite markers, short tandem repeat (STR) analysis, and restriction fragment length polymorphism (RFLP) analysis. Additional methods for assessing copy number of nucleic acid in a sample include, but are not limited to, hybridization-based assays. One method for assessing the copy number of encoding nucleic acid in a sample involves a Southern Blot. In a Southern Blot, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid. Alternatively, a Northern blot may be utilized for assessing the copy number of encoding nucleic acid in a sample. In a Northern blot, mRNA is hybridized to a probe specific for the target region. Comparison of the intensity of the hybridization signal from the probe for the target region with control probe signal from analysis of normal mRNA (e.g., a non-amplified portion of the same or related cell, tissue, organ, etc.) provides an estimate of the relative copy number of the target nucleic acid. Similar methods for assessing copy number can be performed using transcriptional arrays, which are well-known in the art.

Preferred hybridization-based assays include, but are not limited to, traditional “direct probe” methods such as Southern blots or in situ hybridization (e.g., FISH and FISH plus SKY), and “comparative probe” methods such as comparative genomic hybridization (CGH), e.g., cDNA-based or oligonucleotide-based CGH. The methods can be used in a wide variety of formats including, but not limited to, substrate (e.g. membrane or glass) bound methods or array-based approaches.

In CGH methods, a first collection of nucleic acids (e.g., from a sample, such as a squamous cell carcinoma cell) is labeled with a first label, while a second collection of nucleic acids (e.g., a control, e.g., from a healthy cell/tissue) is labeled with a second label. The ratio of hybridization of the nucleic acids is determined by the ratio of the two (first and second) labels binding to each fiber in the array. Where there are chromosomal deletions or multiplications, differences in the ratio of the signals from the two labels will be detected and the ratio will provide a measure of the copy number. Array-based CGH may also be performed with single-color labeling (as opposed to labeling the control and the possible tumor sample with two different dyes and mixing them prior to hybridization, which will yield a ratio due to competitive hybridization of probes on the arrays). In single color CGH, the control is labeled and hybridized to one array and absolute signals are read, and the squamous cell carcinoma sample is labeled and hybridized to a second array (with identical content) and absolute signals are read. Copy number difference is calculated based on absolute signals from the two arrays. Hybridization protocols suitable for use with the methods of the disclosure are described, e.g., in Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In situ Hybridization Protocols, Choo, ed., Humana Press, Totowa, N.J. (1994), etc. In one embodiment, the hybridization protocol of Pinkel, et al. (1998) Nature Genetics 20: 207-211, or of Kallioniemi (1992) Proc. Natl Acad Sci USA 89:5321-5325 (1992) is used.

The methods of the present disclosure are particularly well suited to array-based hybridization formats. Array-based CGH is described in U.S. Pat. No. 6,455,258, the contents of which are incorporated herein by reference. In still another embodiment, amplification-based assays can be used to measure copy number. In such amplification-based assays, the nucleic acid sequences act as a template in an amplification reaction (e.g., Polymerase Chain Reaction (PCR). In a quantitative amplification, the amount of amplification product will be proportional to the amount of template in the original sample. Comparison to appropriate controls, e.g. healthy tissue, provides a measure of the copy number.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y. Measurement of DNA copy number at microsatellite loci using quantitative PCR anlaysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods of the disclosure. In fluorogenic quantitative PCR, quantitation is based on amount of fluorescence signals, e.g., TaqMan and SYBR Green.

Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. USA 86: 1173), self-sustained sequence replication (Guatelli, et al. (1990) Proc. Nat. Acad. Sci. USA 87: 1874), dot PCR, and linker adapter PCR, etc.

In some embodiments, sequencing of individual nucleic molecules (or their amplification products) is performed, as an alternative to hybridization-based assays, using nucleic acid sequencing techniques. In one embodiment, a high throughput parallel sequencing technique that isolates single nucleic acid molecules of a population of nucleic acid molecules prior to sequencing may be used. Such strategies may use so-called “next generation sequencing systems” including, without limitation, sequencing machines and/or strategies well known in the art, such as those developed by Illumina/Solexa (the Genome Analyzer; Bennett et al. (2005) Pharmacogenomics, 6:373-20 382), by Applied Biosystems, Inc. (the SOLiD Sequencer; solid.appliedbiosystems.com), by Roche (e.g., the 454 GS FLX sequencer; Margulies et al. (2005) Nature, 437:376-380; U.S. Pat. Nos. 6,274,320; 6,258,568; 6,210,891), by Heliscope (Registered Trademark) system from Helicos Biosciences (see, e.g., U.S. Patent App. Pub. No. 2007/0070349), and by others. Other sequencing strategies such as stochastic sequencing (e.g., as developed by Oxford Nanopore) may also be used, e.g., as described in International Application No. PCT/GB2009/001690 (Pub. No. WO/2010/004273).

In some embodiments, one or more steps in the assessment and/or reporting of a likelihood of response to treatment with an ERK inhibitor is performed with the aid of a processor, such as with a computer system executing instructions contained in computer-readable media. In one aspect, the disclosure provides a system for of assessing a likelihood of a subject having cancer, such as squamous cell carcinoma, exhibiting a clinically beneficial response to treatment with an ERK inhibitor. In one embodiment, the system comprises (a) a memory unit configured to store information concerning: (i) a first total expression level of at least two genes selected from the group consisting of EGFR, ERK 1 , CCND1, KRAS, ERK2, and HRAS; (ii) a second total expression level of at least two genes selected from the group consisting of DUSP5 , DUSP6, DUSP2 , DUSP4 , SPRY2 , SPRY4, and SPRED1; (iii) a third total expression level of at least two genes selected from the group consisting of CCND1, CRAF , DUSP5 , EGFR, ERK1 , and KRAS; (iv) a copy number profile of at least one MAPK pathway gene; (v) a fourth total expression level of AREG, CDH3, COL17A1 , EGFR, HIF1A, ITGB1, KRT1 , KRT9 , NRG1 , SLC16A1 , SLC22A1 and VEGFA; (vi) a fifth total expression level of DCUN1D1, PIK3CA, PRKCI, SOX2 and TP63; and/or (vii) expression levels of HIF1A and TP63, in a biological sample comprising genomic and/or transcriptomic material from a squamous cell carcinoma cell. In some embodiments, the system further comprises (b) one or more processors alone or in combination programmed to: (1) determine a weighted probability of ERK inhibitor responsiveness based on the first total expression level, the second total expression level, the copy number profile, the third total expression level, the fourth total expression level, the fifth total expression level, and/or the expression levels of HIF1A and TP63; and (2) designate the subject as having a high probability of exhibiting a clinically beneficial response to treatment with the ERK inhibitor if the weighted probability corresponds to at least 1.5 times a baseline probability, wherein the baseline probability represents a likelihood that the subject will exhibit a clinically beneficial response to treatment with the ERK inhibitor before obtaining the weighted probability of (b)(1).

In some embodiments, one or more steps in the assessment and/or reporting of a likelihood of response to treatment with an ERK inhibitor is performed with the aid of a processor, such as with a computer system executing instructions contained in computer-readable media. In one aspect, the disclosure provides a system for assessing a likelihood of a subject having cancer, such as squamous cell carcinoma, exhibiting a clinically beneficial response to treatment with an ERK inhibitor. In some embodiments, the system comprises (a) a memory unit configured to store information concerning a copy number profile and/or expression level of at least one gene located at chromosome 11q13.3-13.4 in a biological sample comprising genomic and/or transcriptomic material from a cancer cell; and (b) one or more processors alone or in combination programmed to (1) determine a weighted probability of ERK inhibitor responsiveness based on the copy number profile and/or the expression level; and (2) designate the subject as having a high probability of exhibiting a clinically beneficial response to treatment with the ERK inhibitor if the weighted probability corresponds to at least 1.5 times a baseline probability, wherein the baseline probability represents a likelihood that the subject will exhibit a clinically beneficial response to treatment with the ERK inhibitor before obtaining the weighted probability of (b)(1). In some embodiments, the at least one gene located at chromosome 11q13.3-13.4 is selected from the group consisting of CCND1, CTTN, FADD, ORAOV1, ANO1, PPFIA1 and SHANK2. In some embodiments, the at least one gene is CCND1 or ANO1 . In some embodiments, the at least one gene comprises CCND1 and ANO1. In some embodiments, the at least one gene located at chromosome 11q13.3-13.4 is selected from the group consisting of CCND1, CTTN, FADD, ORAOV1, ANO1, PPFIA1, SHANK2, FGF3, FGF4 and FGF 19 .

In some embodiments, the expression level is assessed by (a) detecting a level of mRNA; (b) detecting a level of cDNA produced from reverse transcription of mRNA; (c) detecting a level of polypeptide; (d) detecting a level of cell-free DNA; and/or (e) a nucleic acid amplification assay, a hybridization assay, sequencing, or a combination thereof In some embodiments, the copy number profile of the at least one gene is assessed by a method selected from the group consisting of in situ hybridization, Southern blot, immunohistochemistry (IHC), polymerase chain reaction (PCR), quantitative PCR (qPCR), quantitative real-time PCR (qRT-PCR), comparative genomic hybridization, microarray-based comparative genomic hybridization, and ligase chain reaction (LCR). In some embodiments, the cancer is selected from squamous cell carcinoma and adenocarcinoma. In some embodiments, the cancer is selected from lung, esophageal, cervical, head and neck, bladder and gastric squamous cell carcinomas. In some embodiments, the cancer is esophageal squamous cell carcinoma. In some embodiments, the cancer is an adenocarcinoma selected from esophageal and pancreatic adenocarcinomas. In some embodiments, the cancer is selected from lung, esophageal, cervical, head and neck, bladder, gastric and pancreatic cancer. In some embodiments, the cancer is selected from breast cancer, pancreatic cancer, lung cancer, thyroid cancer, seminomas, melanoma, bladder cancer, liver cancer, kidney cancer, myelodysplastic syndrome, acute myelogenous leukemia and colorectal cancer.

In some embodiments, a processor or computational algorithm may aid in the assessment of a likelihood of a subject having cancer, such as squamous cell carcinoma, exhibiting a clinically beneficial response to treatment with an ERK inhibitor. For example, one or more steps of methods or systems described herein may be implemented in hardware. Alternatively, one or more steps may be implemented in software stored in, for example, one or more memories or other computer readable medium and implemented on one or more processors. As is known, the processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, a remote server (e.g. the cloud), or other storage medium, as is also known. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc. A computer system may be involved in one or more of sample collection, sample processing, data analysis, expression profile assessment, calculation of weighted probabilities, calculation of baseline probabilities, comparison of a weighted probability to a reference level and/or control sample, determination of a subject's absolute or increased probability, generating a report, and reporting results to a receiver.

A client-server, relational database architecture can be used in embodiments of the disclosure. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers), workstations, or mobile computing devices (e.g., a tablets or smart phones) on which users run applications, as well as example output devices as disclosed herein. Client computers may rely on server computers for resources, such as files, devices, and even processing power. In some embodiments of the disclosure, the server computer handles all of the database functionality. The client computer can have software that handles all the front-end data management and can also receive data input from users.

In some embodiments, the computer system is connected to an analysis system by a network connection. The computer system may be understood as a logical apparatus that can read instructions from media and/or a network port, which can optionally be connected to server having fixed media. The system can include a CPU, disk drives, optional input devices such as keyboard and/or mouse, and optional monitor. Data communication can be achieved through the indicated communication medium to a server at a local or a remote location. The communication medium can include any means of transmitting and/or receiving data. For example, the communication medium can be a network connection, a wireless connection, or an internet connection. Such a connection can provide for communication over the World Wide Web. In some embodiments, a physical report is generated and delivered to a receiver.

In some embodiments, there is provided a computer readable medium encoded with computer executable software that includes instructions for a computer to execute functions associated with the identified biomarkers. Such computer system may include any combination of such codes or computer executable software, depending upon the types of evaluations desired to be completed. The system can have code for calculating a weighted probability of ERK inhibitor responsiveness, and optionally for calculating an aggregated probability based on a plurality of weighted probabilities. In some embodiments, the weighted probability of ERK inhibitor responsiveness is increased if a squamous cell carcinoma cell (1) overexpresses one or more MAPK pathway genes and/or one or more RAS-ERK feedback regulators and/or one or more of AREG, CDH3, COL17A1 , EGFR, HIF1A, ITGB1, KRT1 , KRT9, NRG1 , SLC16A1 , SLC22A1 and VEGFA, (2) underexpresses one or more of DCUN1D1, PIK3CA, PRKCI, SOX2 and TP63, or (3) comprises copy number amplification of at least one MAPK pathway gene. The weighted probability of ERK inhibitor responsiveness may be decreased if a squamous cell carcinoma cell (1) underexpresses one or more MAPK pathway genes and/or one or more RAS-ERK feedback regulators and/or one or more of AREG , CDH3, COL17A1, EGFR, HIF1A, ITGB1, KRT1 , KRT9, NRG1 , SLC16A1 , SLC22A1 and VEGFA, (2) overexpresses one or more of DCUN1D1, PIK3CA, PRKCI, SOX2 and TP63, or (3) does not comprise copy number amplification of at least one MAPK pathway gene. A squamous cell carcinoma cell may express predictors of both sensitivity and resistance. In calculating a weighted probability, the computer system or computational algorithm may consider the expression of 2 or more, 3 or more, 4 or more, 5 or more, 6 or more, 7 or more, 8 or more, 9 or more, 10 or more, 15 or more, or 20 or more biomarkers. For example, expression levels of two or more biomarkers selected from CDK4, CDK6, CRAF, EGFR, ERK1 , CCND1, KRAS, ERK2, HRAS, DUSP2, DUSP4, DUSP5 , DUSP6, SPRY2 , SPRY4 and SPRED1 can be used to generate an expression profile. In calculating a weighted probability, the computer system or computational algorithm may consider the amplification status of 1 or more, 2 or more, 3 or more, 4 or more, or 5 or more biomarkers. For example, the amplification status of at least one biomarker selected from CDK4, CDK6, CRAF, EGFR, ERK 1 , CCND1, KRAS, ERK2, and HRAS can be used to generate a copy number status. The system can further comprise code for conducting genetic analysis based on specific panel(s) of biomarkers chosen. The system can also have code for one or more of the following: conducting, analyzing, organizing, or reporting the results, as described herein. The system can also have code for generating a report. In some embodiments, the test subject may be designated as having a high probability of exhibiting a clinically beneficial response to treatment with an ERK inhibitor if the weighted probability corresponds to at least about 0.55, at least about 0.6, at least about 0.65, at least about 0.7, at least about 0.75, at least about 0.8, at least about 0.85, at least about 0.9, at least about 0.95, or at least about 0.99. In some embodiments, the test subject may be designated as having a low probability of exhibiting a clinically beneficial response to treatment with an ERK inhibitor if the weighted probability corresponds to less than about 0.45, less than about 0.4, less than about 0.35, less than about 0.3, less than about 0.25, less than about 0.2, less than about 0.15, less than about 0.1, less than about 0.05, less than about 0.01.

In some embodiments, there is provided a computer readable medium encoded with computer executable software that includes instructions for a computer to execute functions associated with the identified biomarkers. Such computer system may include any combination of such codes or computer executable software, depending upon the types of evaluations desired to be completed. The system can have code for calculating a weighted probability of ERK inhibitor responsiveness, and optionally for calculating an aggregated probability based on a plurality of weighted probabilities. In some embodiments, the weighted probability of ERK inhibitor responsiveness is increased if a cancer cell (1) overexpresses at least one gene located at chromosome 11q13.3-13.4 and/or (2) comprises copy number amplification of at least one gene located at chromosome 11q13.3-13.4. The weighted probability of ERK inhibitor responsiveness may be decreased if a cancer cell (1) underexpresses at least one gene located at chromosome 11q13.3-13.4 and/or (2) does not comprise copy number amplification of at least one gene located at chromosome 11q13.3-13.4. The weighted probability may further be adjusted based on one or more MAPK pathway genes and/or one or more RAS-ERK feedback regulators as discussed herein above. A cancer cell may express predictors of both sensitivity and resistance. In calculating a weighted probability, the computer system or computational algorithm may consider the expression of 1 or more, 2 or more, 3 or more, 4 or more, 5 or more, 6 or more, 7 or more, 8 or more, 9 or more, 10 or more, 15 or more, or 20 or more biomarkers. For example, expression levels of one or more biomarkers selected from CCND1, CTTN, FADD, ORAOV 1, ANO1, PPFIA1 and SHANK2 can be used to generate an expression profile. In calculating a weighted probability, the computer system or computational algorithm may consider the amplification status of 1 or more, 2 or more, 3 or more, 4 or more, or 5 or more biomarkers. For example, the amplification status of at least one biomarker selected from CCND1, CTTN, FADD, ORAOV1, ANO1 , PPFIA1 and SHANK2 can be used to generate a copy number status. The system can further comprise code for conducting genetic analysis based on specific panel(s) of biomarkers chosen. In some embodiments, the at least one gene is CCND1 or ANO1 . In some embodiments, the at least one gene comprises CCND1 and ANO1. The system can also have code for one or more of the following: conducting, analyzing, organizing, or reporting the results, as described herein. The system can also have code for generating a report. In some embodiments, the test subject may be designated as having a high probability of exhibiting a clinically beneficial response to treatment with an ERK inhibitor if the weighted probability corresponds to at least about 0.55, at least about 0.6, at least about 0.65, at least about 0.7, at least about 0.75, at least about 0.8, at least about 0.85, at least about 0.9, at least about 0.95, or at least about 0.99. In some embodiments, the test subject may be designated as having a low probability of exhibiting a clinically beneficial response to treatment with an ERK inhibitor if the weighted probability corresponds to less than about 0.45, less than about 0.4, less than about 0.35, less than about 0.3, less than about 0.25, less than about 0.2, less than about 0.15, less than about 0.1, less than about 0.05, less than about 0.01.

The system may further comprise code for comparing a weighted probability to a baseline probability, a threshold value, and/or a reference level, and assigning a fold-baseline probability based on whether or not the baseline probability, threshold value, or reference level is exceeded. Assessing a weighted probability, threshold value, or reference level can be linked to at least one recommendation. Exceeding a weighted probability, threshold value, or reference level may be linked to a recommendation of treatment with an ERK inhibitor. In some embodiments, the baseline probability represents the average probability of a subject having cancer, such as squamous cell carcinoma, exhibiting a clinically beneficial response to treatment with an ERK inhibitor, either in general or for a specific population. In some embodiments, the baseline probability represents a pre-test likelihood that a particular subject will exhibit a clinically beneficial response to treatment with an ERK inhibitor before applying a method of the disclosure to determine a post-test risk. A weighted probability above a baseline probability may correspond to a specified fold-baseline probability, whatever the pre-test baseline for the subject may be. In some embodiments, the test subject may be designated as having a high probability of exhibiting a clinically beneficial response to treatment with an ERK inhibitor if the weighted probability corresponds to about or at least about 1.1-times, 1.2-times, 1.3-times, 1.4-times, 1.5-times, 1.8-times, 2-times, 2.5-times, 3-times, 4-times, 5-times, 6-times, 7-times, 8-times, 9-times, 10-times, 25-times, 50-times, or 100-times the baseline probability. In some embodiments, the test subject may be designated as having a low probability of exhibiting a clinically beneficial response to treatment with an ERK inhibitor if the weighted probability corresponds to about or at less than about 0.9-times, 0.8-times, 0.7-times, 0.6-times, 0.5-times, 0.4-times, 0.3-times, 0.2-times, 0.1-times, 0.05-times, 0.01-times the baseline probability.

After performing a calculation, a processor can provide the output, such as from a calculation, back to, for example, the input device or storage unit, to another storage unit of the same or different computer system, or to an output device. Output from the processor can be displayed by data display. A data display can be a display screen (for example, a monitor or a screen on a digital device), a print-out, a data signal (for example, a packet), an alarm (for example, a flashing light or a sound), a graphical user interface (for example, a webpage), or a combination of any of the above. In an embodiment, an output is transmitted over a network (for example, a wireless network) to an output device. The output device can be used by a user to receive the output from the data-processing computer system. After an output has been received by a user, the user can determine a course of action, or can carry out a course of action, such as a medical treatment when the user is medical personnel. In some embodiments, an output device is the same device as the input device. Example output devices include, but are not limited to, a telephone, a wireless telephone, a mobile phone, a PDA, a tablet, a flash memory drive, a light source, a sound generator, a fax machine, a computer, a computer monitor, a printer, an iPod, and a webpage. The user station may be in communication with a printer or a display monitor to output the information processed by the server.

It is envisioned that data relating to the present disclosure can be transmitted over a network or connections for reception and/or review by a receiver. The receiver can be but is not limited to an individual; the subject to whom the report pertains; a health care provider, manager, other healthcare professional, or other caretaker; an oncologist; a genetic counselor; a person or entity that performed and/or ordered the biomarker expression analysis; or a local or remote system for storing such reports (e.g. servers or other systems of a “cloud computing” architecture). In one embodiment, a computer-readable medium includes a medium suitable for transmission of a result of an analysis of a biological sample, such as analysis of one or more biomarkers. The medium can include a result regarding one or more biomarker expression level or amplification status of an individual, probability (such as fold-baseline probability) of having a cancer that is sensitive to treatment with an ERK inhibitor, and/or a treatment plan for the individual, wherein such a result is derived using the methods described herein.

In some embodiments, the subject or a third party (e.g. a heath care provider, health care manager, other health professional, or other caretaker) is alerted if a subject is designated as having a “high probability” of having a beneficial response to treatment with an ERK inhibitor. The analysis generated can be reviewed and further analyzed by a medical professional such as a managing doctor or licensed physician, or other third party. The medical professional or other third party can meet with the subject to discuss the results, analysis, and report. Information provided can include recommendations, such as treatment (e.g., with an ERK inhibitor or an alternative therapy).

In some embodiments, the method further comprises providing a recommendation for treatment based on an assessment of the likelihood that a subject having squamous cell carcinoma will exhibit a clinically beneficial response to treatment with an ERK inhibitor, such as designation as having high probability. A recommendation may form part of a report generated based on biomarker expression or copy number analysis, or may be made by a receiver on the basis of such report. A recommendation may be for further action on the part of the subj ect and/or for a third party, such as a heath care provider, health care manager, other health professional, or other caretaker. Recommendations may include, but are not limited to, treatment with an ERK inhibitor; continued monitoring of the subject; screening exams or laboratory tests that may further characterize the cancer; prescription and/or administration of one or more therapeutic agents that are not ERK inhibitors; discontinued therapy; and treatment with an alternative therapy, e.g. chemotherapy, immunotherapy, radiotherapy, or surgery.

In some embodiments, the disclosure provides a method of categorizing a squamous cell carcinoma status of a subject. The status of the subject may be categorized based on an expression profile of a biological sample from the subject. A cancer status may be categorized as likely sensitive to treatment with an ERK inhibitor or likely resistant to treatment with an ERK inhibitor. The likely sensitive categorization may be assigned to a squamous cell carcinoma having (1) overexpression of one or more MAPK pathway genes and/or one or more RAS-ERK feedback regulators and/or one or more of AREG, CDH3, COL17A1 , EGFR, HIF1A, ITGB1, KRT1, KRT9, NRG1 , SLC16A1 , SLC22A1 and VEGFA, (2) underexpression of one or more of DCUN1D1 , PIK3CA, PRKCI, SOX2 and TP63, and/or (3) copy number amplification of at least one MAPK pathway gene. A “likely resistant” categorization may be assigned to a cancer or cancer cell (1) having underexpression of one or more MAPK pathway genes and/or one or more RAS-ERK feedback regulators and/or one or more of AREG, CDH3, COL17A1 , EGFR, HIF1A, ITGB1, KRT1, KRT9, NRG1 , SLC16A1 , SLC22A1 and VEGFA, (2) having overexpression of one or more of DCUN1D1, PIK3CA, PRKCI, SOX2 and TP63, and/or (3) lacking copy number amplification of at least one MAPK pathway gene. A squamous cell carcinoma may have an expression profile having predictors of both sensitivity and resistance. In some embodiments, a squamous cell carcinoma may be categorized as sensitive if the total expression level of at least 2 or more, 3 or more, 4 or more, 5 or more, 6 or more, 7 or more, 8 or more, 9 or more, 10 or more, 15 or more, or 20 or more biomarkers selected from CDK4, CDK6, CRAF, EGFR, ERK1 , CCND1 , KRAS, ERK2, HRAS, DUSP2 , DUSP4 , DUSP5 , DUSP6, SPRY2 , SPRY4 and SPRED1 is greater than a corresponding reference level. In some embodiments, a squamous cell carcinoma may be categorized as sensitive if an average copy number of at least one of CDK4, CDK6, CRAF, EGFR, ERK1 , CCND1 , KRAS, ERK2, and HRAS is amplified, such as an average copy number of greater than 2, greater than 3, greater than 4, greater than 5, greater than 6, greater than 7, greater than 8, greater than 9, or greater than 10.

In some embodiments, a method of the disclosure provides a reference level above which at least two biomarkers must be expressed to be considered in assessing the likelihood of response to treatment with an ERK inhibitor. The biomarkers may be differentially expressed at least 1.1 fold, at least 1.2 fold, at least 1.3 fold, at least 1.4 fold, at least 1.5 fold, at least 2.0 fold, at least 2.25 fold, at least 2.5 fold, at least 2.75 fold, at least 3.0 fold, at least 3.5 fold, at least 4.0 fold, at least 5.0, or even at least 10 fold higher relative to a reference level to be considered in adjusting the likelihood of response. In some embodiments, the reference level is a numerical range of biomarker expression that is obtained from a statistical sampling from a population of individuals having squamous cell carcinoma that has low sensitivity, such as resistance, to treatment with an ERK inhibitor. In some embodiments, the reference level is a numerical range of biomarker expression that is obtained from a statistical sampling from a population of individuals having cancer that is sensitive to treatment with an ERK inhibitor. The reference level may be a numerical range of biomarker expression that is obtained from a statistical sampling from a population of individuals having cancer, e.g., the same cancer as the test subject. In some embodiments, the reference level is derived by comparison of sensitive and resistant populations. As used herein, low sensitivity to an ERK inhibitor refers to a disease condition that progresses after treatment with an ERK inhibitor. In some examples, low sensitivity to an ERK inhibitor is characterized by tumor growth inhibition of less than 60% following treatment with an ERK inhibitor. A disease condition that responds to treatment with an ERK inhibitor is one that exhibits a therapeutically beneficial response, such as regression or stabilization of a tumor, in response to treatment with an ERK inhibitor. In some examples, tumor growth inhibition of greater than 75% is indicative of a response to treatment with an ERK inhibitor.

In some embodiments, the disclosure provides a method of categorizing a squamous cell carcinoma status of a subject. The status of the subject may be categorized based on an expression profile of a biological sample from the subject. A cancer status may be categorized as likely sensitive to treatment with an ERK inhibitor or likely resistant to treatment with an ERK inhibitor. The likely sensitive categorization may be assigned to a cancer or cancer cell having (1) overexpresses at least one gene located at chromosome 11q13.3-13.4 and/or (2) copy number amplification of at least one gene located at chromosome 11q13.3-13.4. The categorization may further consider an expression profile and/or copy number profile of one or more MAPK pathway genes and/or one or more RAS-ERK feedback regulators as discussed herein above. A cancer may have an expression profile having predictors of both sensitivity and resistance. In some embodiments, a cancer may be categorized as sensitive if the total expression level of at least 1 or more, 2 or more, 3 or more, 4 or more, 5 or more, 6 or more, 7 or more, 8 or more, 9 or more, 10 or more, 15 or more, or 20 or more biomarkers selected from CCND1, CTTN, FADD, ORAOV 1 , ANO1, PPFIA1 and SHANK2 is greater than a corresponding reference level. In some embodiments, a cancer may be categorized as sensitive if an average copy number of at least one of CCND1, CTTN, FADD, ORAOV1, ANO1 , PPFIA1 and SHANK2 is amplified, such as an average copy number of greater than 2, greater than 3, greater than 4, greater than 5, greater than 6, greater than 7, greater than 8, greater than 9, or greater than 10.

In some embodiments, a method of the disclosure provides a reference level above which at least two biomarkers must be expressed to be considered in assessing the likelihood of response to treatment with an ERK inhibitor. The biomarkers may be differentially expressed at least 1.1 fold, at least 1.2 fold, at least 1.3 fold, at least 1.4 fold, at least 1.5 fold, at least 2.0 fold, at least 2.25 fold, at least 2.5 fold, at least 2.75 fold, at least 3.0 fold, at least 3.5 fold, at least 4.0 fold, at least 5.0, or even at least 10 fold higher relative to a reference level to be considered in adjusting the likelihood of response. In some embodiments, the reference level is a numerical range of biomarker expression that is obtained from a statistical sampling from a population of individuals having a particular cancer that has low sensitivity, such as resistance, to treatment with an ERK inhibitor. In some embodiments, the reference level is a numerical range of biomarker expression that is obtained from a statistical sampling from a population of individuals having a cancer that is sensitive to treatment with an ERK inhibitor. The reference level may be a numerical range of biomarker expression that is obtained from a statistical sampling from a population of individuals having cancer, e.g., the same cancer as the test subject. In some embodiments, the reference level is derived by comparison of sensitive and resistant populations. As used herein, low sensitivity to an ERK inhibitor refers to a disease condition that progresses after treatment with an ERK inhibitor. In some examples, low sensitivity to an ERK inhibitor is characterized by tumor growth inhibition of less than 60%for example, in a PDX modelfollowing treatment with an ERK inhibitor. A disease condition that responds to treatment with an ERK inhibitor is one that exhibits a therapeutically beneficial response, such as regression or stabilization of a tumor, in response to treatment with an ERK inhibitor. In some examples, tumor growth inhibition of greater than 75% is indicative of a response to treatment with an ERK inhibitor. Published criteria for evaluating treatment with an ERK inhibitor, such as the Response Evaluation Criteria in Solid Tumors (RECIST) criteria, may be used to evaluate a solid tumor. According to the RECIST criteria, a complete response (CR) is evidenced by disappearance of all target lesions; a partial response (PR) is evidenced by at least a 30% decrease in the sum of the longest diameter (LD) of target lesions, taking as reference the baseline sum LD; a stable disease (SD) is evidenced by neither sufficient shrinkage to qualify for PR nor sufficient increase to qualify for PD, taking as reference the smallest sum LD since the treatment started; and progressive disease (PD) is evidenced by at least a 20% increase in the sum of the LD of target lesions, taking as reference the smallest sum LD recorded since the treatment started or the appearance of one or more new lesions. In some examples, a disease condition is classified as responsive to treatment with an ERK inhibitor if categorized in accordance with the RECIST criteria as a CR, PR or SD in response to treatment with an ERK inhibitor. A disease condition that is resistant to treatment may be classified as a PD by the RECIST criteria.

In a further embodiment, the present disclosure provides a method of treating a cancer condition, such as squamous cell carcinoma, comprising administering an effective dose of an ERK inhibitor. The ERK inhibitor may be effective in one or more of inhibiting proliferation of cancer cells, inhibiting invasion or metastasis of cancer cells, killing cancer cells, increasing the sensitivity of cancer cells to treatment with a second antitumor agent and reducing severity or incidence of symptoms associated with the presence of cancer cells. In some embodiments, said method comprises administering to the cancer cells a therapeutically effective amount of an ERK inhibitor. In some embodiments, the administration takes place in vitro. In other embodiments, the administration takes place in vivo.

An ERK inhibitor suitable for use in the subject methods can be selected from a variety of types of molecules. For example, the ERK inhibitor can be a biological or chemical compound, such as a simple or complex organic or inorganic molecule, peptide, peptido mimetic, protein (e.g., antibody), liposome, or a polynucleotide (e.g., small interfering RNA, microRNA, antisense, aptamer, ribozyme, or triple helix). Some exemplary classes of chemical compounds suitable for use in the subject methods are detailed in the sections below. An ERK inhibitor for use in the present disclosure can be any ERK inhibitor that is known in the art, and can include any chemical entity that, upon administration to a subject, results in inhibition of ERK in the subject. Optionally, an ERK inhibitor for use in the treatment of squamous cell carcinoma is a small molecule. As used herein, the term “small molecule” refers to a low molecular weight organic compound, such as a compound having a molecular weight of less than 800 g/mol.

The term “ERK inhibitor” as used herein refers to compounds capable of fully or partially reducing or inhibiting ERK signaling activity. Inhibition may be effective at the transcriptional level, for example by preventing or reducing or inhibiting mRNA synthesis of key members of the ERK signaling pathway, such as MEK1, MEK2, ERK1 and/or ERK2 mRNA. In some examples, said ERK inhibitor inhibits one or more of MEK1, MEK2, ERK1 or ERK2 kinase activity. Inhibition of ERK can be achieved by a variety of mechanisms, including, but not limited to, binding directly to ERK1 or ERK2, binding directly to MEK1 or MEK2, or inhibiting expression of the ERK or MEK genes.

Any component of the ERK pathway is a potential therapeutic target for inhibition in accordance with the present disclosure. The mechanism of inhibition may be at the genetic level (e.g., interference with transcription or translation) or at the protein level (e.g., binding, competition). Because of their converging function, specific inhibition of MEK1/2 or ERK1/2 is expected to effectively intercept a wide variety of upstream mitogenic signals. Preferably, the ERK inhibitor is a specific inhibitor that either acts on MEK1/2 or ERK1/2 at the genetic level or protein level. Either or both approaches may be used in accordance with the present disclosure. For example, an inhibitor may be utilized that interferes with expression of ERK1 and/or ERK2, or which sequesters ERK1 and/or ERK2 in the cytoplasm of the cell, preventing nuclear translocation.

Exemplary ERK inhibitors include, but are not limited to: ulixertinib, BVD-523 (BioMed Discoveries); RG7842, GDC-0094, GDC-0994 (Array BioPharma, Genentech); CC-90003 (Celgene Corp); LTT-462 (Novartis AG); ASN-007 (Asana BioSciences); AMO-01 (AMO Pharma); KO-947 (Kura Oncology); AEZS-134, AEZS-131, AEZS-140 (AEterna Zentaris); AEZS-136, AEZS-132, D-87503 (AEterna Zentaris); KIN-2118, KIN-4050 analogs (Kinentia Biosciences); RB-1, RB-3 (IRCCS San Raffaele); SCH-722984, SCH-772984 (Merck & Co); MK-8353, SCH-900353 (Merck & Co); FR-180204 (Astellas Pharma); IDN-5491, hyperforin trimethoxybenzoate (Indena SpA); and ERK1-2067, ERK1-23211, ERK1-624 (H Lee Moffitt Cancer Center). In some embodiments, the ERK inhibitor is selected from SCH772984, GDC-0994, CC-90003, BVD-523 and KO-947. Preferably, the ERK inhibitor is KO-947.

In some examples, the ERK inhibitor is a compound selected from

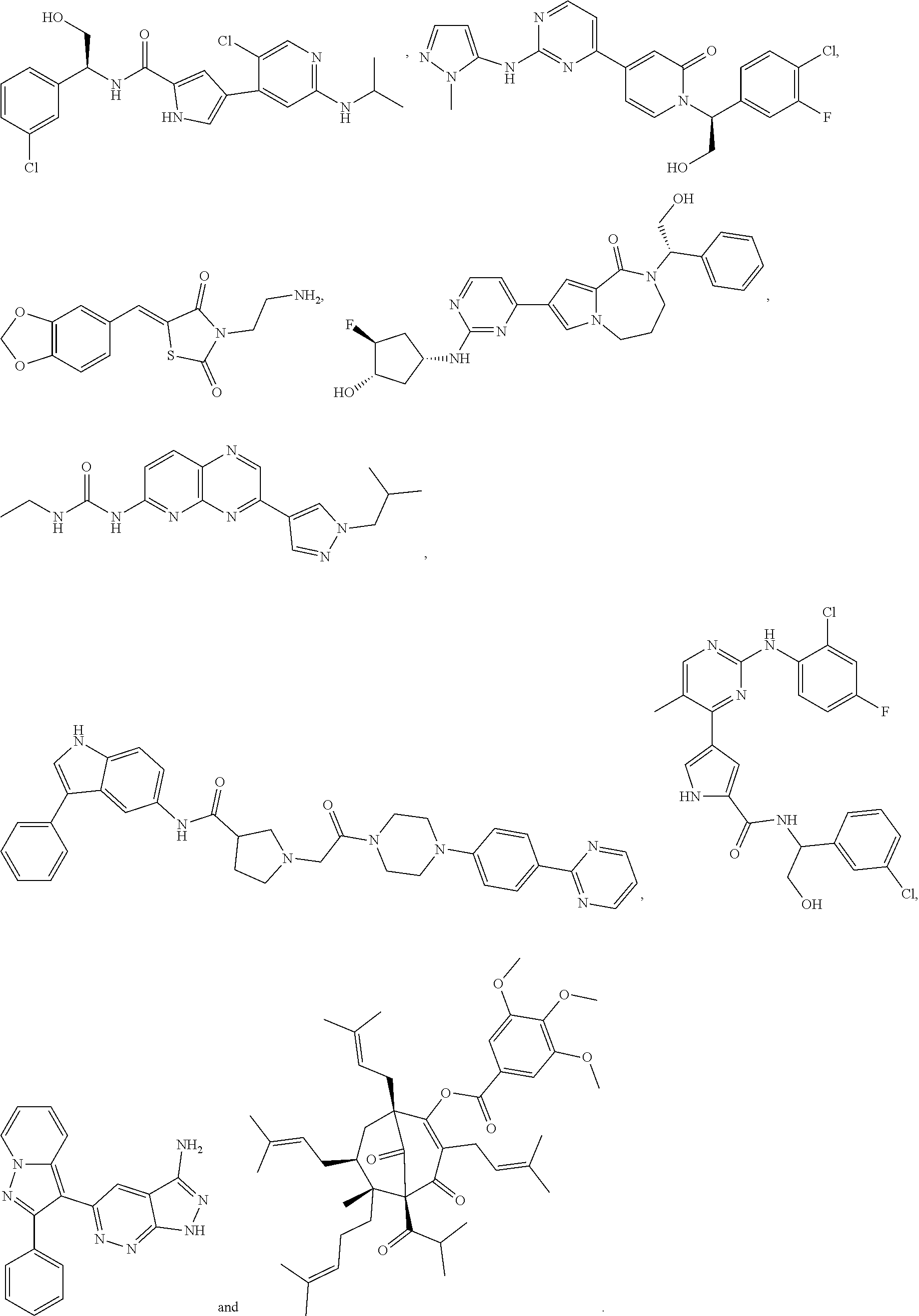

Examples of ERK inhibitors that may be used in accordance with the disclosure include, but are not limited to, Raf-1 inhibitors, such as GW5074, BAY 43-9006, and ISIS 5132 (Lackey, K. et al., Bioorg. Med. Chem. Lett., 2000, 10:223-226; Lyons, J. F. et al., Endocrine-related Cancer, 2001, 8:219-225; and Monia, B. P. et al., Nat. Med., 1996, 2(6):668-675, respectively); and MEK1/2 inhibitors, such as PD98059, PD184352, U0126 (Dudley D. T. et al., Proc. Natl. Acad. Sci. USA, 1995, 92:7686-7689; Sepolt-Leopold J. S. et al., Nat. Med., 1999, 5:810-816; and Favata M. F. et al., J. Biol. Chem., 273:18623-18632, respectively). A series of 3-cyano-4-(phenoxyanilo)quinolines with MEK inhibitory activity has also been developed by Wyeth-Ayerst (Zhang N. et al., Bioorg. Med. Chem. Lett., 2000, 10:2825-2828). Several resorcylic acid lactones having inhibitor activity toward MEK have been isolated from microbial extracts. For example, RO 09-2210, isolated from fungal broth FC2506, and L-783,277, purified from organic extracts of Phoma sp. (ATCC 74403), are competitive with ATP, and the MEK1 inhibition is reversible (Williams D. H. et al., Biochemistry, 1998, 37:9579-9585; and Zhao A. et al., J. Antibiot., 1999, 52:1086-1094). Imidazolium trans-imidazoledimethyl sulfoxide-tetrachlororuthenate (NAMI-A) is a ruthenium-containing inhibitor of the phosphorylation of MEK, the upstream activator of ERK (Pintus G. et al., Eur. J. Biochem., 2002, 269:5861-5870). In some examples, the ERK inhibitor is selected from the group consisting of BVD-523, FR 180204, MK-8353 (SCH900353), pluripotin, SCH772984, VX-11e (ERK-11e; TCS ERK 11e), SL327, hypericin, purvalanol, PD173074, GW5074, BAY 43-9006, AG99, CAY10561, ISIS 5132, apigenin, SP600125, SU4984, SB203580, PD169316, K0947, GDC0994, and AG1478. Other inhibitors include, but are not limited to, chromone and flavone type inhibitors; PD 98059 (Runden E et al, J Neurosci 1998, 18(18) 7296-305); PD0325901 (Pfizer); Selumetinib, a selective MEK inhibitor (AstraZeneca/ Array BioPharma, also known as AZD6244); ARRY-438162 (Array BioPharma); PD198306 (Pfizer); PD0325901 (Pfizer); AZD8330 (AstraZeneca/Array Biopharma, also called ARRY-424704); PD 184352 (Pfizer, also called CI-1040); PD 184161 (Pfizer); a-[Amino[(4-aminophenyl)thio]methylene]-2-(trifluoromethyl)benzeneacetonitrile (SL327);1,4-Diamino-2,3-dicyano-1,4-bis(2-aminophenylthio)butadiene; U0126 (Kohno & Pouyssegur (2003) Prog. Cell. Cyc. Res. 5: 219-224); GW 5074 (Santa Cruz Biotechnology); BAY 43-9006 (Bayer, Sorafenib); RO 09-2210 (Roche, Williams et al, Biochemistry. 1998 Jun 30;37(26):9579-85); FR 1 80204 (Ohori, M. et al. (2005) Biochem. Biophys. Res. Comm. 336: 357-363) ; 3-(2-aminoethyl)-5-))4- ethoxyphenyl)methylene)-2,4-thiazolidinedione (PKI-ERK-005) (Chen, F. et al. (2006) Bioorg. Med. Chem. 16:6281- 6288. 171. Hancock, CN. et al. (2005) J. Med. Chem. 48: 4586- 4595); CAY10561 (CAS 933786-58-4; Cayman Chemical); GSK 120212; RDEA1 19 (Ardea Biosciences); XL518; and ARRY-704 (AstraZeneca).

Other ERK inhibitors and their syntheses have been described in US 5,525,625, US 2003/0060469, US 2004/0048861, US 2004/0082631, WO 98/43960, WO 99/01426, WO 00/41505, WO 00/42002, WO 00/42003, WO 00/41994, WO 00/42022, WO 00/42029, WO 00/68201, WO 01/68619, WO 02/06213, WO 03/077855 and WO 2005/23251. Optionally, the ERK inhibitor is selected from the group consisting of selumetinib, U0126, PD98059, PD0325901, AZD8330 (ARRY-42704), CI-1040 (PD 184352), and PD318088. Preferably, the ERK inhibitor is a compound described in WO/2015051341, the disclosure of which is incorporated by reference herein.

In certain embodiments, the present disclosure provides an ERK inhibitor which is a compound of Formula I:

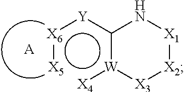

wherein:

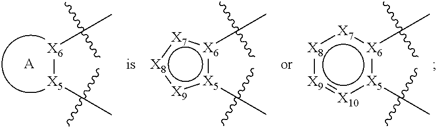

X₁ is C═O, C═S, SO, SO₂, or PO₂⁻; Y is CR₅; W is N or C;

X₂ is NR₁ or CR₁R₁′ and X₃ is null, CR₃R₃′ or C═O; or X₂-X₃ is R₁C═CR₃ or R₁C═N or N═CR₃ or NR₁₂—CR₁₁═CR₃;

X₄ is N or CR₄; X₅ is N or C; X₆ is N or C; X₇ is O, N, NR₇₂ or CR₇₁; X₈ is O, N, NR₈₂ or CR₈₁; X₉ is O, N, NR₂₂ or CR₂₁; X₁₀ is O, N, NR₉₂ or CR₉₁;

R₁ is —C₁₋₁₀alkyl, —C₂₋₁₀kenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —C₁₋₁₀alkyl-C₃₋₁₀aryl, —C₁₋₁₀alkyl-C₁₋₁₀hetaryl, —C₁₋₁₀alkyl-C₃₋₁₀cycloalkyl, —C₁₋₁₀alkyl-C₁₋₁₀heterocyclyl, —C₂₋₁₀alkenyl-C₃₋₁₀aryl, —C₂₋₁₀alkenyl-C₁₋₁₀hetaryl, —C₂₋₁₀alkenyl-C₃₋₁₀cycloalkyl, —C₂₋₁₀alkenyl-C₁₋₁₀heterocyclyl, —C₂₋₁₀alkynyl-C₃₋₁₀aryl, —C₂₋₁₀alkynyl-C₁₋₁₀hetaryl, —C₂₋₁₀alkynyl-C₃₋₁₀cycloalkyl, —C₂₋₁₀alkynyl-C₁₋₁₀heterocyclyl, —C₁₋₁₀heteroalkyl-C₃₋₁₀aryl, —C₁₋₁₀heteroalkyl-C₁₋₁₀hetaryl, —C₁₋₁₀heteroalkyl-C₃₋₁₀cycloalkyl, —C₁₋₁₀heteroalkyl-C₁₋₁₀heterocyclyl, —C₁₋₁₀alkoxy-C₃₋₁₀aryl, —C₁₋₁₀alkoxy-C₁₋₁₀hetaryl, —C₁₋₁₀alkoxy-C₃₋₁₀cycloalkyl, —C₁₋₁₀alkoxy-C₁₋₁₀heterocyclyl, —C₃₋₁₀aryl-C₁₋₁₀alkyl, —C₃₋₁₀aryl-C₂₋₁₀alkenyl, —C₃₋₁₀aryl-C₂₋₁₀alkynyl, —C₃₋₁₀aryl-C₃₋₁₀hetaryl, —C₃₋₁₀aryl-C₃₋₁₀cycloalkyl, —C₃₋₁₀aryl-C₁₋₁₀heterocyclyl, —C₁₋₁₀hetaryl-C₁₋₁₀alkyl, —C₁₋₁₀hetaryl-C₂₋₁₀alkenyl, —C₁₋₁₀hetaryl-C₂₋₁₀alkynyl, —C₃₋₁₀hetaryl-C₃₋₁₀aryl, —C₁₋₁₀hetaryl-C₃₋₁₀cycloalkyl, —C₁₋₁₀hetaryl-C₁₋₁₀heterocyclyl, —C₃₋₁₀cycloalkyl-C₁₋₁₀alkyl, —C₃₋₁₀cycloalkyl-C₂₋₁₀alkenyl, —C₃₋₁₀cycloalkyl-C₂₋₁₀alkynyl, —C₃₋₁₀cycloalkyl-C₃₋₁₀aryl, —C₃₋₁₀cycloalkyl-C₁₋₁₀hetaryl, —C₃₋₁₀cycloalkyl-C₁₋₁₀heterocyclyl, —C₁₋₁₀heterocyclyl-C₁₋₁₀alkyl, —C₁₋₁₀heterocyclyl-C₂₋₁₀alkenyl, —C₁₋₁₀heterocyclyl-C₂₋₁₀alkynyl, —C₁₋₁₀heterocyclyl-C₃₋₁₀aryl, —C₁₋₁₀heterocyclyl-C₁₋₁₀hetaryl, or —C₁₋₁₀heterocyclyl-C₃₋₁₀cycloalkyl, each of which is unsubstituted or substituted by one or more independent R₁₀ or R₁₁ sub stituents;

R₁′ is hydrogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —C₁₋₁₀alkyl-C₃₋₁₀aryl, —C₁₋₁₀alkyl-C₁₋₁₀hetaryl, —C₁₋₁₀alkyl-C₃₋₁₀cycloalkyl, —C₁₋₁₀alkyl-C₁₋₁₀heterocyclyl, —C₂₋₁₀alkenyl-C₃₋₁₀aryl, —C₂₋₁₀alkenyl-C₁₋₁₀hetaryl, —C₂₋₁₀alkenyl-C₃₋₁₀cycloalkyl, —C₂₋₁₀alkenyl-C₁₋₁₀heterocyclyl, —C₂₋₁₀alkynyl-C₃₋₁₀aryl, —C₂₋₁₀alkynyl-C₁₋₁₀hetaryl, —C₂₋₁₀alkynyl-C₃₋₁₀cycloalkyl, —C₂₋₁₀alkynyl-C₁₋₁₀heterocyclyl, —C₁₋₁₀heteroalkyl-C₃₋₁₀aryl, —C₁₋₁₀heteroalkyl-C₁₋₁₀hetaryl, —C₁₋₁₀heteroalkyl-C₃₋₁₀cycloalkyl, —C₁₋₁₀heteroalkyl-C₁₋₁₀heterocyclyl, —C₁₋₁₀alkoxy-C₃₋₁₀aryl, —C₁₋₁₀alkoxy-C₁₋₁₀hetaryl, —C₁₋₁₀alkoxy-C₃₋₁₀cycloalkyl, —C₁₋₁₀alkoxy-C₁₋₁₀heterocyclyl, —C₃₋₁₀aryl-C₁₋₁₀alkyl, —C₁₋₁₀aryl-C₂₋₁₀alkenyl, —C₃₋₁₀aryl-C₂₋₁₀alkynyl, —C₃₋₁₀aryl-C₃₋₁₀hetaryl, —C₃₋₁₀aryl-C₃₋₁₀cycloalkyl, —C₃₋₁₀aryl-C₁₋₁₀heterocyclyl, —C₁₋₁₀hetaryl-C₁₋₁₀alkyl, —C₁₋₁₀hetaryl-C₂₋₁₀alkenyl, —C₁₋₁₀hetaryl-C₂₋₁₀alkynyl, —C₃₋₁₀hetaryl-C₃₋₁₀aryl, —C₁₋₁₀hetaryl-C₃₋₁₀cycloalkyl, —C₁₋₁₀hetaryl-C₁₋₁₀heterocyclyl, —C₃₋₁₀cycloalkyl-C₁₋₁₀alkyl, —C₃₋₁₀cycloalkyl-C₂₋₁₀alkenyl, —C₃₋₁₀cycloalkyl-C₂₋₁₀alkynyl, —C₃₋₁₀cycloalkyl-C₃₋₁₀aryl, —C₃₋₁₀cycloalkyl-C₁₋₁₀hetaryl, —C₃₋₁₀cycloalkyl-C₁₋₁₀heterocyclyl, —C₁₋₁₀heterocyclyl-C₁₋₁₀alkyl, —C₁₋₁₀heterocyclyl-C₂₋₁₀alkenyl, —C₁₋₁₀heterocyclyl-C₂₋₁₀alkynyl, —C₁₋₁₀heterocyclyl-C₃₋₁₀aryl, —C₁₋₁₀heterocyclyl-C₁₋₁₀hetaryl, or —C₁₋₁₀heterocyclyl-C₃₋₁₀cycloalkyl, each of which is unsubstituted or substituted by one or more independent R₁₀ or R₁₁ sub stituents;

R₂₁ is hydrogen, halogen, —OH, —CF₃, —OCF₃, —OR³¹, —NR³¹R³², —C(O)R³¹, —CO₂R³¹, —C(═O)NR³¹, —NO₂, —CN, —S(O)₀₋₂R³¹, —SO₂NR³¹R³², —NR³¹C(═O)R³², —NR³¹C(═O)OR³², —NR³¹C(═O)NR³²R³³, —NR³¹S(═O)₀₋₂R³², —C(═S)OR³¹, —C(═O)SR³¹, —NR³¹C(═NR³²)NR³²R³³, NR³¹C(═NR³²)OR³³, —NR³¹C(═NR³²)SR³³, —OC(═O)OR³³, —OC(═O)NR³¹R³²,—OC(═O)SR³¹, —SC(═O)SR³¹, —P(O)OR⁻OR³², —SC(═O)NR³¹R³², -L-C₂₋₁₀alkenyl, -L-C₂₋₁₀alkynyl, -L-C₁₋₁₀heteroalkyl, -L-C₃₋₁₀aryl, -L-C₁₋₁₀hetaryl, -L-C₃₋₁₀cycloalkyl, -L-C₁₋₁₀heterocyclyl, -L-C₁₋₁₀alkyl-C₃₋₁₀aryl , -L-C₁₋₁₀alkyl-C₁₋₁₀hetaryl, -L-C₁₋₁₀alkyl-C₃₋₁₀cycloalkyl, -L-C₁₋₁₀alkyl-C₁₋₁₀heterocyclyl, -L-C₂₋₁₀alkenyl-C₃₋₁₀aryl, -L-C₂₋₁₀alkenyl-C₁₋₁₀hetaryl, -L-C₂₋₁₀alkenyl-C₃₋₁₀cycloalkyl, -L-C₂₋₁₀alkenyl-C₁₋₁₀heterocyclyl, -L-C₂₋₁₀alkynyl-C₃₋₁₀aryl, -L-C₂₋₁₀alkynyl-C₁₋₁₀hetaryl, -L-C₂₋₁₀alkynyl-C₃₋₁₀cycloalkyl, -L-C₂₋₁₀alkynyl-C₁₋₁₀heterocyclyl, -L-C₁₋₁₀heteroalkyl-C₃₋₁₀aryl, -L-C₁₋₁₀heteroalkyl-C₁₋₁₀hetaryl, -L-C₁₋₁₀heteroalkyl-C₃₋₁₀cycloalkyl, -L-C₁₋₁₀heteroalkyl-C₁₋₁₀heterocyclyl, -L-C₁₋₁₀alkoxy-C₃₋₁₀aryl, -L-C₁₋₁₀alkoxy-C₁₋₁₀hetaryl, -L-C₁₋₁₀alkoxy-C₃₋₁₀cycloalkyl, -L-C₁₋₁₀alkoxy-C₁₋₁₀heterocyclyl, -L-C₃₋₁₀aryl-C₁₋₁₀a;lu;. -L-C₃₋₁₀aryl-C₂₋₁₀alkenyl, -L-C₃₋₁₀aryl-C₂₋₁₀alkynyl, -L-C₃₋₁₀aryl-C₁₋₁₀hetaryl, -L-C₃₋₁₀aryl-C₃₋₁₀cycloalkyl, -L-C₃₋₁₀aryl-C₁₋₁₀heterocyclyl, -L-C₁₋₁₀hetaryl-C₁₋₁₀alkyl, -L-C₁₋₁₀hetaryl-C₂₋₁₀alkenyl, -L-C₁₋₁₀hetaryl-C₂₋₁₀alkynyl, -L-C₁₋₁₀hetaryl-C₃₋₁₀aryl, -L-C₁₋₁₀hetaryl-C₃₋₁₀cycloalkyl, -L-C₁₋₁₀hetaryl-C₁₋₁₀heterocyclyl, -L-C₃₋₁₀cycloalkyl-C₁₋₁₀alkyl, -L-C₃₋₁₀cycloalkyl-C₂₋₁₀alkenyl, -L-C₃₋₁₀cycloalkyl-C₂₋₁₀alkynyl, -L-C₃₋₁₀cycloalkyl-C₃₋₁₀aryl, -L-C₃₋₁₀cycloalkyl-C₁₋₁₀hetaryl, -L-C₃₋₁₀cycloalkyl-C₁₋₁₀heterocyclyl, -L-C₁₋₁₀heterocyclyl-c₁₋₁₀alkyl, -L-C₁₋₁₀heterocyclyl-C₂₋₁₀alkenyl, -L-C₁₋₁₀heterocyclyl-C₂₋₁₀alkynyl, -L-C₁₋₁₀heterocyclyl-C₃₋₁₀aryl, -L-C₁₋₁₀heterocyclyl-C₁₋₁₀hetaryl, or -L-C₁₋₁₀heterocyclyl-C₃₋₁₀cycloalkyl, each of which is unsubstituted or substituted by one or more independent R₁₂ substituents;

R₂₂ is hydrogen, —OH, —CF₃, —C(O)R³¹, —CO₂R³¹, —C(═O)NR³¹, —S(O)₀₋₂R³¹, —C(═S)OR³¹, —C(═O)SR³¹, -L-C₁₋₁₀alkyl, -L-C₂₋₁₀alkenyl, -L-C₂₋₁₀alkynyl, -L-C₁₋₁₀heteroalkyl, -L-C₃₋₁₀aryl, -L-C₁₋₁₀hetaryl, -L-C₃₋₁₀cycloalkyl, -L-C₁₋₁₀heterocyclyl, -L-C₁₋₁₀alkyl-C₃₋₁₀aryl, -L-C₁₋₁₀alkyl-C₁₋₁₀hetaryl, -L-C₁₋₁₀alkyl-C₃₋₁₀cycloalkyl, -L-C₁₋₁₀alkyl-C₁₋₁₀heterocyclyl, -L-C₂₋₁₀alkenyl-C₃₋₁₀aryl, -L-C₂₋₁₀alkenyl-C₁₋₁₀hetaryl, -L-C₂₋₁₀alkenyl-C₃₋₁₀cycloalkyl, -L-C₂₋₁₀alkenyl-C₁₋₁₀heterocyclyl, -L-C₂₋₁₀alkynyl-C₃₋₁₀aryl, -L-C₂₋₁₀alkynyl-C₁₋₁₀hetaryl, -L-C₂₋₁₀alkynyl-C₃₋₁₀cycloalkyl, -L-C₂₋₁₀ alkynyl-C₁₋₁₀heterocyclyl, -L-C₁₋₁₀heteroalkyl-C₃₋₁₀aryl, -L-C₁₋₁₀heteroalkyl-C₁₋₁₀hetaryl, -L-C₁₋₁₀heteroalkyl-C₃₋₁₀cycloalkyl, -L-C₁₋₁₀heteroalkyl-C₁₋₁₀heterocyclyl, -L-C₁₋₁₀alkoxy-C₃₋₁₀aryl, -L-C₁₋₁₀alkoxy-C₁₋₁₀hetaryl, -L-C₁₋₁₀alkoxy-C₃₋₁₀cycloalkyl, -L-C₁₋₁₀alkoxy-C₁₋₁₀heterocyclyl, -L-C₃₋₁₀aryl-C₁₋₁₀alkyl, -L-C₃₋₁₀aryl-C₂₋₁₀alkenyl, -L-C₃₋₁₀aryl-C₂₋₁₀alkynyl, -L-C₃₋₁₀aryl-C₁₋₁₀hetaryl, -L-C₃₋₁₀aryl-C₃₋₁₀cycloalkyl, -L-C₃₋₁₀aryl-C₁₋₁₀heterocyclyl, -L-C₁₋₁₀hetaryl-C₁₋₁₀alkyl, -L-C₁₋₁₀hetaryl-C₂₋₁₀alkenyl, -L-C₁₋₁₀hetaryl-C₂₋₁₀alkynyl, -L-C₁₋₁₀hetaryl-C₃₋₁₀aryl, -L-C₁₋₁₀hetaryl-C₃₋₁₀cycloalkyl, -L-C₁₋₁₀hetaryl-C₁₋₁₀heterocyclyl, -L-C₃₋₁₀cycloalkyl-C₁₋₁₀alkyl, -L-C₃₋₁₀cycloalkyl-C₂₋₁₀alkenyl, -L-C₃₋₁₀cycloalkyl-C₂₋₁₀alkynyl, -L-C₃₋₁₀cycloalkyl-C₃₋₁₀aryl, -L-C₃₋₁₀cycloalkyl-C₁₋₁₀hetaryl, -L-C₃₋₁₀cycloalkyl-C₁₋₁₀heterocyclyl, -L-C₁₋₁₀heterocyclyl-C₁₋₁₀alkyl, -L-C₁₋₁₀heterocyclyl-C₂₋₁₀alkenyl, -L-C₁₋₁₀heterocyclyl-C₂₋₁₀alkynyl, -L-C₁₋₁₀heterocyclyl-C₃₋₁₀aryl, -L-C₁₋₁₀heterocyclyl-C₁₋₁₀hetaryl, or -L-C₁₋₁₀heterocyclyl-C₃₋₁₀cycloalkyl, each of which is unsubstituted or substituted by one or more independent R₁₂ substituents;

L is a bond, —O—, —N(R³¹)—, —S(O)₀₋₂—, —C(═O)—, —C(═O)O—, —OC(═O)—, —C(═O)N(R³¹)—, —N(R³¹)C(═O)—, —NR³¹C(═O)O—, —NR³¹C(═O)NR³²—, —NR³¹S(O)₀₋₂—, —S(O)₀₋₂N(R³¹)—, —C(═S)O—, —C(═O)S—, —NR³¹C(═NR³²)NR³², —NR³¹C(═NR³²)O—, —NR³¹C(═NR³²)S—, —OC(═O)—, —OC (═O)NR³¹—, —OC(═O)S—, —SC(═O)S—, —P(O)OR³¹O—, —SC(═O)NR³¹—;

each of R₃, R₃′ and R₄ is independently hydrogen, halogen, —OH, —CF₃, —OCF₃, —OR³¹, —NR³¹R³², —C(O)R³¹, —CO₂R⁻, —C(═O)NR³¹, —NO₂, —CN, —S(O)₀₋₂R³¹, —SO₂NR³¹R³², —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³²R³³, —NR³¹C(═NR³²)OR³³, —NR³¹C(═NR³²)SR³³, —OC(═O)OR³³, —OC(═O)NR³¹R³², —OC(═O)SR³¹, —SC(═O)SR³¹, —P(O)OR³¹OR³², —SC (═O)NR³¹R³², —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —C₁₋₁₀alkyl-C₃₋₁₀aryl, —C₁₋₁₀alkyl-C₁₋₁₀hetaryl, —C₁₋₁₀alkyl-C₃₋₁₀cycloalkyl, —C₁₋₁₀alkyl-C₁₋₁₀heterocyclyl, —C₂₋₁₀alkenyl-C₃₋₁₀aryl, —C₂₋₁₀alkenyl-C₁₋₁₀hetaryl, —C₂₋₁₀alkenyl-C₃₋₁₀cycloalkyl, —C₂₋₁₀alkenyl-C₁₋₁₀heterocyclyl, —C₂₋₁₀alkynyl-C₃₋₁₀aryl, —C₂₋₁₀alkynyl-C₁₋₁₀hetaryl, —C₂₋₁₀alkynyl-C₃₋₁₀cycloalkyl, —C₂₋₁₀alkynyl-C₁₋₁₀heterocyclyl, —C₁₋₁₀heteroalkyl-C₃₋₁₀aryl, —C₁₋₁₀heteroalkyl-C₁₋₁₀hetaryl, —C₁₋₁₀heteroalkyl-C₃₋₁₀cycloalkyl, —C₁₋₁₀heteroalkyl-C₁₋₁₀heterocyclyl, —C₁₋₁₀alkoxy-C₃₋₁₀aryl, —C₁₋₁₀alkoxy-C₁₋₁₀hetaryl, —C₁₋₁₀alkoxy-C₃₋₁₀cycloalkyl, —C₁₋₁₀alkoxy-C₁₋₁₀heterocyclyl, —C₃₋₁₀aryl-C₁₋₁₀alkyl, —C₃₋₁₀aryl-C₂₋₁₀alkenyl, —C₃₋₁₀aryl-C₂₋₁₀alkynyl, —C₃₋₁₀aryl-C₃₋₁₀hetaryl, —C₃₋₁₀aryl-C₃₋₁₀cycloalkyl, —C₃₋₁₀aryl-C₁₋₁₀heterocyclyl, —C₁₋₁₀hetaryl-C₁₋₁₀alkyl, —C₁₋₁₀hetaryl-C₂₋₁₀alkenyl, —C₁₋₁₀hetaryl-C₂₋₁₀alkynyl, —C₃₋₁₀hetaryl-C₃₋₁₀aryl, —C₁₋₁₀hetaryl-C₃₋₁₀cycloalkyl, —C₁₋₁₀hetaryl-C₁₋₁₀heterocyclyl, —C₃₋₁₀cycloalkyl-C₁₋₁₀alkyl, —C₃₋₁₀cycloalkyl-C₂₋₁₀alkenyl, —C₃₋₁₀cycloalkyl-C₂₋₁₀alkynyl, —C₃₋₁₀cycloalkyl-C₃₋₁₀aryl, —C₃₋₁₀cycloalkyl-C₁₋₁₀hetaryl, —C₃₋₁₀cycloalkyl-C₁₋₁₀heterocyclyl, —C₁₋₁₀heterocyclyl-C₁₋₁₀alkyl, —C₁₋₁₀heterocyclyl-C₂₋₁₀alkenyl, —C₁₋₁₀heterocyclyl-C₂₋₁₀alkynyl, —C₁₋₁₀heterocyclyl-C₃₋₁₀aryl, —C₁₋₁₀heterocyclyl-C₁₋₁₀hetaryl, or —C₁₋₁₀heterocyclyl-C₃₋₁₀cycloalkyl, each of which is unsubstituted or substituted by one or more independent R₁₃ substituents; or R₃′ is —OR⁶, —NR⁶R³⁴, —S(O)₀₋₂R⁶, —C(═O)R⁶, —C(═O)OR⁶, —OC(═O)R⁶, —C(═O)N(R³⁴)R⁶, or —N(R³⁴)C(═O)R⁶, wherein R⁶ together with R³⁴ can optionally form a heterocyclic ring; or R₃′ is —OR⁶, —NR⁶R³⁴, —S(O)₀₋₂R⁶, —C(═O)R⁶, —C(═O)OR⁶, —OC(═O)R⁶, —C(═O)N(R³⁴)R⁶, or —N(R³⁴)C(═O)R⁶, wherein R⁶ together with R³⁴ can optionally form a heterocyclic ring;

each of R₅, R₇₁, R₈₁ and R₉₁ is independently hydrogen, halogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —OH, —CF₃, —OCF₃, —OR³¹, —NR³¹R³², —C(═O)R³¹, —CO₂R³¹, —C(═O)NR³¹, —NO₂, —CN, —S(O)₀₋₂R³¹, —SO₂NR³¹, —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³²R³³, —NR³¹C(═NR³²)OR³³, —NR³¹C(═NR³²)SR³³, —OC(═O)OR³³, —OC(═O)NR³¹R³², —OC(═O)SR³¹, —SC(═O)SR³¹, —P(O)OR⁻OR³², or —SC(═O)NR³¹NR³²;

R₆ is hydrogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —C₁₋₁₀alkyl-C₃₋₁₀aryl, —C₁₋₁₀alkyl-C₁₋₁₀hetaryl, —C₁₋₁₀alkyl-C₃₋₁₀cycloalkyl, —C₁₋₁₀alkyl-C₁₋₁₀heterocyclyl, —C₂₋₁₀alkenyl-C₃₋₁₀aryl, —C₂₋₁₀alkenyl-C₁₋₁₀hetaryl, —C₂₋₁₀alkenyl-C₃₋₁₀cycloalkyl, —C₂₋₁₀alkenyl-C₁₋₁₀heterocyclyl, —C₂₋₁₀alkynyl-C₃₋₁₀aryl, —C₂₋₁₀alkynyl-C₁₋₁₀hetaryl, —C₂₋₁₀alkynyl-C₃₋₁₀cycloalkyl, —C₂₋₁₀alkynyl-C₁₋₁₀heterocyclyl, —C₁₋₁₀heteroalkyl-C₃₋₁₀aryl, —C₁₋₁₀heteroalkyl-C₁₋₁₀hetaryl, —C₁₋₁₀heteroalkyl-C₃₋₁₀cycloalkyl, —C₁₋₁₀heteroalkyl-C₁₋₁₀heterocyclyl, —C₁₋₁₀alkoxy-C₃₋₁₀aryl, —C₁₋₁₀alkoxy-C₁₋₁₀hetaryl, —C₁₋₁₀alkoxy-C₃₋₁₀cycloalkyl, —C₁₋₁₀alkoxy-C₁₋₁₀heterocyclyl, —C₃₋₁₀aryl-C₁₋₁₀alkyl, —C₃₋₁₀aryl-C₂₋₁₀alkenyl, —C₃₋₁₀aryl-C₂₋₁₀alkynyl, —C₃₋₁₀aryl-C₃₋₁₀hetaryl, —C₃₋₁₀aryl-C₃₋₁₀cycloalkyl, —C₃₋₁₀aryl-C₁₋₁₀heterocyclyl, —C₁₋₁₀hetaryl-C₁₋₁₀alkyl, —C₁₋₁₀hetaryl-C₂₋₁₀alkenyl, —C₁₋₁₀hetaryl-C₂₋₁₀alkynyl, —C₃₋₁₀hetaryl-C₃₋₁₀aryl, —C₁₋₁₀hetaryl-C₃₋₁₀cycloalkyl, —C₁₋₁₀hetaryl-C₁₋₁₀heterocyclyl, —C₃₋₁₀cycloalkyl-C₁₋₁₀alkyl, —C₃₋₁₀cycloalkyl-C₂₋₁₀alkenyl, —C₃₋₁₀cycloalkyl-C₂₋₁₀alkynyl, —C₃₋₁₀cycloalkyl-C₃₋₁₀aryl, —C₃₋₁₀cycloalkyl-C₁₋₁₀hetaryl, —C₃₋₁₀cycloalkyl-C₁₋₁₀heterocyclyl, —C₁₋₁₀heterocyclyl-C₁₋₁₀alkyl, —C₁₋₁₀heterocyclyl-C₂alkenyl, —C₁₋₁₀heterocyclyl-C₂₋₁₀alkynyl, —C₁₋₁₀heterocyclyl-C₃₋₁₀aryl, —C₁₋₁₀heterocyclyl-C₁₋₁₀hetaryl, or —C₁₋₁₀heterocyclyl-C₃₋₁₀cycloalkyl, each of which is unsubstituted or substituted by one or more independent R₁₄ or R₁₅ substituents;

each of R₇₂, R₈₂ and R₉₂ is independently hydrogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —OH, —CF₃, —C(O)R³¹, —CO₂R³¹, —C(═O)NR³¹, —S(O)₀₋₂R³¹, —C(═S)OR³¹, —C(═O)SR³¹;

each of R₁₀and R₁₄ is independently —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, optionally substituted by one or more independent R₁₁ substituents;

each of R₁₁, R_{12,} R₁₃ and R₁₅ is independently hydrogen, halogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —OH, —CF₃, —OCF₃, —OR³¹, —NR³¹R³², —C(O)R³¹, —CO₂R³¹, —C(═O)NR³¹, —NO₂, —CN, —S(O)₀₋₂R³¹, —SO₂NR³¹R³², —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³²R³³, —NR³¹C(═NR³²)OR³³, —NR³¹C(═NR³²)SR³³, —OC(═O)OR³³, —OC(═O)NR³¹R³², —OC(═O)SR³¹, —SC(═O)SR³¹, —P(O)OR⁻OR³², or —SC(═O)NR³¹NR³²;

each of R³¹, R³², R³³ and R³⁴ is independently hydrogen, halogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, or wherein R³¹ together with R³² form a heterocyclic ring;

wherein ring A comprises one or more heteroatoms selected from N, O, or S; and

wherein if X₇ is O or X₂-X₃ is R₁C═CR₃, ring A comprises at least two heteroatoms selected from N, O, or S; and

wherein if X₂-X₃ is R₁C═N, at least one of X₇ or X₉ is not N.

In some embodiments of Formula I, X₁ is C═O, X₂ is NR₁ or CR₁R₁′ and X₃ is CR₃R₃′. In some embodiments, X₁ is C═O, X₂ is R₁, and X₃ is C═O. In some embodiments, W is C, Y is CR₅, X₄ is CR₄, X₅ is C and X₆ is C. In some embodiments, X₇ is NH, X₈ is N and X₉ is CR₂₁. In some embodiments, X₇ is CR₇₁, X₈ is N and X₉ is NR₂₂. In some embodiments, X₁ is C═O, X₂ is R₁ or CR₁R_{1′}, X₃ is CR₃R₃′, W is C, Y is CR₅, X₄ is N or CR₄, X₅ is N or C, X₆ is C, X₇ is NR₇₂ or CR₇₁, X₈ is N, and X₉ is NR₂₂ or CR₂₁. In some embodiments, X₁ is C═O, X₂ is NR₁, X₃ is CR₃R₃′, W is C, Y is CR₅, X₄ is CR₄, X₅ is C, X₆ is C, X₇ is NR₇₂, X₈ is N, and X₉ 1S CR₂₁.

In some embodiments of Formula I, X₁ is C═O, X₂ is NR₁ or CR₁R₁′, X₃ is CR₃R₃′ or C═O, W is C, Y is CR₅, X₄ is N or CR₄, X₅ is N or C, X₆ is C, X₇ is N or NR₇₂ or CR₇₁, X₈ is N or CR₈₁, X₉ is NR₂₂ or CR₂₁, and X₁₀ is N or CR₉₁;

R₁ is -C₁₋₁₀alkyl, —C₃₋₁₀aryl, —C₁₋₁₀hetaryl, —C₃₋₁₀cycloalkyl, —C₁₋₁₀heterocyclyl, —C₁₋₁₀alkyl-C₃₋₁₀aryl, —C₁₋₁₀alkyl-C₁₋₁₀hetaryl, —C ₁₀alkyl-C₃₋₁₀cycloalkyl, —C₁₋₁₀alkyl-C₁₋₁₀heterocyclyl, —C₃₋₁₀cycloalkyl-C ₁₋₁₀alkyl, —C₃₋₁₀cycloalkyl-C₃₋₁₀aryl, —C₃₋₁₀ cycloalkyl-C₁₋₁₀hetaryl, —C₃₋₁₀cycloalkyl-C₁₋₁₀heterocyclyl, —C ₁₋₁₀heterocyclyl -C₁₋₁₀alkyl, —C₁₋₁₀heterocyclyl-C₃₋₁₀aryl, —C₁₋₁₀heterocyclyl-C₁₋₁₀hetaryl, or —C₁₋₁₀heterocyclyl-C₃₋₁₀cycloalkyl, each of which is unsubstituted or substituted by one or more independent R₁₀ or R_{H} substituents;

R₁′ is hydrogen, —C₁₋₁₀alkyl, —C₃₋₁₀aryl, —C₁₋₁₀hetaryl, —C₃₋₁₀cycloalkyl, —C₁₋₁₀heterocyclyl, —C₁₋₁₀alkyl-C₃₋₁₀aryl, —C₁₋₁₀alkyl-C₁₋₁₀hetaryl, —C₁₋₁₀alkyl-C₃₋₁₀cycloalkyl, —C₁₋₁₀alkyl-C₁₋₁₀heterocyclyl, —C₃₋₁₀cycloalkyl-C₁₋₁₀alkyl, —C₃₋₁₀cycloalkyl-C₃₋₁₀aryl, —C₃₋₁₀cycl oalkyl-C₁₋₁₀hetaryl, —C₃₋₁₀cycloalkyl-C₁₋₁₀heterocyclyl, —C₁₋₁₀heterocyclyl-C₁₋₁₀alkyl, —C₁₋₁₀heterocyclyl-C₃₋₁₀aryl, —C₁₋₁₀heterocyclyl-C₁₋₁₀hetaryl, or —C₁₋₁₀heterocyclyl-C₃₋₁₀cycloalkyl, each of which is unsubstituted or substituted by one or more independent R₁₀ or R₁₁ substituents;

R₂₁ is halogen, —OH, —CF₃, —OCF₃, —OR³¹, —NR³¹R³², —C(O)R³¹, —CO₂R⁻, —C(═O)NR³¹, —NO₂, —CN, —S(O)₀₋₂R³¹, —NR³¹C(═O)R³², -L-C₁₋₁₀alkyl, -L-C₂₋₁₀alkenyl, -L-C₂₋₁₀alkynyl, -L-C₁₋₁₀heteroalkyl, -L-C₃₋₁₀aryl, -L-C₁₋₁₀hetaryl, -L-C₃₋₁₀cycloalkyl, or -L-C₁₋₁₀heterocyclyl, each of which is unsubstituted or substituted by one or more independent R₁₂ substituents;

R₂₂ is —OH, —CF₃, —C(O)R³¹, —CO₂R³¹, —C(═O)NR³¹, —S(O)₀₋₂R³¹, -L-C₁₋₁₀alkyl, -L-C₂₋₁₀alkenyl, -L-C₂₋₁₀alkynyl, -L-C₁₋₁₀heteroalkyl, -L-C₃₋₁₀aryl, -L-C₁₋₁₀hetaryl, -L-C₃₋₁₀cycloalkyl, or -L-C₁₋₁₀heterocyclyl, each of which is unsubstituted or substituted by one or more independent R₁₂ substituents;

L is a bond, —O—, —N(R³¹)—, —S(O)₀₋₂—, —C(═O)—, —C(═O)O—, —OC(═O)—, —C(═O)N(R³¹)—, —N(R³¹)C(═O)—, —NR³¹C(═O)O—, —NR³¹C(═O)NR³²—, —NR³¹S(O)₀₋₂—, or —S(O)₀₋₂N(R³¹)—;

each of R₃, R₃′ and R₄ is independently hydrogen, halogen, —OH, —CF₃, —OCF₃, —OR³¹, —NR³¹R³², —C(O)R³¹, —CO₂R³¹, —C(O)NR³¹, —NO₂, —CN, —S(O)₀₋₂R³¹, —C₁₋₁₀alkyl, C₂₋₁₀alkenyl, —C₂₋₁₀alkynyl, - L-C₁₋₁₀heteroalkyl, —C₃₋₁₀aryl, —C₁₋₁₀hetaryl, —C₃₋₁₀cycloalkyl, or —C₁₋₁₀heterocyclyl, each of which is unsubstituted or substituted by one or more independent R₁₃ substituents; or R₃′ is —OR⁶, —NR⁶R³⁴, —S(O)₀₋₂R⁶, —C(═O)R⁶, —C(═O)OR⁶, —OC(═O)R⁶, —C(═O)N(R³⁴)R⁶, or —N(R³⁴)C(═O)R⁶, wherein R⁶ together with R³⁴ can optionally form a heterocyclic ring;

each of R₅, R₇₁, and R₈₁ is independently hydrogen, halogen, —C₁₋₁₀alkyl —C₃₋₁₀aryl, —C₁₋₁₀hetaryl, —C₃₋₁₀cycloalkyl, —C₁₋₁₀heterocyclyl, —OH, —CF₃, —OCF₃, —OR³¹, —NR³¹R³², —C(O)R³¹, —CO₂R³¹, —C(═O)NR³¹, —NO₂, —CN, —S(O)₀₋₂R³¹ or —NR³¹C(═O)R³²;

R₆ is —C₁₋₁₀alkyl, —C₃₋₁₀aryl, —C₁₋₁₀hetaryl, —C₃₋₁₀cycloalkyl, —C₁₋₁₀heterocyclyl, —C₁₋₁₀alkyl-C₃₋₁₀aryl, C₁₋₁₀alkyl-C₁₋₁₀hetaryl, —C₁₋₁₀alkyl-C₃₋₁₀cycloalkyl, —C₁₋₁₀heterocyclyl, —C₁₋₁₀alkly-C₃₋₁₀cycloalkyl-C₁₋₁₀alkyl, —C₃₋₁₀cycloalkyl-C₃₋₁₀aryl, —C₃₋₁₀cycloalkyl-C₁₋₁₀hetaryl, —C₃₋₁₀cycloalkyl-C₁₋₁₀heterocyclyl, —C₁₋₁₀heterocyclyl-C₁₋₁₀alkyl, —C₁₋₁₀heterocyclyl-C₃₋₁₀aryl, —C₁₋₁₀heterocyclyl-C₁₋₁₀hetaryl, or —C₁₋₁₀heterocyclyl-C₃₋₁₀cycloalkyl, each of which is unsubstituted or substituted by one or more independent R₁₄ or R₁₅ substituents;

R₇₂ is hydrogen, —C₁₋₁₀alkyl, —C₃₋₁₀aryl, —C₁₋₁₀hetaryl, —C₃₋₁₀cycloalkyl, —C₁₋₁₀heterocyclyl, —OH, —CF₃, —C(O)R³¹, —CO₂R³¹, —C(═O)NR³¹, or —S(O)₀₋₂R³¹;

each of R₁₀and R₁₄ is independently —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, or —C₁₋₁₀heterocyclyl, optionally substituted by one or more independent R₁₁ substituents;

each of R₁₁, R_{12,} R₁₃ and R₁₅ is independently hydrogen, halogen, —C₁₋₁₀alkyl, —C₃₋₁₀aryl, —C₃₋₁₀cycloalkyl, —OH, —CF₃, —OCF₃, —OR³¹, —NR³¹R³², —C(O)R³¹, —CO₂R³¹, —C(═O)NR³¹, —NO₂, —CN, —S(O)₀₋₂R^{3l} or —NR³¹C(═O)R³²;

each of R³¹, R³² and R³⁴ is independently hydrogen, —C₁₋₁₀alkyl, —C₃₋₁₀aryl, or —C₃₋₁₀cycloalkyl, or wherein R³¹ together with R³² form a heterocyclic ring; and

wherein ring A comprises one or more heteroatoms selected from N, O, or S.

In some embodiments of Formula I, X₁ is C═O, X₂ is NR₁ or CR₁R₁′, X₃ is CR₃R₃^{′}, W is C, Y is CR₅, X₄ is N or CR₄, X₅ is N or C, X₆ is C, X₇ is NR₇₂ or CR₇₁, X₈ is N, X₉ is NR₂₁ or CR₂₁, and X₁₀ is N or CR₉₁;

R₁ is —C₁₋₁₀alkyl, —C₁₋₁₀heterocyclyl, —C₁₋₁₀alkyl-C₃₋₁₀aryl, —C₁₋₁₀alkyl-C₁₋₁₀hetaryl, —C₁₋₁₀alkyl-C₃₋₁₀cycloalkyl, —C₁₋₁₀alkyl-C₁₋₁₀heterocyclyl, —C₁₋₁₀heterocyclyl-C₁₋₁₀alkyl, or —C₁₋₁₀heterocyclyl-C₃₋₁₀aryl, each of which is unsubstituted or substituted by one or more independent R₁₀ or R₁₁ substituents;

R₁′ is hydrogen —C₁₋₁₀alkyl, —C₁₋₁₀heterocyclyl, —C₁₋₁₀alkyl-C₃₋₁₀aryl, —C₁₋₁₀alkyl-C₁₋₁₀hetaryl, —C₁₋₁₀alkyl-C₃₋₁₀cycloalkyk, —C₁₋₁₀alkyl-C₁₋₁₀heterocyclyl, —C₁₋₁₀heterocyclyl-C₁₋₁₀alkyl, or —C₁₋₁₀heterocyclyl-C₃₋₁₀aryl, each of which is unsubstituted or substituted by one or more independent R₁₀ or R₁₁ substituents;

R₂₁ is halogen, —OH, —CF₃, —OCF₃, —OR³¹, —NR³¹R³², —C(O)R³¹, —CO₂R³¹, _C(═O)NR³¹, —NO₂, —CN, —S(O)₀₋₂R³¹, —NR³¹C(═O)R³², -L-C₁₋₁₀alkyl, -L-C₃₋₁₀aryl, -L-C₁₋₁₀hetaryl, -L-C₃₋₁₀cycloalkyl, or -L-C₁₋₁₀heterocyclyl, each of which is unsubstituted or substituted by one or more independent R₁₂ substituents;

R₂₂ is —OH, —CF₃, —C(O)R³¹, —CO₂R³¹, —C(═O)NR³¹, —S(O)₀₋₂R³¹, -L-C₁₋₁₀alkyl, -L-C₃₋₁₀aryl, -L-C₁₋₁₀hetaryl, -L-C₃₋₁₀cycloalkyl, or -L-C₁₋₁₀heterocyclyl each of which is unsubstituted or substituted by one or more independent R₁₂ substituents;

L is a bond, —O—, —N(R³¹)—, —S(O)₀₋₂—, —C(═O)—, —C(═O)O—, —OC(═O)—, —C(═O)N(R³¹)—, or —N(R³¹)C(═O)—;

each of R₃, R₃′ and R₄ is independently hydrogen, halogen, —OH, —CF₃, —OCF₃, —OR³¹, —NR³¹R³², —C(O)R³¹, —CO₂R³¹, —C(O)NR³¹, —NO₂, —CN, —S(O)₀₋₂R³¹, —C₁₋₁₀alkyl, —C₂₋₁₀alkenyl, or —C₂₋₁₀alkynyl, each of which is unsubstituted or substituted by one or more independent R₁₃ substituents; or R₃′ is —OR⁶, —NR⁶R³⁴, —C(O)N(R³⁴)R⁶, or —N(R³⁴)C(═O)R⁶, wherein R⁶ together with R³⁴ can optionally form a heterocyclic ring;

each of R₅ and R₇₁ is independently hydrogen, halogen, —C₁₋₁₀alkyl, —C₃₋₁₀aryl, —C₃₋₁₀cycloalkyl, —OH, —CF₃, —OR³¹, —NR³¹R³², —C(O)R31, —CO₂R³¹, —C(═O)NR³¹, —NO₂, —CN, —S(O)₀₋₂, R³¹, or —NR³¹C(═O)R³²;

R₆ is —C₁₋₁₀alkyl, —C₁₋₁₀heterocyclyl, —C₁₋₁₀alkyl-C₃₋₁₀aryl, —C₁₋₁₀alkyl-C₁₋₁₀hetaryl, —C₁₋₁₀alkyl-C₃₋₁₀cycloalkyl, —C₁₋₁₀alkyl-C₁₋₁₀heterocyclyl, —C₁₋₁₀heterocyclyl-C₁₋₁₀alkyl, or —C₁₋₁₀heterocyclyl-C₃₋₁₀aryl, each of which is unsubstituted or substituted by one or more independent R₁₄ or R₁₅ substituents;

R₇₂ is hydrogen, —C₁₋₁₀alkyl, —C₃₋₁₀aryl, —C₃₋₁₀cycloalkyl, —OH, —CF₃, —C(O)R³¹, —CO₂R³¹, —C(═O)NR³¹, or —S(O)₀₋₂R³¹;

each of R₁₀and R₁₄ independently —C₁₋₁₀alkyl, —C₃₋₁₀aryl, —C₁₋₁₀hetaryl, —C₃₋₁₀cycloalkyl, or —C₁₋₁₀heterocyclyl, optionally substituted by one or more independent R₁ₗ substituents;

each of R₁₁, R₁₂, R₁₃ and R₁₅ is independently hydrogen, halogen, —C₁₋₁₀alkyl, —OH, —CF₃, —OR³¹, —NR³¹R³², —C(O)R³¹, —CO₂R³¹, —C(═O)NR³¹, —NO₂, —CN, —S(O)₀₋₂R³¹ or —NR³¹C(═O)R³²;

each of R³¹, R³² and R³⁴ is independently hydrogen or —C₁₋₁₀alkyl, or wherein R³¹ together with R³² form a heterocyclic ring; and

wherein ring A comprises one or more heteroatoms selected from N, O, or S.

In some embodiments of Formula I, X₁ is C═O, X₂ is NR₁, X₃ is CR₃R₃′, W is C, Y is CR₅, X₄ is CR₄, X₅ is C, X₆ is C, X₇ is NR₇₂, X₈ is N, X₉ is CR₂₁, and X₁₀ is N or CR₉₁;

R₁ is —C₁₋₁₀alkyl, —C₁₋₁₀heterocyclyl, —C₁₋₁₀alkyl-C₃₋₁₀aryl, —C₁₋₁₀heterocyclyl-C₁₋₁₀alkyl, or —C₁₋₁₀heterocyclyl-C₃₋₁₀aryl, each of which is unsubstituted or substituted by one or more independent R₁₀ or R₁₁ substituents;

R₂₁ is halogen, —OH, —CF₃, —OR³¹, —NR³¹R³², —C(O)R³¹, —CO₂R³¹, —C(═O)NR³¹, —NO₂, —CN, -L-C₁₋₁₀alkyl, -L-C₃₋₁₀aryl, -L-C₁₋₁₀hetaryl, -L-C₃₋₁₀cycloalkyl, or -L-C₁₋₁₀heterocyclyl, each of which is unsubstituted or substituted by one or more independent Rᵤ substituents;

L is a bond, —N(R³¹)—, —C(═O)N(R³¹)—, or —N(R³¹)C(═O)—;

each of R₃, R₃′ and R₄ is independently hydrogen, halogen, —OH, —CF₃, —OCF₃, —OR³¹, —NR³¹R³², —NO₂, —CN, —S(O)₀₋₂R³¹, —C₁₋₁₀alkyl, —C₂₋₁₀alkenyl, or —C₂₋₁₀alkynyl; or R₃′ is —OR⁶, —NR⁶R³⁴, —C(═O)N(R³⁴)R⁶, or —N(R³⁴)C(═O)R⁶, wherein R⁶ together with R³⁴ can optionally form a heterocyclic ring;

R₅ is hydrogen, halogen, or —C₁₋₁₀alkyl;

R₆ is —C₁₋₁₀alkyl, —C₁₋₁₀heterocyclyl, —C₁₋₁₀alkyl-C₃₋₁₀aryl, —C₁₋₁₀heterocyclyl-C₁₋₁₀alkyl, or —C₁₋₁₀heterocyclyl-C₃₋₁₀aryl, each of which is unsubstituted or substituted by one or more independent R₁₄ or R₁₅ substituents;

R₇₂ is hydrogen, —C₁₋₁₀alkyl, —OH, —CF₃, —C(O)R³¹, —CO₂R³¹, —C(═O)NR³¹, or —S(O)₀₋₂R³¹;

each of R₁₀and R₁₄ is independently —C₁₋₁₀alkyl, —C₃₋₁₀aryl, —C₁₋₁₀hetaryl, or —C₁₋₁₀heterocyclyl, optionally substituted by one or more independent R,, substituents;

each of R₁₁, R₁₂ and R₁₅ is independently hydrogen, halogen, —C₁₋₁₀alkyl, —OH, —CF₃, —OR³, —NR³¹R³², —NO₂, —CN, or —S(O)₀₋₂R³¹;

each of R³¹, R³² and R³⁴ is independently hydrogen or —C₁₋₁₀alkyl, or wherein R³¹ together with R³² form a heterocyclic ring; and

wherein ring A comprises one or more heteroatoms selected from N, O, or S.

In some embodiments of Formula I, X₁ is C═O, X₂ is NR₁, X₃ is CR₃R₃′, W is C, Y is CR₅, X₄ is CR₄, X₅ is C, X₆ is C, X₇ is NR₇₂, X₈ is N, X₉ is CR₂₁, and X₁₀ is N;

R₁ is —C₁₋₁₀alkyl, —C₁₋₁₀heterocyclyl, —C₁₋₁₀alkyl-C₃₋₁₀aryl, —C₁₋₁₀heterocyclyl-C₁₋₁₀alkyl, or —C₁₋₁₀heterocyclyl-C₃₋₁₀aryl, each of which is unsubstituted or substituted by one or more independent R₁₀ or R₁₁ substituents;

R₂₁ is halogen, —CN, -L-C₁₋₁₀alkyl, -L-C₃₋₁₀aryl, -L-C₁₋₁₀hetaryl, -L-C₃₋₁₀cycloalkyl, or -L-C₁₋₁₀heterocyclyl, each of which is unsubstituted or substituted by one or more independent R₁₂ substituents;

L is a bond, —N(R³¹), or —C(═O)N(R³¹)—; or R₃′ is —OR⁶ or —NR⁶R³⁴, wherein R⁶ together with R³⁴ can optionally form a heterocyclic ring;

each of R₃, R₃′ and R₄ is independently hydrogen, halogen, —OH, —CF₃, or —C₁₋₁₀alkyl; or R₃′ is —OR⁶ or —NR⁶R³⁴, wherein R⁶ together with R³⁴ can optionally form a heterocyclic ring;

R₅ is hydrogen;

R₆ is —C₁₋₁₀alkyl, —C₁₋₁₀heterocyclyl, —C₁₋₁₀alkyl-C₃₋₁₀aryl, —C₁₋₁₀heterocyclyl-C₁₋₁₀alkyl, or —C₁₋₁₀heterocyclyl-C₃₋₁₀aryl, each of which is unsubstituted or substituted by one or more independent R₁₄ or R₁₅ substituents;

R₇₂ is hydrogen, —C₁₋₁₀alkyl, —OH, —CF₃, —C(O)R³¹, CO₂R³¹, —C(═O)NR³¹, or —S(O)₀₋₂R³¹;

each of R₁₀and R₁₄ is independently C₁₋₁₀alkyl, —C₃₋₁₀aryl, —C₁₋₁₀hetaryl, or —C₁₋₁₀heterocyclyl, optionally substituted by one or more independent R₁₁ substituents;

each of R₁₁, R₁₂ and R₁₅ is independently hydrogen, halogen, —C₁₋₁₀alkyl, -OH or CF₃;

each of R³¹ and R³⁴ is independently hydrogen or —C₁₋₁₀alkyl; and

wherein ring A comprises one or more heteroatoms selected from N, O, or S.

In certain embodiments, the present disclosure provides an ERK inhibitor which is a compound of Formula I-A:

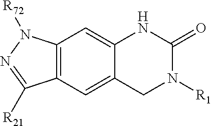

or a pharmaceutically acceptable salt or prodrug thereof, and wherein the substituents are as defined above.

In some embodiments of Formula I-A, R₁ is —C₁₋₁₀alkyl, —C₃₋₁₀aryl, —C₁₋₁₀hetaryl, —C₃₋₁₀cycloalkyl, —C₁₋₁₀heterocyclyl, —C₁₋₁₀alkyl-C₃₋₁₀aryl, —C₁₋₁₀alkyl-C₁₋₁₀hetaryl, —C₁₋₁₀alkyl-C₃₋₁₀cycloalkyl, —C₁₋₁₀alkyl-C₁₋₁₀heterocyclyl, —C₃₋₁₀cycloalkyl-C₁₋₁₀alkyl, —C₃₋₁₀cycloalkyl-C₃₋₁₀aryl, —C₃₋₁₀cycloalkyl-C₁₋₁₀hetaryl, —C₃₋₁₀cycloalkyl-C₁₋₁₀heterocyclyl, —C₁₋₁₀heterocyclyl-C₁₋₁₀alkyl, —C₁₋₁₀heterocyclyl-C₃₋₁₀aryl, —C₁₋₁₀heterocyclyl-C₁₋₁₀hetaryl, or —C₁₋₁₀heterocyclyl-C₃₋₁₀cycloalkyl, each of which is unsubstituted or substituted by one or more independent R₁₀ or R₁₁ substituents. In some embodiments, R₁ is —C₁₋₁₀alkyl, —C₁₋₁₀heterocyclyl, —C₁₋₁₀alkyl-C₃₋₁₀aryl, —C₁₋₁₀alkyl-C₁₋₁₀hetaryl, —C₁₋₁₀alkyl-C₃₋₁₀cycloalkyl, —C₁₋₁₀alkyl-C₁₋₁₀heterocyclyl, —C₁₋₁₀heterocyclyl-C₁₋₁₀alkyl, or —C₁₋₁₀heterocyclyl-C₃₋₁₀aryl, each of which is unsubstituted or substituted by one or more independent R₁₀ or R₁₁ substituents. In some embodiments, R₁ is —C₁₋₁₀alkyl, —C₁₋₁₀heterocyclyl, —C₁₋₁₀alkyl-C₃₋₁₀aryl, —C₁₋₁₀heterocyclyl-C₁₋₁₀alkyl, or —C₁₋₁₀heterocyclyl-C₃₋₁₀aryl, each of which is unsubstituted or substituted by one or more independent R₁₀ or R₁₁ substituents. In some embodiments, R₁ is —C₁₋₁₀heterocyclyl-C₁₋₁₀alkyl, unsubstituted or substituted by one or more independent R₁₀ or R₁₁ substituents.

In some embodiments of Formula I-A, R₂₁ is hydrogen, halogen, —OH, —CF₃, —OCF₃, —OR³¹, —NR³¹R³², —C(O)R³¹, —CO₂R³¹, —C(═O)NR³¹, —NO₂, —CN, —S(O)₀₋₂R³¹, —NR³¹C(═O)R³², -L-C₁₋₁₀alkyl, -L-C₂₋₁₀alkenyl, -L-C₂₋₁₀alkynyl, -L-C₁₋₁₀heteroalkyl, -L-C₃₋₁₀aryl, -L-C₁₋₁₀hetaryl, -L-C₃₋₁₀cycloalkyl, or -L-C₁₋₁₀heterocyclyl, each of which is unsubstituted or substituted by one or more independent R₁₂ substituents. In some embodiments, R₂₁ is halogen, —OH, —CF₃, —OCF₃, —OR³¹, —NR³¹R³², —C(O)R³¹, —CO₂R³¹, —C(O)NR³¹, —NO₂, —CN, —S(O)₀₋₂R³¹, —NR³¹C(═O)R³², -L-C₁₋₁₀alkyl, -L-C₃₋₁₀aryl, -L-C₁₋₁₀hetaryl, -L-C₃₋₁₀cycloalkyl, or -L-C₁₋₁₀heterocyclyl, each of which is unsubstituted or substituted by one or more independent R₁₂ substituents. In some embodiments, R₂₁ is halogen, —OH, —CF₃, —OR³¹, —NR³¹R³², —C(O)R³¹, —CO₂R³¹, —C(O)NR³¹, —NO₂, —CN, -L-C₁₋₁₀alkyl, -L-C₃₋₁₀aryl, -L-C₁₋₁₀hetaryl, -L-C₃₋₁₀cycloalkyl, or -L-C₁₋₁₀heterocyclyl, each of which is unsubstituted or substituted by one or more independent R₁₂ substituents. In some embodiments, R₂₁ is halogen, —CN, -L-C₁₋₁₀alkyl, -L-C₃₋₁₀aryl, -L-C₁₋₁₀hetaryl, -L-C₃₋₁₀cycloalkyl, or -L-C₁₋₁₀heterocyclyl, each of which is unsubstituted or substituted by one or more independent R₁₂ substituents.

In some embodiments of Formula I-A, R₂₁ is -L-C₁₋₁₀hetaryl unsubstituted or substituted by one or more independent R₁₂ substituents; wherein the C₁₋₁₀hetaryl of R₂₁ comprises one or more nitrogen atoms; each R₁₂ substituent, when present, is independently selected from the group consisting of —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —OH, —CF₃, —OCF₃, —OR³¹; wherein each R₃₁ is independently hydrogen or —C₁₋₁₀alkyl; L is a bond; and R₁ is —C₁₋₁₀alkyl-C₃₋₁₀aryl, —C₁₋₁₀alkyl-C₁₋₁₀hetaryl, —C₁₋₁₀heterocyclyl-C₁₋₁₀alkyl, or —C₁₋₁₀heterocyclyl-C₃₋₁₀aryl, unsubstituted or substituted by one or more independent R₁₀ or R₁₁ sub stituents.

In some embodiments of Formula I-A, R₂₁ is -L-C₁₋₁₀hetaryl unsubstituted or substituted by one or more independent R₁₂ substituents; wherein the C₁₋₁₀hetaryl of R₂₁ comprises one or more nitrogen atoms; each R₁₂ substituent, when present, is independently selected from the group consisting of C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —OH, —CF₃, —OCF₃, —OR³¹; wherein each R₃₁ is independently hydrogen or —C₁₋₁₀alkyl; L is a bond; and R₁ is

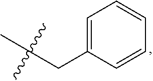 [SMILES: *Cc1ccccc1]

unsubstituted or substituted by one or more independent R₁₀ or R₁₁ substituents.

In some embodiments of Formula I-A, R₂₁ is -L-C₁₋₁₀hetaryl unsubstituted or substituted by one or more independent R₁₂ substituents; wherein the C₁₋₁₀hetaryl of R₂₁ comprises one or more nitrogen atoms; each R₁₂ substituent, when present, is independently selected from the group consisting of —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —OH, —CF₃, —OCF₃, —OR³¹; wherein each R₃₁ is independently hydrogen or —C₁₋₁₀alkyl; L is a bond; and R₁ is

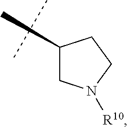

unsubstituted or substituted by one or more independent R₁₀ or R₁₁ substituents.

In some embodiments of Formula I-A, R₂₁ is -L-C₁₋₁₀hetaryl unsubstituted or substituted by one or more independent R₁₂ substituents; wherein the C₁₋₁₀hetaryl of R₂₁ comprises one or more nitrogen atoms; each R₁₂ substituent, when present, is independently selected from the group consisting of C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —OH, —CF₃, —OCF₃, —OR³¹; wherein each R₃₁ is independently hydrogen or —C₁₋₁₀alkyl; L is a bond; and R₁ is

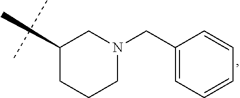

unsubstituted or substituted by one or more independent R₁₀ or R₁₁ substituents.

In some embodiments of Formula I-A, R₂₁ is -L-C₁₋₁₀hetaryl unsubstituted or substituted by one or more independent R₁₂ substituents; the C₁₋₁₀hetaryl of R₂₁ is selected from the group consisting of pyrazolyl, pyridinyl, pyrazinyl, pyrimidinyl, and pyridazinyl; each R₁₂ substituent, when present, is independently selected from the group consisting—Me, -Et, -i-Pr, -n-Pr, OH, -OMe, -OEt, -OPr; L is a bond; and R₁ is —C₁₋₁₀alkyl-C₃₋₁₀aryl, —C₁₋₁₀alkyl-C₁₋₁₀hetaryl, —C₁₋₁₀heterocyclyl-C₁₋₁₀alkyl, or —C₁₋₁₀heterocyclyl-C₃₋₁₀aryl, unsubstituted or substituted by one or more independent R₁₀ or R₁₁ substituents.

In some embodiments of Formula I-A, R₂₁ is -L-C₁₋₁₀hetaryl unsubstituted or substituted by one or more independent R₁₂ substituents; the C₁₋₁₀hetaryl of R₂₁ is selected from the group consisting of pyrazolyl, pyridinyl, pyrazinyl, pyrimidinyl, and pyridazinyl; each R₁₂ substituent, when present, is independently selected from the group consisting—Me, -Et, -i-Pr, -n-Pr, OH, -OMe, -OEt, -OPr; L is a bond; and R₁ is

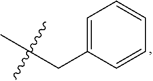 [SMILES: *Cc1ccccc1]

unsubstituted or substituted by one or more independent R₁₀ or R₁₁ sub stituents.

In some embodiments of Formula I-A, R₂₁ is -L-C₁₋₁₀hetaryl unsubstituted or substituted by one or more independent R₁₂ substituents; the C₁₋₁₀hetaryl of R₂₁ is selected from the group consisting of pyrazolyl, pyridinyl, pyrazinyl, pyrimidinyl, and pyridazinyl; each R₁₂ substituent, when present, is independently selected from the group consisting—Me, -Et, -i-Pr, -n-Pr, OH, -OMe, -OEt, -OPr; L is a bond; and R₁ is

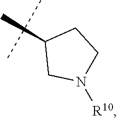

unsubstituted or substituted by one or more independent R₁₀ or R₁₁ sub stituents.

In some embodiments of Formula I-A, R₂₁ is -L-C₁₋₁₀hetaryl unsubstituted or substituted by one or more independent R₁₂ substituents; the C₁₋₁₀hetaryl of R₂₁ is selected from the group consisting of pyrazolyl, pyridinyl, pyrazinyl, pyrimidinyl, and pyridazinyl; each R₁₂ substituent, when present, is independently selected from the group consisting—Me, -Et, -i-Pr, -n-Pr, OH, -OMe, -OEt, -OPr; L is a bond; and R₁ is

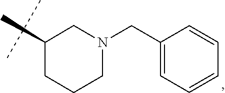

unsubstituted or substituted by one or more independent R₁₀ or R₁₁ sub stituents.

In some embodiments of Formula I-A, L is a bond, O, N(R³¹), S(O)₀₋₂, C(═O), —C(═O)O—, —OC(═O)—, —C(═O)N(R³¹)—, —N(R³¹)C(═O)—, —NR³¹C(═O)O—, —NR³¹C(═O)NR³²—, —NR³¹S(O)₀₋₂—, or —S(O)₀₋₂N(R³¹)—. In some embodiments, L is a bond, —O—, —N(R³¹)—, —S(O)₀₋₂—, —C(═O)—, —C(═O)O—, —OC(═O), —C(═O)N(R³¹), or —N(R³¹)C(═O)—. In some embodiments, L is a bond, —N(R³¹), —C(═O)N(R³¹)—, or —N(R³¹)C(═O). In some embodiments, L is a bond, —N(R³¹)—, or —C(═O)N(R³¹)—.

In some embodiments of Formula I-A, R₇₂ is hydrogen, —C₁₋₁₀alkyl, —C₃₋₁₀aryl, —C₃₋₁₀cycloalkyl, —C₁₋₁₀heterocyclyl, —OH, —CF₃, —C(O)R³¹, CO₂R³¹, —C(═O)NR³¹, or —S(O)₀₋₂R³¹. In some embodiments, R₇₂ is independently hydrogen, —C₁₋₁₀alkyl, —C₃₋₁₀aryl, —C₃₋₁₀cycloalkyl, —C(O)R³¹, —CO₂R³¹, —C(═O)NR³¹, or —S(O)₀₋₂R³¹. In some embodiments, R₇₂ is independently hydrogen or —C₁₋₁₀alkyl. In some embodiments, R₇₂ is independently hydrogen.

In some embodiments of Formula I-A, each of R₁₀ independently is —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, optionally substituted by one or more independent R₁₁ substituents. In some embodiments, each of R₁₀ is independently —C₁₋₁₀alkyl, —C₃₋₁₀aryl, —C₃₋₁₀cycloalkyl, or —C₁₋₁₀heterocyclyl, optionally substituted by one or more independent R₁₁ substituents. In some embodiments, each of R₁₀ is independently —C₁₋₁₀alkyl, —C₃₋₁₀aryl, —C₁₋₁₀hetaryl, or —C₁₋₁₀heterocyclyl, optionally substituted by one or more independent R₁₁ substituents.

In some embodiments of Formula I-A, each of R₁₁, R₁₂, and R₁₃ is independently hydrogen, halogen, —C₁₋₁₀alkyl, —C₃₋₁₀aryl, —C₃₋₁₀cycloalkyl, —OH, —CF₃, —OCF₃, —OR³¹, —NR³¹R³², —C(O)R³¹, —CO₂R³¹, —C(═O)NR³¹, —NO₂, —CN, —S(O)₀₋₂R³¹ or —NR³¹C(═O)R³². In some embodiments, each of R₁₁, R_{12,} and R₁₃ is independently hydrogen, halogen, —C₁₋₁₀alkyl, —OH, —CF₃, —OR³¹, —NR³¹R³², —C(O)R³¹, —CO₂R³¹, —C(═O)NR³¹, —NO₂, —CN, —S(O)₀₋₂R³¹ or —NR³¹C(═O)R³². In some embodiments, each of R₁₁, R₁₂, and R₁₃ is independently hydrogen, halogen, —C₁₋₁₀alkyl, —OH, —CF₃, —OR³, —NR³¹R³², —NO₂, —CN, or —S(O)₀₋₂R³¹. In some embodiments, each of R₁₁, R₁₂, and R₁₃ is independently hydrogen, halogen, —C₁₋₁₀alkyl, -OH or -CF₃

In some embodiments of Formula I-A, each of R³¹, R³², and R³³ is independently hydrogen, halogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, or wherein R³¹ together with R³² form a heterocyclic ring. In some embodiments, each of R³¹, R³², and R³³ is independently hydrogen, —C₁₋₁₀alkyl, —C₃₋₁₀aryl, or —C₃₋₁₀cycloalkyl, or wherein R³¹ together with R³² form a heterocyclic ring. In some embodiments, each of R³¹, R³², and R³³ is independently hydrogen or —C₁₋₁₀alkyl, or wherein R³¹ together with R³² form a heterocyclic ring. In some embodiments, each of R³¹, R³², and R³³ is independently hydrogen or —C₁₋₁₀alkyl.

In some embodiments of Formula I-A,

R₁ is-C₁₋₁₀alkyl, —C₃₋₁₀aryl, —C₁₋₁₀hetaryl, —C₃₋₁₀cycloalkyl, —C₁₋₁₀heterocyclyl, —C₁₋₁₀alkyl-C₃₋₁₀aryl, —C₁₋₁₀alkyl-C₁₋₁₀hetaryl, —C₁₋₁₀alkyl-C₃₋₁₀cycloalkyl, —C₁₋₁₀alkyl-C₁₋₁₀heterocyclyl, —C₃₋₁₀cycloalkyl-C₁₋₁₀alkyl, —C₃₋₁₀cycloalkyl-C₃₋₁₀aryl, —C₃₋₁₀cycloalkyl-C₁₋₁₀hetaryl, —C₃₋₁₀cycloalkyl-C₁₋₁₀heterocyclyl, —C₁₋₁₀heterocyclyl-C₁₋₁₀alkyl, —C₁₋₁₀heterocyclyl-C₃₋₁₀aryl, —C₁₋₁₀heterocyclyl-C₁₋₁₀hetaryl, or —C₁₋₁₀heterocyclyl-C₃₋₁₀cycloalkyl, each of which is unsubstituted or substituted by one or more independent R₁₀ or R₁₁ substituents;

R₂₁ is hydrogen, halogen, —OH, —CF₃, —OCF₃, —OR³¹, —NR³¹R³², —C(O)R³¹, —CO₂R³¹, —C(═O)NR³¹, —NO₂, —CN, —S(O)₀₋₂R³¹, —NR³¹C(═O)R³², -L-C₁₋₁₀alkyl, -L-C₂₋₁₀alkenyl, -L-C₂₋₁₀alkynyl, -L-C₃₋₁₀aryl, -L-C₁₋₁₀hetaryl, -L-C₃₋₁₀cycloalkyl, or -L-C₁₋₁₀heterocyclyl, each of which is unsubstituted or substituted by one or more independent R₁₂ substituents;

L is a bond, —O—, —N(R³¹)—, —S(O)₀₋₂—, —C(═O)—, —C(═O)O—, —OC(═O)—, —C(═O)N(R³¹)—, —N(R³¹)C(═O)—, —NR³¹C(═O)O—, —NR³¹C(═O)NR³²—, —NR³¹S(O)₀₋₂—, or —S(O)₀₋₂N(R³¹)—;

R₇₂ is hydrogen, —C₁₋₁₀alkyl, —C₃₋₁₀aryl, —C₁₋₁₀hetaryl, —C₃₋₁₀cycloalkyl, —C₁₋₁₀heterocyclyl, —OH, —CF₃, —C(O)R³¹, —CO₂R³¹, —C(═O)NR³¹, or —S(O)₀₋₂R³¹;

each of R₁₀ is independently -C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, or —C₁₋₁₀heterocyclyl, optionally substituted by one or more independent R₁₁ substituents;

each of R₁₁, R₁₂, and R₁₃ is independently hydrogen, halogen, —C₁₋₁₀alkyl, —C₃₋₁₀aryl, —C₃₋₁₀cycloalkyl, —OH, —CF₃, —OCF₃, —OR³¹, —NR³¹R³², —C(O)R³¹, —C(═O)NR³¹, —NO₂, —CN, —S(O)₀₋₂R^{3l} or —NR³¹C(═O)R³²; and

each of R³¹and R³² is independently hydrogen, —C₁₋₁₀alkyl, —C₃₋₁₀aryl, or —C₃₋₁₀cycloalkyl, or wherein R³¹ together with R³² form a heterocyclic ring.

In some embodiments of Formula I-A,

R₁ is —C₁₋₁₀alkyl, —C₁₋₁₀heterocyclyl, —C₁₋₁₀alkyl-C₃₋₁₀aryl, —C₁₋₁₀alkyl-C₁₋₁₀hetaryl, —C₁₋₁₀alkyl-C₃₋₁₀cycloalkyl, —C₁₋₁₀alkyl-C₁₋₁₀heterocyclyl, —C₁₋₁₀heterocyclyl-C₁₋₁₀alkyl, or —C₁₋₁₀heterocyclyl-C₃₋₁₀aryl, each of which is unsubstituted or substituted by one or more independent R₁₀ or R₁₁ substituents;

R₂₁ is halogen, —OH, —CF₃, —OCF₃, —OR³¹, —NR³¹R³², —C(O)R³¹, —C(═O)NR³¹, —NO₂, —CN, —S(O)₀₋₂R³¹, —NR³¹C(═O)R³², -L-C₁₋₁₀alkyl, -L-C₃₋₁₀aryl, -L-C₁₋₁₀hetaryl, -L-C₃₋₁₀cycloalkyl, or -L-C₁₋₁₀heterocyclyl, each of which is unsubstituted or substituted by one or more independent R₁₂ substituents;

L is a bond, —O—, —N(R³¹)—, —S(O)₀₋₂—, —C(═O)—, —C(═O)O—, —OC(═O)—, —C(═O)N(R³¹)—, or —N(R³¹)C(═O)—;

R₇₂ is hydrogen, —C₁₋₁₀alkyl, —C₃₋₁₀aryl, —C₃₋₁₀cycloalkyl, —C(O)R³¹, —CO₂R³¹, —C(═O)NR³¹, —S(O)₀₋₂R³¹;

R₁₀ is -C₁₋₁₀alkyl, —C₃₋₁₀aryl, —C₃₋₁₀cycloalkyl, or —C₁₋₁₀heterocyclyl, optionally substituted by one or more independent R₁₁ substituents;

each of R₁₁, R_{12,} and R₁₃ is independently hydrogen, halogen, —C₁₋₁₀alkyl, —OH, —CF₃, —OR³¹, —NR³¹R³², —C(O)R³¹, —CO₂R³¹, —C(═O)NR³¹, —NO₂, —CN, —S(O)₀₋₂R³¹ or —NR³¹C(═O)R³²; _{and}

each of R³¹ and R³² is independently hydrogen or —C₁₋₁₀alkyl, or wherein R³¹ together with R³² form a heterocyclic ring.

In some embodiments of Formula I-A,

R₁ is —C₁₋₁₀alkyl, —C₁₋₁₀heterocyclyl, —C₁₋₁₀alkyl-C₃₋₁₀aryl, —C₁₋₁₀alkyl-C₁₋₁₀hetaryl, —C₁₋₁₀alkyl-C₃₋₁₀cycloalkyl, —C₁₋₁₀alkyl-C₁₋₁₀heterocyclyl, —C₁₋₁₀heterocyclyl-C₁₋₁₀alkyl, or C₁₋₁₀heterocyclyl-C₃₋₁₀aryl, each of which is unsubstituted or substituted by one or more independent R₁₀ or R₁₁ substituents;

R₂₁ is halogen, —OH, —CF₃, —OCF₃, —OR³¹, —NR³¹R³², —C(O)R³¹, —CO₂R³¹, —C(═O)NR³¹, —NO₂, —CN, —S(O)₀₋₂R³¹, —NR³¹C(═O)R³², -L-C₁₋₁₀alkyl, -L-C₃₋₁₀aryl, -L-C₁₋₁₀hetaryl, -L-C₃₋₁₀cycloalkyl, or -L-C₁₋₁₀heterocyclyl, each of which is unsubstituted or substituted by one or more independent R₁₂ substituents;

L is a bond, —O—, —N(R³¹)—, —S(O)₀₋₂—, —C(═O)—, —C(═O)O—, —OC(═O)—, —C(═O)N(R³¹)—, or —N(R³¹)C(═O)—;

R₇₂ is hydrogen or —C₁₋₁₀alkyl;

each of R₁₀ is independently C₁₋₁₀alkyl, —C₃₋₁₀aryl, —C₁₋₁₀hetaryl, —C₃₋₁₀cycloalkyl, or —C₁₋₁₀heterocyclyl, optionally substituted by one or more independent R₁₁ substituents;

each of R₁₁ and R₁₂ is independently hydrogen, halogen, —C₁₋₁₀alkyl, —OH, —CF₃, —OR³¹, —NR³¹R³², —C(O)R³¹, —CO₂R³¹, —C(═O)NR31, —NO₂, —CN, —S(O)₀₋₂R³¹ or —NR³¹C(═O)R³²; and

each of R³¹ and R³² is independently hydrogen or —C₁₋₁₀alkyl.

In some embodiments of Formula I-A,

R₁ is —C₁₋₁₀alkyl, —C₁₋₁₀heterocyclyl, —C₁₋₁₀alkyl-C₃₋₁₀aryl, —C₁₋₁₀heterocyclyl-C₁₋₁₀alkyl, or —C₁₋₁₀heterocyclyl-C₃₋₁₀aryl, each of which is unsubstituted or substituted by one or more independent R₁₀ or R₁₁ substituents;

R₂₁ is halogen, —CN, -L-C₁₋₁₀alkyl, -L-C₃₋₁₀aryl, -L-C₁₋₁₀hetaryl, -L-C₃₋₁₀cycloalkyl, or -L-C₁₋₁₀heterocyclyl, each of which is unsubstituted or substituted by one or more independent R₁₂ substituents;

L is a bond, —N(R³¹)—, or —C(═O)N(R³¹)—;

R₇₂ is hydrogen;

each of R₁₀ is independently —C₁₋₁₀alkyl, —C₃₋₁₀aryl, —C₁₋₁₀hetaryl, or —C₁₋₁₀heterocyclyl, optionally substituted by one or more independent R₁₁ substituents;

each of R₁₁ and R₁₂ is independently hydrogen, halogen, —C₁₋₁₀alkyl, —OH, —CF₃, —OR³¹ or —CN; and

each of R³¹ is independently hydrogen or —C₁₋₁₀alkyl.

In some embodiments of Formula I-A,

R₁ is —C₁₋₁₀alkyl, —C₁₋₁₀alkyl-C₃₋₁₀aryl, or —C₁₋₁₀heterocyclyl-C₁₋₁₀alkyl, each of which is unsubstituted or substituted by one or more independent R₁₀ or R₁₁ substituents;

R₂₁ is -L-C₃₋₁₀aryl or LC₁₋₁₀hetaryl, each of which is unsubstituted or substituted by one or more independent R₁₂ substituents;

L is a bond or —N(R³¹)—;

R₇₂ is hydrogen;

each of R₁₀ is independently —C₃₋₁₀aryl, —C₁₋₁₀hetaryl, or —C₁₋₁₀heterocyclyl, optionally substituted by one or more independent R₁₁ substituents;

each of R₁₁ and R₁₂ is independently halogen, —C₁₋₁₀alkyl, —OH, —CF₃ or OR³¹; and

each of R³¹ is independently hydrogen or —C₁₋₁₀alkyl.

In some embodiments of Formula I-A,

R₁ is —C₁₋₁₀heterocyclyl-C₁₋₁₀alkyl, unsubstituted or substituted by one or more independent R₁₁ substituents;

R₂₁ is pyridyl selected from the group consisting of 2-pyridyl, 3-pyridyl and 4-pyridyl, which is unsubstituted or substituted by one or more independent R₁₂ substituents;

L is a bond;

R₇₂ is hydrogen;

each of R₁₁ and R₁₂ is independently halogen, —C₁₋₁₀alkyl, —CF₃ or —OR³¹; and

each of R³¹ is independently hydrogen or —C₁₋₁₀alkyl.

In certain embodiments, for a compound of Formula I or I-A, R₁ is —C₁₋₁₀heterocyclyl-C₁₋₁₀alkyl, which is unsubstituted. In some embodiments, R₁ is —C₁₋₁₀heterocyclyl-C₁₋₁₀alkyl, substitute by one or more independent R₁₀ substituents. In some embodiments, R₁ is —C₁₋₁₀heterocyclyl-C₁₋₁₀alkyl, substituted by one or more independent R₁₁ substituents. In some embodiments, R₁ is —C₁₋₁₀heterocyclyl-C₁₋₁₀alkyl, substituted by one or more independent R₁₀ or R₁₁ substituents. In some embodiments, R₁₀and R₁₁ are selected from aryl, such as phenyl.

In certain embodiments, for a compound of Formula I or I-A, R₁ is —C₁₋₁₀alkyl, —C₁₋₁₀heterocyclyl, —C₁₋₁₀alkyl-C₃₋₁₀heterocyclyl, —C₁₋₁₀alkyl C₁₋₁₀hetaryl, —C₁₋₁₀heterocyclyl-C₁₋₁₀alkyl, or —C₁₋₁₀heterocyclyl-C₃₋₁₀aryl, unsubstituted or substituted by one or more independent R₁₀ or R₁₁ substituents. In other embodiments, R₁ is —C₁₋₁₀alkyl, —C₁₋₁₀heterocyclyl, —C₁₋₁₀alkyl-C₃₋₁₀heterocyclyl, —C₁₋₁₀alkyl-C₃₋₁₀aryl, —C₁₋₁₀alkyl, —C₁₋₁₀hetaryl, —C₁₋₁₀heterocyclyl-C₁₋₁₀alkyl, or —C₁₋₁₀heterocyclyl-C₃₋₁₀aryl, unsubstituted or substituted by one or more independent R₁₀ or R₁₁ substituents. In yet other embodiments, R₁ is —C₁₋₁₀alkyl-C₃₋₁₀aryl, —C₁₋₁₀alkyl-C₁₋₁₀hetaryl, —C₁₋₁₀heterocyclyl-C₁₋₁₀alkyl, or —C₁₋₁₀heterocyclyl-C₃₋₁₀aryl, unsubstituted or substituted by one or more independent R₁₀ or R₁₁ substituents. In yet other embodiments, R₁ is —C₁₋₁₀alkyl-C₃₋₁₀aryl or —C₁₋₁₀heterocyclyl-C₃₋₁₀aryl, unsubstituted or substituted by one or more independent R₁₀ or R₁₁ substituents. In further embodiments, wherein R₁ is

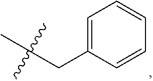 [SMILES: *Cc1ccccc1]

unsubstituted or substituted by one or more independent R₁₀ or R₁₁ substituents. In some embodiments, R1 is R₁ is —C₁₋₁₀heterocyclyl, —C₁₋₁₀heterocyclyl-C₁₋₁₀alkyl, or —C₁₋₁₀heterocyclyl-C₃₋₁₀aryl, unsubstituted or substituted by one or more independent R₁₀ or R₁₁ substituents. In some embodiments, R₁ is

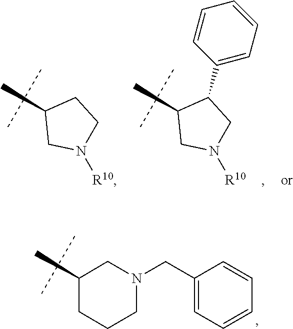

unsubstituted or substituted by one or more independent R₁₀ or R₁₁ sub stituents.

In certain embodiments, for a compound of Formula I or I-A, each of R₁ or R₁′ is independently a substituent as shown below:

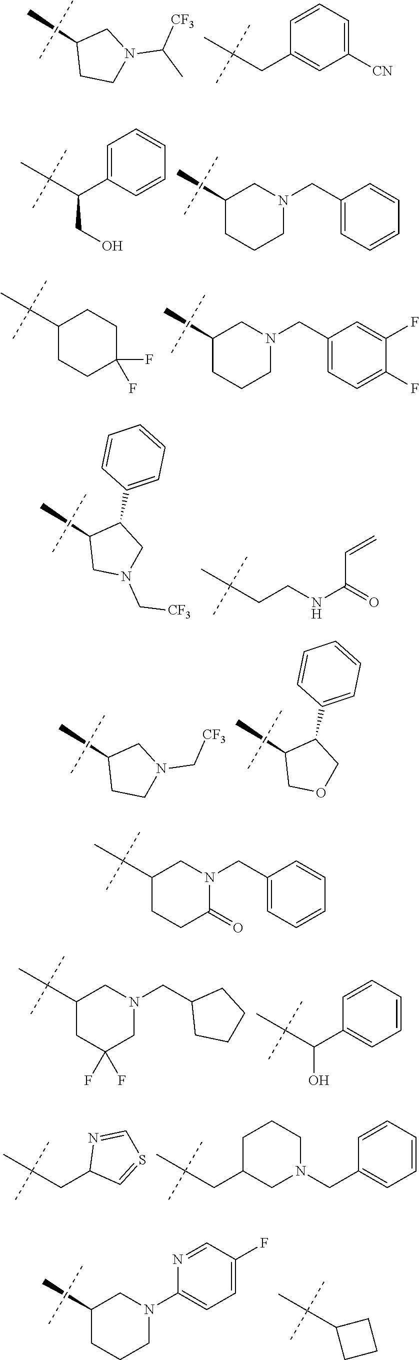

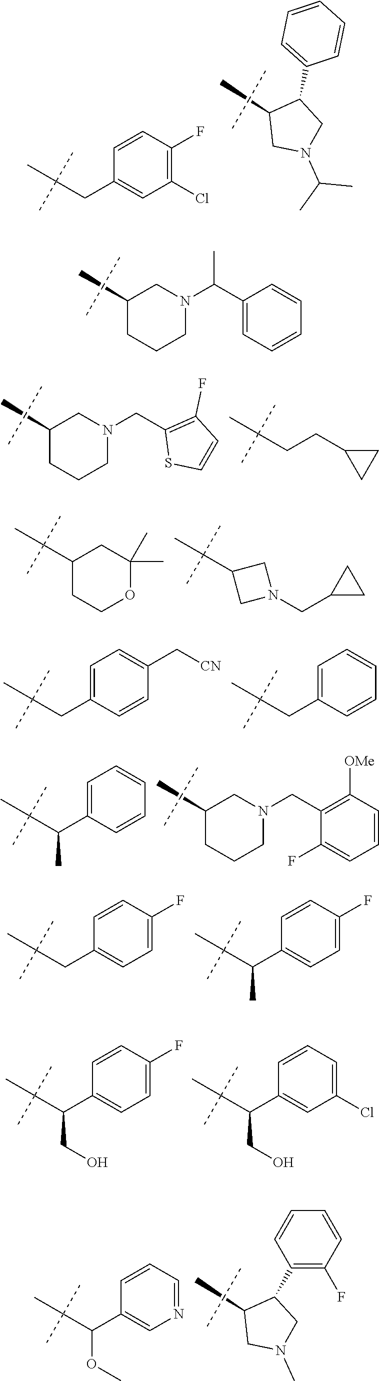

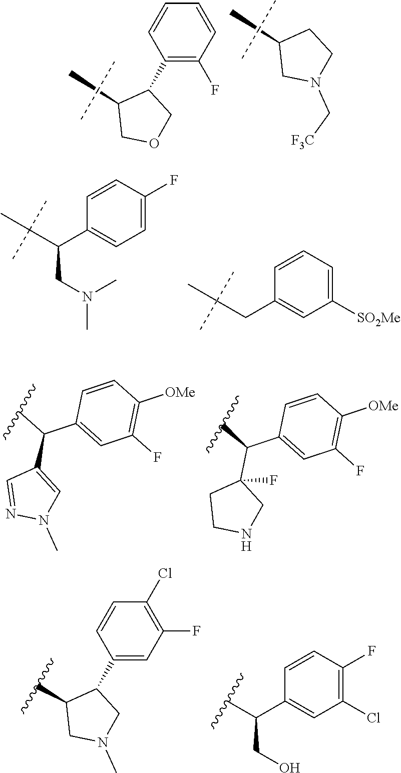

In certain embodiments, the present disclosure provides an ERK inhibitor which is a compound selected from the group consisting of:

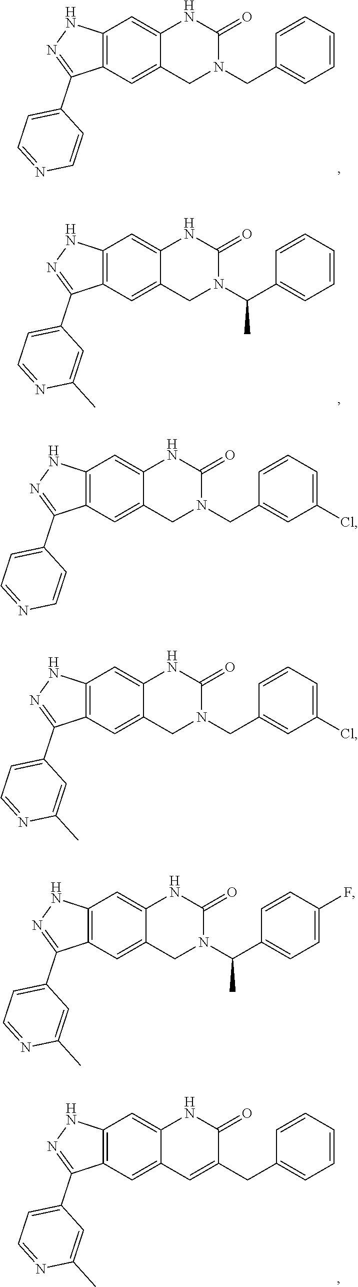 [SMILES: O=C1Nc2cc3[nH]nc(-c4ccncc4)c3cc2CN1Cc1ccccc1 ; Cc1cc(-c2n[nH]c3cc4c(cc23)CN([C@H](C)c2ccccc2)C(=O)N4)ccn1 ; O=C1Nc2cc3[nH]nc(-c4ccncc4)c3cc2CN1Cc1cccc(Cl)c1 ; Cc1cc(-c2n[nH]c3cc4c(cc23)CN(Cc2cccc(Cl)c2)C(=O)N4)ccn1 ; Cc1cc(-c2n[nH]c3cc4c(cc23)CN([C@H](C)c2ccc(F)cc2)C(=O)N4)ccn1 ; Cc1cc(-c2n[nH]c3cc4[nH]c(=O)c(Cc5ccccc5)cc4cc23)ccn1]

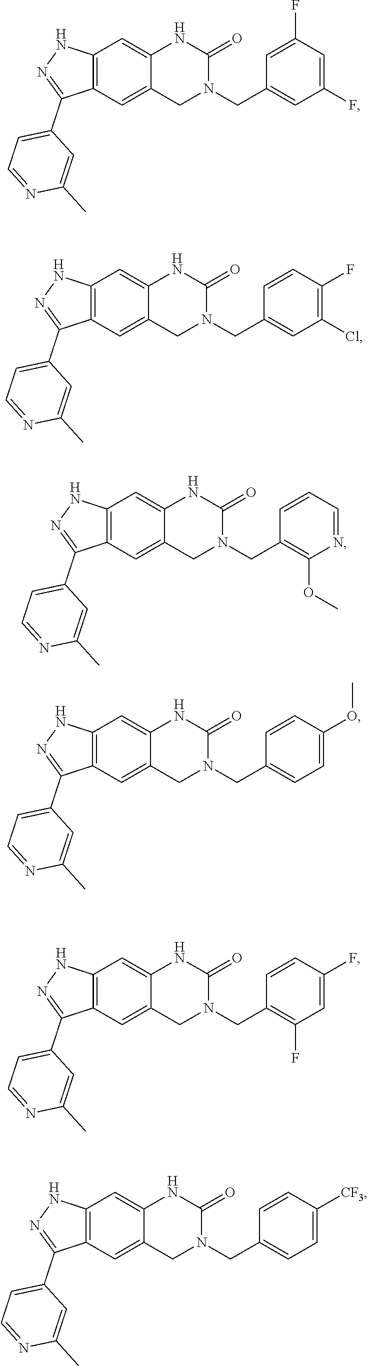 [SMILES: Cc1cc(-c2n[nH]c3cc4c(cc23)CN(Cc2cc(F)cc(F)c2)C(=O)N4)ccn1 ; Cc1cc(-c2n[nH]c3cc4c(cc23)CN(Cc2ccc(F)c(Cl)c2)C(=O)N4)ccn1 ; COc1ncccc1CN1Cc2cc3c(-c4ccnc(C)c4)n[nH]c3cc2NC1=O ; COc1ccc(CN2Cc3cc4c(-c5ccnc(C)c5)n[nH]c4cc3NC2=O)cc1 ; Cc1cc(-c2n[nH]c3cc4c(cc23)CN(Cc2ccc(F)cc2F)C(=O)N4)ccn1 ; Cc1cc(-c2n[nH]c3cc4c(cc23)CN(Cc2ccc(C(F)(F)F)cc2)C(=O)N4)ccn1]

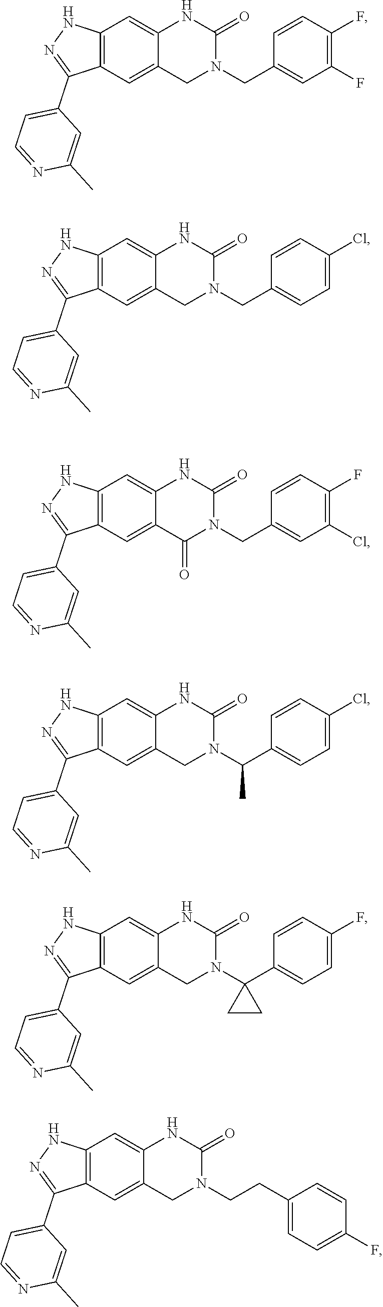 [SMILES: Cc1cc(-c2n[nH]c3cc4c(cc23)CN(Cc2ccc(F)c(F)c2)C(=O)N4)ccn1 ; Cc1cc(-c2n[nH]c3cc4c(cc23)CN(Cc2ccc(Cl)cc2)C(=O)N4)ccn1 ; Cc1cc(-c2n[nH]c3cc4[nH]c(=O)n(Cc5ccc(F)c(Cl)c5)c(=O)c4cc23)ccn1 ; Cc1cc(-c2n[nH]c3cc4c(cc23)CN([C@H](C)c2ccc(Cl)cc2)C(=O)N4)ccn1 ; Cc1cc(-c2n[nH]c3cc4c(cc23)CN(C2(c3ccc(F)cc3)CC2)C(=O)N4)ccn1 ; Cc1cc(-c2n[nH]c3cc4c(cc23)CN(CCc2ccc(F)cc2)C(=O)N4)ccn1]

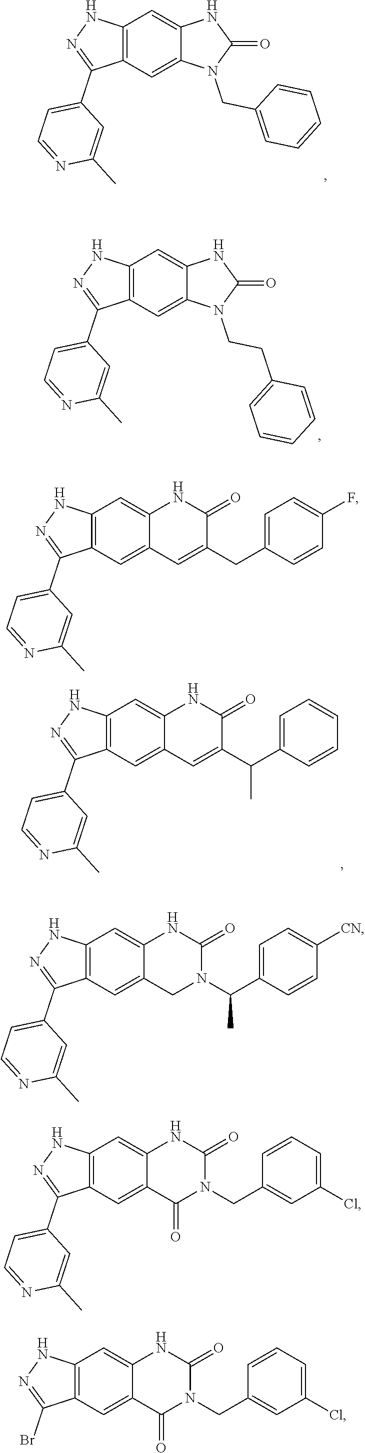

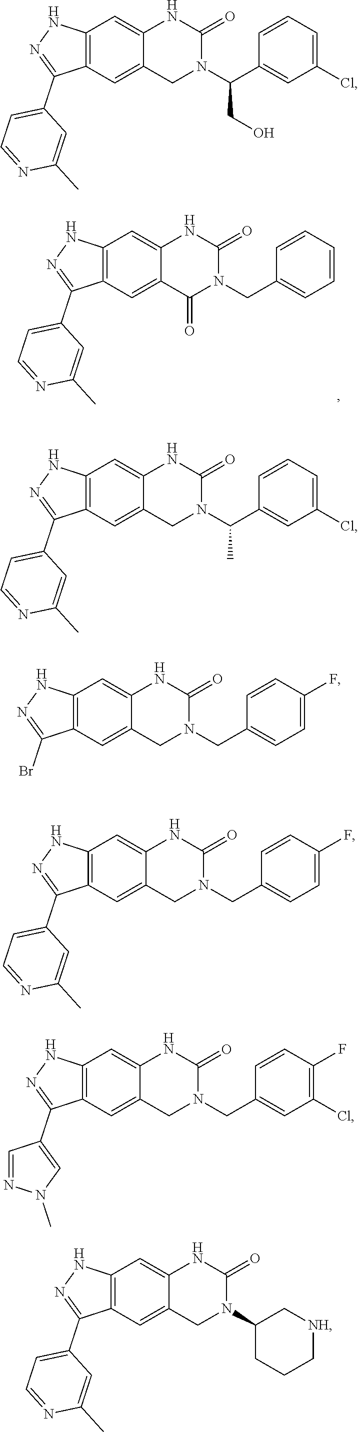

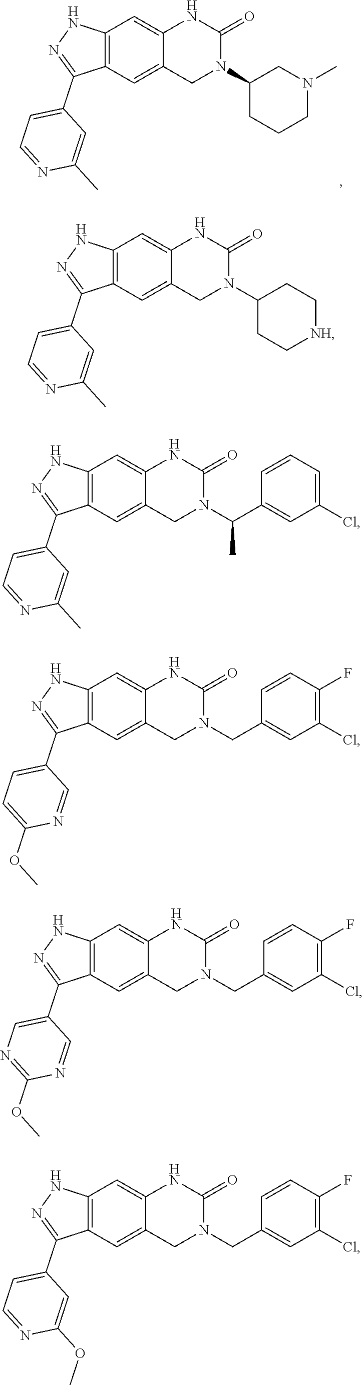 [SMILES: Cc1cc(-c2n[nH]c3cc4c(cc23)CN([C@@H]2CCCN(C)C2)C(=O)N4)ccn1 ; Cc1cc(-c2n[nH]c3cc4c(cc23)CN(C2CCNCC2)C(=O)N4)ccn1 ; Cc1cc(-c2n[nH]c3cc4c(cc23)CN([C@H](C)c2cccc(Cl)c2)C(=O)N4)ccn1 ; COc1ccc(-c2n[nH]c3cc4c(cc23)CN(Cc2ccc(F)c(Cl)c2)C(=O)N4)cn1 ; COc1ncc(-c2n[nH]c3cc4c(cc23)CN(Cc2ccc(F)c(Cl)c2)C(=O)N4)cn1 ; COc1cc(-c2n[nH]c3cc4c(cc23)CN(Cc2ccc(F)c(Cl)c2)C(=O)N4)ccn1]

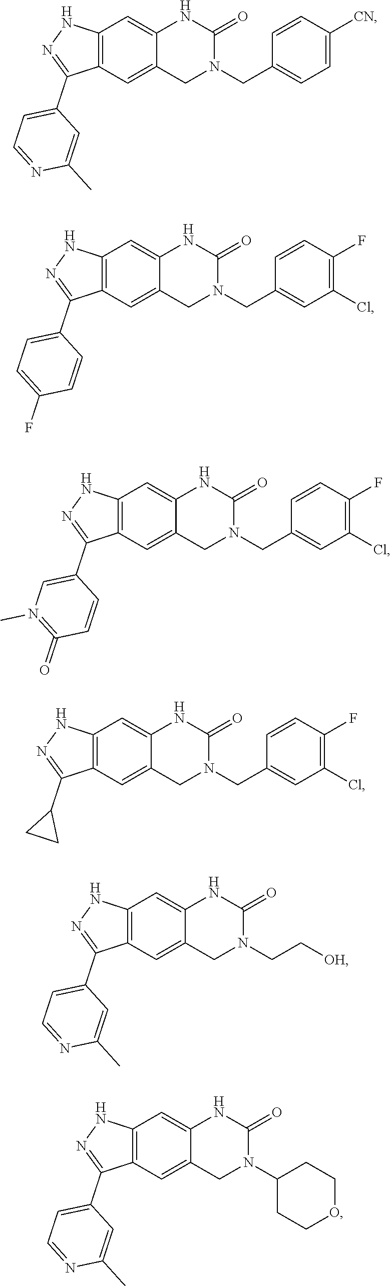

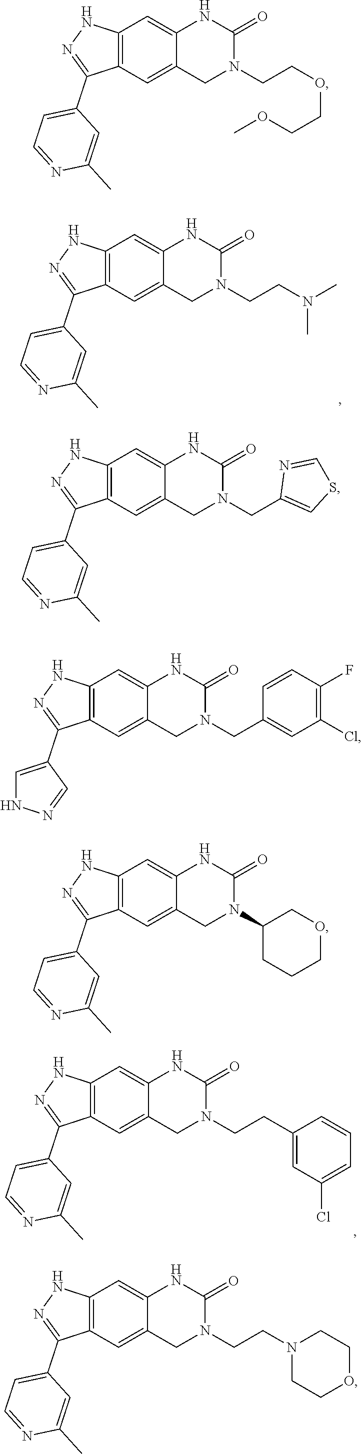

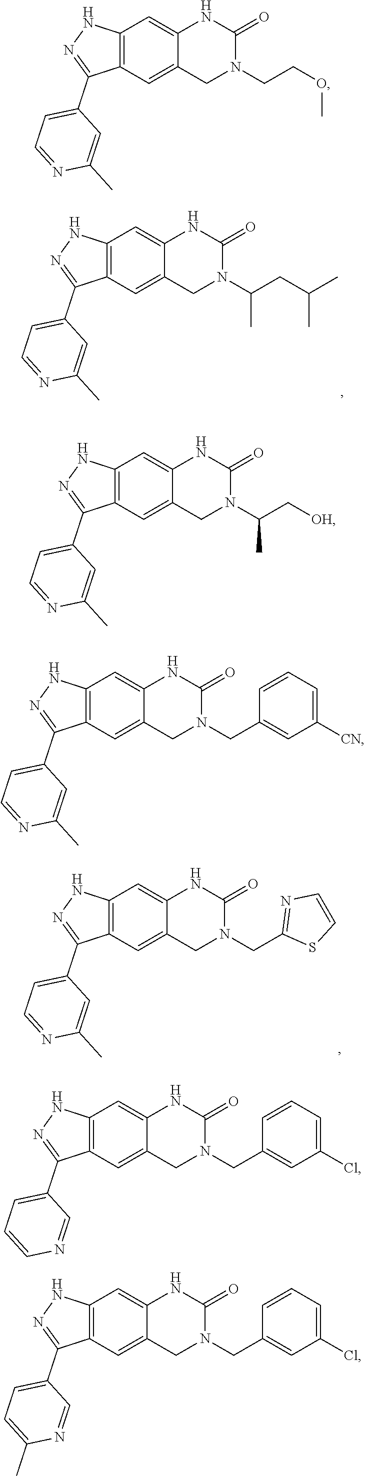

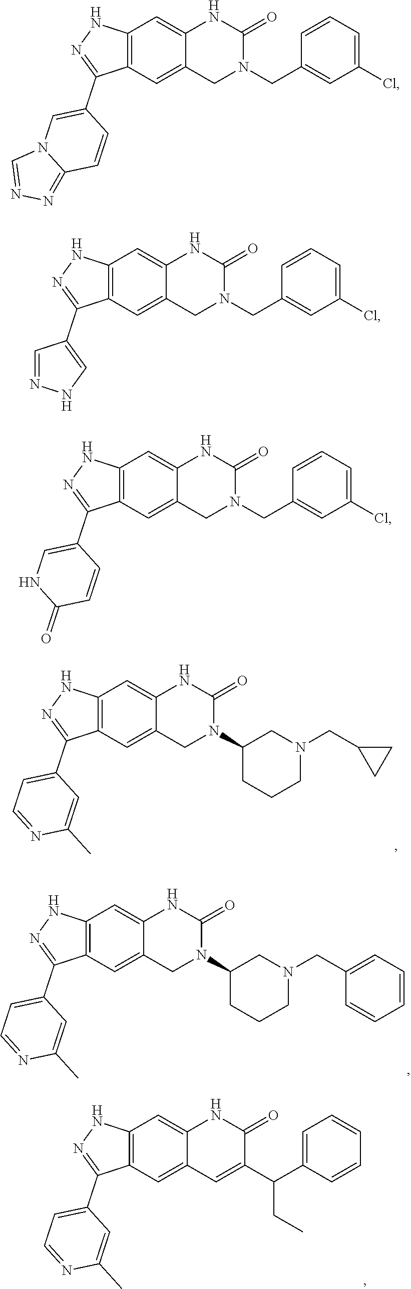 [SMILES: O=C1Nc2cc3[nH]nc(-c4ccc5nncn5c4)c3cc2CN1Cc1cccc(Cl)c1 ; O=C1Nc2cc3[nH]nc(-c4cn[nH]c4)c3cc2CN1Cc1cccc(Cl)c1 ; O=C1Nc2cc3[nH]nc(-c4ccc(=O)[nH]c4)c3cc2CN1Cc1cccc(Cl)c1 ; Cc1cc(-c2n[nH]c3cc4c(cc23)CN([C@@H]2CCCN(CC3CC3)C2)C(=O)N4)ccn1 ; Cc1cc(-c2n[nH]c3cc4c(cc23)CN([C@@H]2CCCN(Cc3ccccc3)C2)C(=O)N4)ccn1 ; CCC(c1ccccc1)c1cc2cc3c(-c4ccnc(C)c4)n[nH]c3cc2[nH]c1=O]

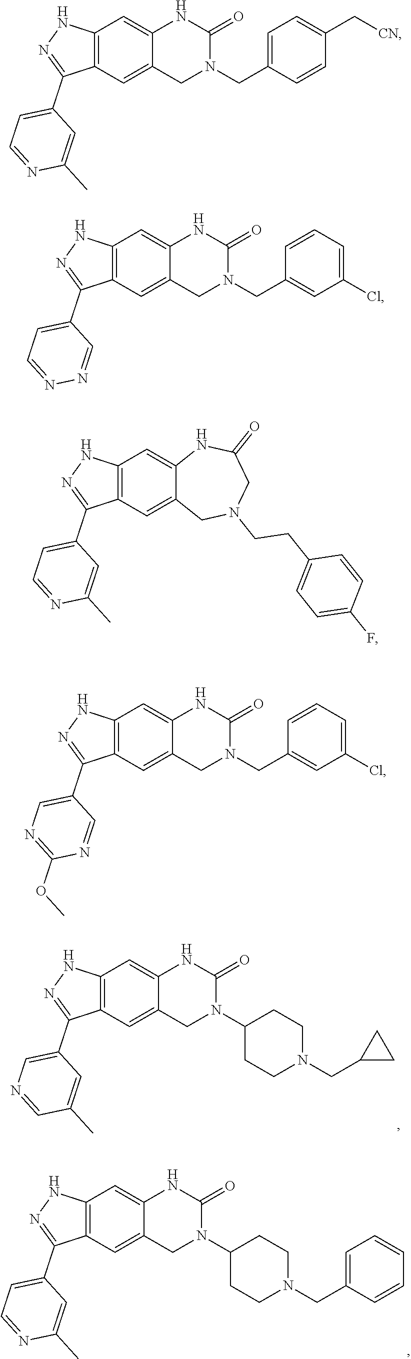 [SMILES: Cc1cc(-c2n[nH]c3cc4c(cc23)CN(Cc2ccc(CC#N)cc2)C(=O)N4)ccn1 ; O=C1Nc2cc3[nH]nc(-c4ccnnc4)c3cc2CN1Cc1cccc(Cl)c1 ; Cc1cc(-c2n[nH]c3cc4c(cc23)CN(CCc2ccc(F)cc2)CC(=O)N4)ccn1 ; COc1ncc(-c2n[nH]c3cc4c(cc23)CN(Cc2cccc(Cl)c2)C(=O)N4)cn1 ; Cc1cncc(-c2n[nH]c3cc4c(cc23)CN(C2CCN(CC3CC3)CC2)C(=O)N4)c1 ; Cc1cc(-c2n[nH]c3cc4c(cc23)CN(C2CCN(Cc3ccccc3)CC2)C(=O)N4)ccn1]

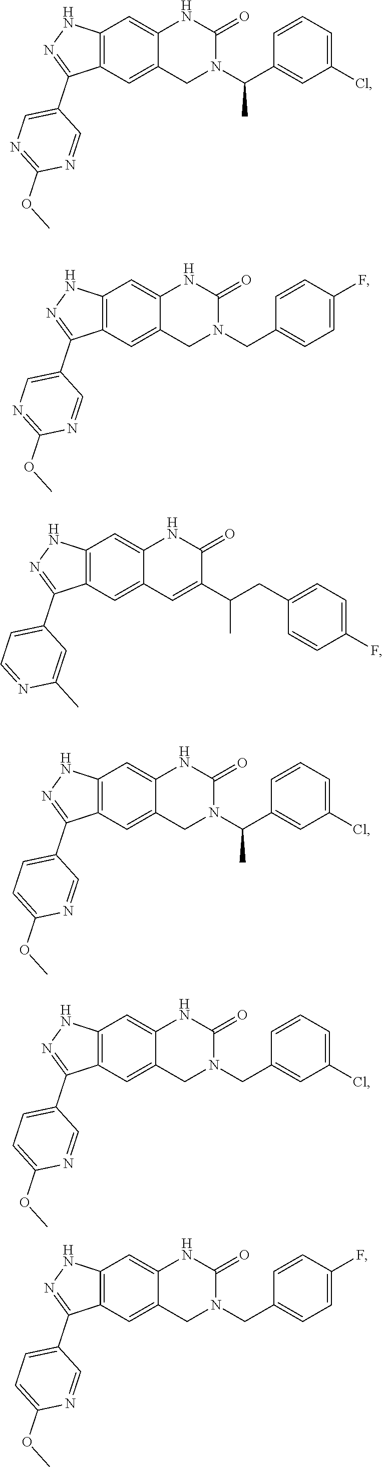

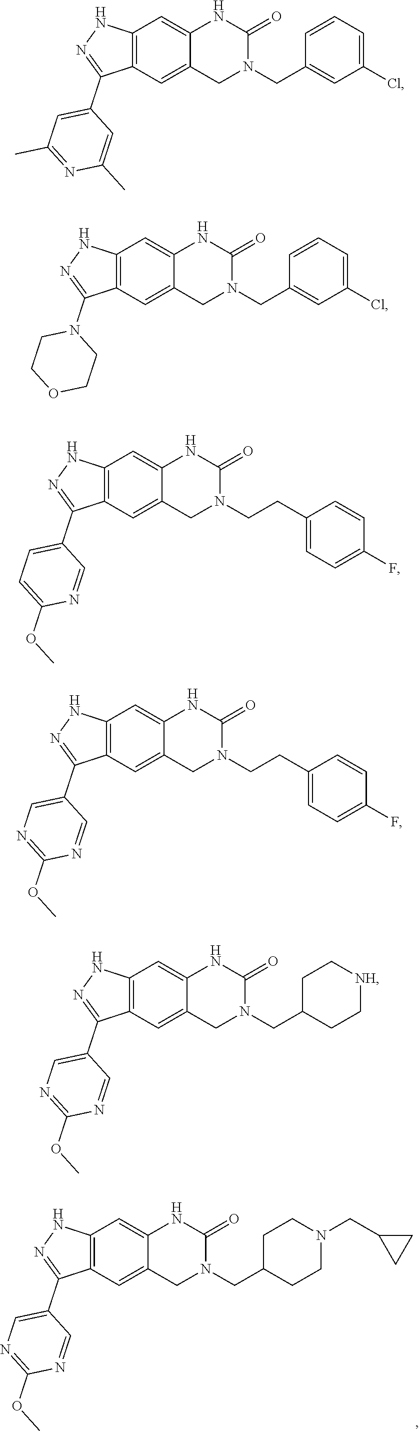 [SMILES: Cc1cc(-c2n[nH]c3cc4c(cc23)CN(Cc2cccc(Cl)c2)C(=O)N4)cc(C)n1 ; O=C1Nc2cc3[nH]nc(N4CCOCC4)c3cc2CN1Cc1cccc(Cl)c1 ; COc1ccc(-c2n[nH]c3cc4c(cc23)CN(CCc2ccc(F)cc2)C(=O)N4)cn1 ; COc1ncc(-c2n[nH]c3cc4c(cc23)CN(CCc2ccc(F)cc2)C(=O)N4)cn1 ; COc1ncc(-c2n[nH]c3cc4c(cc23)CN(CC2CCNCC2)C(=O)N4)cn1 ; COc1ncc(-c2n[nH]c3cc4c(cc23)CN(CC2CCN(CC3CC3)CC2)C(=O)N4)cn1]

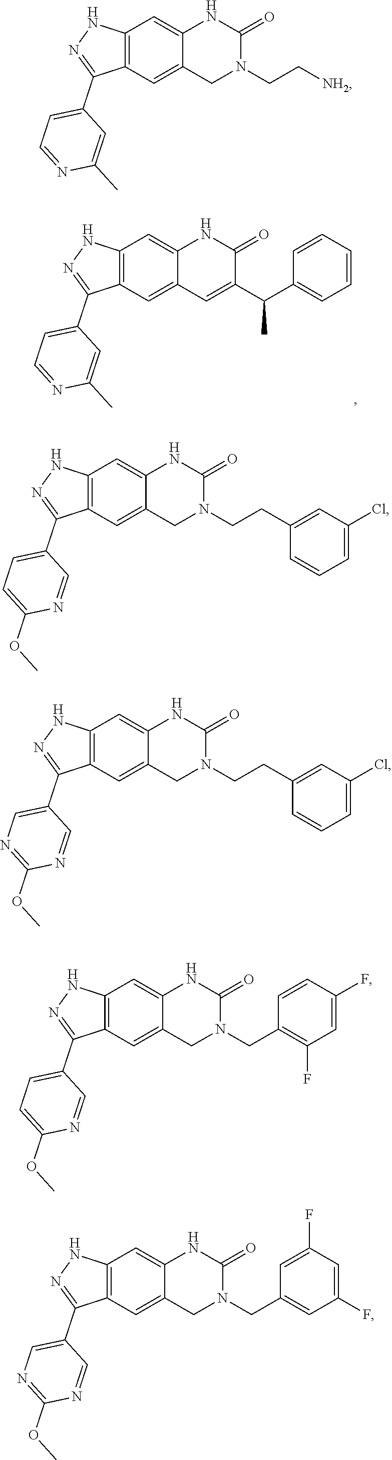 [SMILES: Cc1cc(-c2n[nH]c3cc4c(cc23)CN(CCN)C(=O)N4)ccn1 ; Cc1cc(-c2n[nH]c3cc4[nH]c(=O)c([C@H](C)c5ccccc5)cc4cc23)ccn1 ; COc1ccc(-c2n[nH]c3cc4c(cc23)CN(CCc2cccc(Cl)c2)C(=O)N4)cn1 ; COc1ncc(-c2n[nH]c3cc4c(cc23)CN(CCc2cccc(Cl)c2)C(=O)N4)cn1 ; COc1ccc(-c2n[nH]c3cc4c(cc23)CN(Cc2ccc(F)cc2F)C(=O)N4)cn1 ; COc1ncc(-c2n[nH]c3cc4c(cc23)CN(Cc2cc(F)cc(F)c2)C(=O)N4)cn1]

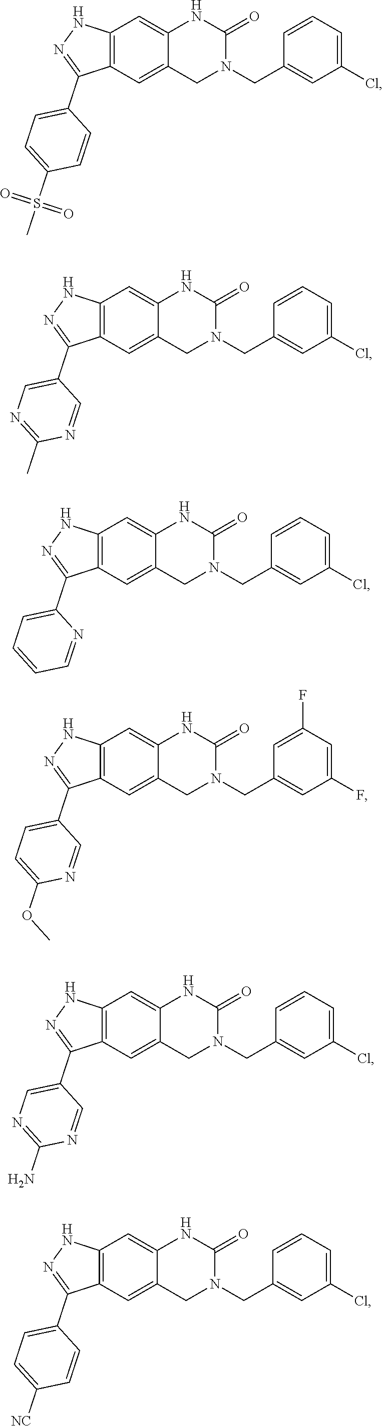 [SMILES: CS(=O)(=O)c1ccc(-c2n[nH]c3cc4c(cc23)CN(Cc2cccc(Cl)c2)C(=O)N4)cc1 ; Cc1ncc(-c2n[nH]c3cc4c(cc23)CN(Cc2cccc(Cl)c2)C(=O)N4)cn1 ; O=C1Nc2cc3[nH]nc(-c4ccccn4)c3cc2CN1Cc1cccc(Cl)c1 ; COc1ccc(-c2n[nH]c3cc4c(cc23)CN(Cc2cc(F)cc(F)c2)C(=O)N4)cn1 ; Nc1ncc(-c2n[nH]c3cc4c(cc23)CN(Cc2cccc(Cl)c2)C(=O)N4)cn1 ; N#Cc1ccc(-c2n[nH]c3cc4c(cc23)CN(Cc2cccc(Cl)c2)C(=O)N4)cc1]

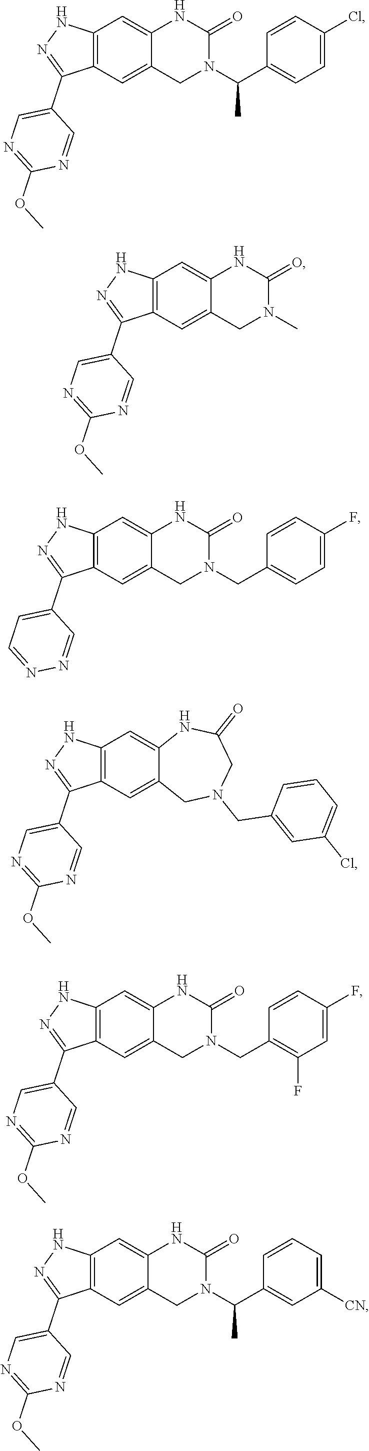

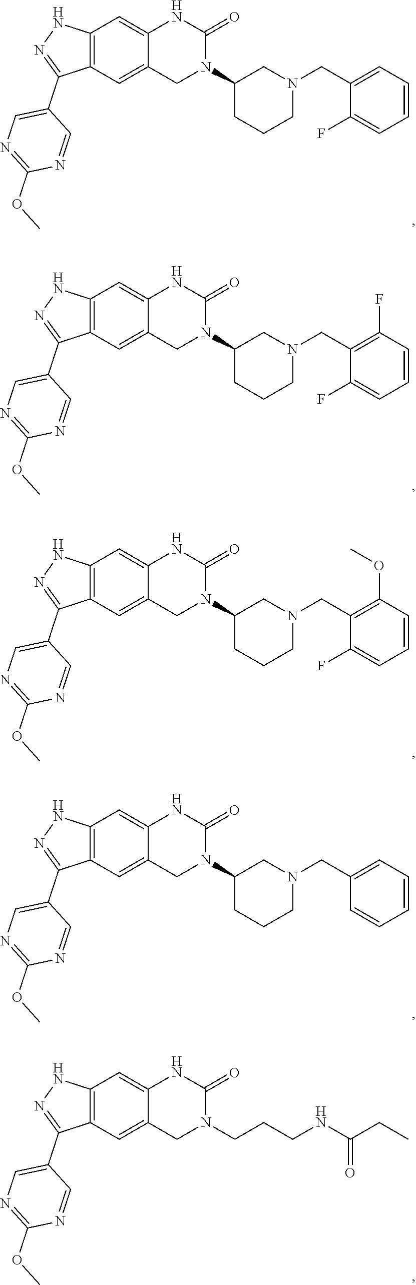 [SMILES: COc1ncc(-c2n[nH]c3cc4c(cc23)CN([C@@H]2CCCN(Cc3ccccc3F)C2)C(=O)N4)cn1 ; COc1ncc(-c2n[nH]c3cc4c(cc23)CN([C@@H]2CCCN(Cc3c(F)cccc3F)C2)C(=O)N4)cn1 ; COc1ncc(-c2n[nH]c3cc4c(cc23)CN([C@@H]2CCCN(Cc3c(F)cccc3OC)C2)C(=O)N4)cn1 ; COc1ncc(-c2n[nH]c3cc4c(cc23)CN([C@@H]2CCCN(Cc3ccccc3)C2)C(=O)N4)cn1 ; CCC(=O)NCCCN1Cc2cc3c(-c4cnc(OC)nc4)n[nH]c3cc2NC1=O]

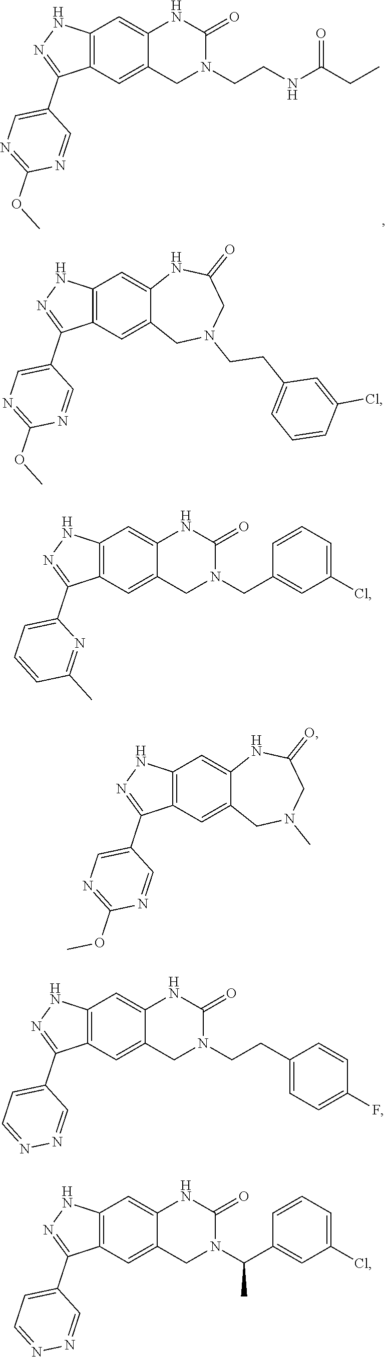

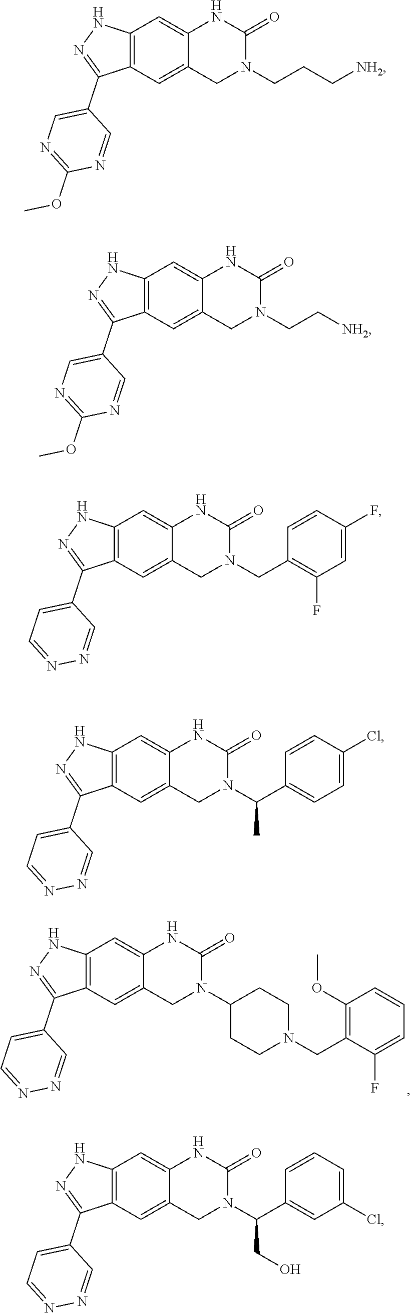 [SMILES: COc1ncc(-c2n[nH]c3cc4c(cc23)CN(CCCN)C(=O)N4)cn1 ; COc1ncc(-c2n[nH]c3cc4c(cc23)CN(CCN)C(=O)N4)cn1 ; O=C1Nc2cc3[nH]nc(-c4ccnnc4)c3cc2CN1Cc1ccc(F)cc1F ; C[C@H](c1ccc(Cl)cc1)N1Cc2cc3c(-c4ccnnc4)n[nH]c3cc2NC1=O ; COc1cccc(F)c1CN1CCC(N2Cc3cc4c(-c5ccnnc5)n[nH]c4cc3NC2=O)CC1 ; O=C1Nc2cc3[nH]nc(-c4ccnnc4)c3cc2CN1[C@H](CO)c1cccc(Cl)c1]

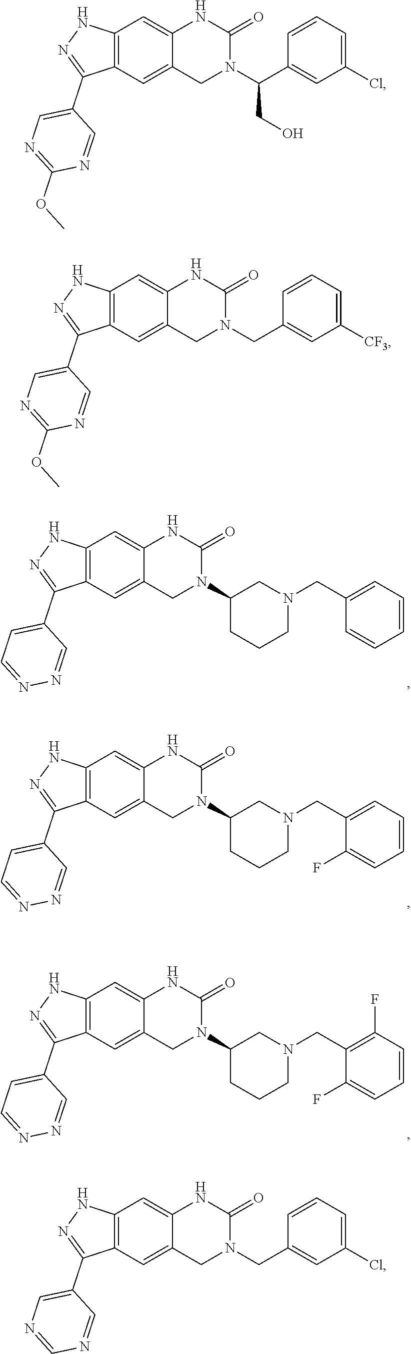 [SMILES: COc1ncc(-c2n[nH]c3cc4c(cc23)CN([C@H](CO)c2cccc(Cl)c2)C(=O)N4)cn1 ; COc1ncc(-c2n[nH]c3cc4c(cc23)CN(Cc2cccc(C(F)(F)F)c2)C(=O)N4)cn1 ; O=C1Nc2cc3[nH]nc(-c4ccnnc4)c3cc2CN1[C@@H]1CCCN(Cc2ccccc2)C1 ; O=C1Nc2cc3[nH]nc(-c4ccnnc4)c3cc2CN1[C@@H]1CCCN(Cc2ccccc2F)C1 ; O=C1Nc2cc3[nH]nc(-c4ccnnc4)c3cc2CN1[C@@H]1CCCN(Cc2c(F)cccc2F)C1 ; O=C1Nc2cc3[nH]nc(-c4cncnc4)c3cc2CN1Cc1cccc(Cl)c1]

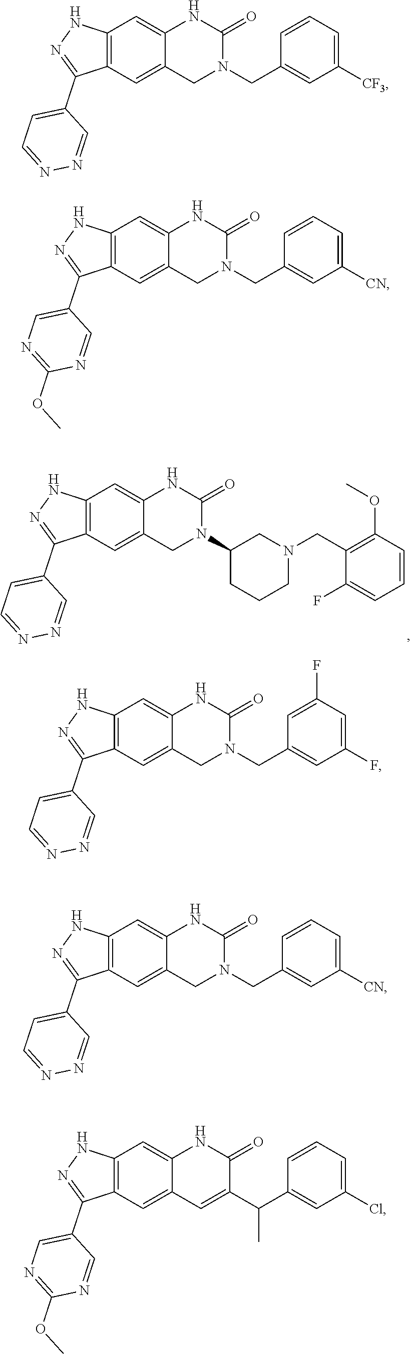 [SMILES: O=C1Nc2cc3[nH]nc(-c4ccnnc4)c3cc2CN1Cc1cccc(C(F)(F)F)c1 ; COc1ncc(-c2n[nH]c3cc4c(cc23)CN(Cc2cccc(C#N)c2)C(=O)N4)cn1 ; COc1cccc(F)c1CN1CCC[C@@H](N2Cc3cc4c(-c5ccnnc5)n[nH]c4cc3NC2=O)C1 ; O=C1Nc2cc3[nH]nc(-c4ccnnc4)c3cc2CN1Cc1cc(F)cc(F)c1 ; N#Cc1cccc(CN2Cc3cc4c(-c5ccnnc5)n[nH]c4cc3NC2=O)c1 ; COc1ncc(-c2n[nH]c3cc4[nH]c(=O)c(C(C)c5cccc(Cl)c5)cc4cc23)cn1]

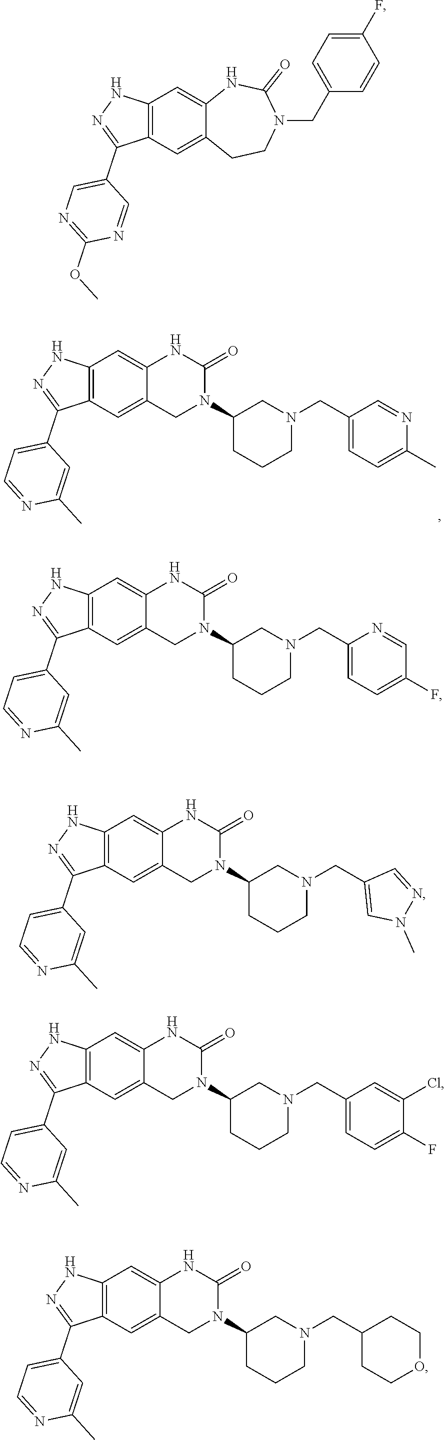 [SMILES: COc1ncc(-c2n[nH]c3cc4c(cc23)CCN(Cc2ccc(F)cc2)C(=O)N4)cn1 ; Cc1ccc(CN2CCC[C@@H](N3Cc4cc5c(-c6ccnc(C)c6)n[nH]c5cc4NC3=O)C2)cn1 ; Cc1cc(-c2n[nH]c3cc4c(cc23)CN([C@@H]2CCCN(Cc3ccc(F)cn3)C2)C(=O)N4)ccn1 ; Cc1cc(-c2n[nH]c3cc4c(cc23)CN([C@@H]2CCCN(Cc3cnn(C)c3)C2)C(=O)N4)ccn1 ; Cc1cc(-c2n[nH]c3cc4c(cc23)CN([C@@H]2CCCN(Cc3ccc(F)c(Cl)c3)C2)C(=O)N4)ccn1 ; Cc1cc(-c2n[nH]c3cc4c(cc23)CN([C@@H]2CCCN(CC3CCOCC3)C2)C(=O)N4)ccn1]

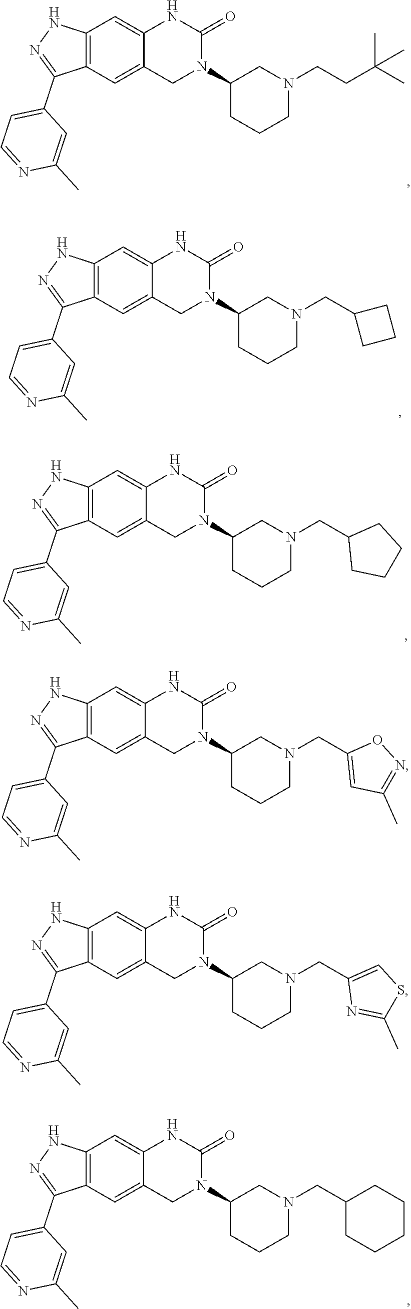 [SMILES: Cc1cc(-c2n[nH]c3cc4c(cc23)CN([C@@H]2CCCN(CCC(C)(C)C)C2)C(=O)N4)ccn1 ; Cc1cc(-c2n[nH]c3cc4c(cc23)CN([C@@H]2CCCN(CC3CCC3)C2)C(=O)N4)ccn1 ; Cc1cc(-c2n[nH]c3cc4c(cc23)CN([C@@H]2CCCN(CC3CCCC3)C2)C(=O)N4)ccn1 ; Cc1cc(-c2n[nH]c3cc4c(cc23)CN([C@@H]2CCCN(Cc3cc(C)no3)C2)C(=O)N4)ccn1 ; Cc1cc(-c2n[nH]c3cc4c(cc23)CN([C@@H]2CCCN(Cc3csc(C)n3)C2)C(=O)N4)ccn1 ; Cc1cc(-c2n[nH]c3cc4c(cc23)CN([C@@H]2CCCN(CC3CCCCC3)C2)C(=O)N4)ccn1]

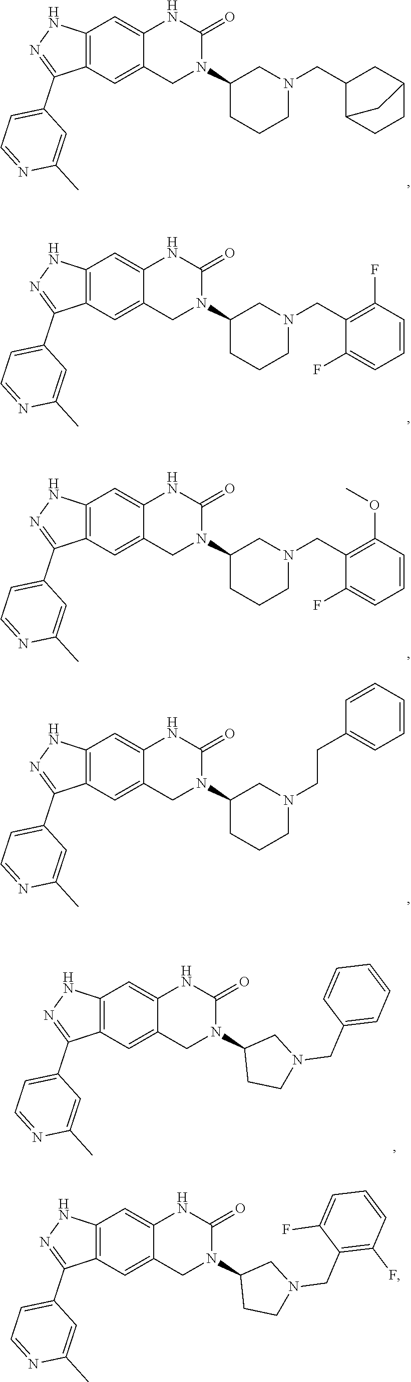 [SMILES: Cc1cc(-c2n[nH]c3cc4c(cc23)CN([C@@H]2CCCN(CC3CC5CCC3C5)C2)C(=O)N4)ccn1 ; Cc1cc(-c2n[nH]c3cc4c(cc23)CN([C@@H]2CCCN(Cc3c(F)cccc3F)C2)C(=O)N4)ccn1 ; COc1cccc(F)c1CN1CCC[C@@H](N2Cc3cc4c(-c5ccnc(C)c5)n[nH]c4cc3NC2=O)C1 ; Cc1cc(-c2n[nH]c3cc4c(cc23)CN([C@@H]2CCCN(CCc3ccccc3)C2)C(=O)N4)ccn1 ; Cc1cc(-c2n[nH]c3cc4c(cc23)CN([C@@H]2CCN(Cc3ccccc3)C2)C(=O)N4)ccn1 ; Cc1cc(-c2n[nH]c3cc4c(cc23)CN([C@@H]2CCN(Cc3c(F)cccc3F)C2)C(=O)N4)ccn1]

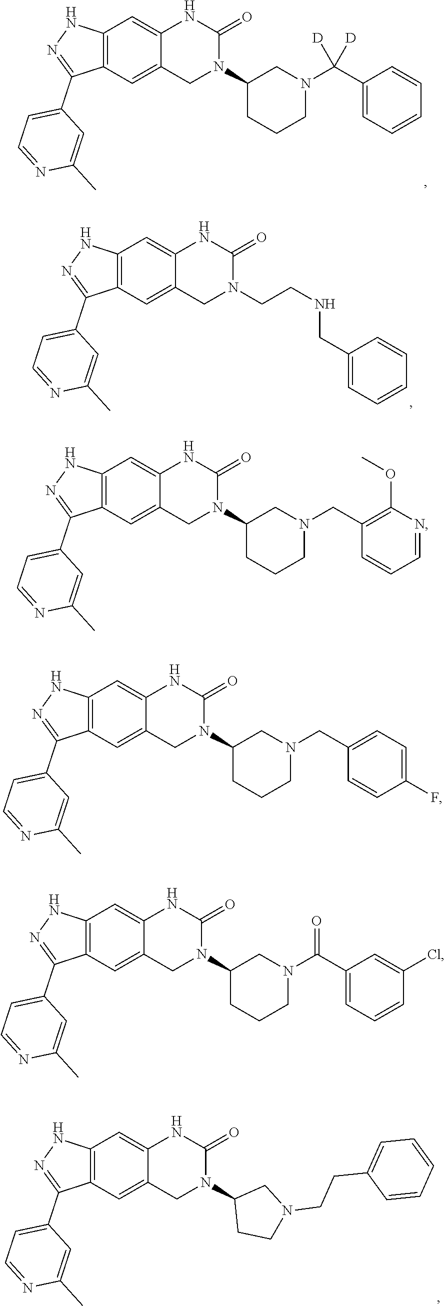 [SMILES: [2H]C([2H])(c1ccccc1)N1CCC[C@@H](N2Cc3cc4c(-c5ccnc(C)c5)n[nH]c4cc3NC2=O)C1 ; Cc1cc(-c2n[nH]c3cc4c(cc23)CN(CCNCc2ccccc2)C(=O)N4)ccn1 ; COc1ncccc1CN1CCC[C@@H](N2Cc3cc4c(-c5ccnc(C)c5)n[nH]c4cc3NC2=O)C1 ; Cc1cc(-c2n[nH]c3cc4c(cc23)CN([C@@H]2CCCN(Cc3ccc(F)cc3)C2)C(=O)N4)ccn1 ; Cc1cc(-c2n[nH]c3cc4c(cc23)CN([C@@H]2CCCN(C(=O)c3cccc(Cl)c3)C2)C(=O)N4)ccn1 ; Cc1cc(-c2n[nH]c3cc4c(cc23)CN([C@@H]2CCN(CCc3ccccc3)C2)C(=O)N4)ccn1]

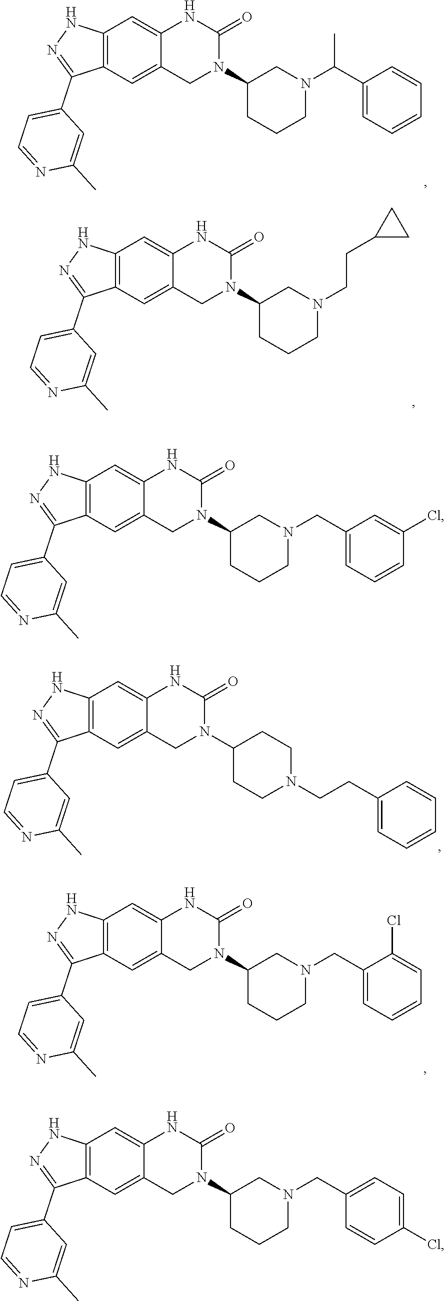 [SMILES: Cc1cc(-c2n[nH]c3cc4c(cc23)CN([C@@H]2CCCN(C(C)c3ccccc3)C2)C(=O)N4)ccn1 ; Cc1cc(-c2n[nH]c3cc4c(cc23)CN([C@@H]2CCCN(CCC3CC3)C2)C(=O)N4)ccn1 ; Cc1cc(-c2n[nH]c3cc4c(cc23)CN([C@@H]2CCCN(Cc3cccc(Cl)c3)C2)C(=O)N4)ccn1 ; Cc1cc(-c2n[nH]c3cc4c(cc23)CN(C2CCN(CCc3ccccc3)CC2)C(=O)N4)ccn1 ; Cc1cc(-c2n[nH]c3cc4c(cc23)CN([C@@H]2CCCN(Cc3ccccc3Cl)C2)C(=O)N4)ccn1 ; Cc1cc(-c2n[nH]c3cc4c(cc23)CN([C@@H]2CCCN(Cc3ccc(Cl)cc3)C2)C(=O)N4)ccn1]

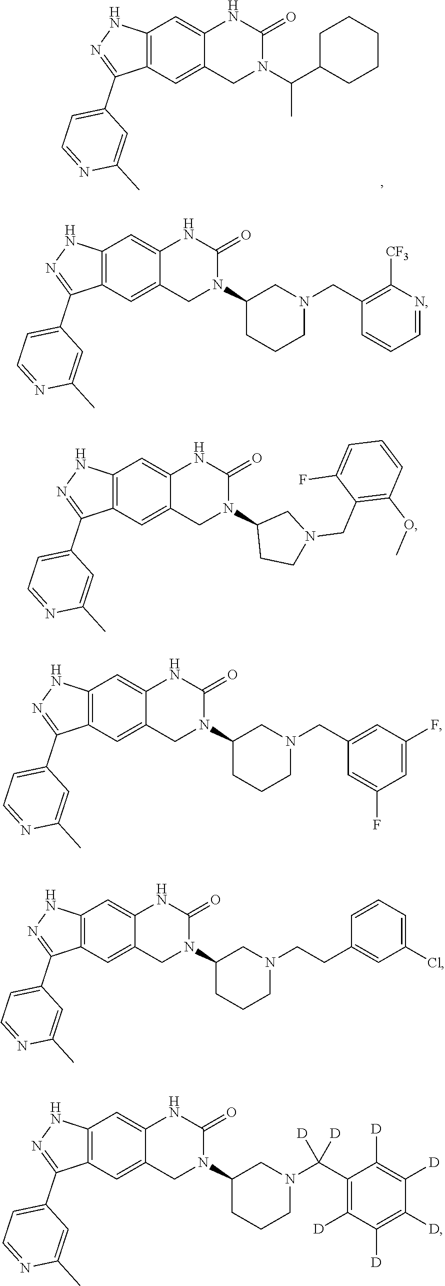

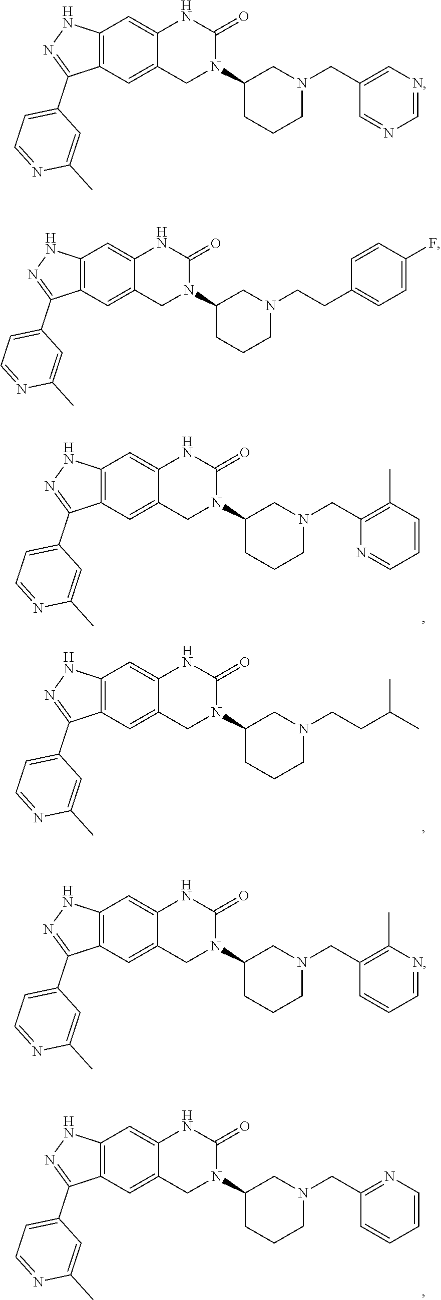 [SMILES: Cc1cc(-c2n[nH]c3cc4c(cc23)CN([C@@H]2CCCN(Cc3cncnc3)C2)C(=O)N4)ccn1 ; Cc1cc(-c2n[nH]c3cc4c(cc23)CN([C@@H]2CCCN(CCc3ccc(F)cc3)C2)C(=O)N4)ccn1 ; Cc1cc(-c2n[nH]c3cc4c(cc23)CN([C@@H]2CCCN(Cc3ncccc3C)C2)C(=O)N4)ccn1 ; Cc1cc(-c2n[nH]c3cc4c(cc23)CN([C@@H]2CCCN(CCC(C)C)C2)C(=O)N4)ccn1 ; Cc1cc(-c2n[nH]c3cc4c(cc23)CN([C@@H]2CCCN(Cc3cccnc3C)C2)C(=O)N4)ccn1 ; Cc1cc(-c2n[nH]c3cc4c(cc23)CN([C@@H]2CCCN(Cc3ccccn3)C2)C(=O)N4)ccn1]

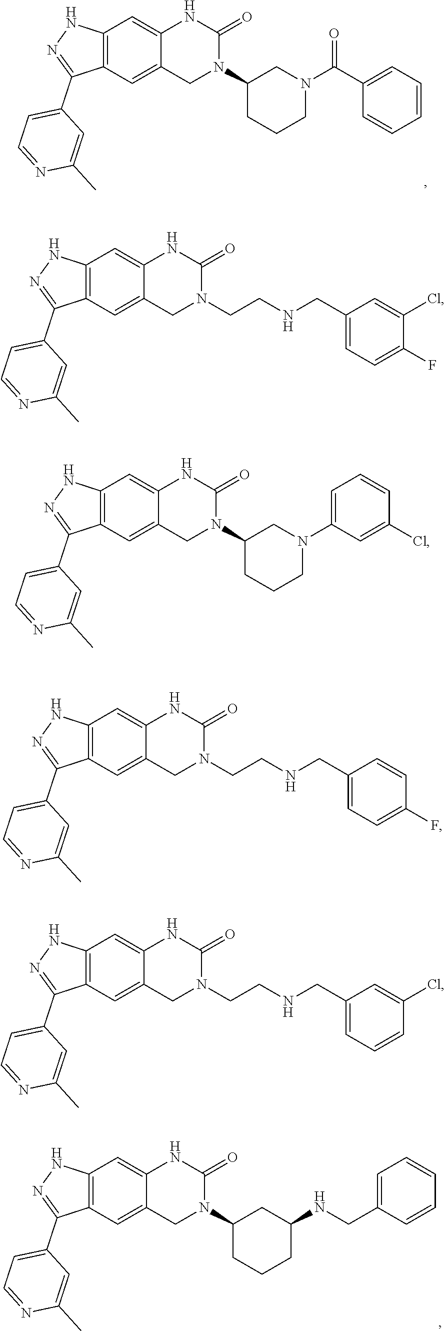 [SMILES: Cc1cc(-c2n[nH]c3cc4c(cc23)CN([C@@H]2CCCN(C(=O)c3ccccc3)C2)C(=O)N4)ccn1 ; Cc1cc(-c2n[nH]c3cc4c(cc23)CN(CCNCc2ccc(F)c(Cl)c2)C(=O)N4)ccn1 ; Cc1cc(-c2n[nH]c3cc4c(cc23)CN([C@@H]2CCCN(c3cccc(Cl)c3)C2)C(=O)N4)ccn1 ; Cc1cc(-c2n[nH]c3cc4c(cc23)CN(CCNCc2ccc(F)cc2)C(=O)N4)ccn1 ; Cc1cc(-c2n[nH]c3cc4c(cc23)CN(CCNCc2cccc(Cl)c2)C(=O)N4)ccn1 ; Cc1cc(-c2n[nH]c3cc4c(cc23)CN([C@@H]2CCC[C@H](NCc3ccccc3)C2)C(=O)N4)ccn1]

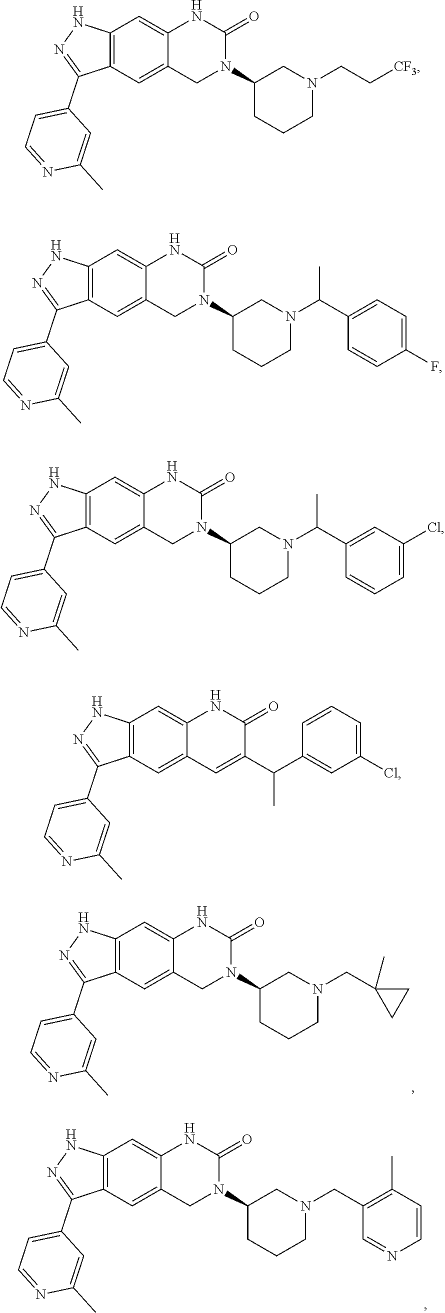 [SMILES: Cc1cc(-c2n[nH]c3cc4c(cc23)CN([C@@H]2CCCN(CCC(F)(F)F)C2)C(=O)N4)ccn1 ; Cc1cc(-c2n[nH]c3cc4c(cc23)CN([C@@H]2CCCN(C(C)c3ccc(F)cc3)C2)C(=O)N4)ccn1 ; Cc1cc(-c2n[nH]c3cc4c(cc23)CN([C@@H]2CCCN(C(C)c3cccc(Cl)c3)C2)C(=O)N4)ccn1 ; Cc1cc(-c2n[nH]c3cc4[nH]c(=O)c(C(C)c5cccc(Cl)c5)cc4cc23)ccn1 ; Cc1cc(-c2n[nH]c3cc4c(cc23)CN([C@@H]2CCCN(CC3(C)CC3)C2)C(=O)N4)ccn1 ; Cc1cc(-c2n[nH]c3cc4c(cc23)CN([C@@H]2CCCN(Cc3cnccc3C)C2)C(=O)N4)ccn1]

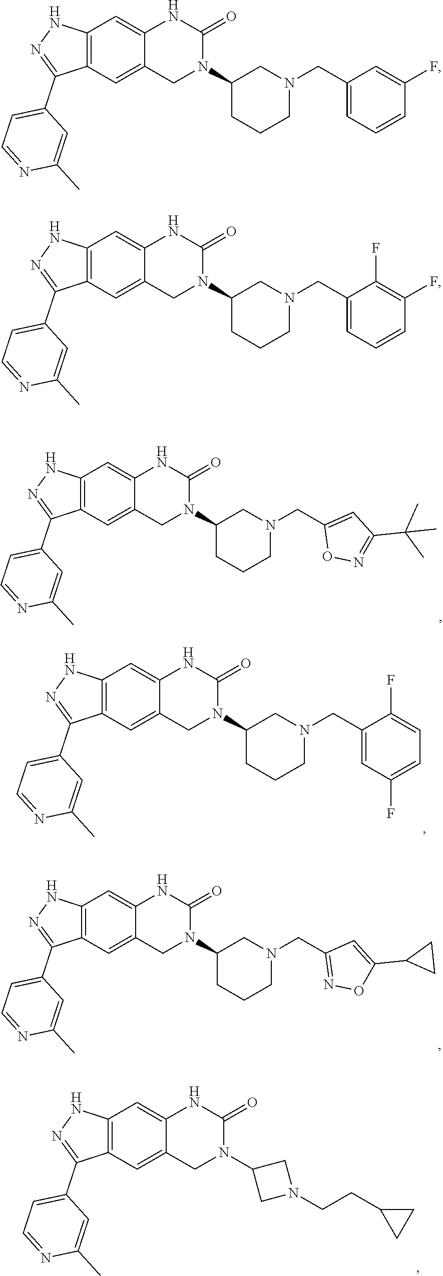

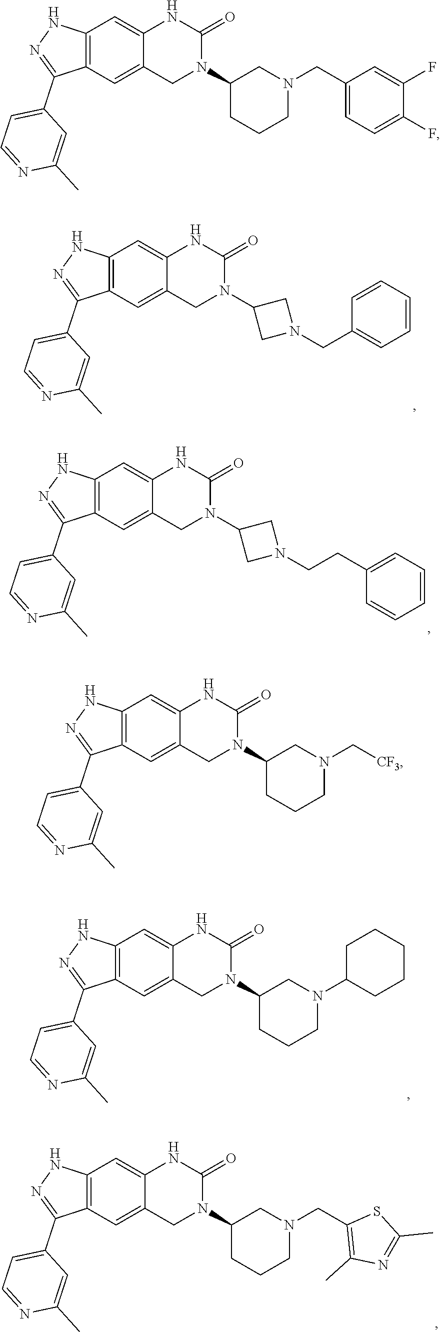 [SMILES: Cc1cc(-c2n[nH]c3cc4c(cc23)CN([C@@H]2CCCN(Cc3ccc(F)c(F)c3)C2)C(=O)N4)ccn1 ; Cc1cc(-c2n[nH]c3cc4c(cc23)CN(C2CN(Cc3ccccc3)C2)C(=O)N4)ccn1 ; Cc1cc(-c2n[nH]c3cc4c(cc23)CN(C2CN(CCc3ccccc3)C2)C(=O)N4)ccn1 ; Cc1cc(-c2n[nH]c3cc4c(cc23)CN([C@@H]2CCCN(CC(F)(F)F)C2)C(=O)N4)ccn1 ; Cc1cc(-c2n[nH]c3cc4c(cc23)CN([C@@H]2CCCN(C3CCCCC3)C2)C(=O)N4)ccn1 ; Cc1cc(-c2n[nH]c3cc4c(cc23)CN([C@@H]2CCCN(Cc3sc(C)nc3C)C2)C(=O)N4)ccn1]

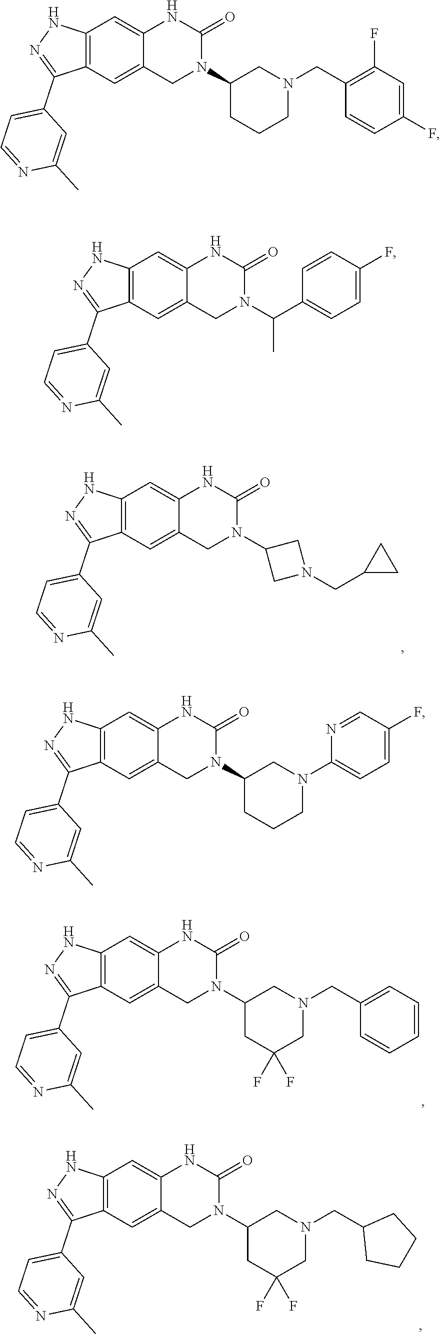 [SMILES: Cc1cc(-c2n[nH]c3cc4c(cc23)CN([C@@H]2CCCN(Cc3ccc(F)cc3F)C2)C(=O)N4)ccn1 ; Cc1cc(-c2n[nH]c3cc4c(cc23)CN(C(C)c2ccc(F)cc2)C(=O)N4)ccn1 ; Cc1cc(-c2n[nH]c3cc4c(cc23)CN(C2CN(CC3CC3)C2)C(=O)N4)ccn1 ; Cc1cc(-c2n[nH]c3cc4c(cc23)CN([C@@H]2CCCN(c3ccc(F)cn3)C2)C(=O)N4)ccn1 ; Cc1cc(-c2n[nH]c3cc4c(cc23)CN(C2CN(Cc3ccccc3)CC(F)(F)C2)C(=O)N4)ccn1 ; Cc1cc(-c2n[nH]c3cc4c(cc23)CN(C2CN(CC3CCCC3)CC(F)(F)C2)C(=O)N4)ccn1]

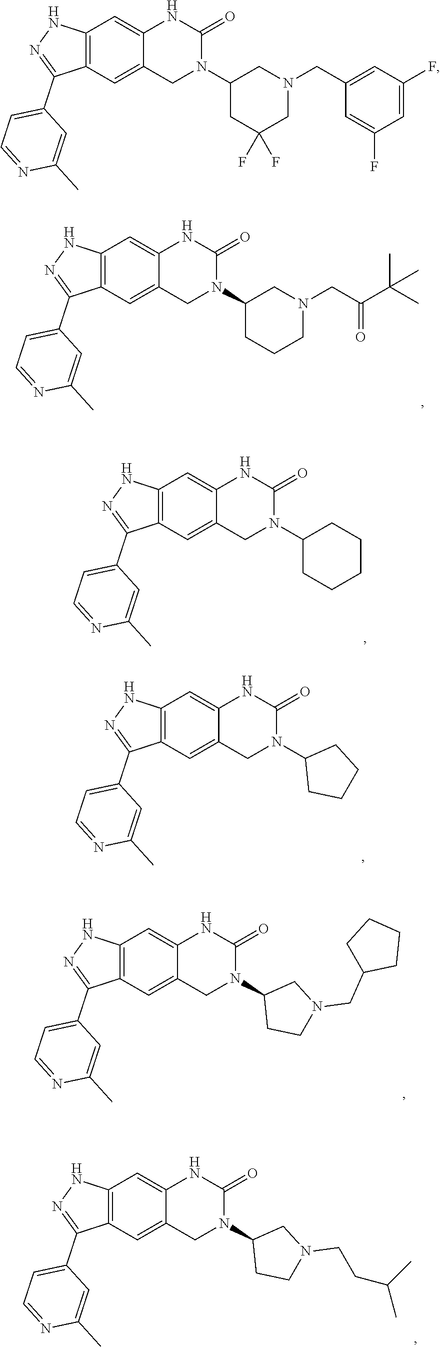 [SMILES: Cc1cc(-c2n[nH]c3cc4c(cc23)CN(C2CN(Cc3cc(F)cc(F)c3)CC(F)(F)C2)C(=O)N4)ccn1 ; Cc1cc(-c2n[nH]c3cc4c(cc23)CN([C@@H]2CCCN(CC(=O)C(C)(C)C)C2)C(=O)N4)ccn1 ; Cc1cc(-c2n[nH]c3cc4c(cc23)CN(C2CCCCC2)C(=O)N4)ccn1 ; Cc1cc(-c2n[nH]c3cc4c(cc23)CN(C2CCCC2)C(=O)N4)ccn1 ; Cc1cc(-c2n[nH]c3cc4c(cc23)CN([C@@H]2CCN(CC3CCCC3)C2)C(=O)N4)ccn1 ; Cc1cc(-c2n[nH]c3cc4c(cc23)CN([C@@H]2CCN(CCC(C)C)C2)C(=O)N4)ccn1]

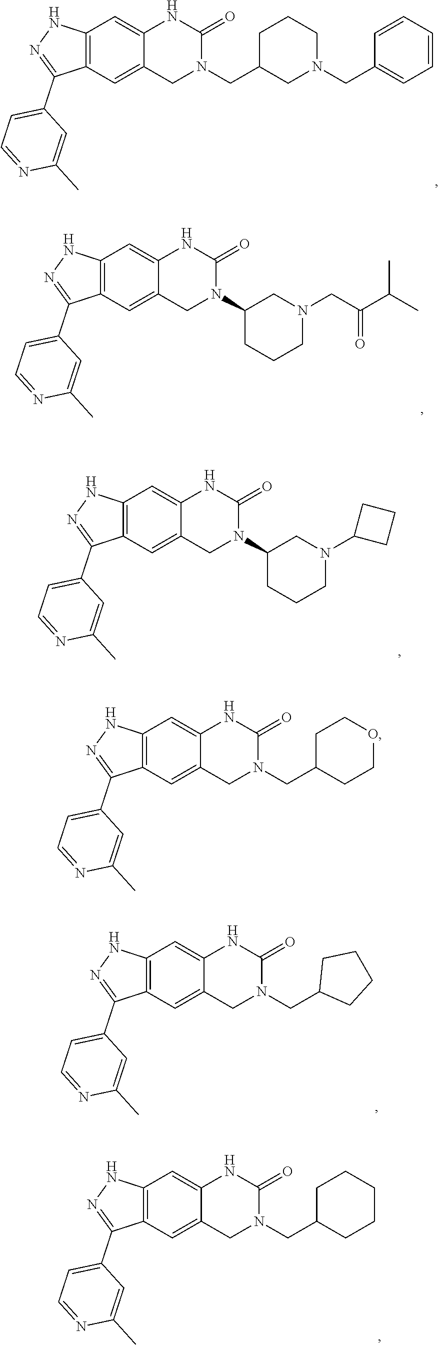 [SMILES: Cc1cc(-c2n[nH]c3cc4c(cc23)CN(CC2CCCN(Cc3ccccc3)C2)C(=O)N4)ccn1 ; Cc1cc(-c2n[nH]c3cc4c(cc23)CN([C@@H]2CCCN(CC(=O)C(C)C)C2)C(=O)N4)ccn1 ; Cc1cc(-c2n[nH]c3cc4c(cc23)CN([C@@H]2CCCN(C3CCC3)C2)C(=O)N4)ccn1 ; Cc1cc(-c2n[nH]c3cc4c(cc23)CN(CC2CCOCC2)C(=O)N4)ccn1 ; Cc1cc(-c2n[nH]c3cc4c(cc23)CN(CC2CCCC2)C(=O)N4)ccn1 ; Cc1cc(-c2n[nH]c3cc4c(cc23)CN(CC2CCCCC2)C(=O)N4)ccn1]

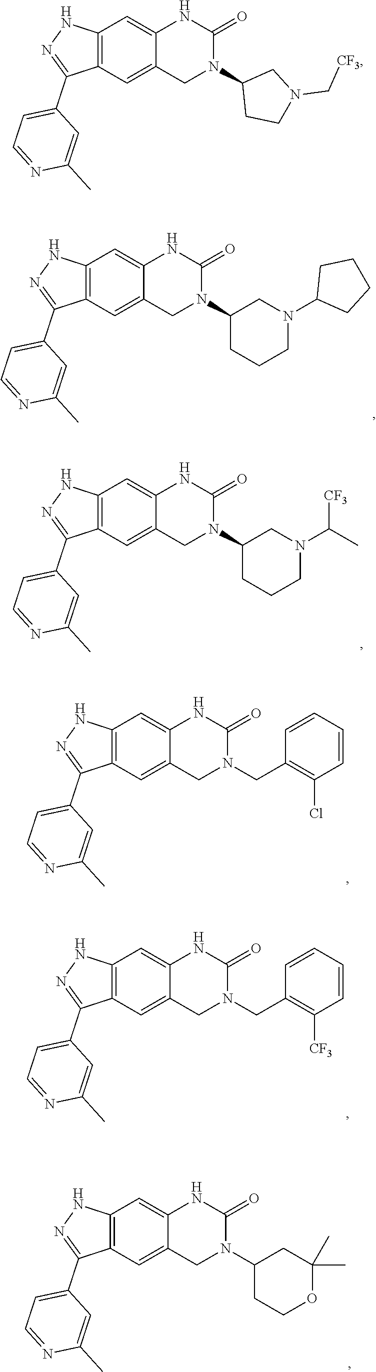 [SMILES: Cc1cc(-c2n[nH]c3cc4c(cc23)CN([C@@H]2CCN(CC(F)(F)F)C2)C(=O)N4)ccn1 ; Cc1cc(-c2n[nH]c3cc4c(cc23)CN([C@@H]2CCCN(C3CCCC3)C2)C(=O)N4)ccn1 ; Cc1cc(-c2n[nH]c3cc4c(cc23)CN([C@@H]2CCCN(C(C)C(F)(F)F)C2)C(=O)N4)ccn1 ; Cc1cc(-c2n[nH]c3cc4c(cc23)CN(Cc2ccccc2Cl)C(=O)N4)ccn1 ; Cc1cc(-c2n[nH]c3cc4c(cc23)CN(Cc2ccccc2C(F)(F)F)C(=O)N4)ccn1 ; Cc1cc(-c2n[nH]c3cc4c(cc23)CN(C2CCOC(C)(C)C2)C(=O)N4)ccn1]

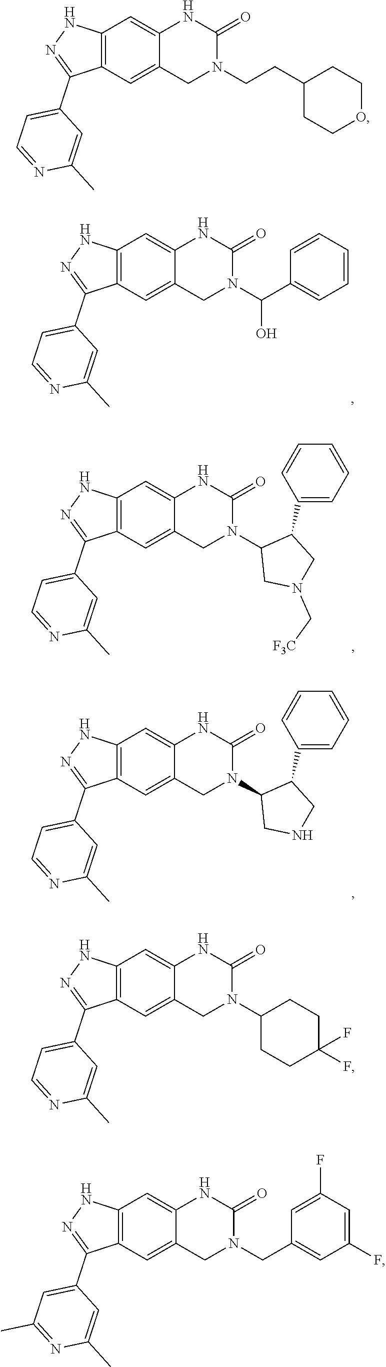

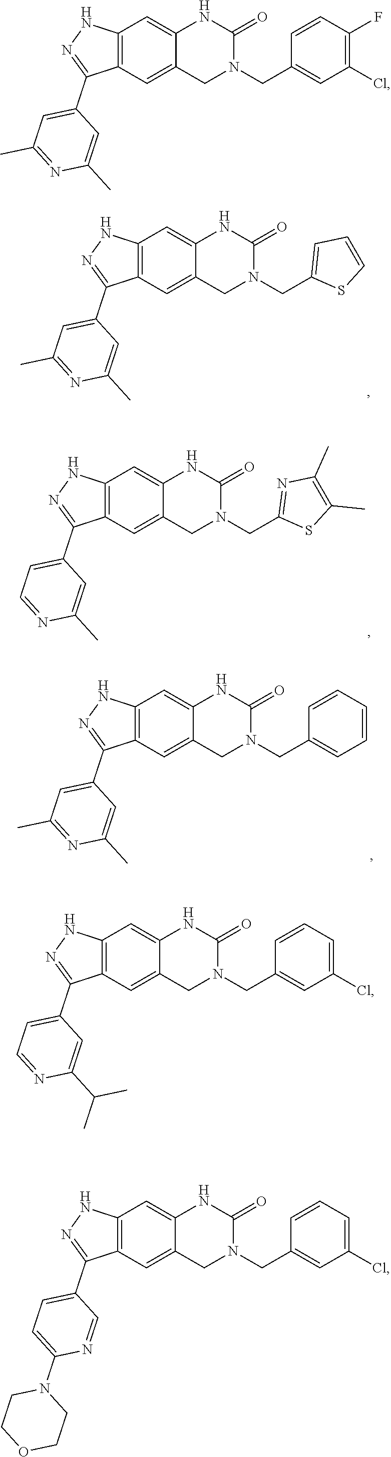

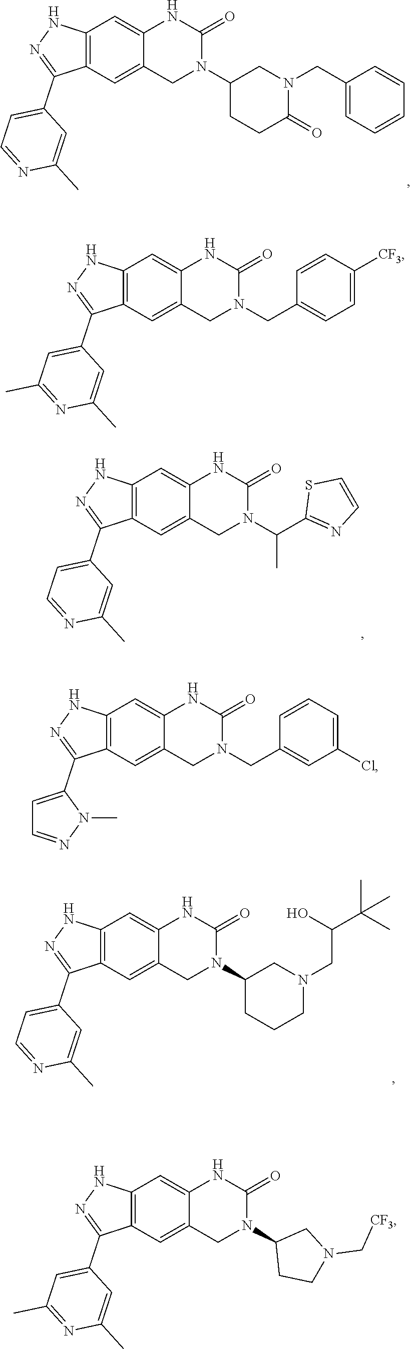

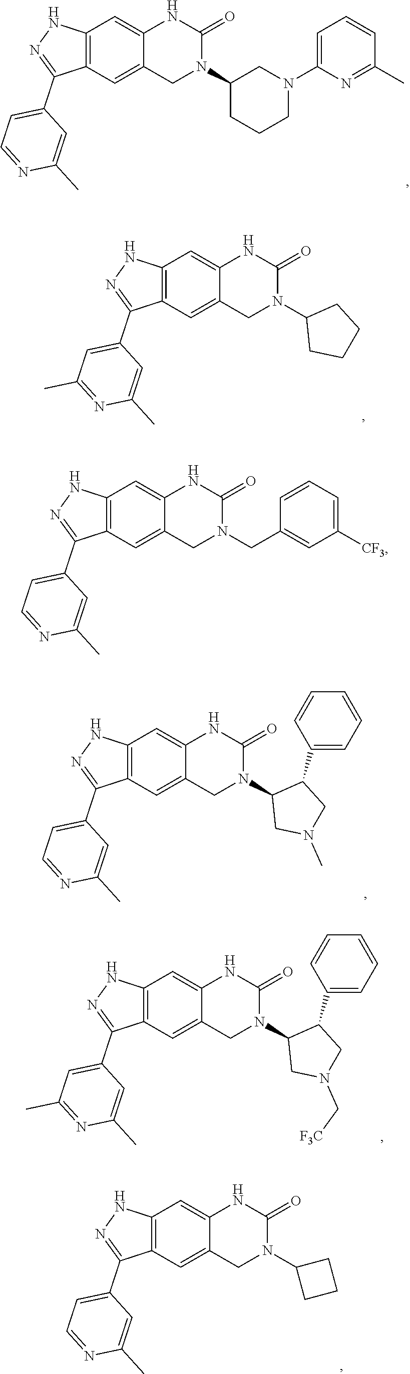

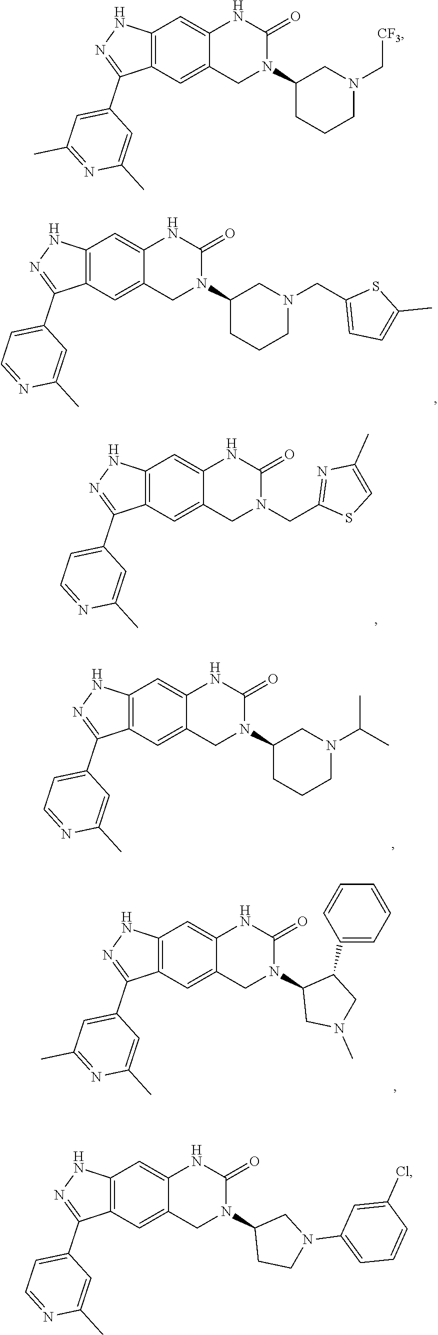

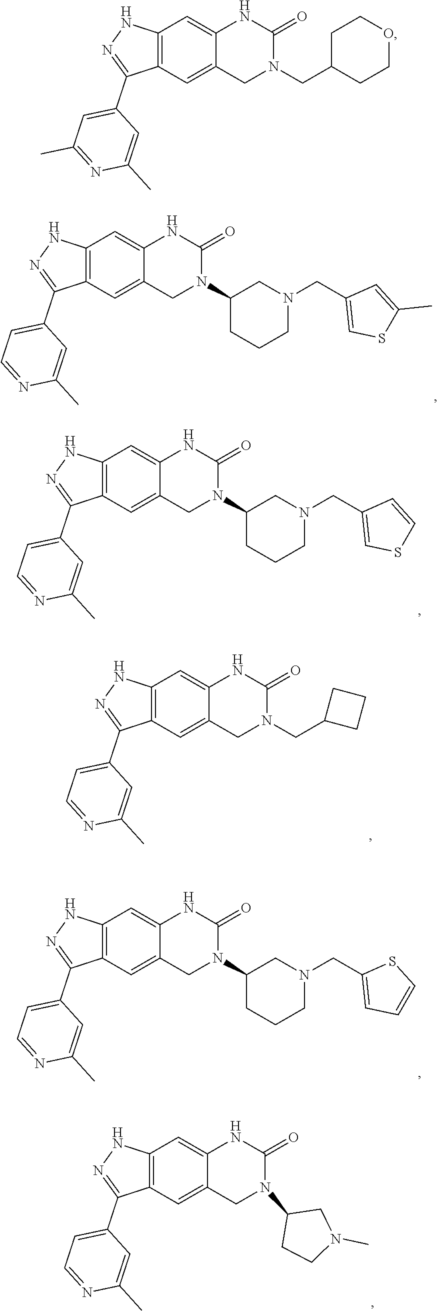 [SMILES: Cc1cc(-c2n[nH]c3cc4c(cc23)CN(CC2CCOCC2)C(=O)N4)cc(C)n1 ; Cc1cc(-c2n[nH]c3cc4c(cc23)CN([C@@H]2CCCN(Cc3csc(C)c3)C2)C(=O)N4)ccn1 ; Cc1cc(-c2n[nH]c3cc4c(cc23)CN([C@@H]2CCCN(Cc3ccsc3)C2)C(=O)N4)ccn1 ; Cc1cc(-c2n[nH]c3cc4c(cc23)CN(CC2CCC2)C(=O)N4)ccn1 ; Cc1cc(-c2n[nH]c3cc4c(cc23)CN([C@@H]2CCCN(Cc3cccs3)C2)C(=O)N4)ccn1 ; Cc1cc(-c2n[nH]c3cc4c(cc23)CN([C@@H]2CCN(C)C2)C(=O)N4)ccn1]

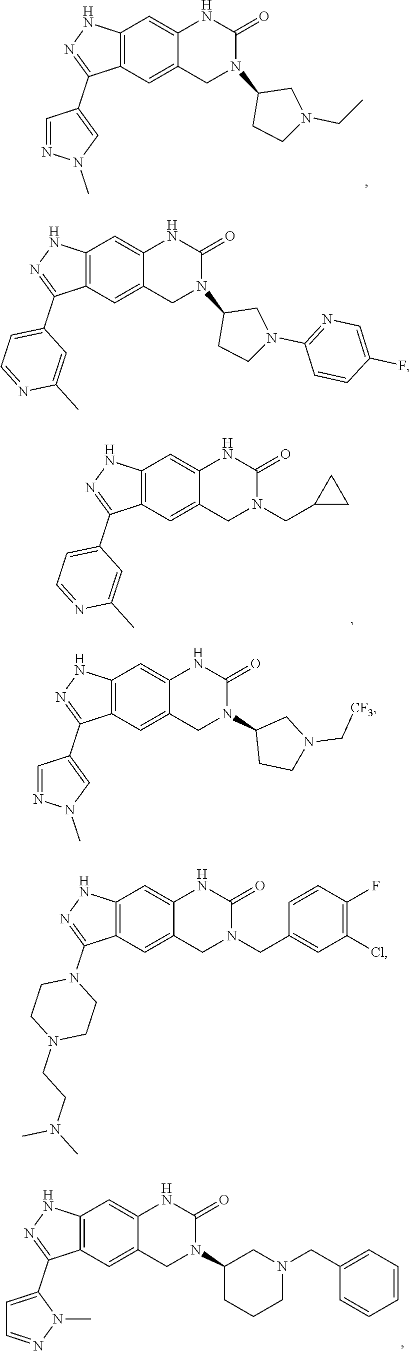

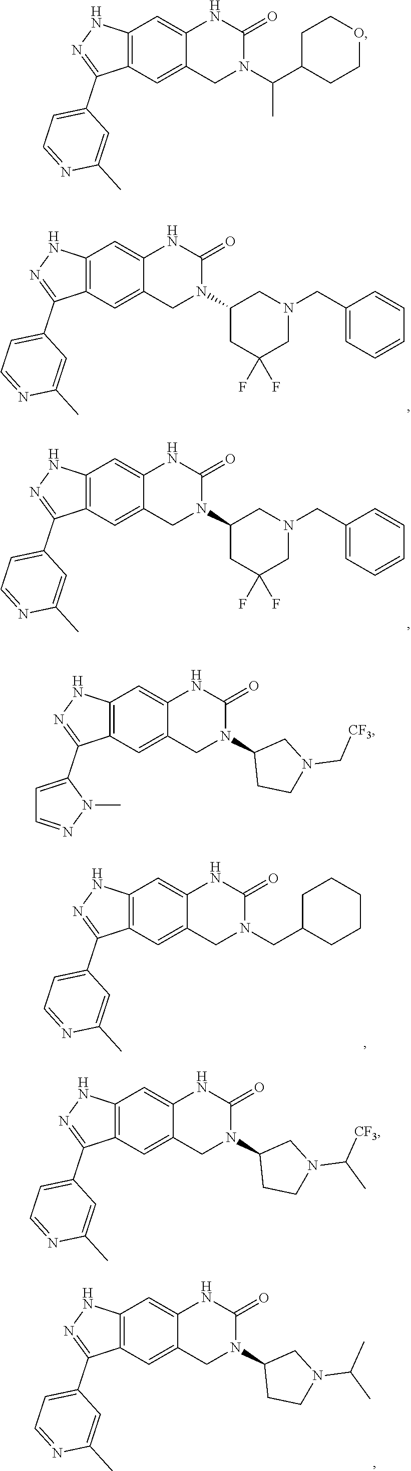

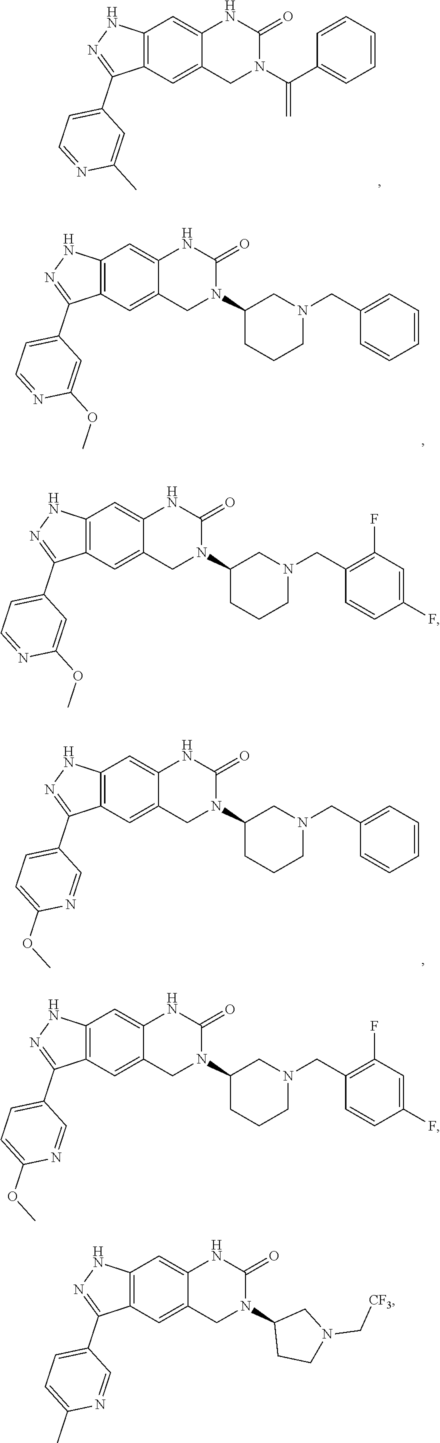 [SMILES: C=C(c1ccccc1)N1Cc2cc3c(-c4ccnc(C)c4)n[nH]c3cc2NC1=O ; COc1cc(-c2n[nH]c3cc4c(cc23)CN([C@@H]2CCCN(Cc3ccccc3)C2)C(=O)N4)ccn1 ; COc1cc(-c2n[nH]c3cc4c(cc23)CN([C@@H]2CCCN(Cc3ccc(F)cc3F)C2)C(=O)N4)ccn1 ; COc1ccc(-c2n[nH]c3cc4c(cc23)CN([C@@H]2CCCN(Cc3ccccc3)C2)C(=O)N4)cn1 ; COc1ccc(-c2n[nH]c3cc4c(cc23)CN([C@@H]2CCCN(Cc3ccc(F)cc3F)C2)C(=O)N4)cn1 ; Cc1ccc(-c2n[nH]c3cc4c(cc23)CN([C@@H]2CCN(CC(F)(F)F)C2)C(=O)N4)cn1]

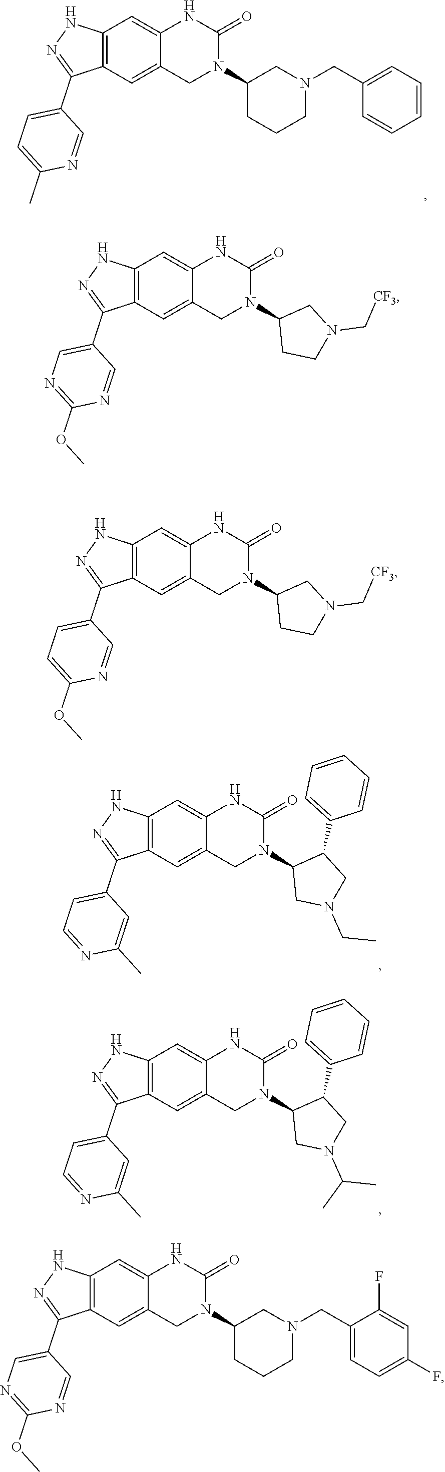 [SMILES: Cc1ccc(-c2n[nH]c3cc4c(cc23)CN([C@@H]2CCCN(Cc3ccccc3)C2)C(=O)N4)cn1 ; COc1ncc(-c2n[nH]c3cc4c(cc23)CN([C@@H]2CCN(CC(F)(F)F)C2)C(=O)N4)cn1 ; COc1ccc(-c2n[nH]c3cc4c(cc23)CN([C@@H]2CCN(CC(F)(F)F)C2)C(=O)N4)cn1 ; CCN1C[C@@H](N2Cc3cc4c(-c5ccnc(C)c5)n[nH]c4cc3NC2=O)[C@H](c2ccccc2)C1 ; Cc1cc(-c2n[nH]c3cc4c(cc23)CN([C@@H]2CN(C(C)C)C[C@H]2c2ccccc2)C(=O)N4)ccn1 ; COc1ncc(-c2n[nH]c3cc4c(cc23)CN([C@@H]2CCCN(Cc3ccc(F)cc3F)C2)C(=O)N4)cn1]

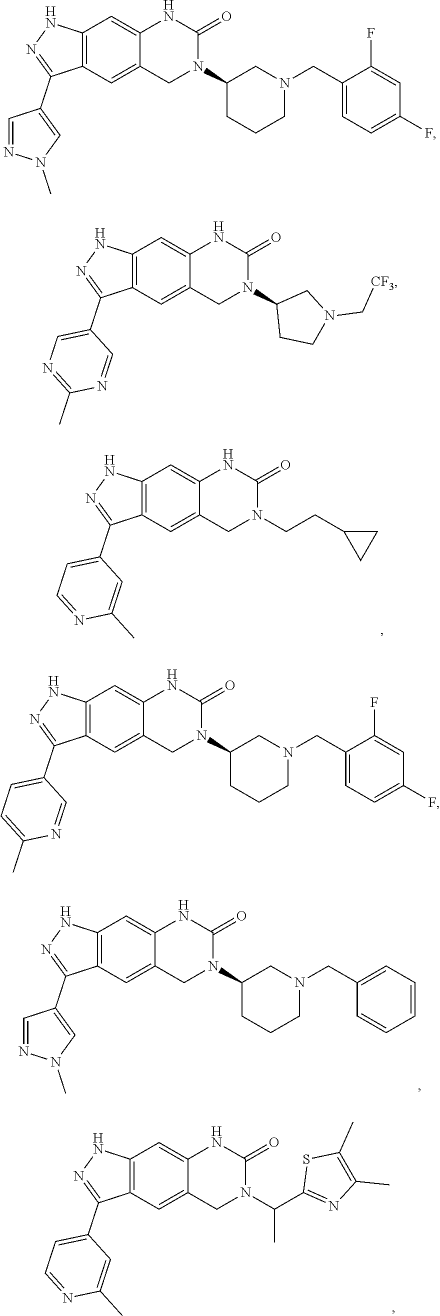 [SMILES: Cn1cc(-c2n[nH]c3cc4c(cc23)CN([C@@H]2CCCN(Cc3ccc(F)cc3F)C2)C(=O)N4)cn1 ; Cc1ncc(-c2n[nH]c3cc4c(cc23)CN([C@@H]2CCN(CC(F)(F)F)C2)C(=O)N4)cn1 ; Cc1cc(-c2n[nH]c3cc4c(cc23)CN(CCC2CC2)C(=O)N4)ccn1 ; Cc1ccc(-c2n[nH]c3cc4c(cc23)CN([C@@H]2CCCN(Cc3ccc(F)cc3F)C2)C(=O)N4)cn1 ; Cn1cc(-c2n[nH]c3cc4c(cc23)CN([C@@H]2CCCN(Cc3ccccc3)C2)C(=O)N4)cn1 ; Cc1cc(-c2n[nH]c3cc4c(cc23)CN(C(C)c2nc(C)c(C)s2)C(=O)N4)ccn1]

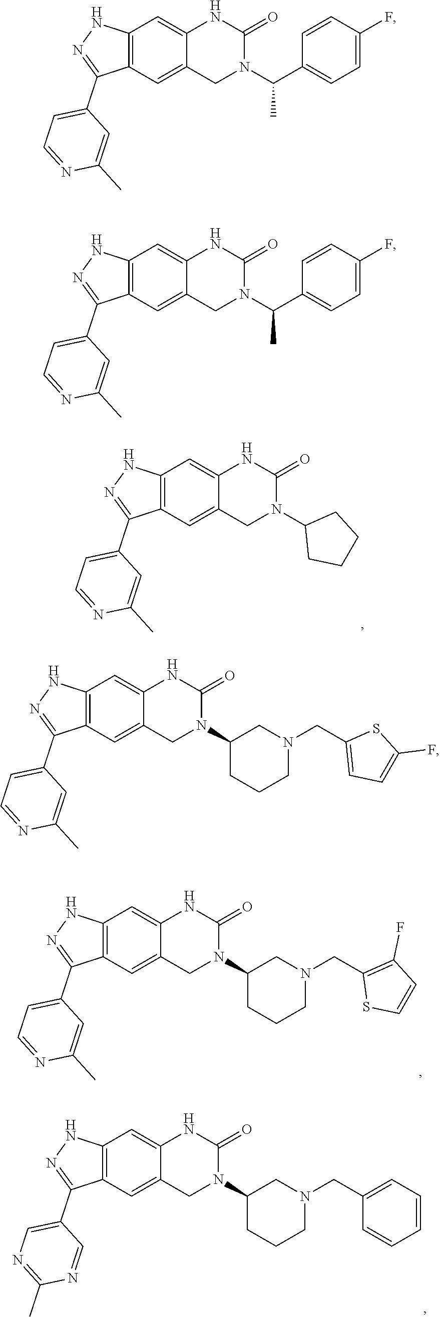 [SMILES: Cc1cc(-c2n[nH]c3cc4c(cc23)CN([C@@H](C)c2ccc(F)cc2)C(=O)N4)ccn1 ; Cc1cc(-c2n[nH]c3cc4c(cc23)CN([C@H](C)c2ccc(F)cc2)C(=O)N4)ccn1 ; Cc1cc(-c2n[nH]c3cc4c(cc23)CN(C2CCCC2)C(=O)N4)ccn1 ; Cc1cc(-c2n[nH]c3cc4c(cc23)CN([C@@H]2CCCN(Cc3ccc(F)s3)C2)C(=O)N4)ccn1 ; Cc1cc(-c2n[nH]c3cc4c(cc23)CN([C@@H]2CCCN(Cc3sccc3F)C2)C(=O)N4)ccn1 ; Cc1ncc(-c2n[nH]c3cc4c(cc23)CN([C@@H]2CCCN(Cc3ccccc3)C2)C(=O)N4)cn1]

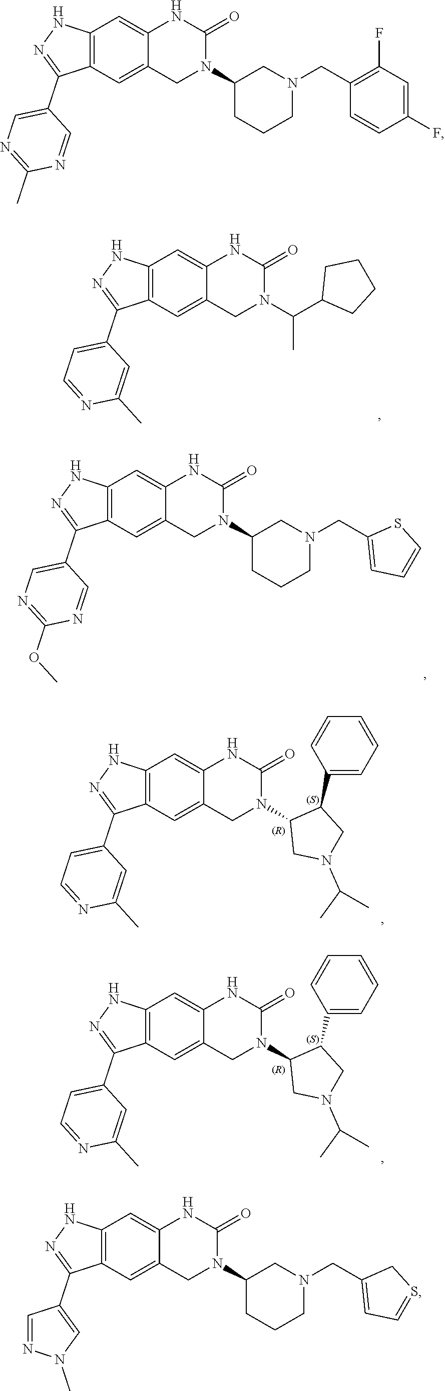

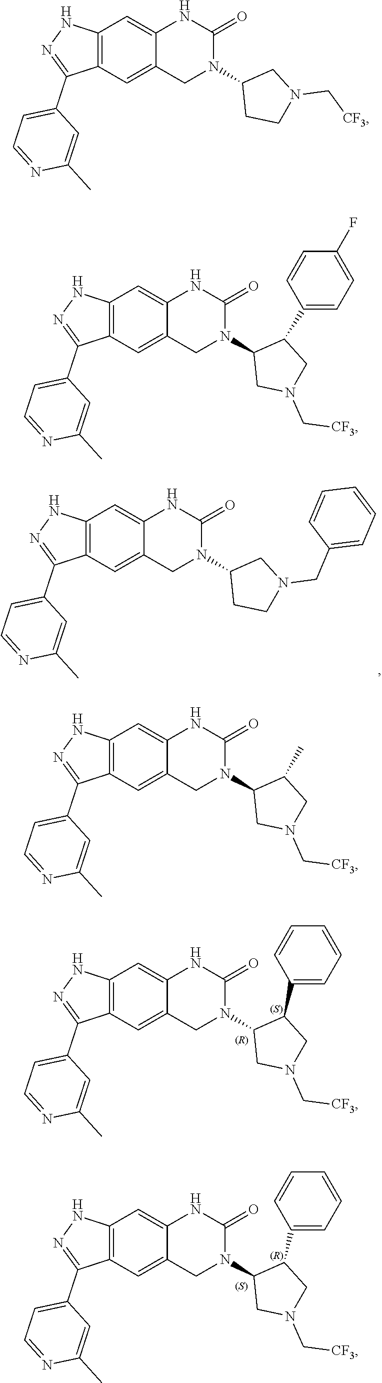

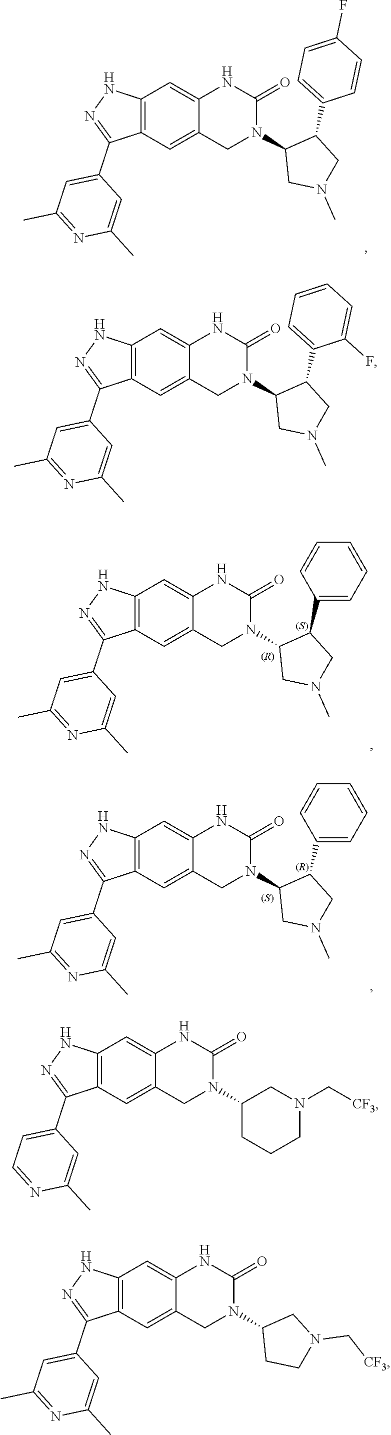

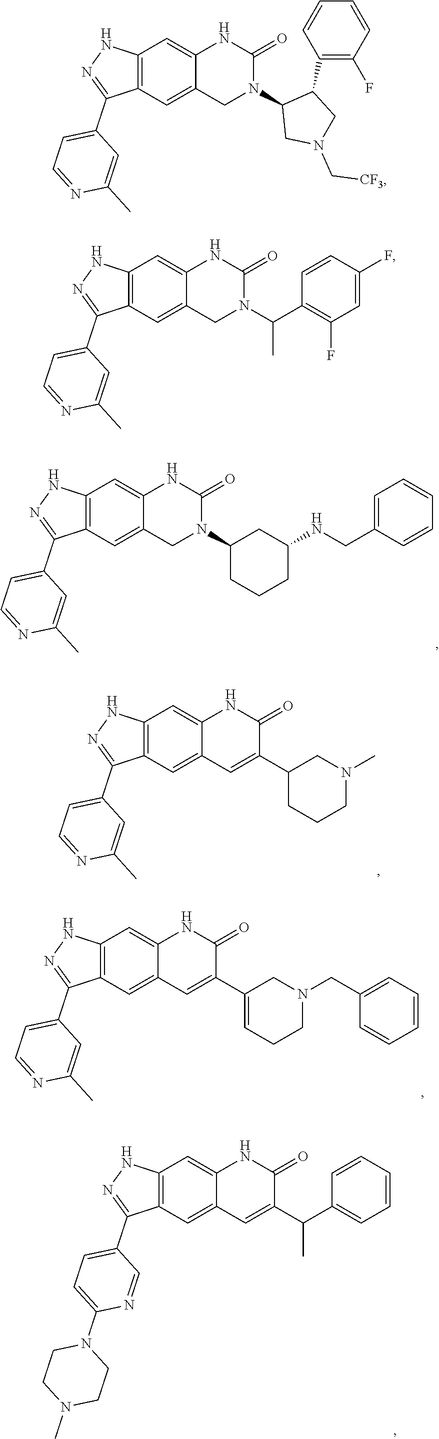 [SMILES: Cc1cc(-c2n[nH]c3cc4c(cc23)CN([C@@H]2CN(CC(F)(F)F)C[C@H]2c2ccccc2F)C(=O)N4)ccn1 ; Cc1cc(-c2n[nH]c3cc4c(cc23)CN(C(C)c2ccc(F)cc2F)C(=O)N4)ccn1 ; Cc1cc(-c2n[nH]c3cc4c(cc23)CN([C@@H]2CCC[C@@H](NCc3ccccc3)C2)C(=O)N4)ccn1 ; Cc1cc(-c2n[nH]c3cc4[nH]c(=O)c(C5CCCN(C)C5)cc4cc23)ccn1 ; Cc1cc(-c2n[nH]c3cc4[nH]c(=O)c(C5=CCCN(Cc6ccccc6)C5)cc4cc23)ccn1 ; CC(c1ccccc1)c1cc2cc3c(-c4ccc(N5CCN(C)CC5)nc4)n[nH]c3cc2[nH]c1=O]

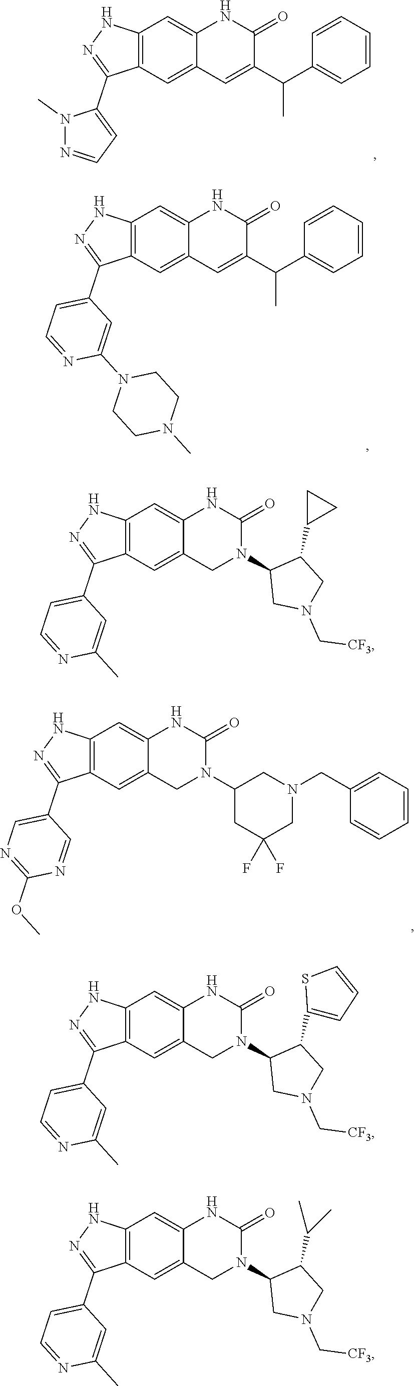

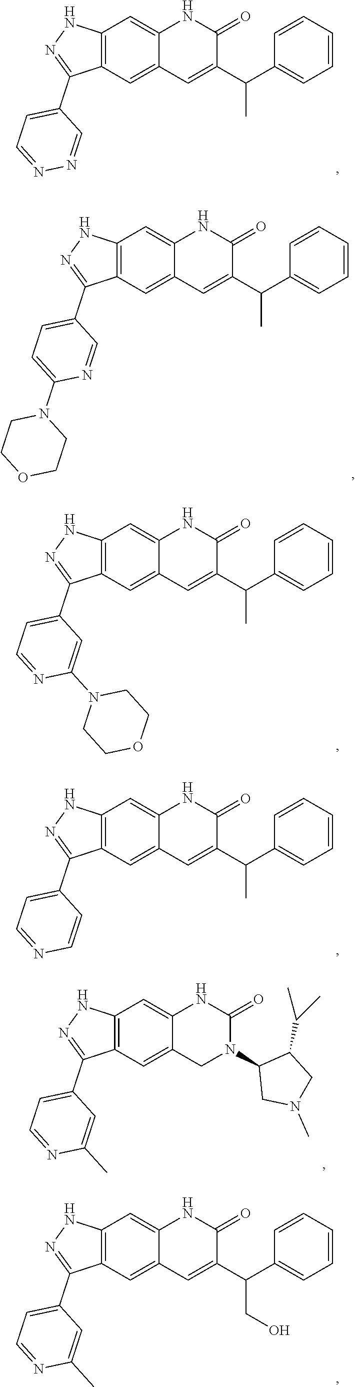 [SMILES: CC(c1ccccc1)c1cc2cc3c(-c4ccnnc4)n[nH]c3cc2[nH]c1=O ; CC(c1ccccc1)c1cc2cc3c(-c4ccc(N5CCOCC5)nc4)n[nH]c3cc2[nH]c1=O ; CC(c1ccccc1)c1cc2cc3c(-c4ccnc(N5CCOCC5)c4)n[nH]c3cc2[nH]c1=O ; CC(c1ccccc1)c1cc2cc3c(-c4ccncc4)n[nH]c3cc2[nH]c1=O ; Cc1cc(-c2n[nH]c3cc4c(cc23)CN([C@@H]2CN(C)C[C@H]2C(C)C)C(=O)N4)ccn1 ; Cc1cc(-c2n[nH]c3cc4[nH]c(=O)c(C(CO)c5ccccc5)cc4cc23)ccn1]

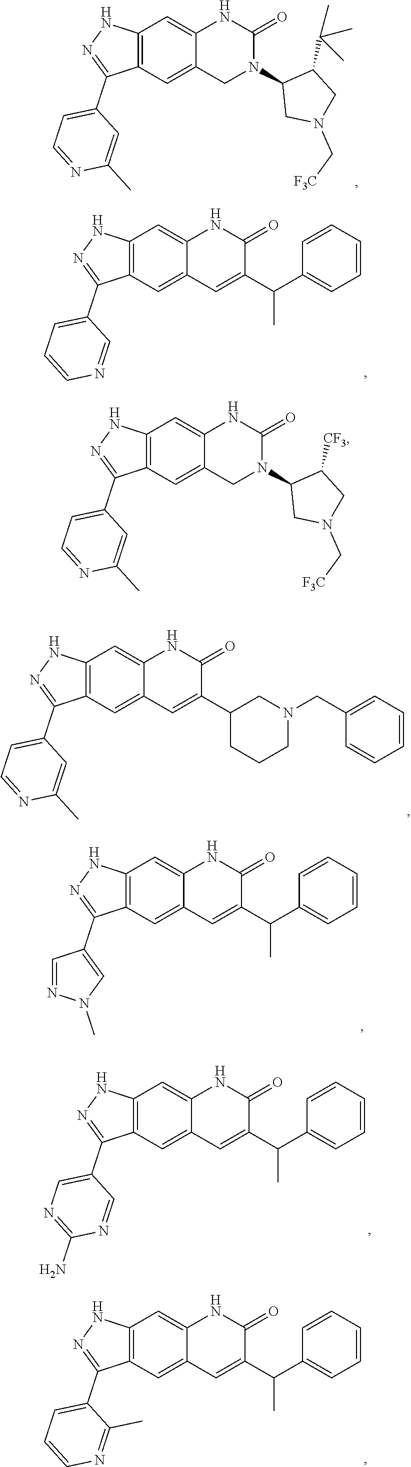

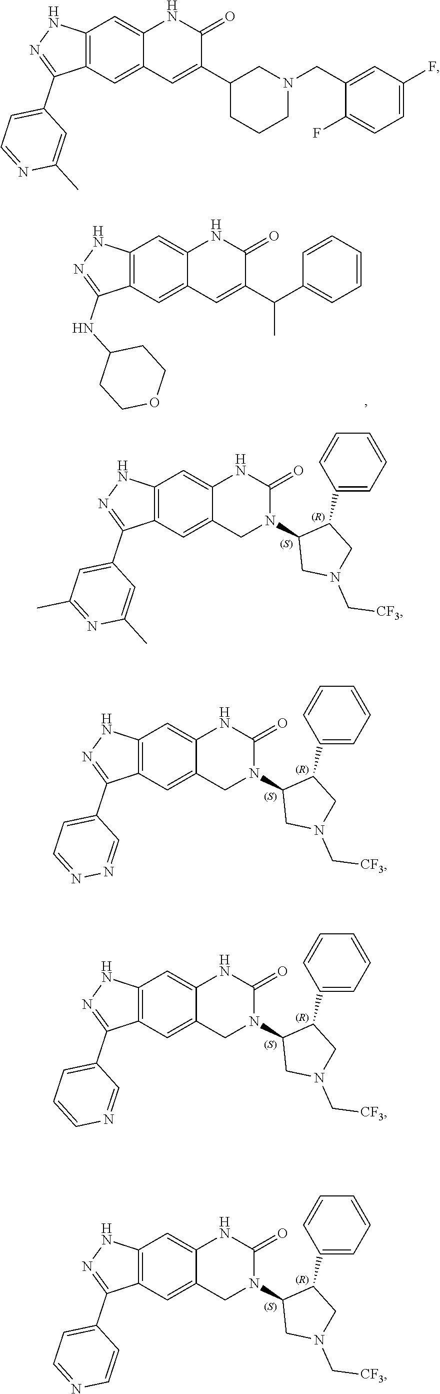

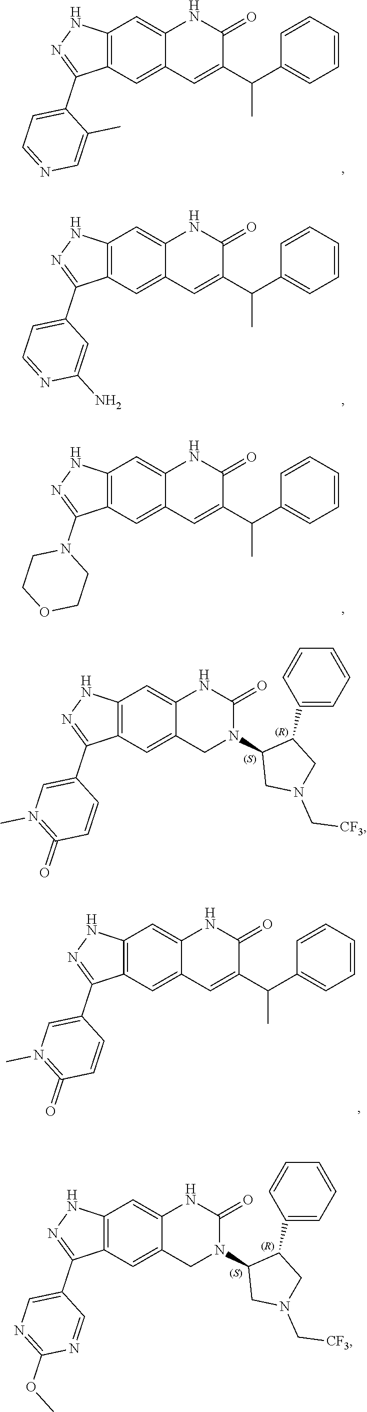

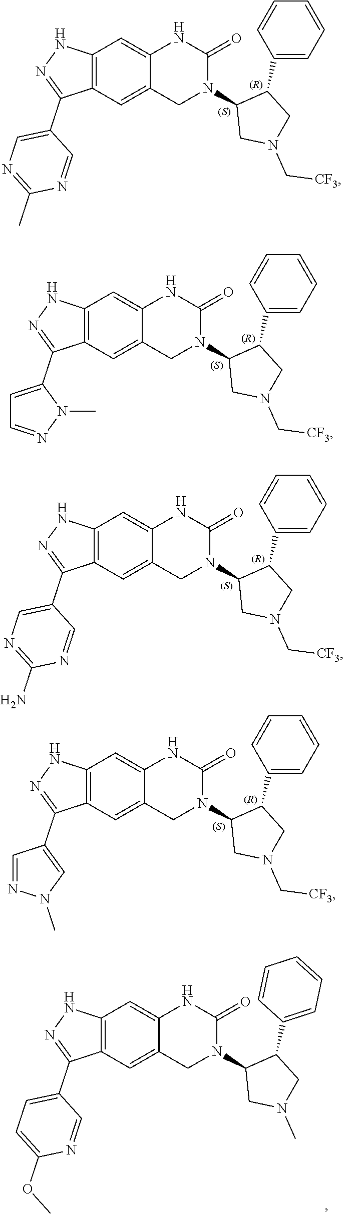

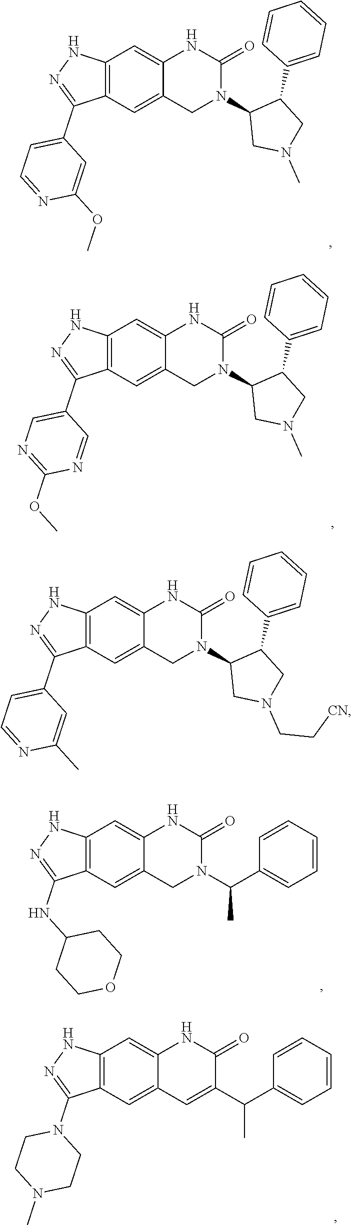 [SMILES: COc1cc(-c2n[nH]c3cc4c(cc23)CN([C@@H]2CN(C)C[C@H]2c2ccccc2)C(=O)N4)ccn1 ; COc1ncc(-c2n[nH]c3cc4c(cc23)CN([C@@H]2CN(C)C[C@H]2c2ccccc2)C(=O)N4)cn1 ; Cc1cc(-c2n[nH]c3cc4c(cc23)CN([C@@H]2CN(CCC#N)C[C@H]2c2ccccc2)C(=O)N4)ccn1 ; C[C@H](c1ccccc1)N1Cc2cc3c(NC4CCOCC4)n[nH]c3cc2NC1=O ; CC(c1ccccc1)c1cc2cc3c(N4CCN(C)CC4)n[nH]c3cc2[nH]c1=O]

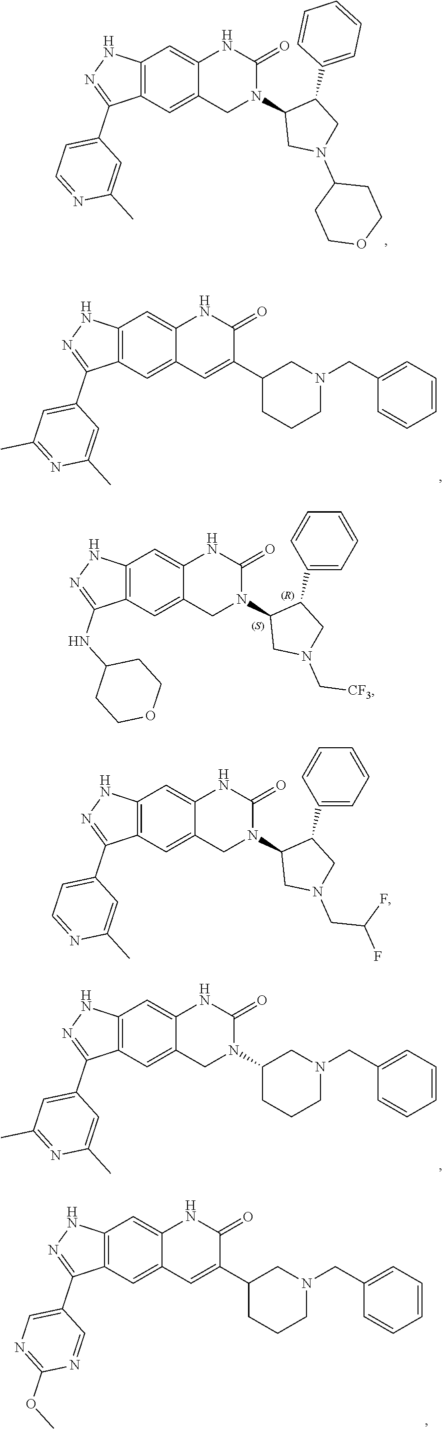

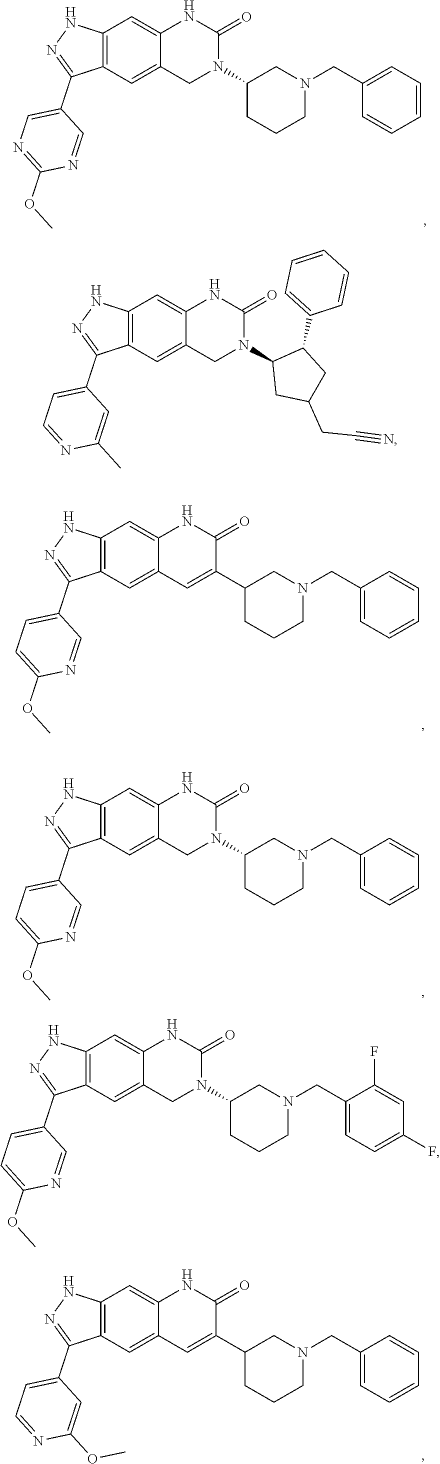

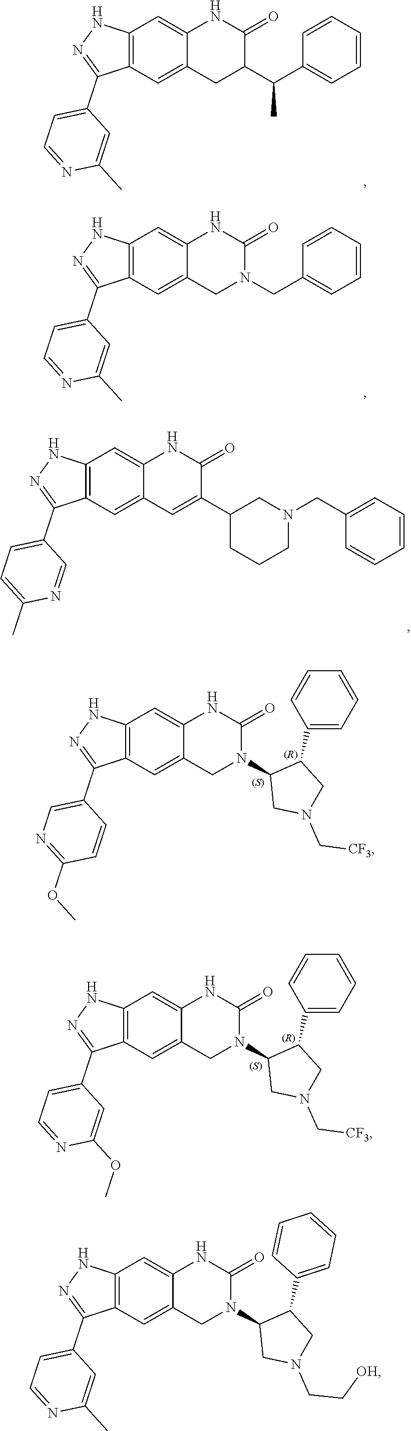

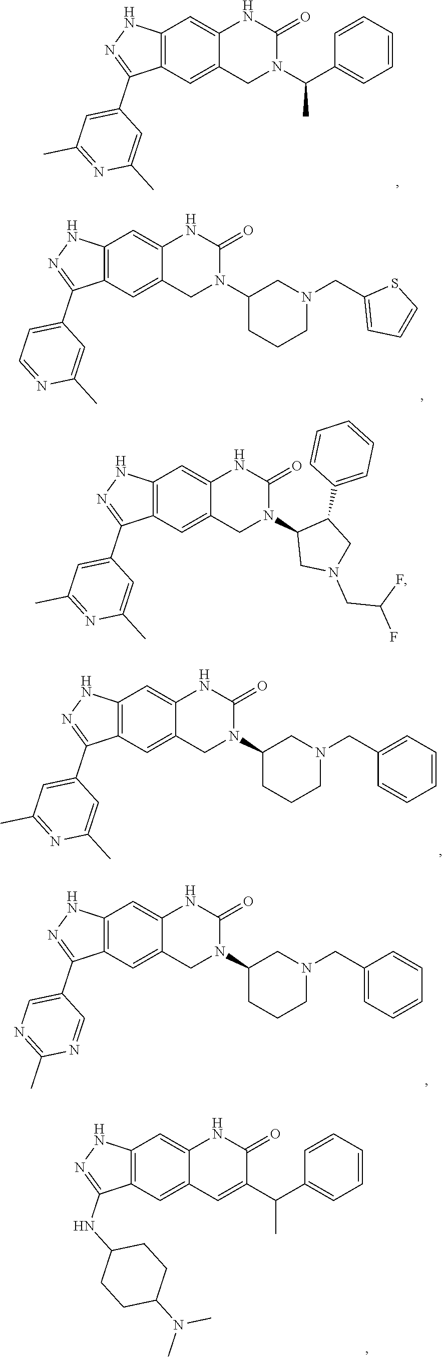

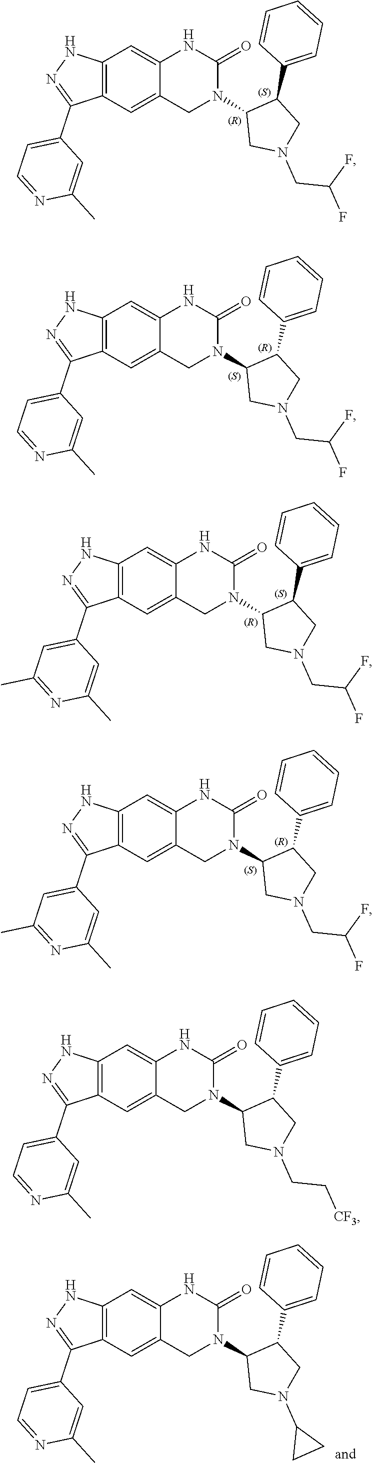

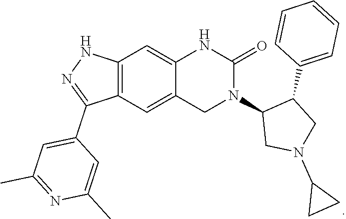 [SMILES: Cc1cc(-c2n[nH]c3cc4c(cc23)CN([C@@H]2CN(C3CC3)C[C@H]2c2ccccc2)C(=O)N4)cc(C)n1]

In certain embodiments, the present disclosure provides an ERK inhibitor which is a compound selected from the group consisting of:

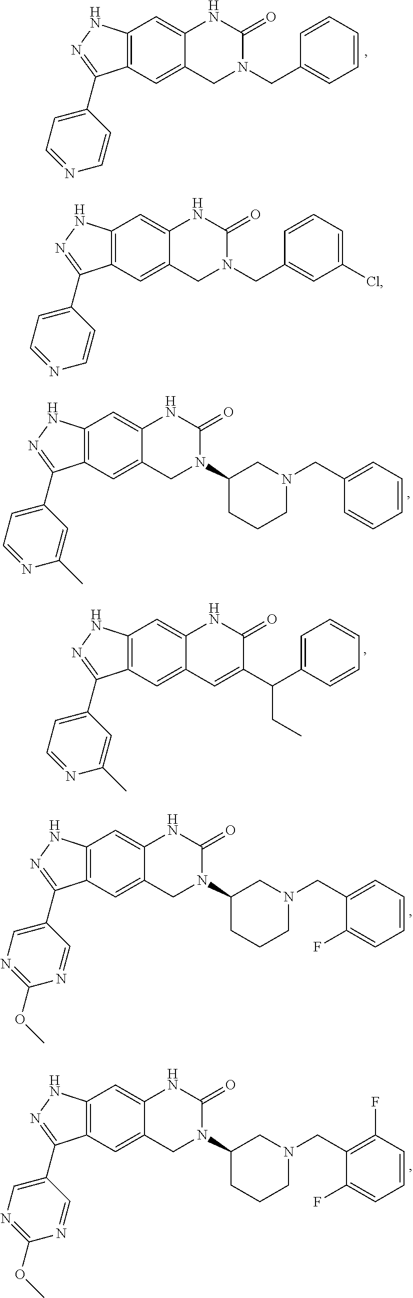

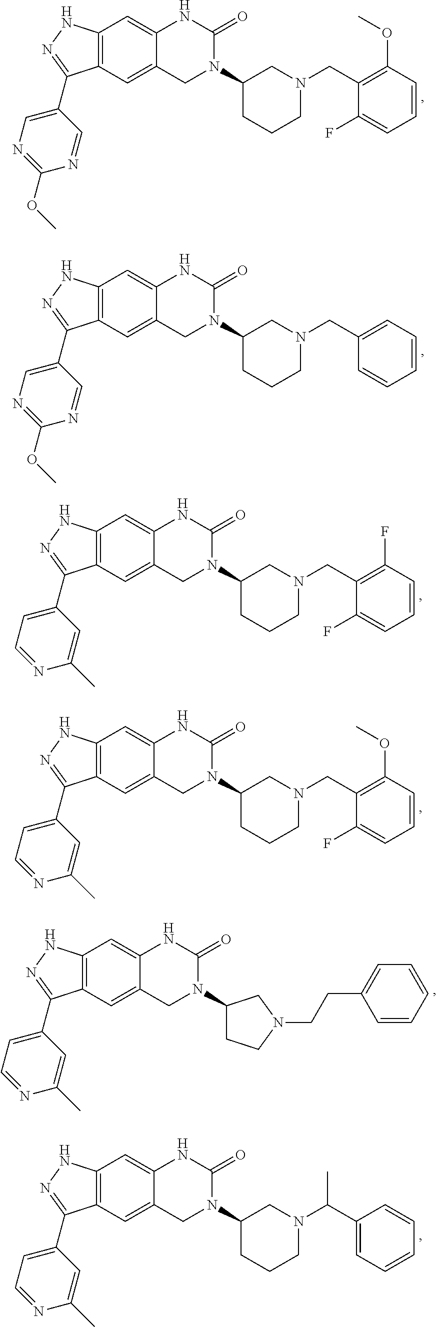

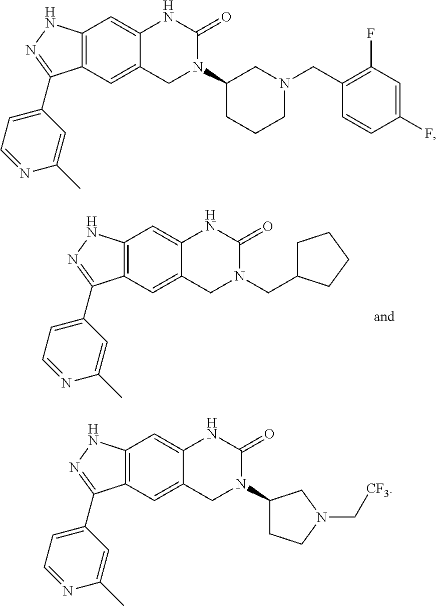

In certain embodiments, the present disclosure provides an ERK inhibitor which is a compound selected from the group consisting of:

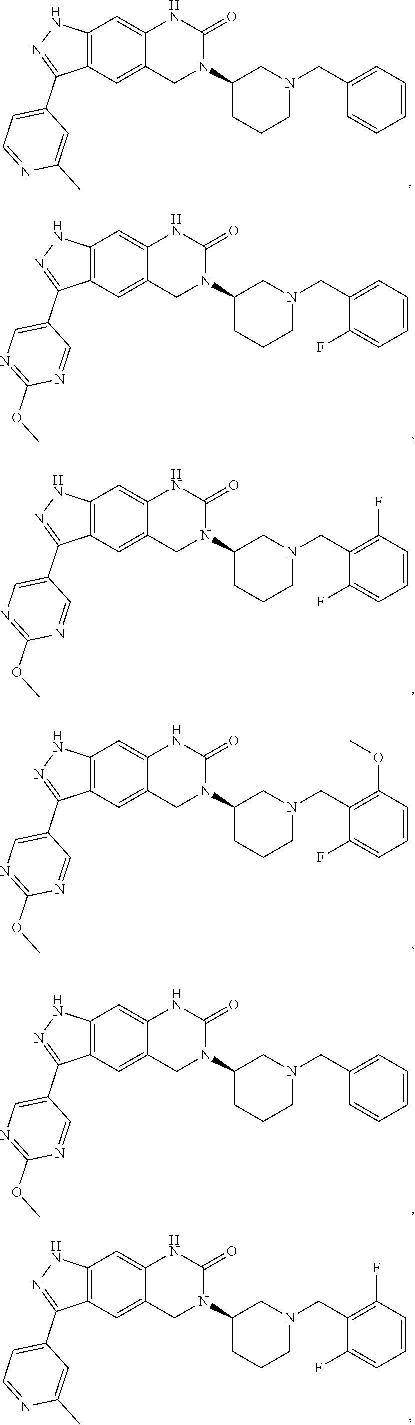

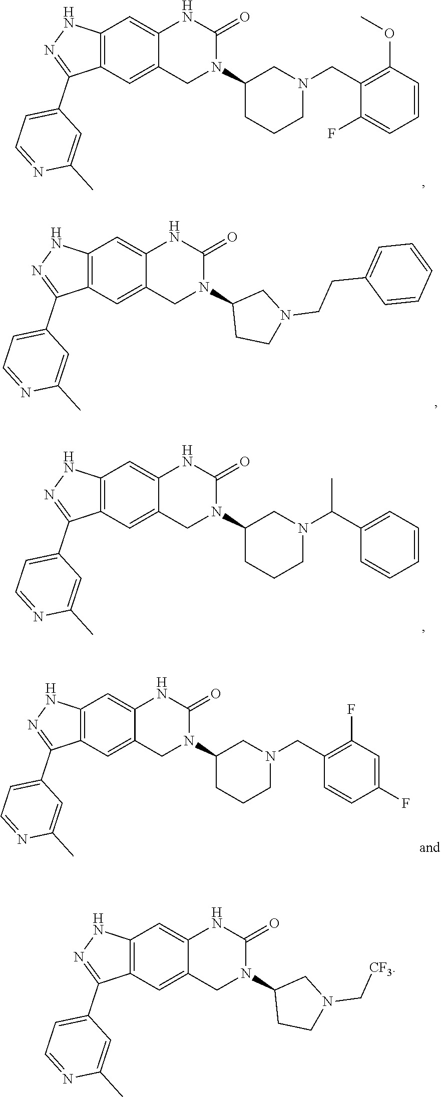 [SMILES: COc1cccc(F)c1CN1CCC[C@@H](N2Cc3cc4c(-c5ccnc(C)c5)n[nH]c4cc3NC2=O)C1 ; Cc1cc(-c2n[nH]c3cc4c(cc23)CN([C@@H]2CCN(CCc3ccccc3)C2)C(=O)N4)ccn1 ; Cc1cc(-c2n[nH]c3cc4c(cc23)CN([C@@H]2CCCN(C(C)c3ccccc3)C2)C(=O)N4)ccn1 ; Cc1cc(-c2n[nH]c3cc4c(cc23)CN([C@@H]2CCCN(Cc3ccc(F)cc3F)C2)C(=O)N4)ccn1 ; Cc1cc(-c2n[nH]c3cc4c(cc23)CN([C@@H]2CCN(CC(F)(F)F)C2)C(=O)N4)ccn1]

Compounds of the present disclosure also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹ H (protium), ²H (deuterium), and ³H (tritium). Protium is the most abundant isotope of hydrogen in nature. Enriching for deuterium may afford certain therapeutic advantages, such as increased in vivo half-life and/or exposure, or may provide a compound useful for investigating in vivo routes of drug elimination and metabolism. Isotopically-enriched compounds may be prepared by conventional techniques well known to those skilled in the art.

“Isomers” are different compounds that have the same molecular formula. “Stereoisomers” are isomers that differ only in the way the atoms are arranged in space. “Enantiomers” are a pair of stereoisomers that are non superimposable mirror images of each other. A 1:1 mixture of a pair of enantiomers is a “racemic: mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, chemical entities described herein are intended to include all Z-, E- and tautomeric forms as well.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

“Optional” or “optionally” means that the subsequently described event of circumstances may or may not occur, and that the description includes instances where the event or circumstance occurs and instances in which it does not. For example, “optionally substituted aryl” means that the aryl group may or may not be substituted and that the description includes both substituted aryl groups and aryl groups having no substitution.

“Pharmaceutically acceptable carrier, diluent or excipient” includes without limitation any adjuvant, carrier, excipient, glidant, sweetening agent, diluent, preservative, dye, colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

Any combination of the groups described above for the various variables is contemplated herein. Throughout the specification, groups and substituents thereof can be chosen to provide stable moieties and compounds.

The chemical entities described herein can be synthesized according to one or more illustrative schemes herein and/or techniques known in the art, for example, as described in PCT/US2014/059197, the disclosure of which is incorporated by reference herein. Materials used herein are either commercially available or prepared by synthetic methods generally known in the art.

The present disclosure provides a method of inhibiting the activity of one or more kinases of ERK (including ERK1 and ERK2) in a cell, comprising contacting the cell with an effective amount of one or more compounds disclosed herein. Inhibition of kinase activity can be assessed and demonstrated by a wide variety of ways known in the art. Non-limiting examples include (a) immunoblotting and immunoprecipitation with antibodies such as anti-phosphotyrosine, anti-phosphoserine or anti-phosphothreonine antibodies that recognize phosphorylated proteins; (b) using antibodies that specifically recognize a particular phosphorylated form of a kinase substrate (e.g. anti-phospho ERK); (c) cell proliferation assays, such as but not limited to tritiated thymidine uptake assays, BrdU (5′-bromo-2′-deoxyuridine) uptake (kit marketed by Calibochem), MTS uptake (kit marketed by Promega), MTT uptake (kit marketed by Cayman Chemical), CyQUANT® dye uptake (marketed by Invitrogen).

Selective PI3Kαinhibition may also be determined by expression levels of the PI3Kαgenes, its downstream signaling genes (for example by RT-PCR), or expression levels of the proteins (for example by immunocytochemistry, immunohistochemistry, Western blots) as compared to other PI3-kinases or protein kinases.

In some embodiments, the practice of a subject method involves a contacting step taking place in vitro. In other embodiments, the contacting step takes place in vivo.

Any of the compounds shown above may show a biological activity in an ERK inhibition assay of between about 1 pM and 25 μM (IC50).

In some embodiments, one or more compounds of the disclosure may bind specifically to an ERK (MAPK) kinase or a protein kinase selected from the group consisting of Ras, Raf, JNK, ErbB-1 (EGFR), Her2 (ErbB-2), Her 3 (ErbB-3), Her 4 (ErbB-4), MAP2K1 (MEK1), MAP2K2 (MEK2), MAP2K3 (MEK3), MAP2K4 (MEK4), MAP2K5 (MEK5), MAP2K6 (MEK6), MAP2K7 (MEK7), CDK1, CDK2, CDK3, CDK4, CDK5, CDK6, CDK7, CDK8, CDK9, CDK11 and any other protein kinases listed in the appended tables and figures, as well as any functional mutants thereof

In some embodiments, the IC50 of a compound of the disclosure for ERK1 and/or ERK2 is less than about 1μM, less than about 100 nM, less than about 50 nM, less than about 10 nM, less than 1 nM or even less than about 0.5 nM. In some embodiments, the IC50 of a compound of the disclosure for ERK is less than about 1μM, less than about 100 nM, less than about 50 nM, less than about 10 nM, less than 1 nM or even less than about 0.5 nM. In some embodiments, one or more compounds of the disclosure exhibit dual binding specificity and are capable of inhibiting an ERK kinase (e.g., ERK-1 kinase, ERK-2 kinase, etc.) as well as a protein kinase (e.g., Ras, Raf, Her-2, MEK1, etc.) with an IC50 value less than about 1 μM, less than about 100 nM, less than about 50 nM, less than about 10 nM, less than 1 nM or even less than about 0.5 nM. In some embodiments, one or more compounds of the disclosure may be capable of inhibiting kinases involved in the Ras-Raf-MEK-ERK pathway including, for example, Ras, Raf, INK, ErbB-1 (EGFR), Her2 (ErbB-2), Her3 (ErbB-3), Her4 (ErbB-4), MAP2K1 (MEK1), MAP2K2 (MEK2), MAP2K3 (MEK3), MAP2K4 (MEK4), MAP2K5 (MEK5), MAP2K6 (MEK6), MAP2K7 (MEK7), CDK1, CDK2, CDK3, CDK4, CDK5, CDK6, CDK7, CDK8, CDK9, CDK11, and functional mutants thereof. In some embodiments, the kinase is Ras, Raf, INK, ErbB-1 (EGFR), Her2 (ErbB-2), MAP2K1 (MEK1), CDK1, CDK2, CDK3, CDK4, CDK5, CDK6, or any other kinases listed in the tables and figures herein.

In still another embodiment, the compounds of the disclosure selectively inhibit ERK 1 and/or ERK2 activity relative to one or more protein kinases including but not limited to serine/threonine kinase such as DNA-PK and mTor. Such selective inhibition can be evidenced by, e.g., the IC50 value of the compound of the disclosure that can be ½, ⅓^{rd}, ¼^{th}, ⅕^{th}, 1/7^{th}, 1/10^{th}, 1/20^{th}, 1/25^{th}, 1/50^{th}, 1/100^{th}, 1/200^{th}, 1/300^{th}, 1/400^{th}, 1/500^{th}, 1/1000^{th}, 1/2000^{th} or less as compared to that of a reference protein kinase. In some instances, the compounds of the disclosure lack substantial cross-reactivity with at least about 100, 200, 300, or more protein kinases other than ERK1 or ERK2. The lack of substantial cross-reactivity with other non-ERK protein kinases can be evidenced by, e.g., at least 50%, 60%, 70%, 80%, 90% or higher kinase activity retained when the compound of the disclosure is applied to the protein kinase at a concentration of 1 μM, 5 μM, 10 μM or higher.

In some embodiments, one or more compounds of the disclosure selectively inhibits both ERK1 and ERK2 activity with an IC50 value of about 100 nM, 50 nM, 10 nM, 5 nM, 100 pM, 10 pM or even 1 pM, or less as ascertained in an in vitro kinase assay.

In some embodiments, one or more compounds of the disclosure competes with ATP for binding to ATP-binding site on ERK1 and/or ERK2. In some embodiments, one or more compounds of the disclosure binds to ERK1 and/or ERK2 at a site other than the ATP-binding site.

In some embodiments, one or more compounds of the disclosure is capable of inhibiting and/or otherwise modulating cellular signal transduction via one or more protein kinases or lipid kinases disclosed herein. For example, one or more compounds of the disclosure is capable of inhibiting or modulating the output of a signal transduction pathway. Output of signaling transduction of a given pathway can be measured by the level of phosphorylation, dephosphorylation, fragmentation, reduction, oxidation of a signaling molecule in the pathway of interest. In another specific embodiment, the output of the pathway may be a cellular or phenotypic output (e.g. modulating/inhibition of cellular proliferation, cell death, apoptosis, autophagy, phagocytocis, cell cycle progression, metastases, cell invasion, angiogenesis, vascularization, ubiquitination, translation, transcription, protein trafficking, mitochondrial function, golgi function, endoplasmic reticular function, etc). In some embodiments, one or more compounds of the disclosure is capable of, by way of example, causing apoptosis, causing cell cycle arrest, inhibiting cellular proliferation, inhibiting tumor growth, inhibiting angiogenesis, inhibiting vascularization, inhibiting metastases, and/or inhibiting cell invasion.

In some embodiments, one or more compounds of the disclosure causes apoptosis of said cell or cell cycle arrest. Cell cycle can be arrested at the G0/G1 phase, S phase, and/or G2/M phase by the subject compounds.

In some embodiments, one or more compounds of the disclosure including but not limited to the compounds listed above are capable of inhibiting cellular proliferation. For example, in some cases, one or more compounds of the disclosure may inhibit proliferation of tumor cells or tumor cell lines with a wide range of genetic makeup. In some cases, the compounds of the disclosure may inhibit PC3 cell proliferation in vitro or in an in vivo model such as a xenograft mouse model. In some cases, in vitro cultured PC3 cell proliferation may be inhibited with an IC50 of less than 100 nM, 75 nM, 50 nM, 25 nM, 15 nM, 10 nM, 5 nM, 3 nM, 2 nM, 1 nM, 0.5 nM, 0.1 nM or less by one or more compounds of the disclosure.

In some embodiments, proliferation of primary tumors derived from subjects (e.g. cancer patients) can be inhibited by a compound of the disclosure as shown by in vitro assays, or in vivo models (e.g. using the subjects' tumor cells for generating a xenograft mode). In some cases primary tumor cell line proliferation may be inhibited with an IC50 of less than 100 nM, 75 nM, 50 nM, 25 nM, 15 nM, 10 nM, 5 nM, 3 nM, 2 nM, 1 nM, 0.5 nM, 0.1 nM or even less by one or more compounds of the disclosure. In some cases, the average IC50 of a compound of the disclosure for inhibiting a panel 10, 20, 30, 40, 50, 100 or more primary tumor cells may be about 200 nM, 100 nM, 75 nM, 50 nM, 25 nM, 15 nM, 10 nM, 5 nM, 3 nM, 2 nM, 1 nM, 0.5 nM, 0.1 nM or even less. The tumor cells that can be inhibited by the compounds of the present disclosure include but are not limited to squamous cell carcinomas, such as squamous cell carcinomas of the lung, esophagus, head and neck, and cervix.

In some embodiments, the compounds of the disclosure are effective in blocking cell proliferation signals in cells. In some cases, cell proliferation signaling may be inhibited by one or more compounds of the disclosure as evidenced by Western blot analysis of phosphorylation of proteins such as FOXO1 (phosphorylation at T24/3a T32), GSK3I3(phosphorylation at S9), PRAS40 (phosphorylation at T246), or MAPK phosphorylation. In some cases, the compounds of the disclosure can inhibit phosphorylation of signaling proteins and suppress proliferation of cells containing these signaling proteins but are resistant to existing chemotherapeutic agents including but not limited to rapamycin, Gleevec, dasatinib, alkylating agents, antimetabolites, anthracyclines, plant alkaloids, topoisomerase inhibitors and other antitumor agents disclosed herein.

In some embodiments, one or more compounds of the disclosure may cause cell cycle arrest. In some cases, cells treated with one or more compounds of the disclosure may arrest or take longer to proceed through one or more cell cycle stages such as G0/G1, S, or G2/M. For example, cells treated with one or more compounds of the disclosure may arrest or take longer to proceed through the G0/G1 cell cycle stage. In some cases, about 35%, 40%, 50%, 55%, 60%, 65%, 70% or more of cells treated with one or more compounds of the disclosure may be in the G0/G1 cell cycle stage. In some cases, cells exhibiting cell cycle arrest in the G0/G1 cell cycle stage in response to treatment with the compounds of the disclosure are tumor cells or rapidly dividing cells. In some cases, the compounds of the disclosure affect a comparable or a greater degree of G0/G1 arrest as compared to doxorubicin.

In some embodiments, a method of the present disclosure relates to the treatment of a disease or a condition that is resistant to a Ras, Raf and/or MEK inhibitor. For example, the disease can be a squamous cell carcinoma that is resistant to a B-Raf and/or MEK inhibitor.

In certain aspects, the present disclosure provides a method of treating cancer in a subject in need thereof, comprising administering an effective dose of an inhibitor of an extracellular signal-regulated kinase (ERK) to the subject, wherein the subject exhibits resistance to a treatment with a Ras, Raf or MEK inhibitor. Optionally, the method comprises screening the subject, or a cancer cell isolated from the subject, for resistance to a treatment with a Ras, Raf or MEK inhibitor. In some embodiments, the method comprises administering an ERK inhibitor to the subject if the subject or cancer cell isolated from the subject is determined to be resistant to a treatment with the Ras, Raf or MEK inhibitor.

In some embodiments, the subject exhibits resistance to a treatment with a B-Raf inhibitor. The B-Raf inhibitor may be selected from vemurafenib, GDC-0879, PLX-4720, PLX-3603, PLX-4032, RAF265, XL281, AZ628, sorafenib, dabrafenib and LGX818, such as vemurafenib. In some embodiments, the subject exhibits resistance to a treatment with an MEK inhibitor. The MEK inhibitor may be selected from trametinib, cobimetinib, binimetinib, selumetinib, PD-325901, CI-1040, PD-035901, TAK-733, PD98059, PD184352, U0126, RDEA119, AZD8330, RO4987655, RO4927350, R05068760, AS703026 and E6201, such as trametinib.

In some embodiments, a cancer of the subject methods comprises a B-Raf or N-Ras mutation. The cancer may be selected from breast cancer, pancreatic cancer, lung cancer, thyroid cancer, seminomas, melanoma, bladder cancer, liver cancer, kidney cancer, myelodysplastic syndrome, acute myelogenous leukemia and colorectal cancer. In some embodiments, the cancer is selected from pancreatic cancer, lung cancer, melanoma and colorectal cancer, such as melanoma.

In certain aspects, the present disclosure provides a method of inhibiting growth of a cancer cell, the method comprising administering to the cell an ERK inhibitor, wherein the cell exhibits resistance to a treatment with a Ras, Raf or MEK inhibitor. In some embodiments, the cell exhibits resistance to a treatment with a B-Raf inhibitor. In some embodiments, the cell exhibits resistance to a treatment with an MEK inhibitor. Exemplary B-Raf and MEK inhibitors of the subject methods are provided above, including, for example, trametinib and vemurafenib. In some embodiments, the cell comprises a B-Raf or N-Ras mutation. The cell may be selected from a pancreatic cancer cell, a lung cancer cell, a melanoma cell and a colorectal cancer cell, such as a melanoma cell.

The term “resistance” refers to a decreased response of a subject or cell to a standard dose of a particular therapeutic agent or to a standard treatment protocol. Resistance of a subject or cell to a particular treatment may be characterized by a lack of a desired response, wherein a desired response in the treatment of cancer may include one or more of inhibition of tumor cell proliferation, inhibition of tumor cell growth, inhibition of tumor vascularization, eradication of tumor cells, reduction in the rate of growth of a tumor, reduction in the size of at least one tumor, and/or eradication or amelioration of one or more physiological symptoms associated with the cancer. A subject or cancer cell that exhibits resistance to a treatment may be nonresponsive or exhibit a reduced or limited response to the treatment, such as having a reduced response to the treatment by 25% or more, for example, 30%, 40%, 50%, 60%, 70%, 80%, or more, to 2-fold, 3-fold, 4-fold, 5-fold, 10-fold, 15-fold, 20-fold or more. The resistance can be mediated by a B-Raf or N-Ras mutation (e.g., BRAF V600E or NRAS Q61R) or by other mechanisms.

The disclosure further provides methods of modulating ERK kinase activity by contacting the kinase with an effective amount of a compound of the disclosure. Modulation can be inhibiting or activating kinase activity. In some embodiments, the disclosure provides methods of inhibiting kinase activity by contacting the kinase with an effective amount of a compound of the disclosure in solution. In some embodiments, the disclosure provides methods of inhibiting the kinase activity by contacting a cell, tissue, organ that expresses the kinase of interest. In some embodiments, the disclosure provides methods of inhibiting kinase activity in subject including but not limited to rodents and mammal (e.g., human) by administering into the subject an effective amount of a compound of the disclosure. In some embodiments, the percentage of inhibiting exceeds 25%, 30%, 40%, 50%, 60%, 70%, 80%, or 90%.

In some embodiments, the kinase is selected from the group consisting of ERK, including different isoforms such as ERK1 and ERK2; Ras; Raf; JNK; ErbB-1 (EGFR); Her2 (ErbB-2); Her 3 (ErbB-3); Her 4 (ErbB-4); MAP2K1 (MEK1); MAP2K2 (MEK2); MAP2K3 (MEK3); MAP2K4 (MEK4); MAP2K5 (MEK5); MAP2K6 (MEK6); MAP2K7 (MEK7); CDK1; CDK2; CDK3; CDK4; CDKS; CDK6; CDK7; CDK8; CDK9; CDK11.

The disclosure further provides methods of modulating ERK activity by contacting ERK with an amount of a compound of the disclosure sufficient to modulate the activity of ERK. Modulate can be inhibiting or activating ERK activity. In some embodiments, the disclosure provides methods of inhibiting ERK by contacting ERK with an amount of a compound of the disclosure sufficient to inhibit the activity of ERK. In some embodiments, the disclosure provides methods of inhibiting ERK activity in a solution by contacting said solution with an amount of a compound of the disclosure sufficient to inhibit the activity of ERK in said solution. In some embodiments, the disclosure provides methods of inhibiting ERK activity in a cell by contacting said cell with an amount of a compound of the disclosure sufficient to inhibit the activity of ERK in said cell. In some embodiments, the disclosure provides methods of inhibiting ERK activity in a tissue by contacting said tissue with an amount of a compound of the disclosure sufficient to inhibit the activity of ERK in said tissue. In some embodiments, the disclosure provides methods of disclosure ERK activity in an organism by contacting said organism with an amount of a compound of the disclosure sufficient to inhibit the activity of ERK in said organism. In some embodiments, the disclosure provides methods of inhibiting ERK activity in an animal by contacting said animal with an amount of a compound of the disclosure sufficient to inhibit the activity of ERK in said animal. In some embodiments, the disclosure provides methods of inhibiting ERK activity in a mammal by contacting said mammal with an amount of a compound of the disclosure sufficient to inhibit the activity of ERK in said mammal. In some embodiments, the disclosure provides methods of inhibiting ERK activity in a human by contacting said human with an amount of a compound of the disclosure sufficient to inhibit the activity of ERK in said human. The present disclosure provides methods of treating a disease mediated by ERK activity in a subject in need of such treatment.

In some embodiments, a method of the disclosure provides an effective dose of an ERK inhibitor. An effective dose refers to an amount sufficient to effect the intended application, including but not limited to, disease treatment, as defined herein. Also contemplated in the subject methods is the use of a sub-therapeutic amount of an ERK inhibitor for treating an intended disease condition.

The amount of the ERK inhibitor administered may vary depending upon the intended application (in vitro or in vivo), or the subject and disease condition being treated, e.g., the weight and age of the subject, the severity of the disease condition, the manner of administration and the like, which can readily be determined by one of ordinary skill in the art.

A subject being treated with an ERK inhibitor may be monitored to determine the effectiveness of treatment, and the treatment regimen may be adjusted based on the subject's physiological response to treatment. For example, if inhibition of a biological effect of ERK inhibition is above or below a threshold, the dosing amount or frequency may be decreased or increased, respectively. The methods can further comprise continuing the therapy if the therapy is determined to be efficacious. The methods can comprise maintaining, tapering, reducing, or stopping the administered amount of a compound in the therapy if the therapy is determined to be efficacious. The methods can comprise increasing the administered amount of a compound in the therapy if it is determined not to be efficacious. Alternatively, the methods can comprise stopping therapy if it is determined not to be efficacious. In some embodiments, treatment with an ERK inhibitor is discontinued if inhibition of the biological effect is above or below a threshold, such as in a lack of response or an adverse reaction. The biological effect may be a change in any of a variety of physiological indicators.

The effectiveness of treatment (or, alternatively, “therapeutic efficacy” or “clinically beneficial response”) is measured based on an effect of treating a cancer. In general, therapeutic efficacy of the methods of the disclosure, with regard to the treatment of a cancer (whether benign or malignant), may be measured by the degree to which the methods and compositions promote inhibition of tumor cell proliferation, the inhibition of tumor vascularization, the eradication of tumor cells, the reduction in the rate of growth of a tumor, and/or a reduction in the size of at least one tumor. Several parameters to be considered in the determination of therapeutic efficacy are discussed herein. The proper combination of parameters for a particular situation can be established by the clinician. The progress of the inventive method in treating cancer (e.g., reducing tumor size or eradicating cancerous cells) can be ascertained using any suitable method, such as those methods currently used in the clinic to track tumor size and cancer progress. The primary efficacy parameter used to evaluate the treatment of cancer by the disclosed methods and compositions preferably is a reduction in the size of a tumor. Tumor size can be figured using any suitable technique, such as measurement of dimensions, or estimation of tumor volume using available computer software, such as FreeFlight software developed at Wake Forest University that enables accurate estimation of tumor volume. Tumor size can be determined by tumor visualization using, for example, CT, ultrasound, SPECT, spiral CT, Mill, photographs, and the like. In embodiments where a tumor is surgically resected after completion of the therapeutic period, the presence of tumor tissue and tumor size can be determined by gross analysis of the tissue to be resected, and/or by pathological analysis of the resected tissue.

Several parameters as described herein may be considered by the clinician in determining if a subject having cancer exhibits a clinically beneficial response. In some desirable embodiments, the growth of a tumor is stabilized (i.e., one or more tumors do not increase more than 1%, 5%, 10%, 15%, or 20% in size, and/or do not metastasize) as a result of the subject methods and compositions. In some embodiments, a tumor is stabilized for at least about 1, 2, 3, 4, 5, 6, 7, 8, 9, 10, 11, 12, or more weeks. In some embodiments, a tumor is stabilized for at least about 1, 2, 3, 4, 5, 6, 7, 8, 9, 10, 11, 12, or more months. In some embodiments, a tumor is stabilized for at least about 1, 2, 3, 4, 5, 6, 7, 8, 9, 10, or more years. Preferably, the inventive method reduces the size of a tumor at least about 5% (e.g., at least about 10%, 15%, 20%, or 25%). More preferably, tumor size is reduced at least about 30% (e.g., at least about 35%, 40%, 45%, 50%, 55%, 60%, or 65%). Even more preferably, tumor size is reduced at least about 70% (e.g., at least about 75%, 80%, 85%, 90%, or 95%). Most preferably, the tumor is completely eliminated, or reduced below a level of detection. In some embodiments, a subject remains tumor free (e.g. in remission) for at least about 1, 2, 3, 4, 5, 6, 7, 8, 9, 10, 11, 12, or more weeks following treatment. In some embodiments, a subject remains tumor free for at least about 1, 2, 3, 4, 5, 6, 7, 8, 9, 10, 11, 12, or more months following treatment. In some embodiments, a subject remains tumor free for at least about 1, 2, 3, 4, 5, 6, 7, 8, 9, 10, or more years after treatment.

In some embodiments, the efficacy of the disclosed methods in reducing tumor size can be determined by measuring the percentage of necrotic (i.e., dead) tissue of a surgically resected tumor following completion of the therapeutic period. In some further embodiments, a treatment is therapeutically effective if the necrosis percentage of the resected tissue is greater than about 20% (e.g., at least about 30%, 40%, 50%, 60%, 70%, 80%, 90%, or 100%), more preferably about 90% or greater (e.g., about 90%, 95%, or 100%). Most preferably, the necrosis percentage of the resected tissue is 100%, that is, no tumor tissue is present or detectable.

The efficacy of the disclosed methods can be determined by a number of secondary parameters. Examples of secondary parameters include, but are not limited to, detection of new tumors, detection of tumor antigens or markers, biopsy, surgical downstaging (i.e., conversion of the surgical stage of a tumor from unresectable to resectable), PET scans, survival, disease progression-free survival, time to disease progression, quality of life assessments such as the Clinical Benefit Response Assessment, and the like, all of which can point to the overall progression (or regression) of cancer in a human. Biopsy is particularly useful in detecting the eradication of cancerous cells within a tissue. Radioimmunodetection (RAID) is used to locate and stage tumors using serum levels of markers (antigens) produced by and/or associated with tumors (“tumor markers” or “tumor-associated antigens”), and can be useful as a pre-treatment diagnostic predicate, a post-treatment diagnostic indicator of recurrence, and a post-treatment indicator of therapeutic efficacy. Examples of tumor markers or tumor-associated antigens that can be evaluated as indicators of therapeutic efficacy include, but are not limited to, carcinembryonic antigen (CEA), prostate-specific antigen (PSA), erythropoietin (EPO), CA-125, CA19-9, ganglioside molecules (e.g., GM2, GD2, and GD3), MART-1, heat shock proteins (e.g., gp96), siaryl Tn (STn), tyrosinase, MUC-1, HER-2/neu, c-erb-B2, KSA, PSMA, p53, RAS, EGF-R, VEGF, MAGE, and gp100. Other tumor-associated antigens are known in the art. RAID technology in combination with endoscopic detection systems also can efficiently distinguish small tumors from surrounding tissue (see, for example, U.S. Pat. No. 4,932,412).

In additional desirable embodiments, the treatment of cancer in a human patient in accordance with the disclosed methods is evidenced by one or more of the following results: (a) the complete disappearance of a tumor (i.e., a complete response), (b) about a 25% to about a 50% reduction in the size of a tumor for at least four weeks after completion of the therapeutic period as compared to the size of the tumor before treatment, (c) at least about a 50% reduction in the size of a tumor for at least four weeks after completion of the therapeutic period as compared to the size of the tumor before the therapeutic period, and (d) at least a 2% decrease (e.g., about a 5%, 10%, 20%, 30%, 40%, 50%, 60%, 70%, 80% or 90% decrease) in a specific tumor-associated antigen level at about 4-12 weeks after completion of the therapeutic period as compared to the tumor-associated antigen level before the therapeutic period. While at least a 2% decrease in a tumor-associated antigen level is preferred, any decrease in the tumor-associated antigen level is evidence of treatment of a cancer in a patient by the inventive method.

With respect to quality of life assessments, such as the Clinical Benefit Response Criteria, the therapeutic benefit of the treatment in accordance with the disclosure can be evidenced in terms of pain intensity, analgesic consumption, and/or the Karnofsky Performance Scale score. The treatment of cancer in a human patient alternatively, or in addition, is evidenced by (a) at least a 50% decrease (e.g., at least a 60%, 70%, 80%, 90%, or 100% decrease) in pain intensity reported by a patient, such as for any consecutive four week period in the 12 weeks after completion of treatment, as compared to the pain intensity reported by the patient before treatment, (b) at least a 50% decrease (e.g., at least a 60%, 70%, 80%, 90%, or 100% decrease) in analgesic consumption reported by a patient, such as for any consecutive four week period in the 12 weeks after completion of treatment as compared to the analgesic consumption reported by the patient before treatment, and/or (c) at least a 20 point increase (e.g., at least a 30 point, 50 point, 70 point, or 90 point increase) in the Karnofsky Performance Scale score reported by a patient, such as for any consecutive four week period in the 12 weeks after completion of the therapeutic period as compared to the Karnofsky Performance Scale score reported by the patient before the therapeutic period.

In some embodiments, tumor size is reduced as a result of the inventive method preferably without significant adverse events in the subject. Adverse events are categorized or “graded” by the Cancer Therapy Evaluation Program (CTEP) of the National Cancer Institute (NCI), with Grade 0 representing minimal adverse side effects and Grade 4 representing the most severe adverse events. Desirably, the disclosed methods are associated with minimal adverse events, e.g. Grade 0, Grade 1, or Grade 2 adverse events, as graded by the CTEP/NCI. However, as discussed herein, reduction of tumor size, although preferred, is not required in that the actual size of tumor may not shrink despite the eradication of tumor cells. Eradication of cancerous cells is sufficient to realize a therapeutic effect. Likewise, any reduction in tumor size is sufficient to realize a therapeutic effect.

Detection, monitoring and rating of various cancers in a human are further described in Cancer Facts and FIGS. 2001, American Cancer Society, New York, N.Y., and International Patent Application WO 01/24684. Accordingly, a clinician can use standard tests to determine the efficacy of the various embodiments of the inventive method in treating cancer. However, in addition to tumor size and spread, the clinician also may consider quality of life and survival of the patient in evaluating efficacy of treatment.

In some embodiments, the disclosure provides a pharmaceutical composition comprising an amount of an ERK inhibitor formulated for administration to a subject in need thereof In some embodiments, the pharmaceutical composition comprises between about 0.0001-500 g, 0.001-250 g, 0.01-100 g, 0.1-50 g, or 1-10 g of the ERK inhibitor. In some embodiments, the pharmaceutical composition comprises about or more than about 0.0001 g, 0.001 g, 0.01g, 0.1, 0.5 g, 1 g, 2 g, 3 g, 4 g, 5 g, 6 g, 7 g, 8 g, 9 g, 10 g, 15 g, 20 g, 25 g, 50g, 100 g, 200 g, 250 g, 300 g, 350 g, 400 g, 450 g, 500 g, or more of the ERK inhibitor. In some embodiments, the pharmaceutical composition comprises between 0.001-2 g of an ERK inhibitor in a single dose. In some embodiments, the therapeutic amount can be an amount between about 0.001-0.1 g of an ERK inhibitor. In some embodiments, the therapeutic amount can be an amount between about 0.01-30 g of an ERK inhibitor. In some embodiments, the therapeutic amount can be an amount between about 0.45 mg/kg/week to 230.4 mg/kg/week of an ERK inhibitor. In some embodiments, the ERK inhibitor is given as an intravenous infusion once per week. Preferably, the ERK inhibitor is given as an intravenous infusion once per week at a dose of about 0.45 mg/kg/week to about 1000 mg/kg/week, such as about 10 mg/kg/week to about 50 mg/kg/week. In some embodiments, the ERK inhibitor is given as an intravenous infusion once per week at a dose of about 5 mg/kg/week, about 10 mg/kg/week, about 20 mg/kg/week, about 30 mg/kg/week, about 40 mg/kg/week, or about 50 mg/kg/week, such as about 20 mg/kg/week.

In some embodiments, the ERK inhibitor can be administered as part of a therapeutic regimen that comprises administering one or more second agents (e.g. 1, 2, 3, 4, 5, or more second agents), either simultaneously or sequentially with the ERK inhibitor. When administered sequentially, the ERK inhibitor may be administered before or after the one or more second agents. When administered simultaneously, the ERK inhibitor and the one or more second agents may be administered by the same route (e.g. injections to the same location; tablets taken orally at the same time), by a different route (e.g. a tablet taken orally while receiving an intravenous infusion), or as part of the same combination (e.g. a solution comprising an ERK inhibitor and one or more second agents). In some embodiments, the ERK inhibitor is administered in combination with anti-EGFR therapy.

The present disclosure also provides methods for combination therapies in which an agent known to modulate other pathways, or other components of the same pathway, or even overlapping sets of target enzymes are used in combination with a compound of the present disclosure, or a pharmaceutically acceptable salt, ester, prodrug, solvate, hydrate or derivative thereof In one aspect, such therapy includes but is not limited to the combination of one or more compounds of the disclosure with chemotherapeutic agents, therapeutic antibodies, and radiation treatment, to provide a synergistic or additive therapeutic effect.

In another aspect, the disclosure also relates to methods and pharmaceutical compositions for inhibiting abnormal cell growth in a mammal which comprises an amount of a compound of the disclosure, or a pharmaceutically acceptable salt, ester, prodrug, solvate, hydrate or derivative thereof, in combination with an amount of an anti-cancer agent (e.g. a chemotherapeutic agent). Many chemotherapeutics are presently known in the art and can be used in combination with the compounds of the disclosure. In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

Non-limiting examples are chemotherapeutic agents, cytotoxic agents, and non-peptide small molecules such as Gleevec® (Imatinib Mesylate), Velcade® (bortezomib), Casodex (bicalutamide), Iressa® (gefitinib), and Adriamycin as well as a host of chemotherapeutic agents. Non-limiting examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK.R™; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL™, Bristol-Myers Squibb Oncology, Princeton, N.J.) and docetaxel (TAXOTERE™, Rhone-Poulenc Rorer, Antony, France); retinoic acid; esperamicins; capecitabine; and pharmaceutically acceptable salts, acids or derivatives of any of the above. Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO). Where desired, the compounds or pharmaceutical composition of the present disclosure can be used in combination with commonly prescribed anti-cancer drugs such as Herceptin®, Avastin®, Erbitux®, Rituxan®, Taxol®, Arimidex®, Taxotere®, ABVD, AVICINE, Abagovomab, Acridine carboxamide, Adecatumumab, 17—N-Allylamino-17-demethoxygeldanamycin, Alpharadin, Alvocidib, 3-Aminopyridine-2-carboxaldehyde thiosemicarbazone, Amonafi de, Anthracenedione, Anti-CD22 immunotoxins, Antineoplastic, Antitumorigenic herbs, Apaziquone, Atiprimod, Azathioprine, Belotecan, Bendamustine, BMW 2992, Biricodar, Brostallicin, Bryostatin, Buthionine sulfoximine, CBV (chemotherapy), Calyculin, cell-cycle nonspecific antineoplastic agents, cetuximab, cisplatin, Dichloroacetic acid, Discodermolide, Elsamitrucin, Enocitabine, Epothilone, Eribulin, erlotinib, Everolimus, Exatecan, Exisulind, Ferruginol, Forodesine, Fosfestrol, gemcitabine, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, palbociclib, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, ZD6126, and Zosuquidar.

In certain embodiments, the present disclosure provides a method of treating squamous cell carcinoma in a subject in need thereof, comprising administering to said subject an ERK inhibitor and a second therapeutic agent. In practicing any of the subject methods, the second therapeutic agent may be selected from gemcitabine, cisplatin, an EGFR inhibitor and a CDK inhibitor. In some embodiments, the second therapeutic agent is selected from gemcitabine, cisplatin, cetuximab, erlotinib and palbociclib. In some embodiments, the second therapeutic agent is selected from gemcitabine, cisplatin, cetuximab. In some embodiments, the second therapeutic agent is an EGFR inhibitor, such as cetuximab or erlotinib. In some embodiments, the second therapeutic agent is a CDK inhibitor, preferably a CDK4/6 inhibitor, such as palbociclib. In some embodiments, the second therapeutic agent is selected from gemcitabine, cisplatin, cetuximab, wherein the squamous cell carcinoma is squamous cell carcinoma of the lung. In some embodiments, the second therapeutic agent is cetuximab, wherein the squamous cell carcinoma is squamous cell carcinoma of the esophagus or head and neck. In some embodiments, the second therapeutic agent is erlotinib, wherein the squamous cell carcinoma is squamous cell carcinoma of the lung.

In practicing any of the subject methods, the second therapeutic agent may be selected from osimertinib, olmutinib, icotinib hydrochloride, afatinib, necitumumab, lapatinib, pertuzumab, vandetanib, BV—NSCLC-001, nimotuzumab, panitumumab, erlotinib, gefitinib, cetuximab, brigatinib, naquotinib mesylate, anti-EGFR antibody , depatuxizumab mafodotin, tesevatinib , dacomitinib, neratinib, anti-EGFR CART cell therapy, PF-06747775, AP-32788, AZD-3759, nazartinib, entinostat +erlotinib , allitinib tosylate, tarloxotinib bromide, S-222611, pyrroltinib maleate , poziotinib, second generation cetuximab , RXDX-105, futuximab, seribantumab, varlitinib, icotinib hydrochloride , SYN-004 (Synermore Biologics), anti-EGFR CAR-T therapy, durvalumab+osimertinib, LY-3164530, tremelimumab+gefitinib, durvalumab+gefitinib , GC-1118, JNJ-61186372, Pirotinib, SKLB-1028, PB-357, BGB-283, SCT-200, QLNC-120, TAS-121, Hemay-020, Hemay-022, theliatinib, NRC-2694-A, epitinib succinate, MM-151, simotinib hydrochloride, depatuxizumab, AFM-24, HTI-1511, EGFR/Axl dual inhibitors, RC-68, EGFRvIII CAR T cell therapy, UBP-1215, LL-067, Probody T cell-engaging bispecific targeting CD3 and EGFR, YH-25448, SKLB-287, AFM-22 (Affimed), AK-568, panitumumab biosimilar, RJS-013, RJS-012, recombinant EGF/CRM-197 vaccine, recombinant fully human anti-EGFR mAb, nimotuzumab biosimilar, EGFR-targeted siRNA therapeutics, anti-EGFR recombinant Fc engineered IgA2m antibody, sirotinib malate, anti-EGFR targeting mAbs, anti-EGFR/anti-CD3 bispecific antibody, alpha-c-Met/EGFR-0286 bispecific antibody drug conjugate, small molecule therapeutic, HLX-07, JHL-1189, KN-023, panitumumab biosimilar, anti-EGFR monoclonal antibody, FV-225, EGFR T790M inhibitors (Beta Pharma), cetuximab biosimilar, MP-0274, EGFR T790M inhibitor (Genentech/Argenta), STI-A020X, KL-ON113, neratinib, 18F-afatinib, PMIP, DBPR-112, SKI-O-751, PTZ-09, bi-specific anti-Her3 Zybodies (Zyngenia), SHR-1258, G5-7, bispecific centyrins (Janssen), AG-321, kahalalide F, E-10C, JRP-980, JRP-890, MED-1007, LA22-MMC, NT-004, NT-113, Sym-013, anti-Her-2/anti-Ang2 mAb (Zyngenia), MT-062, trastuzumab biosimilar, AFM-21, NT-219, ANG-MAB (AngioChem), ISU-101, and VRCTC-310. In some embodiments, the second therapeutic agent is selected from osimertinib, olmutinib, icotinib hydrochloride, afatinib, necitumumab, lapatinib, pertuzumab, vandetanib, BV—NSCLC-001, nimotuzumab, panitumumab, erlotinib, gefitinib, cetuximab, brigatinib, naquotinib mesylate, anti-EGFR antibody , depatuxizumab mafodotin, tesevatinib , dacomitinib, neratinib, anti-EGFR CART cell therapy, PF-06747775, AP-32788, AZD-3759, nazartinib, entinostat +erlotinib , allitinib tosylate, tarloxotinib bromide, A-222611, pyrroltinib maleate , poziotinib, second generation cetuximab , RXDX-105, futuximab, seribantumab, and varlitinib. In some embodiments, the second therapeutic agent is selected from palbociclib, abemaciclib, ribociclib, G1T-28, AT-7519, alvocidib, FLX-925, G1T-38, GZ-38-1, ON-123300 and voruciclib. In some embodiments, the second therapeutic agent is selected from palbociclib, abemaciclib, ribociclib, G1T-28, AT-7519 and alvocidib. In some embodiments, the second therapeutic agent is selected from palbociclib, osimertinib, olmutinib, icotinib hydrochloride, afatinib, necitumumab, lapatinib, pertuzumab, vandetanib, BV—NSCLC-001, nimotuzumab, panitumumab, erlotinib, gefitinib and cetuximab.

This disclosure further relates to a method for using the compounds or pharmaceutical compositions provided herein in combination with radiation therapy for inhibiting abnormal cell growth or treating the hyperproliferative disorder in the mammal. Techniques for administering radiation therapy are known in the art, and these techniques can be used in the combination therapy described herein. The administration of the compound of the disclosure in this combination therapy can be determined as described herein.

Radiation therapy can be administered through one of several methods, or a combination of methods, including without limitation external-beam therapy, internal radiation therapy, implant radiation, stereotactic radiosurgery, systemic radiation therapy, radiotherapy and permanent or temporary interstitial brachytherapy. The term “brachytherapy,” as used herein, refers to radiation therapy delivered by a spatially confined radioactive material inserted into the body at or near a tumor or other proliferative tissue disease site. The term is intended without limitation to include exposure to radioactive isotopes (e.g. At-211, I-131, I-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive micro spheres.

Without being limited by any theory, the compounds of the present disclosure can render abnormal cells more sensitive to treatment with radiation for purposes of killing and/or inhibiting the growth of such cells. Accordingly, this disclosure further relates to a method for sensitizing abnormal cells in a mammal to treatment with radiation which comprises administering to the mammal an amount of a compound of the present disclosure or pharmaceutically acceptable salt, ester, prodrug, solvate, hydrate or derivative thereof, which amount is effective is sensitizing abnormal cells to treatment with radiation. The amount of the compound, salt, or solvate in this method can be determined according to the means for ascertaining effective amounts of such compounds described herein.

The compounds or pharmaceutical compositions of the disclosure can be used in combination with an amount of one or more substances selected from anti-angiogenesis agents, signal transduction inhibitors, antiproliferative agents, glycolysis inhibitors, or autophagy inhibitors.

Anti-angiogenesis agents, such as MMP-2 (matrix-metalloproteinase 2) inhibitors, MMP-9 (matrix-metalloprotienase 9) inhibitors, and COX-11 (cyclooxygenase 11) inhibitors, can be used in conjunction with a compound of the disclosure and pharmaceutical compositions described herein. Anti-angiogenesis agents include, for example, rapamycin, temsirolimus (CCI-779), everolimus (RAD001), sorafenib, sunitinib, and bevacizumab. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931, 788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/M98/01113 (filed Jul. 21, 1998), European Patent Application No. 99302232.1 (filed Mar. 25, 1999), Great Britain Patent Application No. 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference. Preferred MMP-2 and MMP-9 inhibitors are those that have little or no activity inhibiting MMP-1. More preferred, are those that selectively inhibit MMP-2 and/or AMP-9 relative to the other matrix-metalloproteinases (i. e., MAP-1, MMP-3, MMP-4, MMP-5, MMP-6, MMP-7, MMP-8, MMP-10, MMP-11, MMP-12, and MMP-13). Some specific examples of MMP inhibitors useful in the disclosure are AG-3340, RO 32-3555, and RS 13-0830.

Autophagy inhibitors include, but are not limited to chloroquine, 3-methyladenine, hydroxychloroquine (Plaquenil™), bafilomycin Al, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATGS (which are implicated in autophagy), may also be used.

Administration of the compounds of the present disclosure can be effected by any method that enables delivery of the compounds to the site of action. An effective amount of a compound of the disclosure may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer. Preferably, the ERK inhibitor is administered intravenously or orally.

The amount of the compound administered will be dependent on the mammal being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g. by dividing such larger doses into several small doses for administration throughout the day.

When used in combination with a second therapeutic agent, the ERK inhibitor can be administered at a dosage that is the same as the effective amount for that agent when administered as a monotherapy. In some embodiments, the ERK inhibitor is administered in a sub-therapeutic amount in combination with a second therapeutic agent, such as a CDK inhibitor. A sub-therapeutic amount of an agent is an amount less than the effective amount of the agent. For example, the ERK inhibitor, when administered in combination with a second therapeutic agent, can be administered in an amount less than 90% of the effective amount, such as less than 80%, less than 70%, less than 60%, less than 50%, less than 40%, less than 30%, less than 20%, or less than 10% of the effective amount. In some embodiments, a sub-therapeutic amount of the second therapeutic agent is administered in combination with an ERK inhibitor. In some embodiments, sub-therapeutic amounts of both an ERK inhibitor and a second therapeutic agent are administered. An ERK inhibitor described herein, such as a compound provided in Table 3, is expected to produce a synergistic effect when used in combination with a second therapeutic agent, such as a CDK inhibitor. In some embodiments, the synergistic effect is more pronounced when a sub-therapeutic amount of the ERK inhibitor is administered. The individual components of the combination, though one or more is present in a sub-therapeutic amount, synergistically yield an efficacious effect and/or reduced a side effect in an intended application.

In some embodiments, a compound of the disclosure is administered in a single dose. Typically, such administration will be by injection, e.g., intravenous injection, in order to introduce the agent quickly. However, other routes may be used as appropriate. A single dose of a compound of the disclosure may also be used for treatment of an acute condition.

In some embodiments, a compound of the disclosure is administered in multiple doses. Dosing may be about once, twice, three times, four times, five times, six times, or more than six times per day. Dosing may be about once a month, once every two weeks, once a week, or once every other day. In another embodiment a compound of the disclosure and another agent are administered together about once per day to about 6 times per day. In another embodiment the administration of a compound of the disclosure and an agent continues for less than about 7 days. In yet another embodiment the administration continues for more than about 6, 10, 14, 28 days, two months, six months, or one year. In some cases, continuous dosing is achieved and maintained as long as necessary.

Administration of the agents of the disclosure may continue as long as necessary. In some embodiments, an agent of the disclosure is administered for more than 1, 2, 3, 4, 5, 6, 7, 14, or 28 days. In some embodiments, an agent of the disclosure is administered for less than 28, 14, 7, 6, 5, 4, 3, 2, or 1 day. In some embodiments, an agent of the disclosure is administered chronically on an ongoing basis, e.g., for the treatment of chronic effects.

When a compound of the disclosure is administered in a composition that comprises one or more agents, and the agent has a shorter half-life than the compound of the disclosure, unit dose forms of the agent and the compound of the disclosure may be adjusted accordingly.

The compounds described herein can be used in combination with other agents disclosed herein or other suitable agents, depending on the condition being treated. Hence, in some embodiments, the one or more compounds of the disclosure will be co-administered with other agents as described above. In some embodiments, the other agent is an anti-cancer agent. When used in combination therapy, the compounds described herein may be administered with the second agent simultaneously, or separately. The administration in combination can include simultaneous administration of the two agents in the same dosage form, simultaneous administration in separate dosage forms, or separate administration. That is, a compound described herein and any of the agents described above can be formulated together in the same dosage form and administered simultaneously. Alternatively, a compound of the disclosure and any of the agents described above can be simultaneously administered, wherein both the agents are present in separate formulations. In another alternative, a compound of the present disclosure can be administered just followed by and any of the agents described above, or vice versa. In the separate administration protocol, a compound of the disclosure and any of the agents described above may be administered a few minutes apart, or a few hours apart, or a few days apart.

The following examples are given for the purpose of illustrating various embodiments of the disclosure and are not meant to limit the present disclosure in any fashion. The present examples, along with the methods and compositions described herein, are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### EXAMPLES

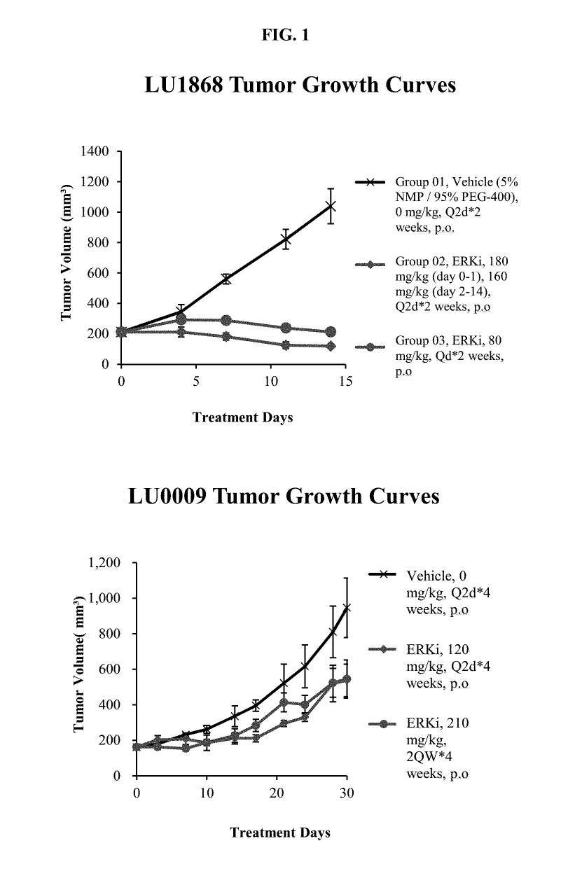

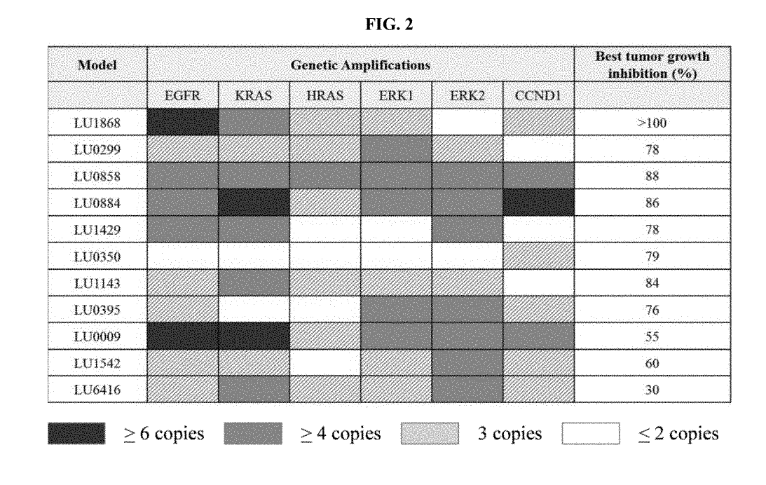

Example 1: Efficacy studies in patient-derived xenograft models of squamous NSCLC. Tumor fragments (2-4 mm in diameter) from stock mice inoculated with LU1868 or LU0009 primary human NSCLC tissues were inoculated subcutaneously into BALB/C nude mice. The mice were stratified into groups when the average tumor size reached about 200 mm³. Animals were treated with vehicle or an ERK inhibitor (KO-947, a compound of Formula I as described herein) at the doses indicated in FIG. 1. Tumor volumes were measured twice weekly in two dimensions using a caliper, with volume expressed in mm³ (mean+/−SEM) using the formula V=0.5(a×b)², where a and b are the long and short diameters of the tumor, respectively. A total of eleven NSCLC patient-derived xenograft (PDX) models were treated in the same manner with either vehicle or the ERK inhibitor. Gene copy number data for the NSCLC models is presented in FIG. 2. Examples of responses of LSCC models to the ERK inhibitor are shown in FIG. 1. In the highly responsive LU1868 model, tumor regression was seen on both daily and Q2D dosing schedules, but only modest inhibition of tumor growth was observed on either Q2D or 2QW schedules in the unresponsive LU0009 model.

As shown in FIG. 2, some apparent copy number increase in many of the six gene MAPK pathway gene set was evident in most of the lung SCC models (10/11 for EGFR, 9/11 for KRAS, ERK1 and ERK2, 8/11 for CCND1 and 7/11 for HRAS). More robust copy number increases were less common, with ≥four copies of KRAS or ERK2 being detected in 7/11 models, EGFR or ERK1 in 5/11 models, CCND1 in 3/11 models, and HRAS in only a single model. Although robust increases in copy number of EGFR, ERK1 and/or KRAS in particular were associated with response to the ERK inhibitor, some unresponsive tumors also displayed this pattern.

Gene copy number data was generated by Affymetrix SNP6.0 array using genomic DNA from the patient-derived xenograft tumor samples, analyzed by PICNIC or PENNCNV software. Gene expression of the samples was profiled by RNAseq. Tumor RNA was extracted in Trizol solution according to the manufacturer's protocol. RNA was evaluated by Agilent Bioanalyzer for quality control. Samples with RIN 7.0 or greater were used for library construction (using Illumina TruSeq kit), and transcriptome sequencing was conducted using Illumina HiSeq systems. MMSEQ software was used to perform the gene expression analysis. The output of MMSEQ software was in the format of Ln(FPKM) and was converted to linear values for signature analyses.

ERK inhibitor responses were classified as “regressions” if tumor growth inhibition (TGI) exceeded 100%, i.e. the tumors were smaller when dosing was completed than at the start of the dosing period. Responses were classified as “tumor stasis” if tumors grew ≤10% during the dosing period. Many SCC patient-derived xenograft models retain comorbid properties of the original tumors from which they were derived, such as induction of cachexia and spontaneous ulceration, that negatively impact the physiology of the host animal and reduce tolerability to exogenous agents, such as therapeutic drugs. In this series of experiments, it was observed that the ERK inhibitor was less well-tolerated in some models, presumably due to tumor-related factors, resulting in body weight loss. Dosing holidays were necessary during periods when the mouse had lost ≥10% of body weight until the weight of the mouse returned to baseline. In models where TGI was ≥75% despite more than a quarter of the doses being missed, it was considered that the true potential activity of the drug was significantly undermined, so these models were considered to be in the same category as “tumor stasis” for some analyses. Where TGI was 70-85% and no doses were missed, activity was classified as “borderline”, and any TGI levels below 70% were classified as “resistant”. For purposes of bioinformatics analyses leading to the identification of genetic and gene expression biomarkers linked to sensitivity or resistance to ERK inhibition, the borderline and resistant groups (i.e., those models displaying less than 85% TGI when no doses were missed) were classified as “inactive”, or, as used in the methods described in the present disclosure, as having “low sensitivity to the ERK inhibitor”. All other models were classified as “active”, or, as used in the methods described herein, as “sensitive to the ERK inhibitor”.

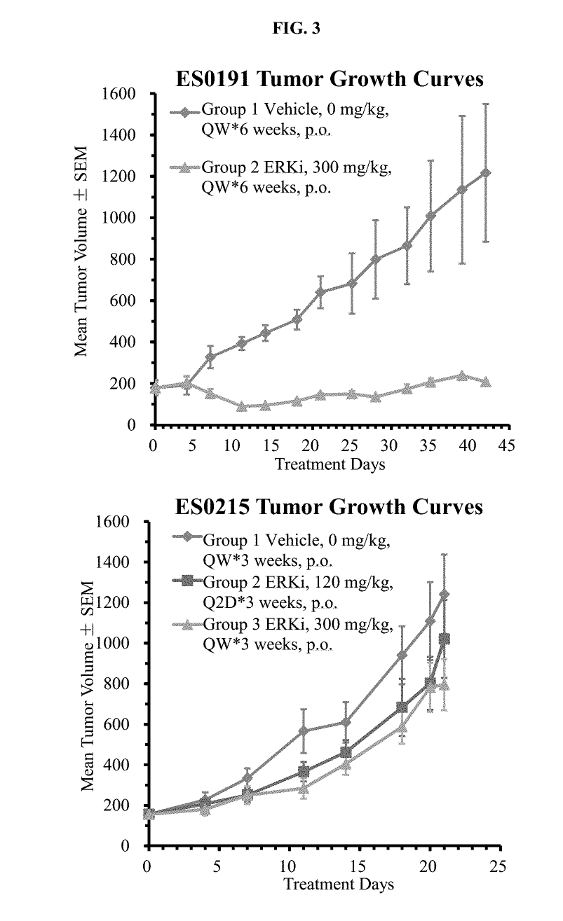

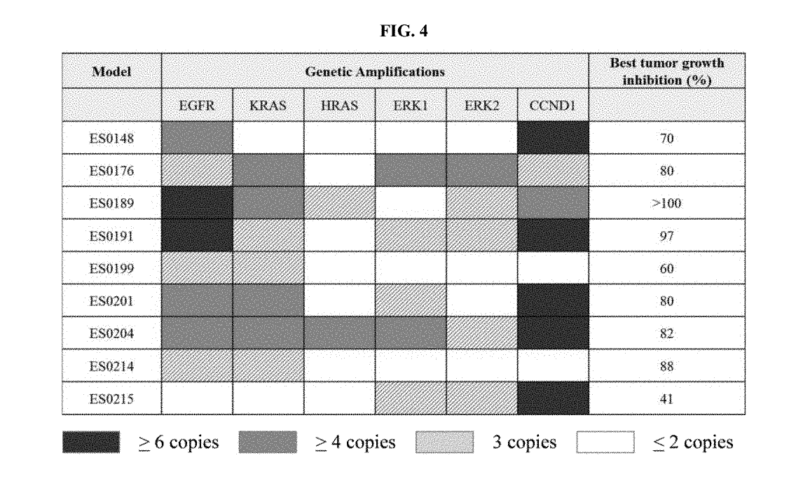

Example 2: Efficacy studies in patient-derived xenograft models of ESCC. The general procedure outline in Example 1 was followed. Briefly, tumor fragments (2-4 mm in diameter) from stock mice inoculated with ES0191 or ES0215 primary human ESCC tissues were inoculated subcutaneously into BALB/C nude mice. The mice were stratified into groups when the average tumor size reached about 180 mm³. Animals were treated with vehicle or an ERK inhibitor (KO-947, a compound of Formula I as described herein) at the doses indicated in FIG. 3. Tumor volumes were measured twice weekly in two dimensions using a caliper, with volume expressed in mm³ (mean+/−SEM) using the formula V=0.5(a×b)², where a and b are the long and short diameters of the tumor, respectively. A total of nine ESCC patient-derived xenograft models were treated in the same manner with either vehicle or the ERK inhibitor. Gene copy number data for the ESCC models is presented in FIG. 4. Gene copy number and gene expression were assessed as described in Example 1. Examples of responses of ESCC models to the ERK inhibitor are shown in FIG. 3. In the highly responsive ES0191 model, tumor regression was initially seen on a weekly schedule, with some regrowth late in the experiment resulting ultimately in tumor stasis. In the unresponsive ES0215 model, only modest inhibition of tumor growth was observed on either Q2D or QW schedules.

As shown in FIG. 4, copy number changes in MAPK pathway gene set were quite common in ESCC models (8/9 for EGFR, 7/9 for KRAS or CCND1, 6/9 for ERK1, ERK2 or both and 2/9 for HRAS). Fewer models showed at least four copies. Among the panel of ESCC models, there was a positive association between a copy number of at least four for one of the MAPK pathway genes EGFR, KRAS, HRAS, ERK1, ERK2, and/or CCND1 and response to treatment with the ERK inhibitor.

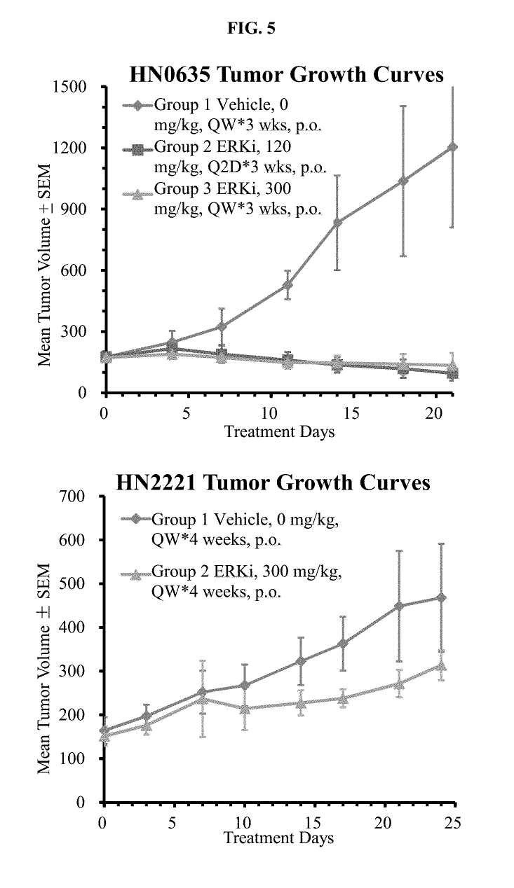

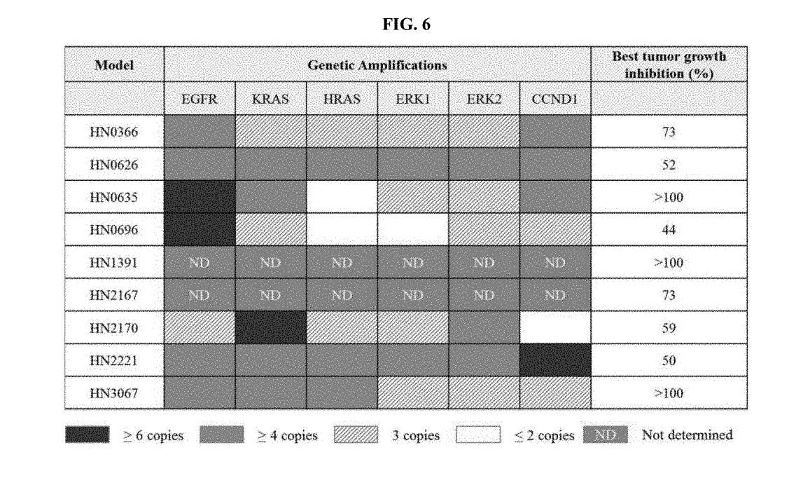

Example 3: Efficacy studies in patient-derived xenograft models of HNSCC. The general procedure outline in Example 1 was followed. Briefly, tumor fragments (2-4 mm in diameter) from stock mice inoculated with HN0635 or HN2221 primary human HNSCC tissues were inoculated subcutaneously into BALB/C nude mice. The mice were stratified into groups when the average tumor size reached about 150-200 mm³. Animals were treated with vehicle or an ERK inhibitor (KO-947, a compound of Formula I as described herein) at the doses indicated in FIG. 5. Tumor volumes were measured twice weekly in two dimensions using a caliper, with volume expressed in mm³ (mean+/−SEM) using the formula V=0.5(a×b)², where a and b are the long and short diameters of the tumor, respectively. A total of nine HNSCC patient-derived xenograft models were treated in the same manner with either vehicle or the ERK inhibitor. Gene copy number data for the HNSCC models is presented in FIG. 6. Gene copy number and gene expression were assessed as described in Example 1. Examples of responses of HNSCC models to the ERK inhibitor are shown in FIG. 5. In the highly responsive HN0635 model, the ERK inhibitor induced tumor regression in 5 of 6 animals treated on Q2D or QW schedules, whereas in the unresponsive HN2221 model, only modest inhibition of tumor growth was achieved on weekly dosing of the ERK inhibitor.

As shown in FIG. 6, 4/7 evaluable models were hyperdiploid for all six MAPK pathway genes in the analysis, and two were hyperdiploid for five of the six genes. Higher order copy number changes were less common, although 6/7 evaluable models possessed at least 4 copies of EGFR, and 4/7 each had at least 4 copies of KRAS or CCND1 .

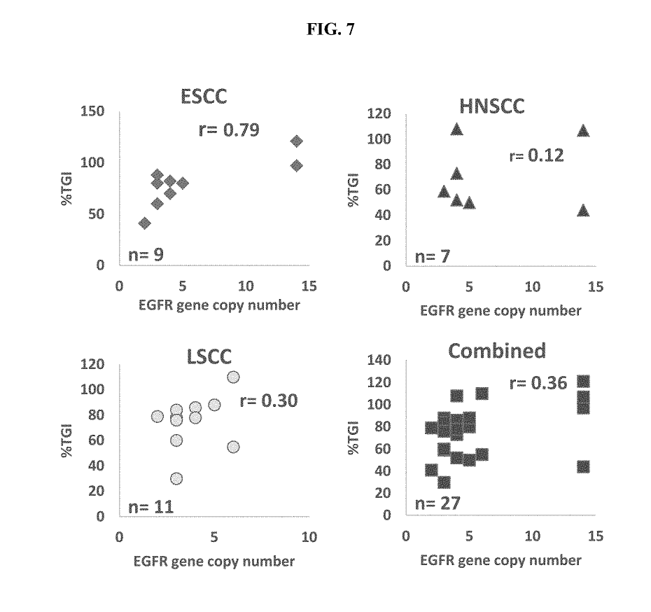

Results of the studies described in Examples 1 to 3 are summarized in Table 1. The correlation between EGFR copy number and tumor growth inhibition in each of the squamous cell carcinoma types tested is plotted in FIG. 7.

TABLE 1
Summary of ERK inhibitor activity in patient-derived xenograft
models of SCC.

| Squamous | Total | Response to treatment (number of models) | | | | |
|---|---|---|---|---|---|---|
| cell | number | | | ≥75% TGI | | |
| carcinoma | of | >100% | 90-100% | (incomplete | 70-85% | <70% |
| type | models | TGI | TGI | dosing) | TGI | TGI |
| Lung | 11 | 1 | 1 | 4 | 2 | 3 |
| Esophagus | 9 | 1 | 2 | 4 | 0 | 2 |
| Head & | 9 | 3 | 0 | 1 | 1 | 4 |
| neck | | | | | | |
| Cervix | 2 | 0 | 1 | 0 | 1 | 0 |

Example 4: Efficacy studies in patient-derived xenograft models of other tumor types. The general procedure outline in Example 1 was followed for 46 different patient-derived xenograft models, representing eleven different tumor types. These eleven tumor types displayed mostly low sensitivity to treatment with the ERK inhibitor, as summarized in Table 2. Strikingly, of the twelve tumor types tested in these Examples, only the squamous cell carcinomas displayed robust responsiveness to treatment with the ERK inhibitor.

TABLE 2
Summary of ERK inhibitor activity in various tumor types.

| | | Response to treatment (number of models) | | | | |
|---|---|---|---|---|---|---|
| | Total | | | ≥75% TGI | | Inactive |
| | number of | | Tumor | incomplete | ≥70% | (<70% |
| Tumor type | models | Regression | stasis | dosing | TGI | TGI) |
| Bladder carcinoma | 2 | 1 | 0 | 0 | 0 | 1 |
| Brain tumors (primary) | 3 | 1 | 0 | 0 | 0 | 2 |
| Breast cancer | 4 | 0 | 0 | 0 | 0 | 4 |
| Cholangiocarcinoma | 3 | 0 | 0 | 0 | 1 | 1 |
| Gall bladder carcinoma | 3 | 0 | 0 | 0 | 0 | 3 |
| Renal cell carcinoma | 2 | 0 | 0 | 0 | 0 | 2 |
| Hepatocellular carcinoma | 8 | 0 | 0 | 0 | 0 | 8 |
| Lung cancer (small cell) | 7 | 0 | 0 | 0 | 0 | 7 |
| Lymphoma | 3 | 0 | 0 | 0 | 0 | 3 |
| Malignant melanoma | 2 | 0 | 1 | 0 | 0 | 1 |
| Ovarian carcinoma | 9 | 0 | 1 | 0 | 0 | 8 |

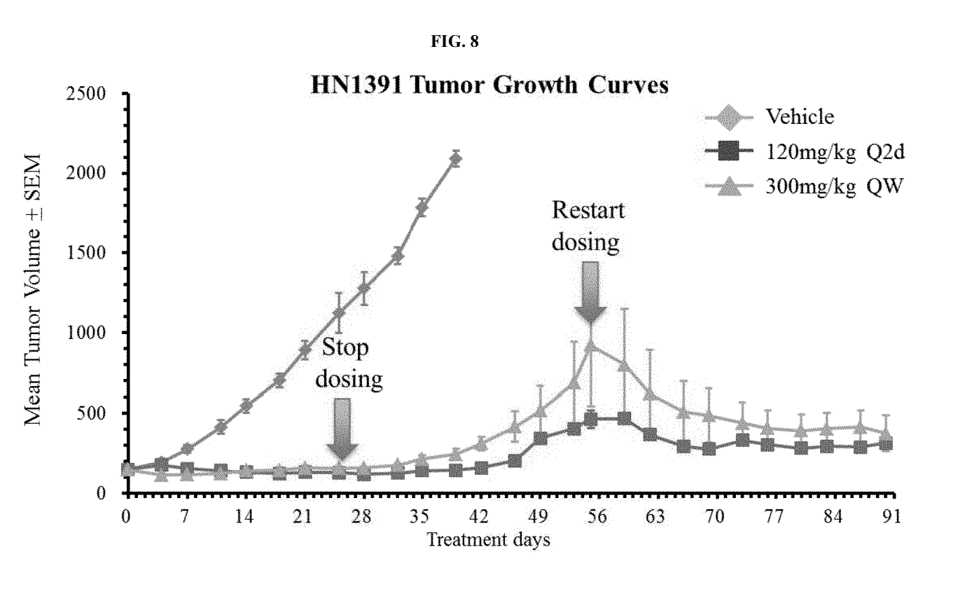

Example 5: Efficacy studies in patient-derived xenograft modes of HNSCC. The general procedure outline in Example 1 was followed. Briefly, tumor fragments (2-4 mm in diameter) from stock mice inoculated with HN1391 primary human HNSCC tissues were inoculated subcutaneously into BALB/C nude mice. The mice were stratified into groups when the average tumor size reached about 150 mm³. Animals were treated with vehicle or an ERK inhibitor (KO-947, a compound of Formula I as described herein) at the doses indicated in FIG. 8 for 25 days. Dosing was discontinued to observe tumor regrowth, then restarted on the same dosing schedule on day 56. Tumor volumes were measured twice weekly in two dimensions using a caliper, with volume expressed in mm³ (mean+−SEM) using the formula V=0.5(a×b)², where a and b are the long and short diameters of the tumor, respectively. As shown in FIG. 8, the ERK inhibitor induced regression and tumor stasis in the HN1391 model when treatment was initiated. This activity was maintained for 25 days on treatment at either 120 mg/kg Q2D or 300 mg/kg QW. When dosing was discontinued, tumor stasis was maintained for 10-20 days, but eventually all of the tumors regrew. Regrowth was allowed to proceed for 2-3 weeks, until some individual tumors exceeded 1600 mm³, then therapy was restarted. Strikingly, all of the retreated tumors regressed, decreasing in size by 27-66% in the subsequent 35 days. Treatment with the ERK inhibitor was actually curative in several cases.

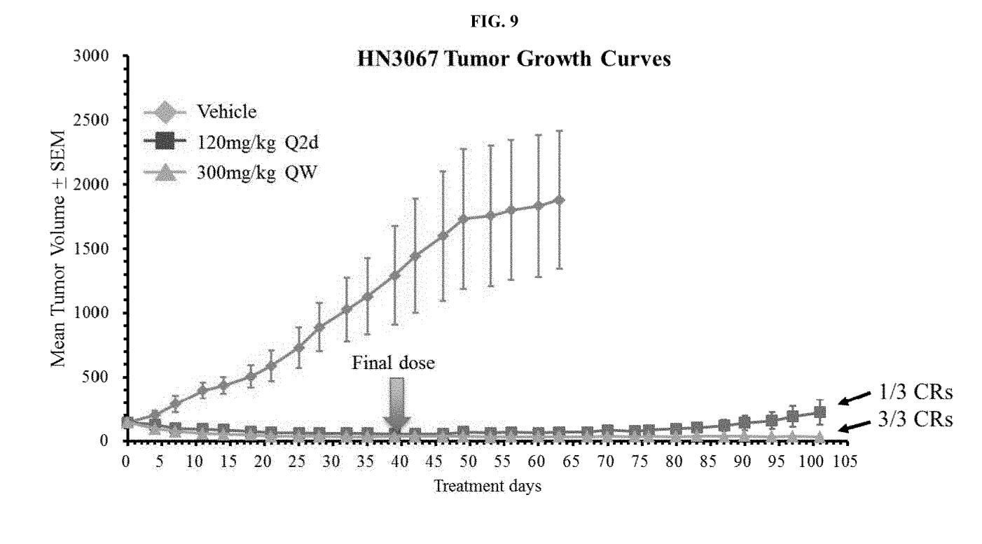

Example 6: Efficacy studies in patient-derived xenograft models of HNSCC. The general procedure outline in Example 1 was followed. Briefly, tumor fragments (2-4 mm in diameter) from stock mice inoculated with HN3067 primary human HNSCC tissues were inoculated subcutaneously into BALB/C nude mice. The mice were stratified into groups when the average tumor size reached about 180 mm³. Animals were treated with vehicle or an ERK inhibitor (KO-947, a compound of Formula I as described herein) at the doses indicated in FIG. 9 for 40 days. Dosing was discontinued to observe tumor regrowth. Tumor volumes were measured twice weekly in two dimensions using a caliper, with volume expressed in mm³ (mean+−SEM) using the formula V=0.5(a×b)², where a and b are the long and short diameters of the tumor, respectively. As shown in FIG. 9, all six animals bearing HN3067 patient-derived xenograft tumors displayed robust tumor regression after treatment at either 120 mg/kg Q2D or 300 mg/kg QW. When dosing was discontinued to observe the rate of tumor regrowth, 4 of 6 animals (including all three treated on the QW schedule), exhibited no evidence of remaining viable tumor even sixty days later, suggesting that these animals had been permanently cured of their disease.

Example 7: Analysis of copy number and gene expression signatures. Results of the copy number analyses described in Examples 1 to 3 revealed that frequent but highly variable copy number changes in certain members of the MAPK pathway arise in squamous cell carcinomas of the lung, esophagus and head and neck, at least as represented by the patient-derived models tested herein, but that clear associations between apparent amplification of particular individual genes and responses to the ERK inhibitor are difficult to discern for the models tested, except for in ESCC. For this reason, a second analytical approach, focused on expression levels of key MAPK pathway genes and RAS-ERK feedback regulators (i.e. their mRNA abundance, as estimated by RNAseq) was employed to generate gene expression signatures that integrated information from multiple genes of interest into a single value, thus enabling the comparison of sensitivity to ERK inhibition with an aggregate readout of either (i) overexpression of commonly amplified pathway components (i.e. a total expression level that is greater than a reference level), and/or (ii) signaling output of the pathway components, as indicated by the abundance of mRNAs for genes in the pathway.

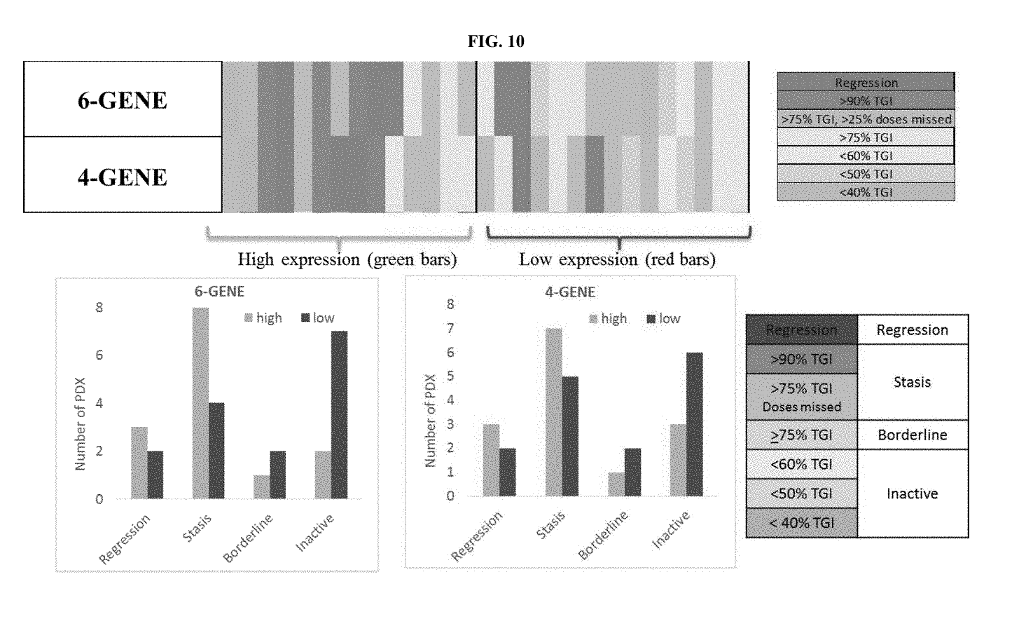

Several gene signatures were evaluated across the panel of 29 lung, esophageal and head and neck squamous cell carcinoma models tested in Examples 1 to 3. Results of the analyses are presented in FIGS. 10-15 as heatmaps that show the relationship between response to treatment with the ERK inhibitor and total mRNA abundance of the genes that comprise the signature. Total expression levels (i.e. total mRNA abundance) are plotted from high to low (left to right) in each of the figures. The cutoff used to distinguish “high expression” from “low expression”, represented by the heavy black line in the heatmaps, is the average expression level of the 29 models. This cutoff is referred to as a reference level in the methods described herein. Therapeutic outcomes were grouped into four categories (regression, stasis, borderline and inactive) as shown in the conversion keys provided for each figure.

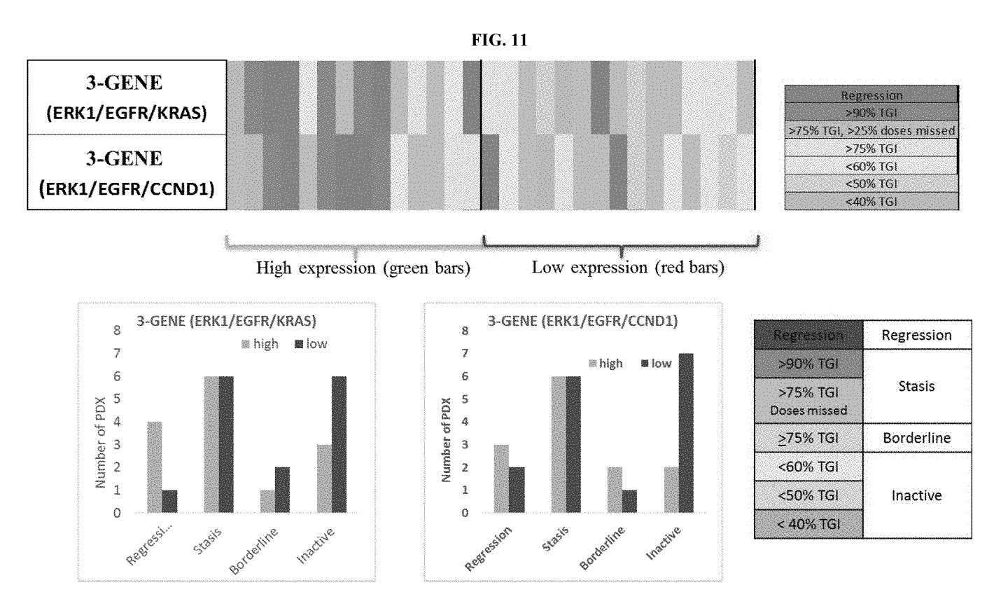

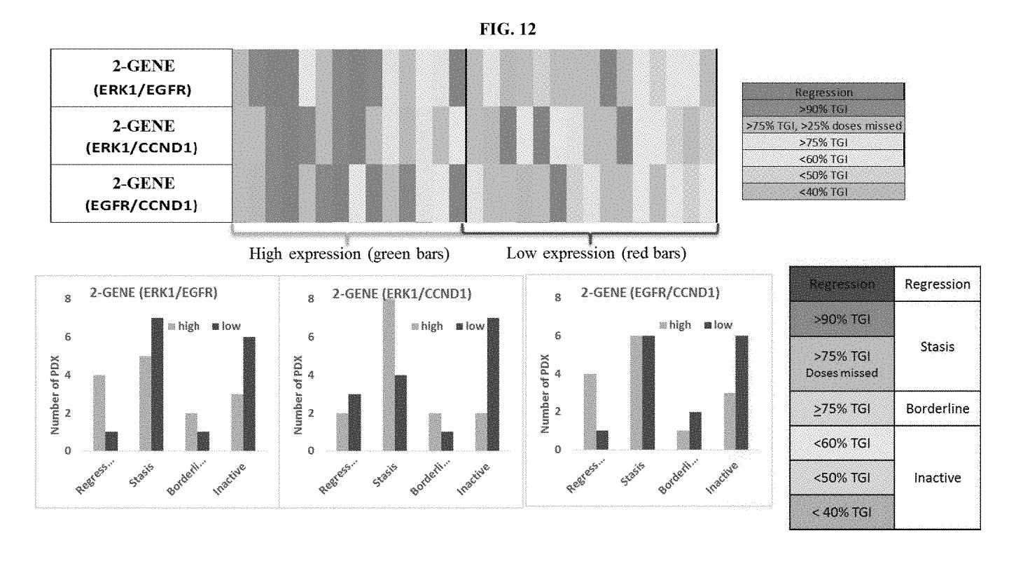

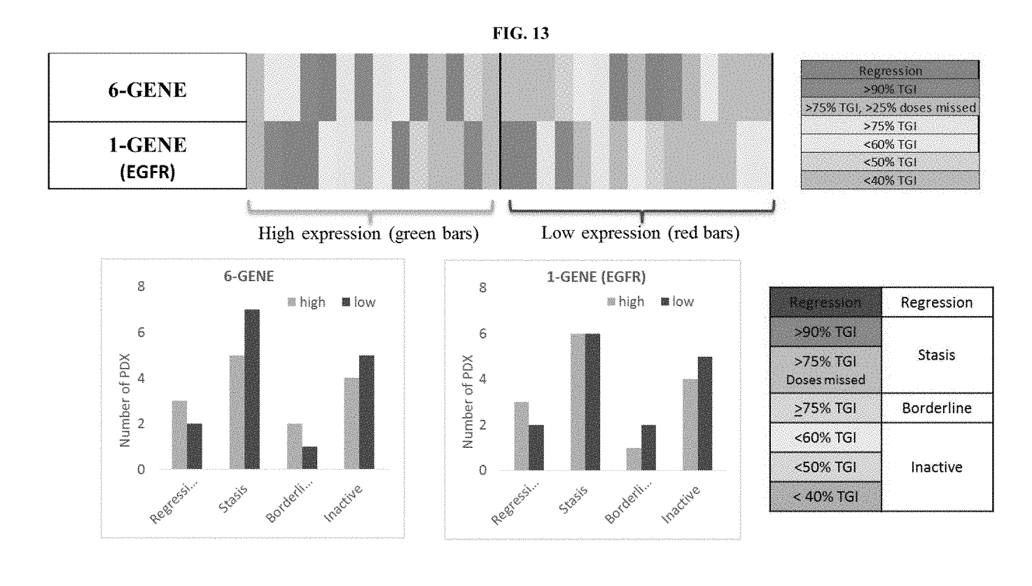

As shown in FIG. 10, there is a clear association between high signals from the gene expression signatures (left side of the heatmap) and positive responses (e.g. regression or stasis in the bar graphs) to treatment with the ERK inhibitor for both MAPK pathway gene signatures. The 6-gene signature of FIG. 10 comprises EGFR, ERK1, ERK2, KRAS, HRAS and CCND1 , and the 4-gene signature comprises EGFR, ERK1, KRAS and CCND1 . Positive predictive power was good, with only 3 or 4 of the 14 models with high readouts failing to respond with stasis or tumor regression. Negative predictive power was slightly less robust, with 6 or 7 of the 15 low readouts showing good therapeutic responses. Interestingly, the signature could be reduced to three key genes (EGFR, ERK1 and KRAS or EGFR, ERK1 and CCND1) without loss of predictive power (FIG. 11). Even two-gene signatures (EGFR and ERK1, ERK1 and CCND1 , or EGFR and CCND1) correctly predicted sensitivity in 9/14, 10/14, and 11/14 high expressors, respectively, and resistance in 7/15, 8/15 and 8/15 low expressors, respectively (FIG. 12). By contrast, single gene signatures of all of the markers tested, or a signature comprising other RAS-ERK pathway components, such as a 6-gene signature comprising NRAS, ARAF , BRAF , CRAF , MEK1 and MEK2 , were largely uninformative (FIG. 13).

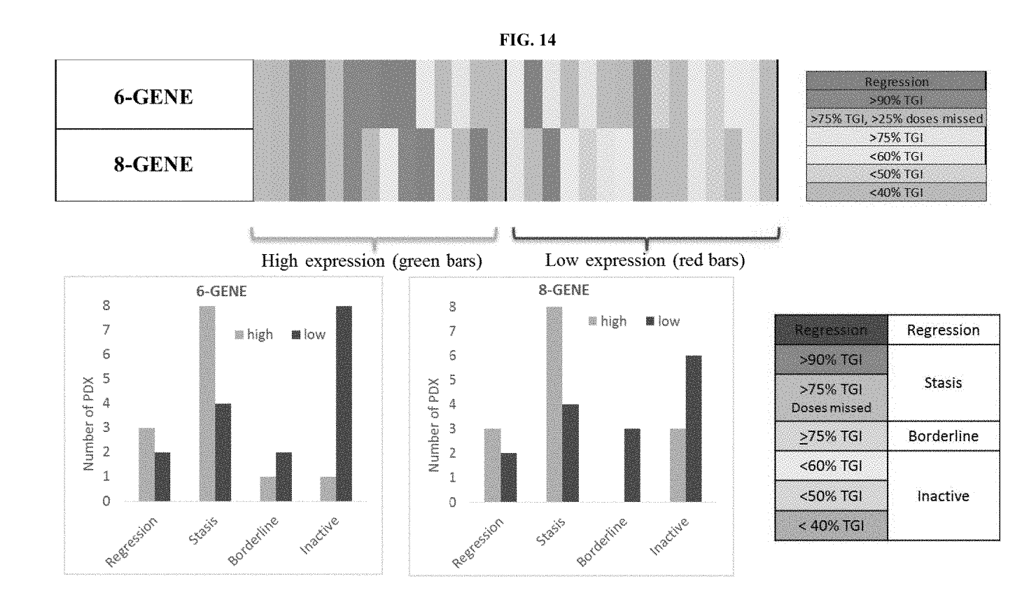

As shown in FIG. 14, a different 6-gene signature comprising both MAPK pathway genes and RAS-ERK feedback regulators (CCND1 , CRAF , DUSP5 , EGFR, ERK 1 and KRAS) correctly predicted sensitivity in 11/14 models with high readout, and resistance in 9/15 models with low readout. An 8-gene signature comprising MAPK pathway genes (EGFR, ERK 1 , ERK2, KRAS, HRAS, CCND1, CDK4 and CDK6) afforded similar predictive power.

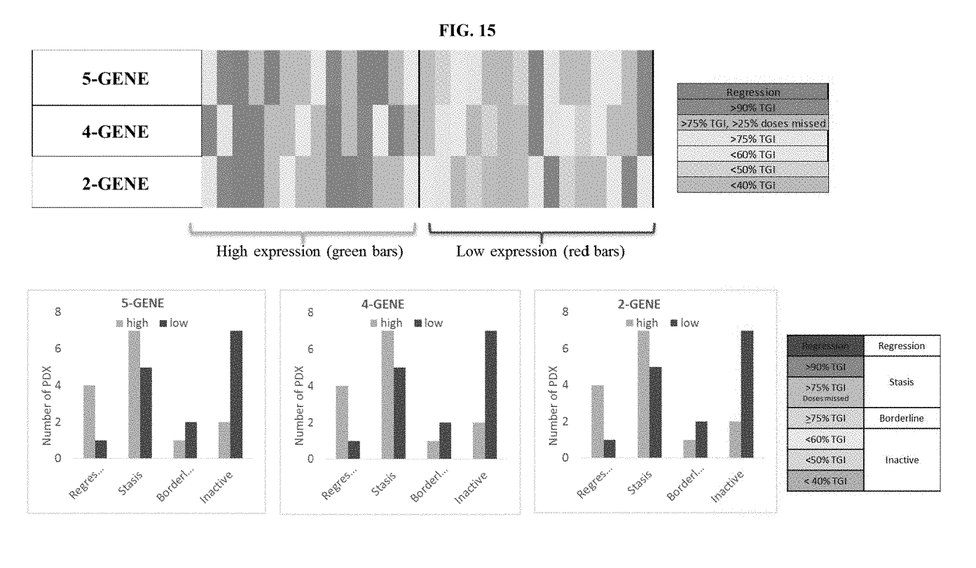

The predictive power of 5-, 4- and 2-gene signatures comprising RAS-ERK feedback regulators, including ERK phosphatases (DUSP2 , DUSP4 , DUSP5 and DUSP6) and RAS inhibitors (SPRY2, SPRY4 and SPRED1), was assessed for their association with sensitivity or resistance to an ERK inhibitor. As shown in FIG. 15, a 5-gene signature comprising DUSP5 , DUSP6, SPRY2 , SPRY4 and SPRED1 gave good predictive value in the series of 29 SCC models, with 11/14 models with high readout correctly predicted to be sensitive, and 9/15 models with low readout correctly predicted to be resistant to treatment with the ERK inhibitor. Because RAS is rarely mutated in SCCs, it was considered possible that the ERK feedback regulators alone may predict sensitivity to ERK inhibition, so a DUSP-specific 4-gene signature was assessed (DUSP2, DUSP4 , DUSP5 and DUSP6) and found to be equally predictive as the 5-gene signature. Remarkably, the full predictive power of both the 5- and 4-gene signatures was retained in a 2-gene signature comprising only DUSP5 and DUSP6, underlining the value of these biomarkers for identifying SCC patients whose tumors are likely to respond to treatment with an ERK inhibitor, such as KO-947. Exemplary ERK inhibitors, including KO-947, are provided in Table 3.

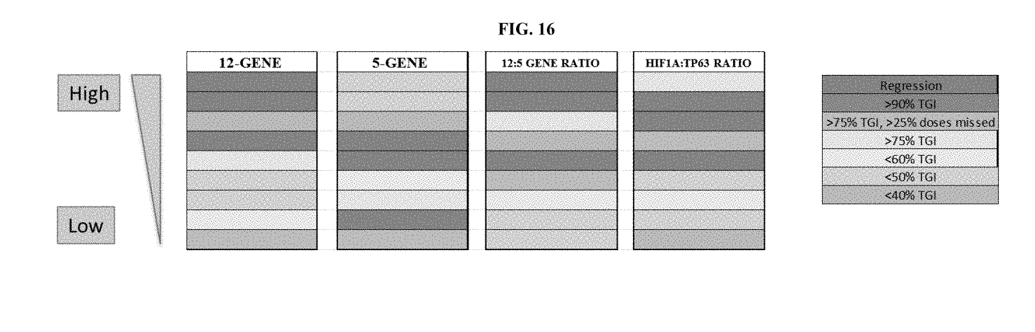

Example 8: Analysis of gene expression signatures in squamous cell carcinomas of the head and neck. Several gene signatures were evaluated across the panel of 9 head and neck squamous cell carcinoma models tested in Example 3. Results of the analyses are presented in FIG. 16 as heatmaps that show the relationship between response to treatment with the ERK inhibitor and total mRNA abundance of the genes that comprise the signature. Total expression levels (i.e. total mRNA abundance) are plotted from high to low (top to bottom) in each of the figures. A 12-gene transcriptional signature comprising AREG, CDH3, COL17A1 , EGFR, HIF1A, ITGB1, KRT1 , KRT9 , NRG1 , SLC16A1 , SLC22A1 and VEGFA correctly predicted a good response to the ERK inhibitor for the high readout models. A 5-gene signature comprising genes located in a region of chromosome 3 that is commonly amplified (Ch3A) in HNSCC (i.e. DCUN1D1, PIK3CA, PRKC 1, SOX2 and TP63) predicted for poor response to ERK inhibition, as shown in FIG. 16. A ratio of the 12-gene signature to the 5-gene signature correctly predicted a good response to ERK inhibition. Remarkably, the ratio of HIF1A to TP63 expression strongly predicted for good response to ERK inhibition.

Example 9: Inhibition Assays of ERK. The inhibition of ERK activity by the compounds disclosed herein was determined using the Z′-LYTE kinase assay kit (Life Technologies) with a Ser/Thr 3 peptide substrate (Life Technologies) according to manufacturer's instructions. The assay was run with an ERK2 enzyme (Life Technologies) concentration of 0.47 ng/μL at 100 μM ATP (approximately the ATP Kₘ for ERK2). The IC50 values for the compounds were determined with 3-fold serial dilutions in duplicate. The compounds were first diluted in 1:3 dilutions in 100% DMSO at 100× the desired concentration, and then further diluted (1:25) in 20 mM HEPES buffer (Invitrogen) to make 4× solutions prior to adding to the enzyme solution. The final DMSO concentration in the assay was 1%. Final reaction volume was 20 pt/well in 384-well plates. Kinase reactions were conducted for 1 hour followed by the assay development reaction (1 hour) in a 384 well plate format (20 μL/well). One or more compounds disclosed herein exhibited an IC50 less than 10 nM when tested in this assay. Results for select compounds are presented in Table 3.

TABLE 3
In vitro Erk2 IC50 data for select compounds
(+++ represents 50 nM to 250 nM, and ++++ represents less than 500 nM).

| | | Erk2 |
|---|---|---|
| | | IC50 |
| No | Chemical Structure | (nM) |
| 1 | 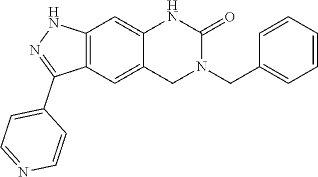 [SMILES: O=C1Nc2cc3[nH]nc(-c4ccncc4)c3cc2CN1Cc1ccccc1] | ++++ |
| 3 | 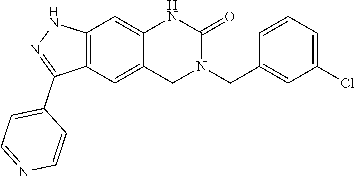 [SMILES: O=C1Nc2cc3[nH]nc(-c4ccncc4)c3cc2CN1Cc1cccc(Cl)c1] | ++++ |
| 4 | 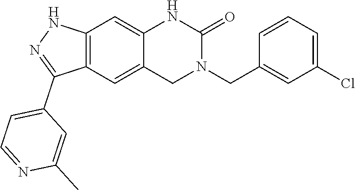 [SMILES: Cc1cc(-c2n[nH]c3cc4c(cc23)CN(Cc2cccc(Cl)c2)C(=O)N4)ccn1] | ++++ |
| 22 | 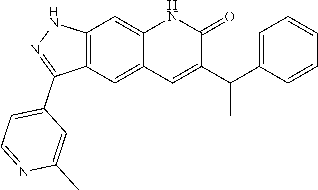 [SMILES: Cc1cc(-c2n[nH]c3cc4[nH]c(=O)c(C(C)c5ccccc5)cc4cc23)ccn1] | ++++ |
| 63 | 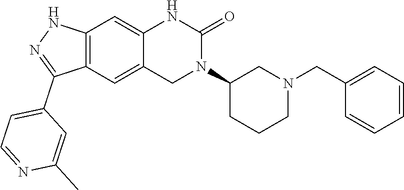 [SMILES: Cc1cc(-c2n[nH]c3cc4c(cc23)CN([C@@H]2CCCN(Cc3ccccc3)C2)C(=O)N4)ccn1] | ++++ |
| 64 | 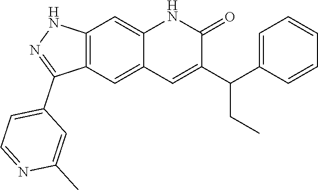 [SMILES: CCC(c1ccccc1)c1cc2cc3c(-c4ccnc(C)c4)n[nH]c3cc2[nH]c1=O] | ++++ |
| 68 | 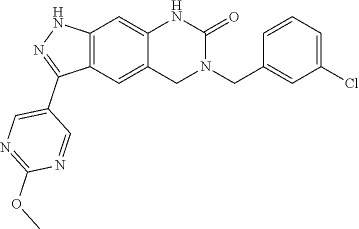 [SMILES: COc1ncc(-c2n[nH]c3cc4c(cc23)CN(Cc2cccc(Cl)c2)C(=O)N4)cn1] | ++++ |
| 84 | 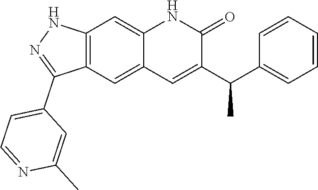 [SMILES: Cc1cc(-c2n[nH]c3cc4[nH]c(=O)c([C@H](C)c5ccccc5)cc4cc23)ccn1] | ++++ |
| 101 | 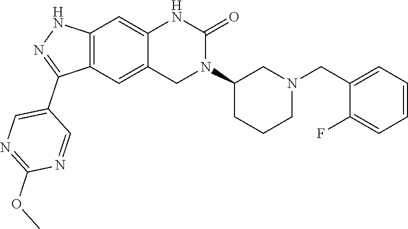 [SMILES: COc1ncc(-c2n[nH]c3cc4c(cc23)CN([C@@H]2CCCN(Cc3ccccc3F)C2)C(=O)N4)cn1] | ++++ |
| 102 | 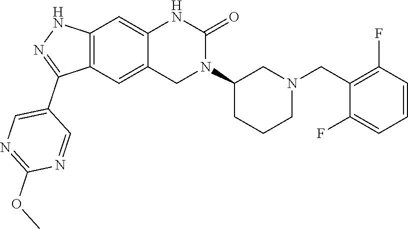 [SMILES: COc1ncc(-c2n[nH]c3cc4c(cc23)CN([C@@H]2CCCN(Cc3c(F)cccc3F)C2)C(=O)N4)cn1] | ++++ |
| 103 | 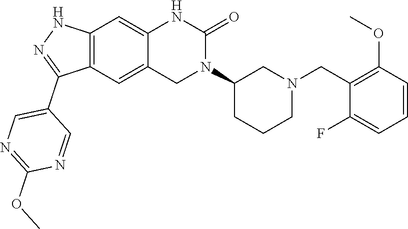 [SMILES: COc1ncc(-c2n[nH]c3cc4c(cc23)CN([C@@H]2CCCN(Cc3c(F)cccc3OC)C2)C(=O)N4)cn1] | ++++ |
| 104 | 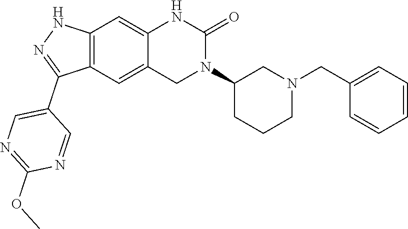 [SMILES: COc1ncc(-c2n[nH]c3cc4c(cc23)CN([C@@H]2CCCN(Cc3ccccc3)C2)C(=O)N4)cn1] | ++++ |
| 134 | 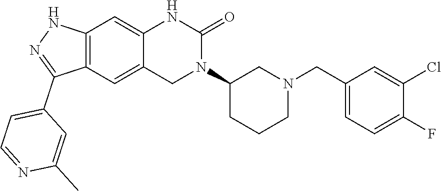 [SMILES: Cc1cc(-c2n[nH]c3cc4c(cc23)CN([C@@H]2CCCN(Cc3ccc(F)c(Cl)c3)C2)C(=O)N4)ccn1] | ++++ |
| 143 | 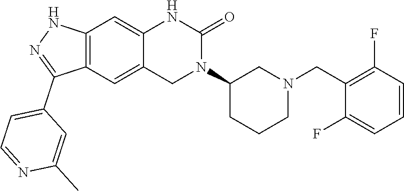 [SMILES: Cc1cc(-c2n[nH]c3cc4c(cc23)CN([C@@H]2CCCN(Cc3c(F)cccc3F)C2)C(=O)N4)ccn1] | ++++ |
| 144 | 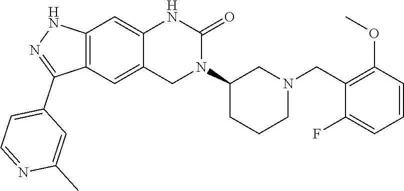 [SMILES: COc1cccc(F)c1CN1CCC[C@@H](N2Cc3cc4c(-c5ccnc(C)c5)n[nH]c4cc3NC2=O)C1] | ++++ |
| 146 | 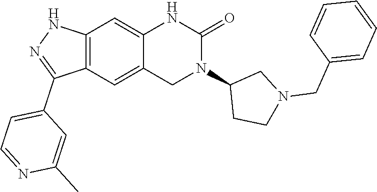 [SMILES: Cc1cc(-c2n[nH]c3cc4c(cc23)CN([C@@H]2CCN(Cc3ccccc3)C2)C(=O)N4)ccn1] | ++++ |
| 148 | 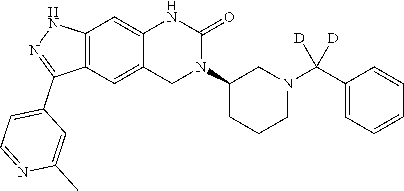 [SMILES: [2H]C([2H])(c1ccccc1)N1CCC[C@@H](N2Cc3cc4c(-c5ccnc(C)c5)n[nH]c4cc3NC2=O)C1] | ++++ |
| 149 | 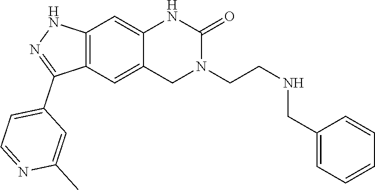 [SMILES: Cc1cc(-c2n[nH]c3cc4c(cc23)CN(CCNCc2ccccc2)C(=O)N4)ccn1] | ++++ |
| 151 | 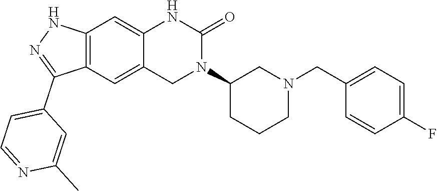 [SMILES: Cc1cc(-c2n[nH]c3cc4c(cc23)CN([C@@H]2CCCN(Cc3ccc(F)cc3)C2)C(=O)N4)ccn1] | ++++ |
| 153 | 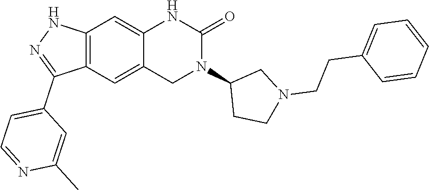 [SMILES: Cc1cc(-c2n[nH]c3cc4c(cc23)CN([C@@H]2CCN(CCc3ccccc3)C2)C(=O)N4)ccn1] | ++++ |
| 154 | 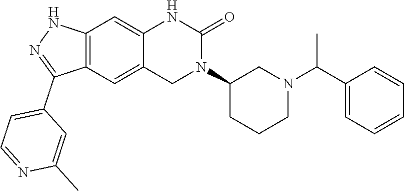 [SMILES: Cc1cc(-c2n[nH]c3cc4c(cc23)CN([C@@H]2CCCN(C(C)c3ccccc3)C2)C(=O)N4)ccn1] | ++++ |
| 156 | 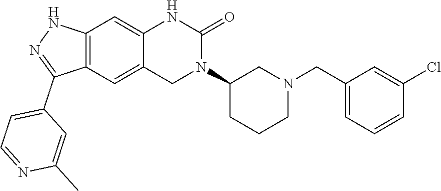 [SMILES: Cc1cc(-c2n[nH]c3cc4c(cc23)CN([C@@H]2CCCN(Cc3cccc(Cl)c3)C2)C(=O)N4)ccn1] | ++++ |
| 158 | 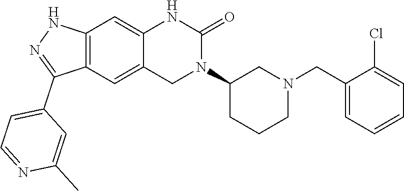 [SMILES: Cc1cc(-c2n[nH]c3cc4c(cc23)CN([C@@H]2CCCN(Cc3ccccc3Cl)C2)C(=O)N4)ccn1] | ++++ |
| 162 | 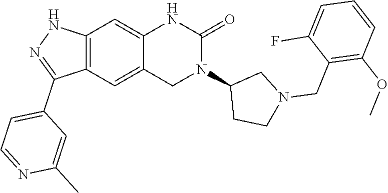 [SMILES: COc1cccc(F)c1CN1CC[C@@H](N2Cc3cc4c(-c5ccnc(C)c5)n[nH]c4cc3NC2=O)C1] | ++++ |
| 165 | 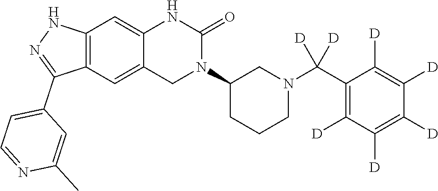 [SMILES: [2H]c1c([2H])c([2H])c(C([2H])([2H])N2CCC[C@@H](N3Cc4cc5c(-c6ccnc(C)c6)n[nH]c5cc4NC3=O)C2)c([2H])c1[2H]] | ++++ |
| 181 | 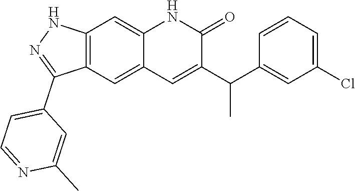 [SMILES: Cc1cc(-c2n[nH]c3cc4[nH]c(=O)c(C(C)c5cccc(Cl)c5)cc4cc23)ccn1] | +++ |
| 184 | 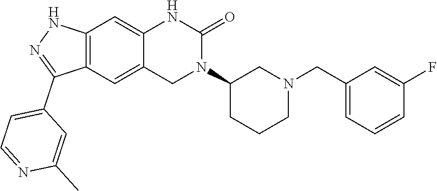 [SMILES: Cc1cc(-c2n[nH]c3cc4c(cc23)CN([C@@H]2CCCN(Cc3cccc(F)c3)C2)C(=O)N4)ccn1] | ++++ |
| 185 | 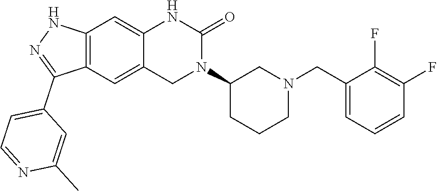 [SMILES: Cc1cc(-c2n[nH]c3cc4c(cc23)CN([C@@H]2CCCN(Cc3cccc(F)c3F)C2)C(=O)N4)ccn1] | ++++ |
| 187 | 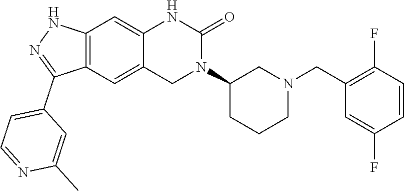 [SMILES: Cc1cc(-c2n[nH]c3cc4c(cc23)CN([C@@H]2CCCN(Cc3cc(F)ccc3F)C2)C(=O)N4)ccn1] | ++++ |
| 190 | 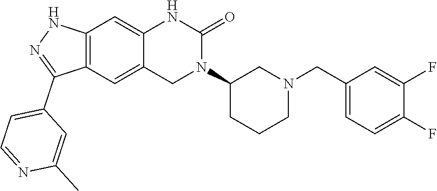 [SMILES: Cc1cc(-c2n[nH]c3cc4c(cc23)CN([C@@H]2CCCN(Cc3ccc(F)c(F)c3)C2)C(=O)N4)ccn1] | ++++ |
| 196 | 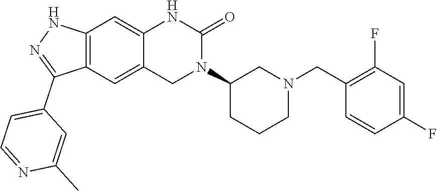 [SMILES: Cc1cc(-c2n[nH]c3cc4c(cc23)CN([C@@H]2CCCN(Cc3ccc(F)cc3F)C2)C(=O)N4)ccn1] | ++++ |
| 197 | 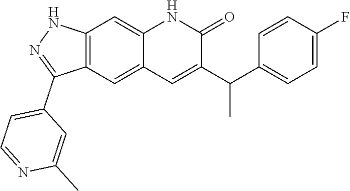 [SMILES: Cc1cc(-c2n[nH]c3cc4[nH]c(=O)c(C(C)c5ccc(F)cc5)cc4cc23)ccn1] | ++++ |
| 212 | 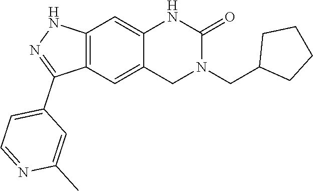 [SMILES: Cc1cc(-c2n[nH]c3cc4c(cc23)CN(CC2CCCC2)C(=O)N4)ccn1] | ++++ |
| 213 | 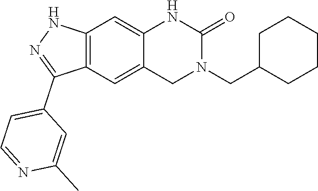 [SMILES: Cc1cc(-c2n[nH]c3cc4c(cc23)CN(CC2CCCCC2)C(=O)N4)ccn1] | ++++ |
| 214 | 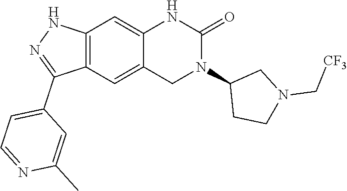 [SMILES: Cc1cc(-c2n[nH]c3cc4c(cc23)CN([C@@H]2CCN(CC(F)(F)F)C2)C(=O)N4)ccn1] | ++++ |
| 225 | 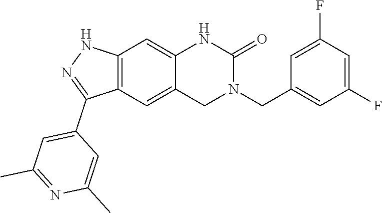 [SMILES: Cc1cc(-c2n[nH]c3cc4c(cc23)CN(Cc2cc(F)cc(F)c2)C(=O)N4)cc(C)n1] | ++++ |
| 237 | 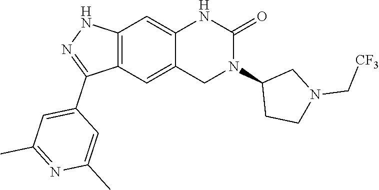 [SMILES: Cc1cc(-c2n[nH]c3cc4c(cc23)CN([C@@H]2CCN(CC(F)(F)F)C2)C(=O)N4)cc(C)n1] | ++++ |
| 239 | 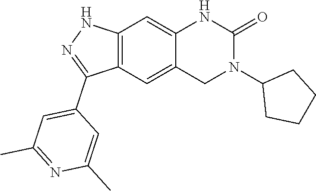 [SMILES: Cc1cc(-c2n[nH]c3cc4c(cc23)CN(C2CCCC2)C(=O)N4)cc(C)n1] | ++++ |
| 240 | 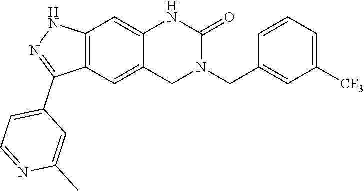 [SMILES: Cc1cc(-c2n[nH]c3cc4c(cc23)CN(Cc2cccc(C(F)(F)F)c2)C(=O)N4)ccn1] | ++++ |
| 241 | 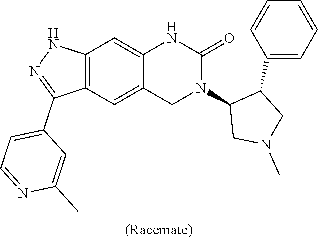 [SMILES: Cc1cc(-c2n[nH]c3cc4c(cc23)CN([C@@H]2CN(C)C[C@H]2c2ccccc2)C(=O)N4)ccn1] | ++++ |
| 243 | 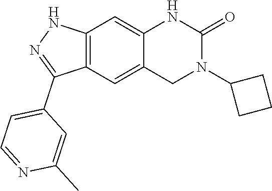 [SMILES: Cc1cc(-c2n[nH]c3cc4c(cc23)CN(C2CCC2)C(=O)N4)ccn1] | ++++ |
| 244 | 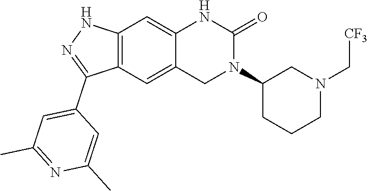 [SMILES: Cc1cc(-c2n[nH]c3cc4c(cc23)CN([C@@H]2CCCN(CC(F)(F)F)C2)C(=O)N4)cc(C)n1] | ++++ |
| 245 | 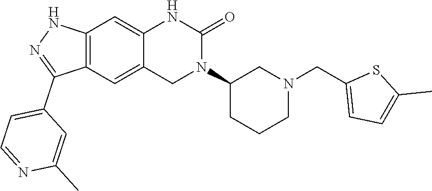 [SMILES: Cc1cc(-c2n[nH]c3cc4c(cc23)CN([C@@H]2CCCN(Cc3ccc(C)s3)C2)C(=O)N4)ccn1] | ++++ |
| 248 | 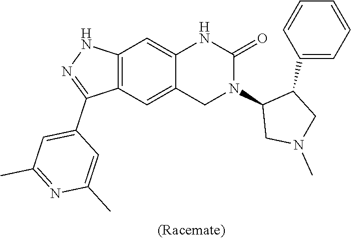 [SMILES: Cc1cc(-c2n[nH]c3cc4c(cc23)CN([C@@H]2CN(C)C[C@H]2c2ccccc2)C(=O)N4)cc(C)n1] | ++++ |
| 251 | 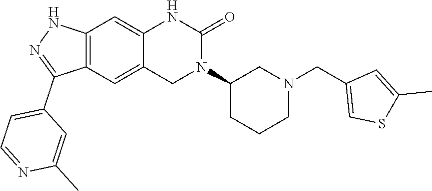 [SMILES: Cc1cc(-c2n[nH]c3cc4c(cc23)CN([C@@H]2CCCN(Cc3csc(C)c3)C2)C(=O)N4)ccn1] | ++++ |
| 252 | 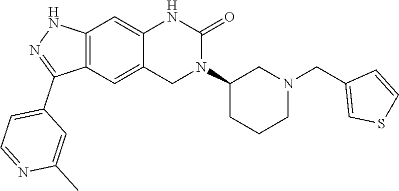 [SMILES: Cc1cc(-c2n[nH]c3cc4c(cc23)CN([C@@H]2CCCN(Cc3ccsc3)C2)C(=O)N4)ccn1] | ++++ |
| 254 | 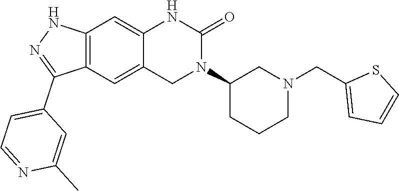 [SMILES: Cc1cc(-c2n[nH]c3cc4c(cc23)CN([C@@H]2CCCN(Cc3cccs3)C2)C(=O)N4)ccn1] | ++++ |
| 264 | 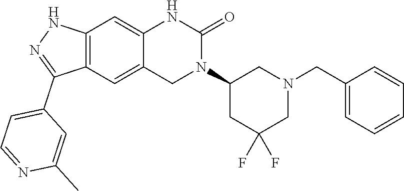 [SMILES: Cc1cc(-c2n[nH]c3cc4c(cc23)CN([C@H]2CN(Cc3ccccc3)CC(F)(F)C2)C(=O)N4)ccn1] | ++++ |
| 267 | 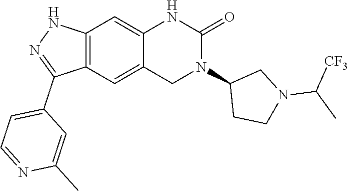 [SMILES: Cc1cc(-c2n[nH]c3cc4c(cc23)CN([C@@H]2CCN(C(C)C(F)(F)F)C2)C(=O)N4)ccn1] | ++++ |
| 269 | 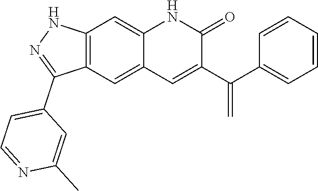 [SMILES: C=C(c1ccccc1)c1cc2cc3c(-c4ccnc(C)c4)n[nH]c3cc2[nH]c1=O] | ++++ |
| 270 | 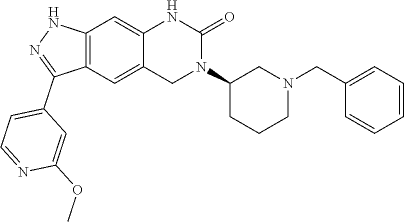 [SMILES: COc1cc(-c2n[nH]c3cc4c(cc23)CN([C@@H]2CCCN(Cc3ccccc3)C2)C(=O)N4)ccn1] | ++++ |
| 271 | 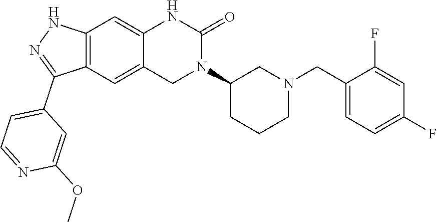 [SMILES: COc1cc(-c2n[nH]c3cc4c(cc23)CN([C@@H]2CCCN(Cc3ccc(F)cc3F)C2)C(=O)N4)ccn1] | ++++ |
| 272 | 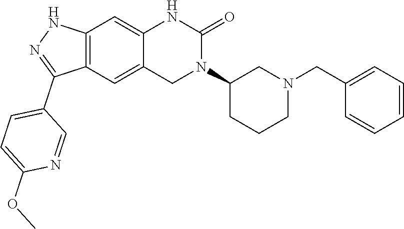 [SMILES: COc1ccc(-c2n[nH]c3cc4c(cc23)CN([C@@H]2CCCN(Cc3ccccc3)C2)C(=O)N4)cn1] | ++++ |
| 273 | 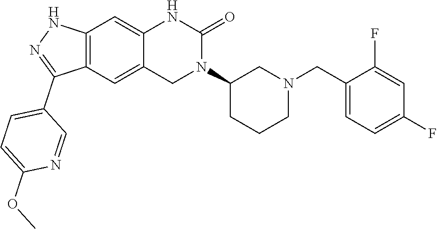 [SMILES: COc1ccc(-c2n[nH]c3cc4c(cc23)CN([C@@H]2CCCN(Cc3ccc(F)cc3F)C2)C(=O)N4)cn1] | ++++ |
| 275 | 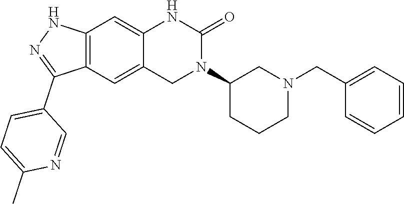 [SMILES: Cc1ccc(-c2n[nH]c3cc4c(cc23)CN([C@@H]2CCCN(Cc3ccccc3)C2)C(=O)N4)cn1] | ++++ |
| 278 | 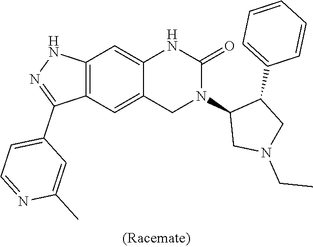 [SMILES: CCN1C[C@@H](N2Cc3cc4c(-c5ccnc(C)c5)n[nH]c4cc3NC2=O)[C@H](c2ccccc2)C1] | ++++ |
| 279 | 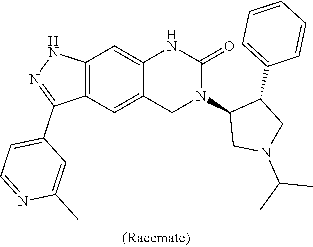 [SMILES: Cc1cc(-c2n[nH]c3cc4c(cc23)CN([C@@H]2CN(C(C)C)C[C@H]2c2ccccc2)C(=O)N4)ccn1] | ++++ |
| 280 | 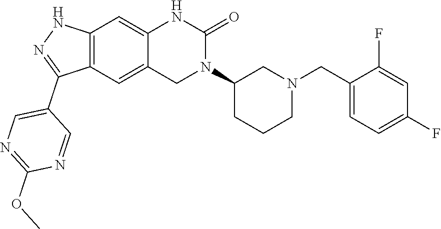 [SMILES: COc1ncc(-c2n[nH]c3cc4c(cc23)CN([C@@H]2CCCN(Cc3ccc(F)cc3F)C2)C(=O)N4)cn1] | ++++ |
| 281 | 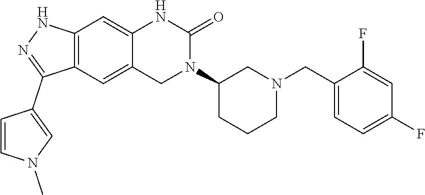 [SMILES: Cn1ccc(-c2n[nH]c3cc4c(cc23)CN([C@@H]2CCCN(Cc3ccc(F)cc3F)C2)C(=O)N4)c1] | ++++ |
| 284 | 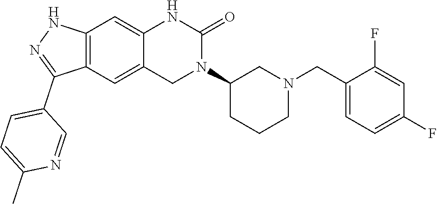 [SMILES: Cc1ccc(-c2n[nH]c3cc4c(cc23)CN([C@@H]2CCCN(Cc3ccc(F)cc3F)C2)C(=O)N4)cn1] | ++++ |
| 285 | 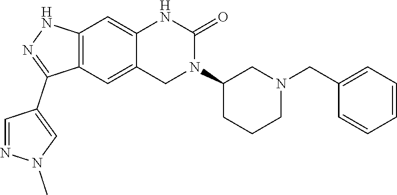 [SMILES: Cn1cc(-c2n[nH]c3cc4c(cc23)CN([C@@H]2CCCN(Cc3ccccc3)C2)C(=O)N4)cn1] | ++++ |
| 288 | 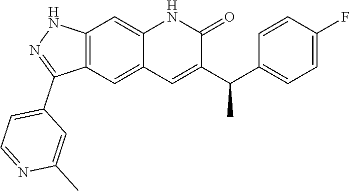 [SMILES: Cc1cc(-c2n[nH]c3cc4[nH]c(=O)c([C@H](C)c5ccc(F)cc5)cc4cc23)ccn1] | ++++ |
| 291 | 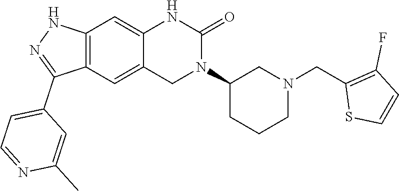 [SMILES: Cc1cc(-c2n[nH]c3cc4c(cc23)CN([C@@H]2CCCN(Cc3sccc3F)C2)C(=O)N4)ccn1] | ++++ |
| 292 | 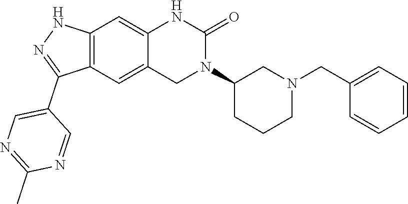 [SMILES: Cc1ncc(-c2n[nH]c3cc4c(cc23)CN([C@@H]2CCCN(Cc3ccccc3)C2)C(=O)N4)cn1] | ++++ |
| 293 | 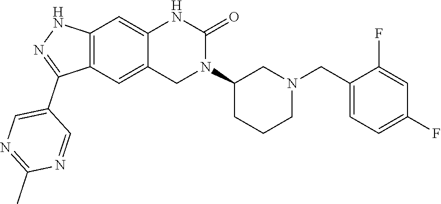 [SMILES: Cc1ncc(-c2n[nH]c3cc4c(cc23)CN([C@@H]2CCCN(Cc3ccc(F)cc3F)C2)C(=O)N4)cn1] | ++++ |
| 304 | 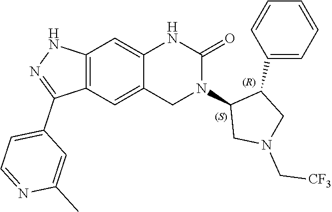 | ++++ |
| 305 | 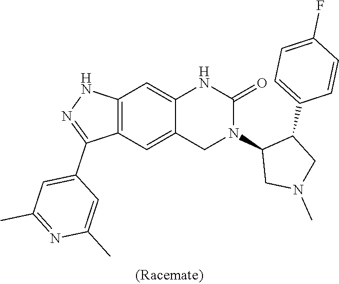 [SMILES: Cc1cc(-c2n[nH]c3cc4c(cc23)CN([C@@H]2CN(C)C[C@H]2c2ccc(F)cc2)C(=O)N4)cc(C)n1] | +++ |
| 306 | 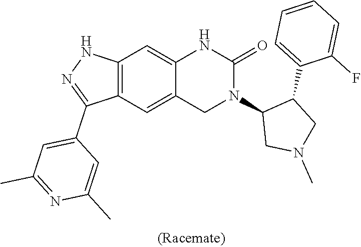 [SMILES: Cc1cc(-c2n[nH]c3cc4c(cc23)CN([C@@H]2CN(C)C[C@H]2c2ccccc2F)C(=O)N4)cc(C)n1] | ++++ |
| 316 | 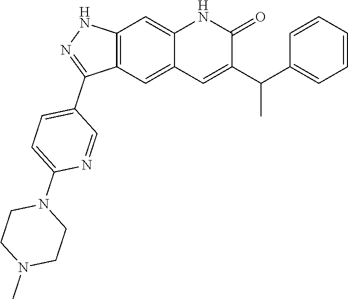 [SMILES: CC(c1ccccc1)c1cc2cc3c(-c4ccc(N5CCN(C)CC5)nc4)n[nH]c3cc2[nH]c1=O] | ++++ |
| 318 | 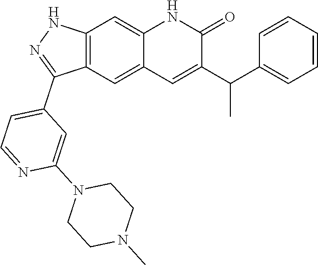 [SMILES: CC(c1ccccc1)c1cc2cc3c(-c4ccnc(N5CCN(C)CC5)c4)n[nH]c3cc2[nH]c1=O] | ++++ |
| 324 | 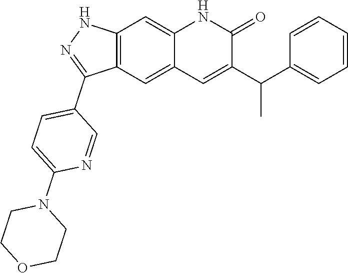 [SMILES: CC(c1ccccc1)c1cc2cc3c(-c4ccc(N5CCOCC5)nc4)n[nH]c3cc2[nH]c1=O] | +++ |
| 325 | 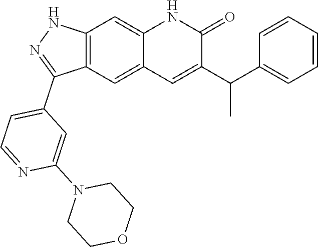 [SMILES: CC(c1ccccc1)c1cc2cc3c(-c4ccnc(N5CCOCC5)c4)n[nH]c3cc2[nH]c1=O] | ++++ |
| 326 | 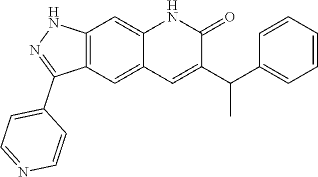 [SMILES: CC(c1ccccc1)c1cc2cc3c(-c4ccncc4)n[nH]c3cc2[nH]c1=O] | ++++ |
| 332 | 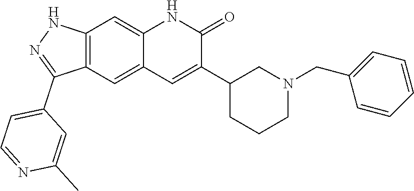 [SMILES: Cc1cc(-c2n[nH]c3cc4[nH]c(=O)c(C5CCCN(Cc6ccccc6)C5)cc4cc23)ccn1] | ++++ |
| 333 | 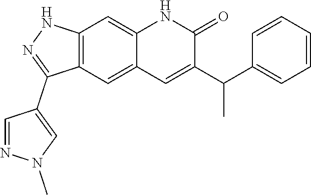 [SMILES: CC(c1ccccc1)c1cc2cc3c(-c4cnn(C)c4)n[nH]c3cc2[nH]c1=O] | ++++ |
| 338 | 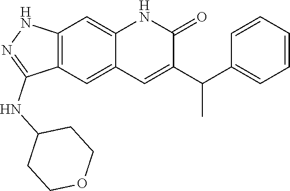 [SMILES: CC(c1ccccc1)c1cc2cc3c(NC4CCOCC4)n[nH]c3cc2[nH]c1=O] | ++++ |
| 339 | 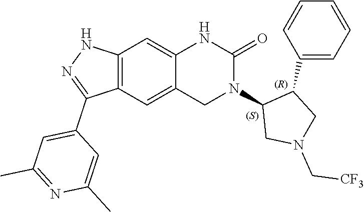 | ++++ |
| 357 | 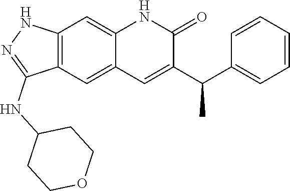 [SMILES: C[C@H](c1ccccc1)c1cc2cc3c(NC4CCOCC4)n[nH]c3cc2[nH]c1=O] | ++++ |
| 362 | 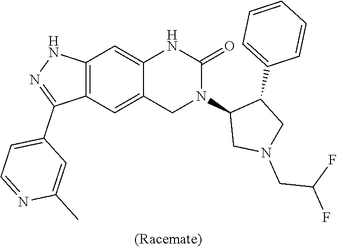 [SMILES: Cc1cc(-c2n[nH]c3cc4c(cc23)CN([C@@H]2CN(CC(F)F)C[C@H]2c2ccccc2)C(=O)N4)ccn1] | ++++ |
| 364 | 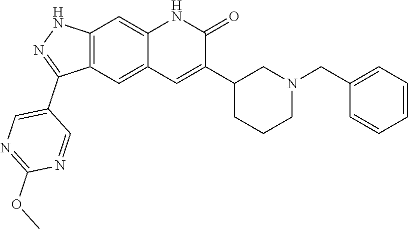 [SMILES: COc1ncc(-c2n[nH]c3cc4[nH]c(=O)c(C5CCCN(Cc6ccccc6)C5)cc4cc23)cn1] | ++++ |

Example 10: Tumor cell line proliferation assay. The ability of one or more compounds of the disclosure to inhibit tumor cell line proliferation was determined according to standard procedures known in the art. For instance, an in vitro cellular proliferation assay was performed to measure the metabolic activity of live cells. A375 cells (ATCC) were grown to near 80% confluence, trypsinized and seeded at 1500 cells/well at volume of 100 μL per well in full growth medium (10% FBS in DMEM or 10% FBS in RPMI) in a 96 well plate. The cells were incubated at 37 ° C. under 5% CO₂ for two hours to allow for attachment to the plates. Compounds were first diluted in 1:3 dilutions in 100% DMSO at 250× the desired concentration, and then further diluted (1:50) in 10% DMEM growth medium. The diluted compounds were added to the cell plate (25 ₜit for a 5× dilution) and the cells incubated with compounds (0.4% DMSO in 10% FBS DMEM) for 96 hours at 37 ° C. under 5% CO₂. The cell control wells were added with vehicle only (0.4% DMSO in 10% FBS DMEM or in 10% FBS RPMI). Each concentration of the compounds was tested in duplicate. After 96 hours of compound treatment, CellTiter Glo reagent (Promega) was added at a 1:5 dilution to each well of the cell plate and the cell plate was placed at room temperature for 30 minutes. The luminescence of the wells was determined using a Tecan plate reader. Each compound presented in Table 3 exhibited an IC50 of 250 nM or less in A375 cells (ATCC) when tested in this assay.

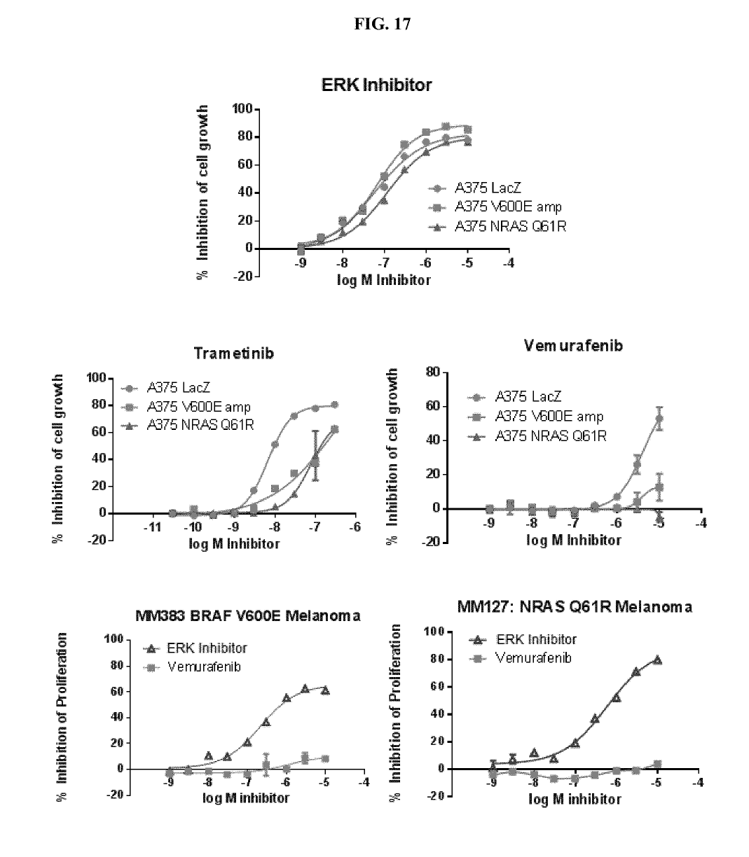

Example 11: Efficacy studies in models of clinical B-Raf and MEK inhibitor resistance. Human melanoma cell lines were obtained from ATCC or DSMZ (e.g., A375, MM383 BRAF V600E and MM127 NRAS Q61R). A375 cells were engineered to overexpress LacZ, BRAF V600E (BRAF V600E amp), or the NRAS mutant NRAS Q61R. The cell lines were grown to confluency, washed with Tumor Cell Media (DMEM+10% FBS or IMDM+20% FBS), and plated in 90 μL Tumor Cell Media at 5,000-10,000 cells/well. Either vemurafenib, trametinib, an ERK inhibitor selected from Table 3, or vehicle was added to each well. Plates were incubated for 72 hours at 37° C. and 5% CO₂. A volume of 100 μL of CellTiter-Glo® reagent was added to each well and plates were mixed for 2 minutes on an orbital shaker. The plates were allowed to stand at room temperature for 20 minutes before measuring the luminescent signal of each well. IC₅₀ values of each compound were calculated for each cell line and are presented in Table 4. Growth inhibition curves are presented in FIG. 17. One or more ERK inhibitors selected from Table 3 was found to potently inhibit cell lines engineered to be resistant to B-Raf and MEK inhibitors (e.g., vemurafenib and trametinib), and also inhibited cell lines with intrinsic resistance to vemurafenib.

TABLE 4
Summary of ERK inhibitor activity in models of
clinical B-Raf and MEK inhibitor resistance.

| | | |
|---|---|---|
| | A375 NRAS | A375 V600E |
| A375 LacZ | Q61R | amp |

| | | | | | | |
|---|---|---|---|---|---|---|
| ERK Inhibitor | 58 | nM | 116 | nM | 66 | nM |
| Vemurafenib | 4200 | nM | >10,000 | nM | >10,000 | nM |
| Trametinib | 7 | nM | 77 | nM | 300 | nM |

Example 12: Efficacy studies in patient-derived xenograft models of ESCC. The general procedure outline in Example 1 was followed. Briefly, tumor fragments (2-4 mm in diameter) from stock mice inoculated with primary human ESCC tissues were inoculated subcutaneously into BALB/C nude mice. The mice were stratified into groups when the average tumor size reached about 180 mm³. Animals were treated with vehicle or 300-350 mg/kg QW PO of an ERK inhibitor (KO-947, a compound of Formula I as described herein). Tumor volumes were measured twice weekly in two dimensions using a caliper, with volume expressed in mm³ (mean+−SEM) using the formula V=0.5(a×b)², where a and b are the long and short diameters of the tumor, respectively.

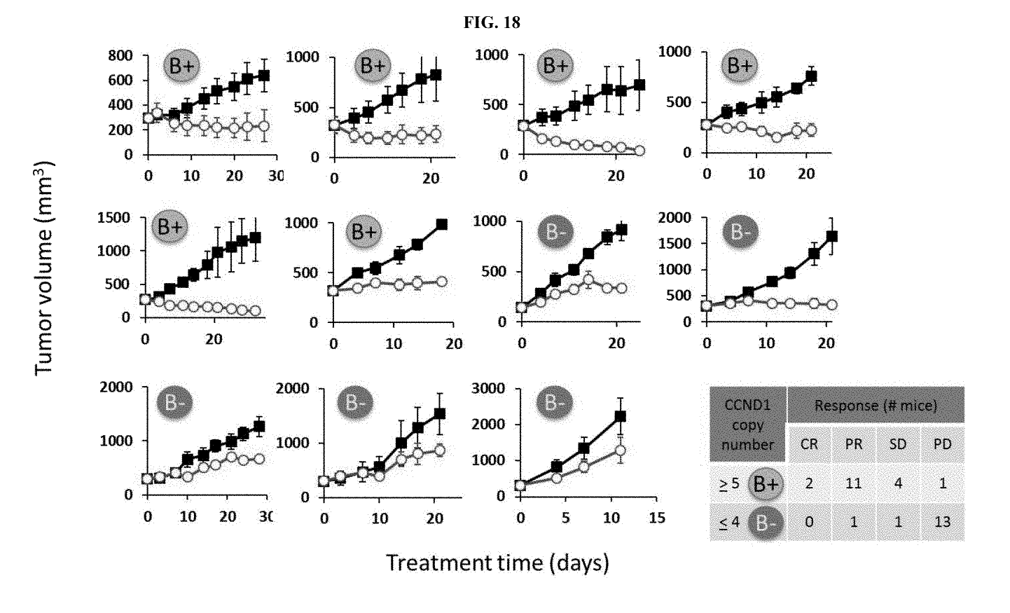

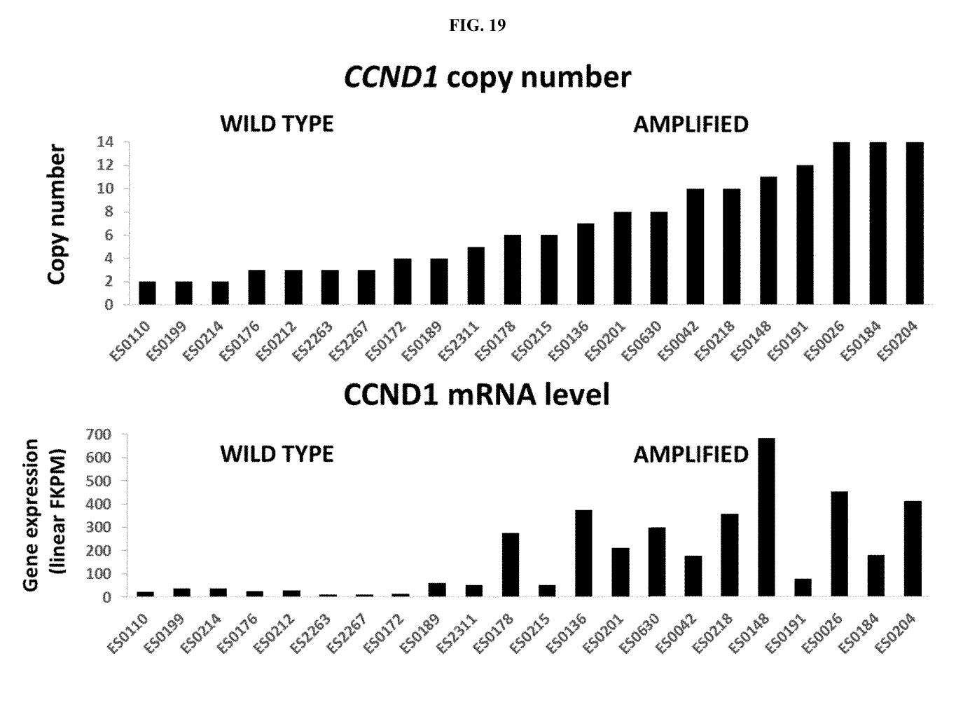

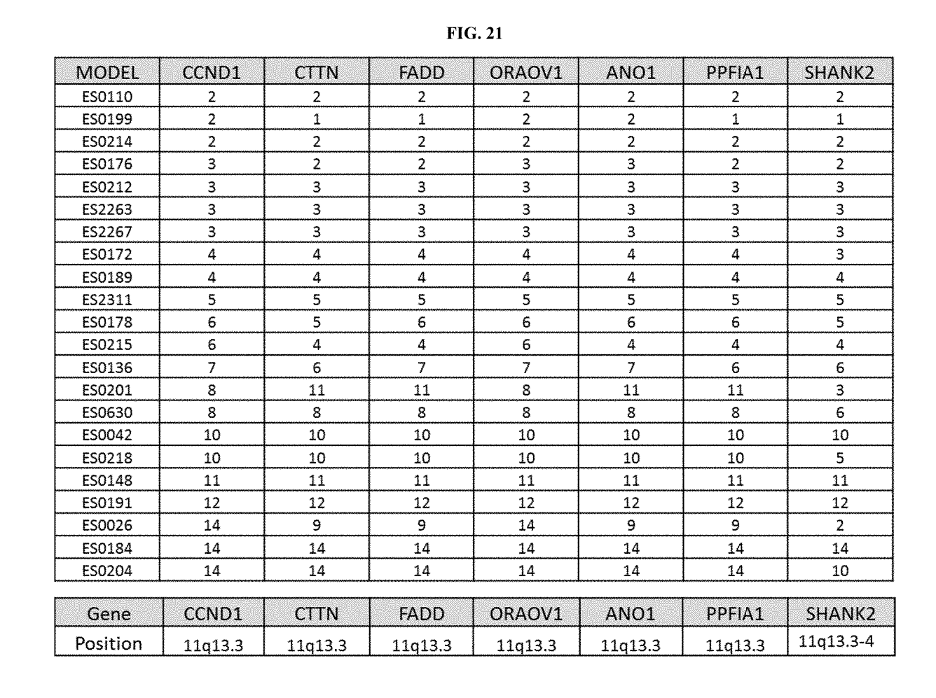

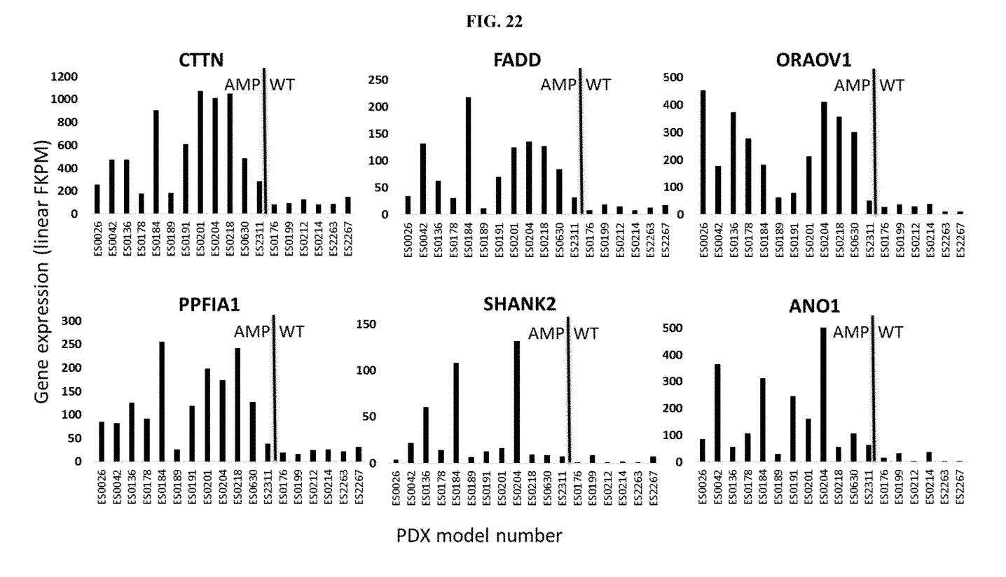

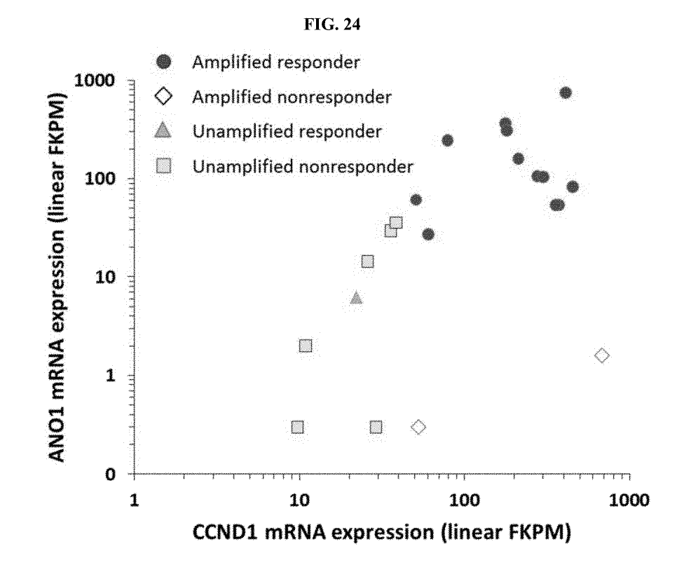

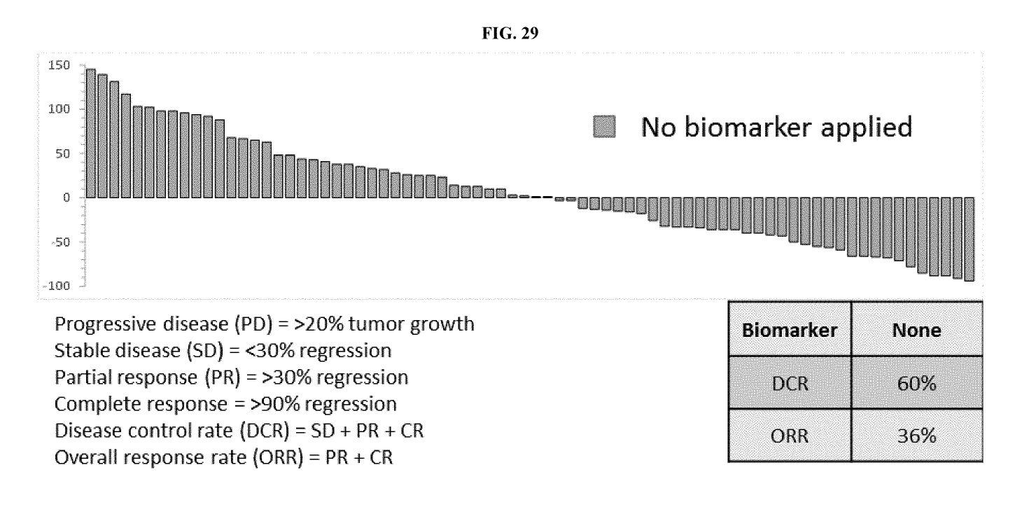

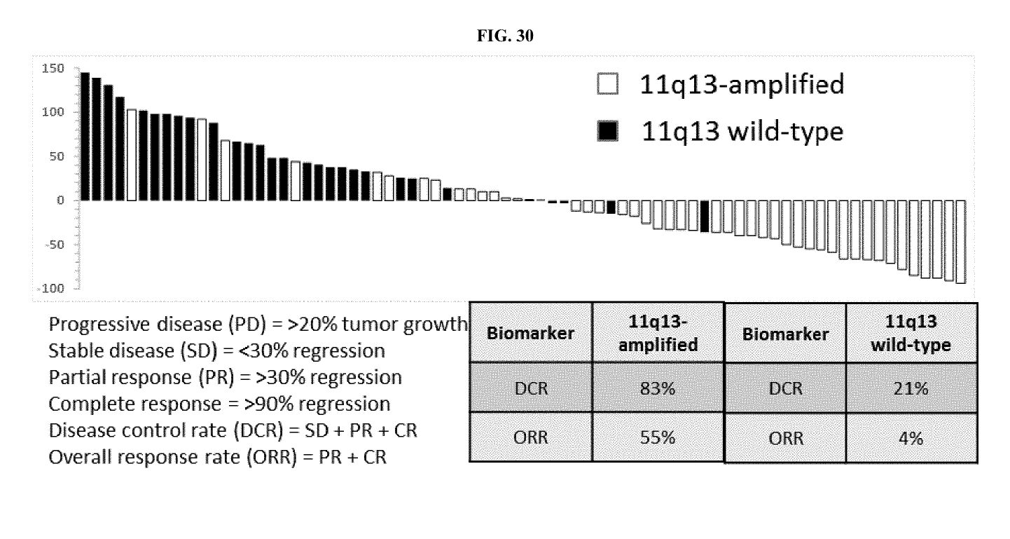

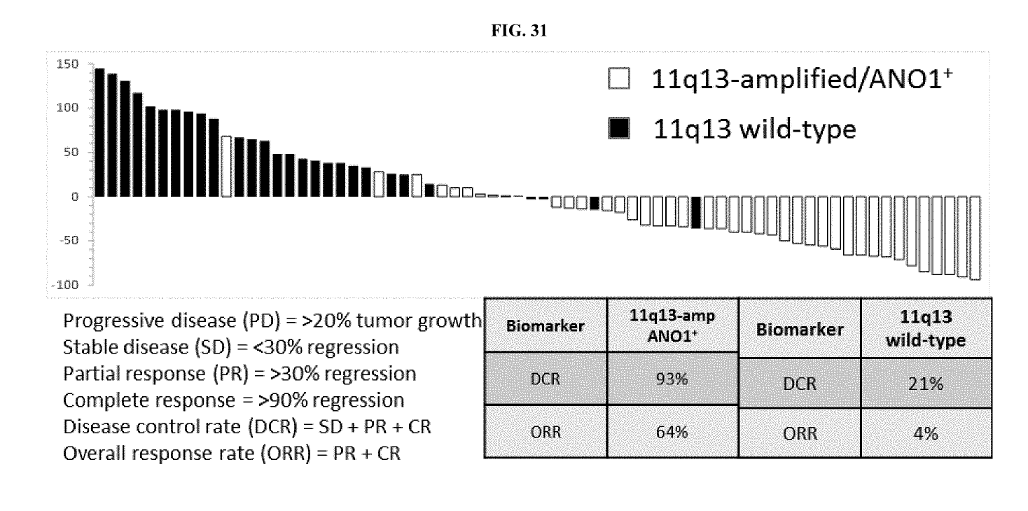

A total of eleven ESCC patient-derived xenograft models were treated in the same manner with either vehicle or the ERK inhibitor as presented in FIG. 18. ESCC models having a CCND1 copy number ≥5 or ≤4 are classified as “B+” or “B−”, respectively, in FIG. 18. CCND1 copy number and mRNA levels for ESCC models are presented in FIG. 19, and copy numbers for genes located at chromosome 11q13.3-13.4 are presented in FIG. 21 for the same ESCC models. Expression levels for six of these genes are presented graphically in FIG. 22. Gene copy number and gene expression were assessed as described in Example 1. As shown in FIG. 24, there was a positive association between ANO1 mRNA expression, CCND1 mRNA expression, ANO1 amplification, CCND1 amplification, and response to treatment with the ERK inhibitor. FIGS. 29-31 illustrate percent tumor growth for all tested ESCC models. When no biomarker is used to predict sensitivity, a disease control rate of 60% is observed (FIG. 29). The disease control rate is increased to 83% for 11q13-amplified models, compared to only 21% for 11q13 wild-type models (FIG. 30). The disease control rate for 11q13-amplified models that are AN01⁺further increased to 93% (FIG. 31).

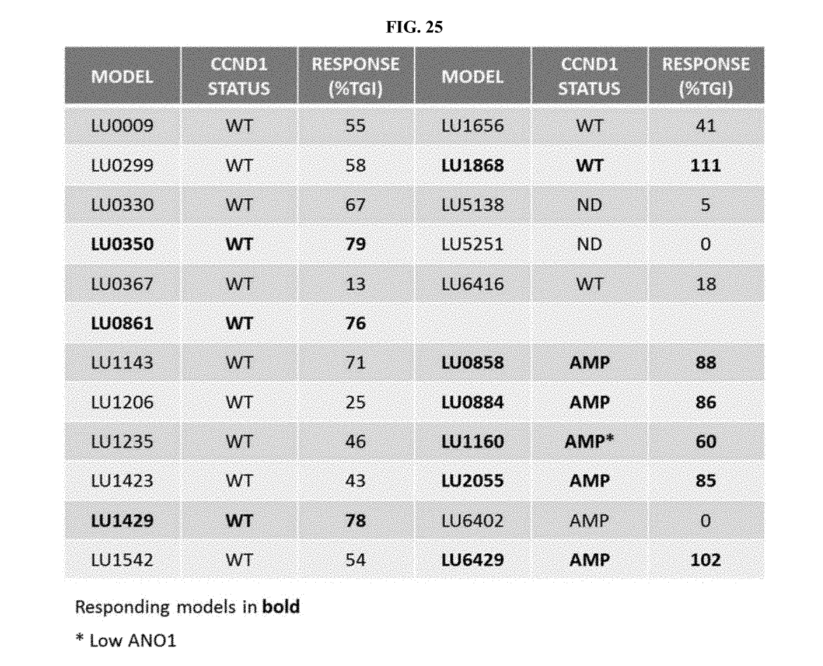

Example 13: Efficacy studies in patient-derived xenograft models of LSCC. The general procedure outline in Example 1 was followed. Briefly, tumor fragments (2-4 mm in diameter) from stock mice inoculated with primary human lung SCC tissues were inoculated subcutaneously into BALB/C nude mice. The mice were stratified into groups when the average tumor size reached about 200 mm³. Animals were treated with vehicle or 300-350 mg/kg QW PO of an ERK inhibitor (KO-947, a compound of Formula I as described herein). Tumor volumes were measured twice weekly in two dimensions using a caliper, with volume expressed in mm³ (mean+−SEM) using the formula V=0.5(a×b)², where a and b are the long and short diameters of the tumor, respectively. A total of 23 LSCC patient-derived xenograft models were treated in the same manner with either vehicle or the ERK inhibitor. Response to treatment with the ERK inhibitor is presented in FIG. 25 as percent tumor growth inhibition. A model may be classified as responsive to treatment with the ERK inhibitor if tumor growth inhibition was greater than or equal to 75%. Models with bold text in FIG. 25 are alternatively classified as responsive to treatment if at least one of the three animals treated achieved tumor stasis or regression.

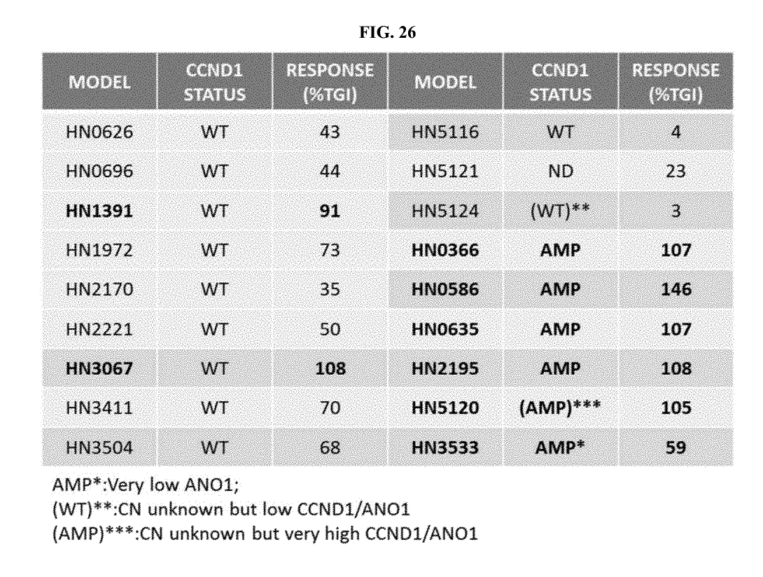

Example 14: Efficacy studies in patient-derived xenograft models of HNSCC. The general procedure outline in Example 1 was followed. Briefly, tumor fragments (2-4 mm in diameter) from stock mice inoculated with primary human HNSCC tissues were inoculated subcutaneously into BALB/C nude mice. The mice were stratified into groups when the average tumor size reached about 180 mm³. Animals were treated with vehicle or 300-350 mg/kg QW PO of an ERK inhibitor (KO-947, a compound of Formula I as described herein). Tumor volumes were measured twice weekly in two dimensions using a caliper, with volume expressed in mm³ (mean+−SEM) using the formula V=0.5(a×b)², where a and b are the long and short diameters of the tumor, respectively. A total of 17 HNSCC patient-derived xenograft models were treated in the same manner with either vehicle or the ERK inhibitor. Response to treatment with the ERK inhibitor is presented in FIG. 26 as percent tumor growth inhibition. Four out of six models exhibiting CCND1 amplification were responsive to treatment with the ERK inhibitor.

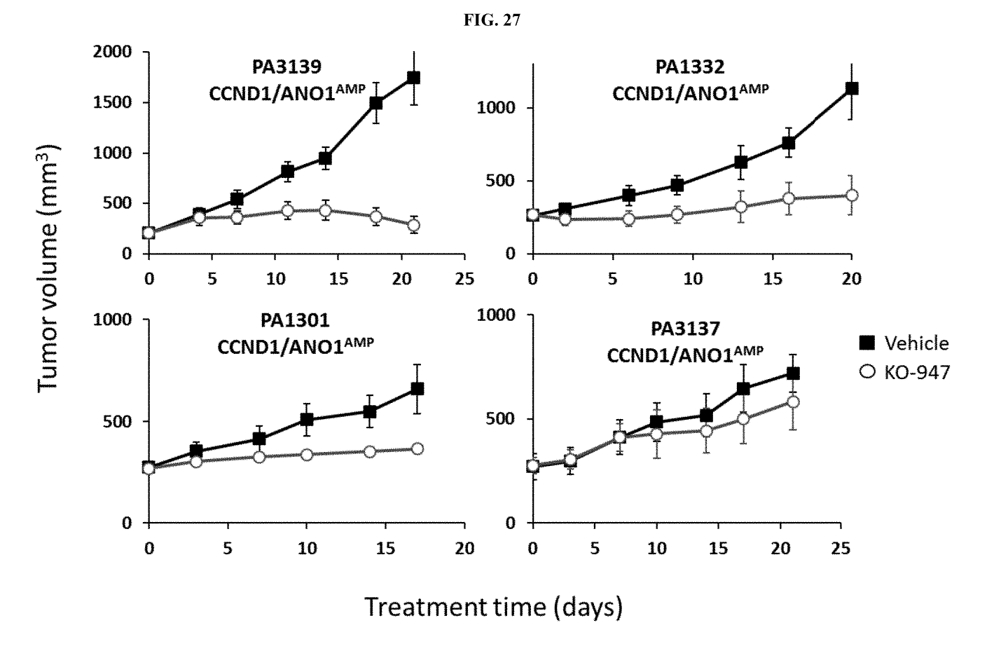

Example 15: Efficacy studies in patient-derived xenograft models of pancreatic cancer. The general procedure outline in Example 1 was followed. Briefly, tumor fragments (2-4 mm in diameter) from stock mice inoculated with primary human pancreatic cancer tissues were inoculated subcutaneously into BALB/C nude mice. The mice were stratified into groups when the average tumor size reached about 180 mm³. Animals were treated with vehicle or 300-350 mg/kg QW PO of an ERK inhibitor (KO-947, a compound of Formula I as described herein). Tumor volumes were measured twice weekly in two dimensions using a caliper, with volume expressed in mm³ (mean+−SEM) using the formula V=0.5(a×b)², where a and b are the long and short diameters of the tumor, respectively. A total of four patient-derived xenograft models were treated in the same manner with either vehicle or the ERK inhibitor as presented in FIG. 27.

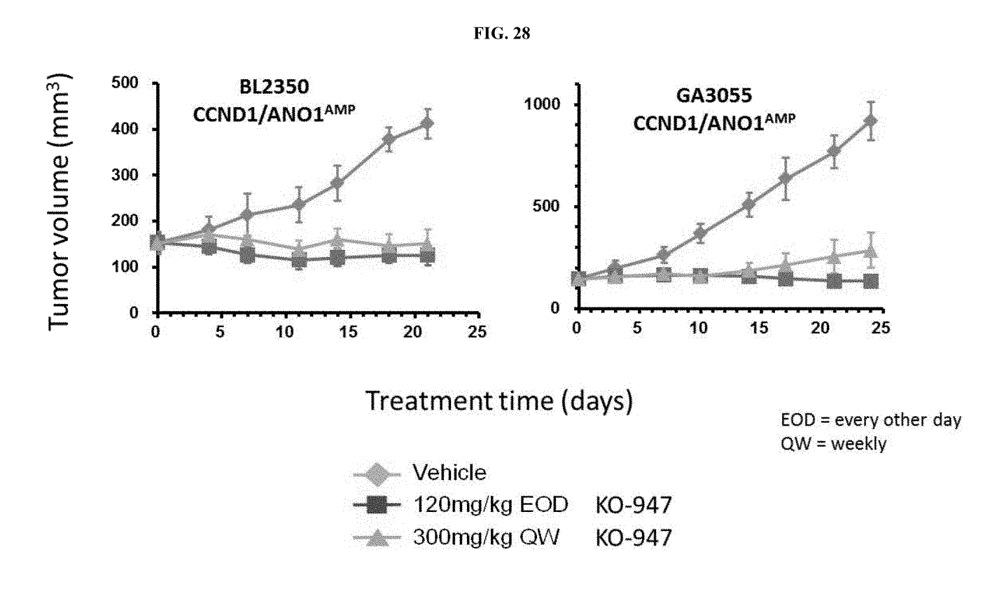

Example 16: Efficacy studies in patient-derived xenograft models of bladder or gastric cancer. The general procedure outline in Example 1 was followed. Briefly, tumor fragments (2-4 mm in diameter) from stock mice inoculated with primary human bladder or gastric cancer tissues were inoculated subcutaneously into BALB/C nude mice. The mice were stratified into groups when the average tumor size reached about 180 mm³. Animals were treated with vehicle, 120 mg/kg EOD of an ERK inhibitor, or 300 mg/kg QW of the ERK inhibitor (KO-947, a compound of Formula I as described herein). Tumor volumes were measured twice weekly in two dimensions using a caliper, with volume expressed in mm³ (mean+−SEM) using the formula V=0.5(a×b)², where a and b are the long and short diameters of the tumor, respectively. Bladder and gastric patient-derived xenograft models were treated in the same manner with either vehicle or the ERK inhibitor as presented in FIG. 28.

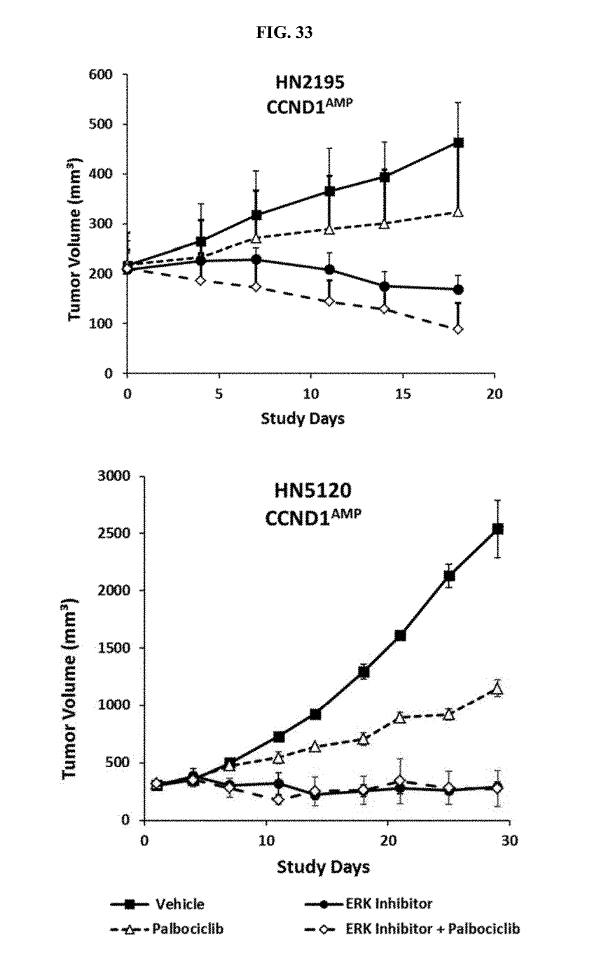

Example 17: Efficacy studies of combination treatments in patient-derived xenograft models of CCND1^{AMP} HNSCC. The general procedure outline in Example 1 was followed. Briefly, tumor fragments (2-4 mm in diameter) from stock mice inoculated with primary human CCND1^{AMP} HNSCC tissues were inoculated subcutaneously into BALB/C nude mice. The mice were stratified into groups when the average tumor size reached about 180 mm³. Animals were treated with vehicle, an ERK inhibitor (KO-947, a compound of Formula I as described herein), palbociclib, or the ERK inhibitor in combination with palbociclib. The ERK inhibitor was administered at a dose of 350 mg/kg QW, with dosing on day 1 of each week. Palbociclib was administered at a dose of 70 mg/kg QD on a 5 on, 2 off schedule, with dosing starting on day 3 of each week. Tumor volumes were measured twice weekly in two dimensions using a caliper, with volume expressed in mm³ (mean+−SEM) using the formula V=0.5(a×b)², where a and b are the long and short diameters of the tumor, respectively. A total of two HNSCC patient-derived xenograft models were treated in the same manner with either vehicle, the ERK inhibitor, palbociclib, or the ERK inhibitor in combination with palbociclib as presented in FIG. 33. The combination of an ERK inhibitor (e.g., KO-947, a compound of Formula I) and a CDK4/6 inhibitor produced a synergistic effect in treating both models.

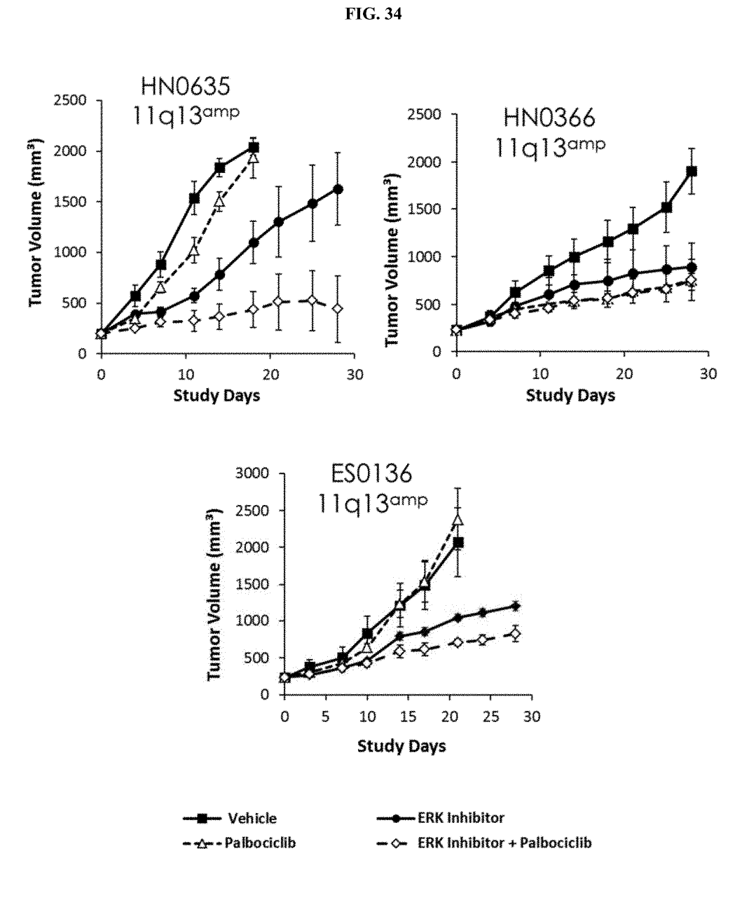

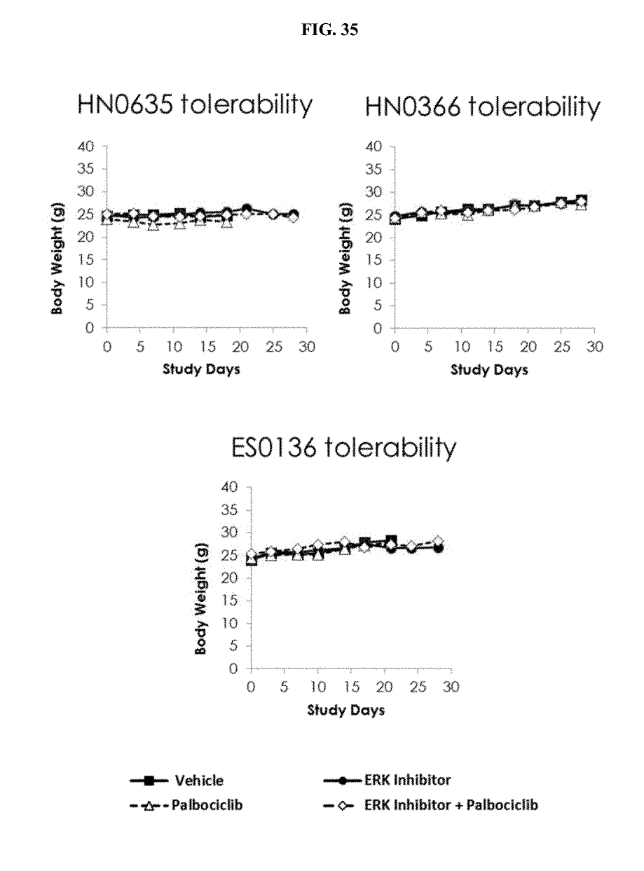

Example 18: Efficacy studies of combination treatments in patient-derived xenograft models of 11q13^{AMP} HNSCC and ESCC. The general procedure outline in Example 1 was followed. Briefly, tumor fragments (2-4 mm in diameter) from stock mice inoculated with primary human 11q13^{AMP} HNSCC or 11q13^{AMP} ESCC tissues were inoculated subcutaneously into BALB/C nude mice. The mice were stratified into groups when the average tumor size reached about 180 mm³. Animals were treated with vehicle, an ERK inhibitor (KO-947, a compound of Formula I as described herein), palbociclib, or the ERK inhibitor in combination with palbociclib. The ERK inhibitor was administered at a dose of 180 mg/kg QW, with dosing on day 1 of each week. Palbociclib was administered at a dose of 70 mg/kg QD on a 5 on, 2 off schedule, with dosing starting on day 3 of each week. Tumor volumes were measured twice weekly in two dimensions using a caliper, with volume expressed in mm³ (mean+−SEM) using the formula V=0.5(a×b)², where a and b are the long and short diameters of the tumor, respectively. An ESCC patient-derived xenograft model and two HNSCC patient-derived xenograft models were treated in the same manner with either vehicle, the ERK inhibitor, palbociclib, or the ERK inhibitor in combination with palbociclib as presented in FIG. 34. The body weights of the treated animals over the study duration are depicted in FIG. 35. The combination of an ERK inhibitor (e.g., KO-947, a compound of Formula I) and a CDK4/6 inhibitor produced a synergistic effect in treating all three models. This effect was more pronounced in this study, which utilized a lower dose of the ERK inhibitor, as compared to Example 17.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the invention. It is intended that the following claims define the scope of the invention and that methods and structures within the scope of these claims and their equivalents be covered thereby.

### FURTHER EMBODIMENTS OF THE INVENTION

1. A method of treating squamous cell carcinoma in a subject in need thereof, comprising administering an effective dose of an inhibitor of an extracellular signal-regulated kinase (ERK) to the subject, said subject comprising a genome that exhibits (1) a first total expression level of at least two mitogen-activated protein kinase (MAPK) pathway genes that is greater than a first reference level, (2) a second total expression level of at least two RAS-ERK feedback regulators that is greater than a second reference level and/or (3) a third total expression level of at least one MAPK pathway gene and at least one RAS-ERK feedback regulator that is greater than a third reference level, wherein the first reference level, the second reference level and the third reference level are each indicative of low sensitivity to the ERK inhibitor.

2. A method of treating a subject having squamous cell carcinoma, comprising:
(a) screening the subject for the presence or absence of a gene signature indicative of sensitivity to an ERK inhibitor; and
(b) administering the ERK inhibitor to the subject if the gene signature is determined to be present.

3. The method of embodiment 2, further comprising applying an alternative therapy to the subject if the gene signature is determined to be absent.

4. The method of embodiment 3, wherein the alternative therapy is selected from the group consisting of chemotherapy, immunotherapy, radiotherapy and surgery.

5. The method of any one of embodiments 2 to 4, wherein the gene signature comprises a first total expression level of at least two MAPK pathway genes that is greater than a first reference level.

6. The method of any one of embodiments 2 to 5, wherein the gene signature comprises a second total expression level of at least two RAS-ERK feedback regulators that is greater than a second reference level.

7. The method of any one of embodiments 2 to 6, wherein the gene signature comprises a third total expression level of at least one MAPK pathway gene and at least one RAS-ERK feedback regulator that is greater than a third reference level.

8. The method of any one of embodiments 2 to 7, wherein the gene signature comprises copy number amplification of at least one MAPK pathway gene.

9. The method of any one of embodiments 2 to 8, wherein the screening comprises performing nucleic acid analysis of a nucleic acid isolated from the subject.

10. The method of embodiment 9, wherein the nucleic acid is from a squamous cell carcinoma cell.

11. A method of downregulating MAPK signaling output in a plurality of squamous cell carcinoma cells with an ERK inhibitor, comprising:
(a) assessing, in a biological sample comprising a nucleic acid from the subject, (1) a first total expression level of at least two MAPK pathway genes, (2) a second total expression level of at least two RAS-ERK feedback regulators and/or (3) a third total expression level of at least one MAPK pathway gene and at least one RAS-ERK feedback regulator; and
(b) administering an effective dose of the ERK inhibitor to the plurality of cells if the first total expression level is greater than a first reference level, the second total expression level is greater than a second reference level and/or the third total expression level is greater than a third reference level, wherein the first reference level, the second reference level and the third reference level are each indicative of low sensitivity to the ERK inhibitor.

12. A method of categorizing a squamous cell carcinoma status of a subject, comprising:
(a) obtaining a biological sample from the subject, the sample comprising genomic and/or transcriptomic material from a squamous cell carcinoma cell of the subject;
(b) assessing (1) a first total expression level of at least two MAPK pathway genes in the sample, (2) a second total expression level of at least two RAS-ERK feedback regulators in the sample, and/or (3) a third total expression level of at least one MAPK pathway gene and at least one RAS-ERK feedback regulator in the sample;
(c) generating an expression profile based on (1) a comparison between the first total expression level and a first reference level, (2) a comparison between the second total expression level and a second reference level, and/or (3) a comparison between the third total expression level and a third reference level, wherein the first reference level, the second reference level and the third reference level are derivable from a reference sample from a different subject having a known squamous cell carcinoma status; and
(d) categorizing the squamous cell carcinoma status of the subject of (a) based on the expression profile.

13. The method of embodiment 12, wherein the squamous cell carcinoma status is categorized as likely sensitive to treatment with an ERK inhibitor if the first total expression level is greater than the first reference level, wherein the first reference level is indicative of low sensitivity to the ERK inhibitor.

14. The method of embodiment 12 or 13, wherein the squamous cell carcinoma status is categorized as likely sensitive to treatment with an ERK inhibitor if the second total expression level is greater than a second reference level, wherein the second reference level is indicative of low sensitivity to the ERK inhibitor.

15. The method of any one of embodiments 12 to 14, wherein the squamous cell carcinoma status is categorized as likely sensitive to treatment with an ERK inhibitor if the third total expression level is greater than a third reference level, wherein the third reference level is indicative of low sensitivity to the ERK inhibitor.

16. The method of any one of embodiments 12 to 15, wherein the known squamous cell carcinoma status of the different subject is categorized as resistant to an ERK inhibitor or sensitive to an ERK inhibitor.

17. The method of embodiment 12, wherein the categorizing step includes calculating, using a computer system, a likelihood of response of the subject to treatment with an ERK inhibitor based on the expression profile, wherein the likelihood is adjusted upward for each fold increase in the first total expression level relative to the first reference level, for each fold increase in the second total expression level relative to the second reference level, and for each fold increase in the third total expression level relative to the third reference level, wherein the first reference level, the second reference level and the third reference level are each indicative of low sensitivity to the ERK inhibitor.

18. The method of embodiment 17, further comprising preparing a report comprising a prediction of the likelihood of response of the subject to treatment with the ERK inhibitor.

19. A method of assessing a likelihood of a subject having squamous cell carcinoma exhibiting a clinically beneficial response to treatment with an ERK inhibitor, the method comprising:
(a) assessing, in a biological sample comprising genomic and/or transcriptomic material from a squamous cell carcinoma cell, (1) a first total expression level of at least two MAPK pathway genes, (2) a second total expression level of at least two RAS-ERK feedback regulators, and/or (3) a third total expression level of at least one MAPK pathway gene and at least one RAS-ERK feedback regulator; and
(b) calculating, using a computer system, a weighted probability of ERK inhibitor responsiveness based on (1) a comparison between the first total expression level and a first reference level, (2) a comparison between the second total expression level and a second reference level, and/or (3) a comparison between the third total expression level and a third reference level, wherein the first reference level, the second reference level and the third reference level are derivable from one or more reference samples.

20. The method of embodiment 19, further comprising designating the subject as having a high probability of exhibiting a clinically beneficial response to treatment with the ERK inhibitor if the weighted probability corresponds to at least 1.5 times a baseline probability, wherein the baseline probability represents a likelihood that the subject will exhibit a clinically beneficial response to treatment with the ERK inhibitor before obtaining the weighted probability of (b).

21. The method of embodiment 20, further comprising transmitting information concerning the likelihood to a receiver.

22. The method of any one of embodiments 19 to 21, further comprising providing a recommendation based on the weighted probability.

23. The method of embodiment 22, wherein the recommendation comprises treating the subject with the ERK inhibitor.

24. The method of embodiment 22, wherein the recommendation comprises discontinuing therapy, chemotherapy, immunotherapy, radiotherapy or surgery.

25. The method of any one of embodiments 19 to 24, further comprising selecting a treatment based on the weighted probability.

26. The method of any one of embodiments 19 to 25, further comprising administering the ERK inhibitor based on the weighted probability.

27. The method of any one of embodiments 1 to 26, wherein the first total expression level, the second total expression level and/or the third total expression level is assessed by detecting a level of mRNA transcribed from: the at least two MAPK pathway genes; the at least two RAS-ERK feedback regulators; and/or the at least one MAPK pathway gene and the at least one RAS-ERK feedback regulator.

28. The method of any one of embodiments 1 to 26, wherein the first total expression level, the second total expression level and/or the third total expression level is assessed by detecting a level of cDNA produced from reverse transcription of mRNA transcribed from: the at least two MAPK pathway genes; the at least two RAS-ERK feedback regulators; and/or the at least one MAPK pathway gene and the at least one RAS-ERK feedback regulator.

29. The method of any one of embodiments 1 to 26, wherein the first total expression level, the second total expression level and/or the third total expression level is assessed by detecting a level of polypeptide encoded by: the at least two MAPK pathway genes; the at least two RAS-ERK feedback regulators; and/or the at least one MAPK pathway gene and the at least one RAS-ERK feedback regulator.

30. The method of embodiment 29, wherein the detecting a level of polypeptide comprises at least one technique selected from the group consisting of immunohistochemistry (IHC), mass spectrometry, Western blotting, enzyme-linked immunosorbent assay (ELISA), immunocytochemistry, immunofluorescence and flow cytometry.

31. The method of any one of embodiments 1 to 26, wherein the first total expression level, the second total expression level and/or the third total expression level is assessed by a nucleic acid amplification assay, a hybridization assay, sequencing, or a combination thereof.

32. The method of embodiment 31, wherein the nucleic acid amplification assay, the hybridization assay, or the sequencing is performed using a nucleic acid sample from the subject.

33. The method of embodiment 32, wherein the nucleic acid sample comprises a nucleic acid selected from the group consisting of genomic DNA, cDNA, ctDNA, cell-free DNA, RNA and mRNA.

34. The method of embodiment 32 or 33, wherein the nucleic acid is from a squamous cell carcinoma cell.

35. The method of any one of embodiments 1 to 26, wherein the first total expression level, the second total expression level and/or the third total expression level is assessed using an nCounter® analysis system.

36. The method of any one of the preceding embodiments, wherein the first reference level, the second reference level and/or the third reference level is obtained by assessing, in a biological sample from a subject having a squamous cell carcinoma exhibiting low sensitivity to treatment with the ERK inhibitor, expression of: the at least two MAPK pathway genes; the at least two RAS-ERK feedback regulators; and/or the at least one MAPK pathway gene and the at least one RAS-ERK feedback regulator.

37. The method of any one of the preceding embodiments, wherein the first reference level represents the average total expression level of the at least two MAPK pathway genes in a plurality of squamous cell carcinoma samples.

38. The method of any one of the preceding embodiments, wherein the second reference level represents the average total expression level of the at least two RAS-ERK feedback regulators in a plurality of squamous cell carcinoma samples.

39. The method of any one of the preceding embodiments, wherein the third reference level represents the average total expression level of the at least one MAPK pathway gene and the at least one RAS-ERK feedback regulator in a plurality of squamous cell carcinoma samples.

40. The method of any one of the preceding embodiments, wherein the at least two MAPK pathway genes consist of four MAPK pathway genes.

41. The method of any one of the preceding embodiments, wherein the at least two MAPK pathway genes consist of six MAPK pathway genes.

42. The method of any one of the preceding embodiments, wherein the at least two MAPK pathway genes consist of eight MAPK pathway genes.

43. The method of any one of the preceding embodiments, wherein the at least two MAPK pathway genes are selected from CDK4, CDK6, EGFR, ERK1, CCND1, KRAS, ERK2, and HRAS.

44. The method of any one of the preceding embodiments, wherein the at least two MAPK pathway genes are selected from EGFR, ERK1, CCND1, KRAS, ERK2, and HRAS.

45. The method of any one of the preceding embodiments, wherein the at least two MAPK pathway genes are selected from EGFR, ERK1, CCND1 and KRAS.

46. The method of any one of the preceding embodiments, wherein the at least two MAPK pathway genes are selected from EGFR, ERK1 and CCND1.

47. The method of any one of the preceding embodiments, wherein the at least two MAPK pathway genes are selected from EGFR, ERK1 and KRAS.

48. The method of any one of the preceding embodiments, wherein the at least two MAPK pathway genes are selected from ERK1 and CCND1.

49. The method of any one of the preceding embodiments, wherein the at least two MAPK pathway genes are selected from ERK1 and EGFR.

50. The method of any one of the preceding embodiments, wherein the at least two MAPK pathway genes are selected from EGFR and CCND1.

51. The method of any one of the preceding embodiments, wherein the at least two RAS-ERK feedback regulators consist of four RAS-ERK feedback regulators.

52. The method of any one of the preceding embodiments, wherein the at least two RAS-ERK feedback regulators consist of five RAS-ERK feedback regulators.

53. The method of any one of the preceding embodiments, wherein the at least two RAS-ERK feedback regulators are selected from DUSP5 , DUSP6, SPRY2 , SPRY4 and SPRED1 .

54. The method of any one of the preceding embodiments, wherein the at least two RAS-ERK feedback regulators are selected from DUSP5 , DUSP6, DUSP2 and DUSP4 .

55. The method of any one of the preceding embodiments, wherein the at least two RAS-ERK feedback regulators are selected from DUSP5 and DUSP6 .

56. The method of any one of the preceding embodiments, wherein the at least one MAPK pathway gene and the at least one RAS-ERK feedback regulator are selected from EGFR, ERK1, CCND1, KRAS, ERK2, HRAS, DUSP5 , DUSP6, DUSP2 , DUSP4 , SPRY2, SPRY4 , SPRED1, and CRAF.

57. The method of any one of the preceding embodiments, wherein the at least one MAPK pathway gene and the at least one RAS-ERK feedback regulator are selected from CCND1, CRAF, DUSP5, EGFR, ERK1 , and KRAS.

58. A method of treating head and neck squamous cell carcinoma in a subject in need thereof, comprising administering an effective dose of an inhibitor of an extracellular signal-regulated kinase (ERK) to the subject, said subject comprising a genome that exhibits (1) a fourth total expression level of AREG, CDH3, COL17A1, EGFR, HIF1A, ITGB1, KRT1, KRT9, NRG1 , SLC16A1, SLC22A1 and VEGFA that is greater than a fourth reference level; (2) a fifth total expression level of DCUN1D1, PIK3CA, PRKCI, SOX2 and TP63 that is less than a fifth reference level; (3) a ratio of the fourth total expression level to the fifth total expression level that is greater than 1; and/or (4) a ratio of HIF1A to TP63 expression levels that is greater than 1, wherein the fourth reference level and the fifth reference level are each indicative of low sensitivity to the ERK inhibitor.

59. A method of treating a subject having head and neck squamous cell carcinoma, comprising:
(a) screening the subject for the presence or absence of a gene signature indicative of sensitivity to an ERK inhibitor; and
(b) administering the ERK inhibitor to the subject if the gene signature is determined to be present.

60. The method of embodiment 59, further comprising applying an alternative therapy to the subject if the gene signature is determined to be absent.

61. The method of embodiment 60, wherein the alternative therapy is selected from the group consisting of chemotherapy, immunotherapy, radiotherapy and surgery.

62. The method of any one of embodiments 59 to 61, wherein the gene signature comprises a fourth total expression level of AREG, CDH3, COL17A1 , EGFR, HIF1A, ITGB1, KRT1, KRT9, NRG1, SLC16A1 , SLC22A1 and VEGFA that is greater than a fourth reference level.

63. The method of any one of embodiments 59 to 62, wherein the gene signature comprises a fifth total expression level of DCUN1D1, PIK3CA, PRKCI, SOX2 and TP63 that is less than a fifth reference level.

64. The method of any one of embodiments 59 to 63, wherein the gene signature comprises a ratio of a fourth total expression level of AREG, CDH3, COL17A1 , EGFR, HIF1A, ITGB1, KRT1, KRT9, NRG1, SLC16A1, SLC22A1 and VEGFA to a fifth total expression level of DCUN1D1, PIK3CA, PRKCI, SOX2 and TP63.

65. The method of any one of embodiments 59 to 64, wherein the gene signature comprises a ratio of HIF1A to TP63 expression levels.

66. The method of any one of embodiments 59 to 64, wherein the gene signature comprises a ratio of HIF1A to TP63 protein levels.

67. The method of any one of embodiments 59 to 65, wherein the screening comprises performing nucleic acid analysis of a nucleic acid isolated from the subject.

68. The method of embodiment 67, wherein the nucleic acid is from a head and neck squamous cell carcinoma cell.

69. A method of downregulating MAPK signaling output in a plurality of head and neck squamous cell carcinoma cells with an ERK inhibitor, comprising:
(a) assessing, in a biological sample comprising a nucleic acid from the subject, (1) a fourth total expression level of AREG, CDH3, COL17A1 , EGFR, HIF1A, ITGB1, KRT1, KRT9, NRG1, SLC16A1 , SLC22A1 and VEGFA; (2) a fifth total expression level of DCUN1D1, PIK3CA, PRKCI, SOX2 and TP63; (3) a ratio of the fourth total expression level to the fifth total expression level; and/or (4) a ratio of HIF1A to TP63 expression levels; and
(b) administering an effective dose of the ERK inhibitor to the plurality of cells if (1) the fourth total expression level is greater than a fourth reference level, (2) the fifth total expression level is less than a fifth reference level, (3) the ratio of the fourth total expression level to the fifth total expression level is greater than 1, and/or (4) the ratio of HIF1A to TP63 is greater than 1, wherein the fourth reference level and the fifth reference level are each indicative of low sensitivity to the ERK inhibitor.

70. A method of categorizing a head and neck squamous cell carcinoma status of a subject, comprising:
(a) obtaining a biological sample from the subject, the sample comprising genomic and/or transcriptomic material from a squamous cell carcinoma cell of the subject;
(b) assessing, in the sample, (1) a fourth total expression level of AREG, CDH3, COL17A1, EGFR, HIF1A, ITGB1, KRT1 , KRT9, NRG1 , SLC16A1 , SLC22A1 and VEGFA; (2) a fifth total expression level of DCUN1D1, PIK3CA, PRKCI, SOX2 and TP63; and/or (3) expression levels of HIF1A and TP63;
(c) generating an expression profile based on (1) a comparison between the fourth total expression level and a fourth reference level, (2) a comparison between the fifth total expression level and a fifth reference level, (3) a comparison between the fourth total expression level to the fifth total expression level, and/or (4) a comparison between expression levels of HIF1A and TP63, wherein the fourth reference level and the fifth reference level are derivable from a reference sample from a different subject having a known squamous cell carcinoma status; and
(d) categorizing the squamous cell carcinoma status of the subject of (a) based on the expression profile.

71. The method of embodiment 70, wherein the squamous cell carcinoma status is categorized as likely sensitive to treatment with an ERK inhibitor if the fourth total expression level is greater than the fourth reference level, wherein the fourth reference level is indicative of low sensitivity to the ERK inhibitor.

72. The method of embodiment 70 or 71, wherein the squamous cell carcinoma status is categorized as likely sensitive to treatment with an ERK inhibitor if the fifth total expression level is less than a fifth reference level, wherein the fifth reference level is indicative of low sensitivity to the ERK inhibitor.

73. The method of any one of embodiments 70 to 72, wherein the squamous cell carcinoma status is categorized as likely sensitive to treatment with an ERK inhibitor if a ratio of the fourth total expression level to the fifth total expression level is greater than 1.

74. The method of any one of embodiments 70 to 73, wherein the squamous cell carcinoma status is categorized as likely sensitive to treatment with an ERK inhibitor if a ratio of HIF1A to TP63 expression levels is greater than 1.

75. The method of embodiment 70, wherein the categorizing step includes calculating, using a computer system, a likelihood of response of the subject to treatment with an ERK inhibitor based on the expression profile, wherein the likelihood is adjusted upward for each fold increase in the fourth total expression level relative to the fourth reference level and downward for each fold increase in the fifth total expression level relative to the fifth reference level, wherein the fourth reference level and the fifth reference level are each indicative of low sensitivity to the ERK inhibitor.

76. The method of embodiment 75, further comprising preparing a report comprising a prediction of the likelihood of response of the subject to treatment with the ERK inhibitor.

77. A method of assessing a likelihood of a subject having head and neck squamous cell carcinoma exhibiting a clinically beneficial response to treatment with an ERK inhibitor, the method comprising:
(a) assessing, in a biological sample comprising genomic and/or transcriptomic material from a squamous cell carcinoma cell, (1) a fourth total expression level of AREG, CDH3 , COL17A1 , EGFR, HIF1A, ITGB1, KRT1, KRT9 , NRG1 , SLC16A1 , SLC22A1 and VEGFA; (2) a fifth total expression level of DCUN1D1, PIK3CA, PRKCI, SOX2 and TP63; and/or (3) expression levels of HIF1A and TP63; and
(b) calculating, using a computer system, a weighted probability of ERK inhibitor responsiveness based on (1) a comparison between the fourth total expression level and a fourth reference level, (2) a comparison between the fifth total expression level and a fifth reference level, (3) a comparison between the fourth total expression level to the fifth total expression level, and/or (4) a comparison between expression levels of HIF1A and TP63, wherein the fourth reference level and the fifth reference level are derivable from one or more reference samples.

78. The method of embodiment 77, further comprising designating the subject as having a high probability of exhibiting a clinically beneficial response to treatment with the ERK inhibitor if the weighted probability corresponds to at least 1.5 times a baseline probability, wherein the baseline probability represents a likelihood that the subject will exhibit a clinically beneficial response to treatment with the ERK inhibitor before obtaining the weighted probability of (b).

79. The method of embodiment 78, further comprising transmitting information concerning the likelihood to a receiver.

80. The method of any one of embodiments 77 to 79, further comprising providing a recommendation based on the weighted probability.

81. The method of embodiment 80, wherein the recommendation comprises treating the subject with the ERK inhibitor.

82. The method of any one of embodiments 77 to 81, further comprising selecting a treatment based on the weighted probability.

83. The method of any one of embodiments 77 to 82, further comprising administering the ERK inhibitor based on the weighted probability.

84. The method of any one of embodiments 58 to 83, wherein the expression levels are assessed by detecting a level of mRNA.

85. The method of any one of embodiments 58 to 83, wherein the expression levels are assessed by detecting a level of cDNA produced from reverse transcription of mRNA.

86. The method of any one of embodiments 58 to 83, wherein the expression levels are assessed by detecting a level of polypeptide.

87. The method of embodiment 86, wherein the detecting a level of polypeptide comprises at least one technique selected from the group consisting of immunohistochemistry (IHC), mass spectrometry, Western blotting, enzyme-linked immunosorbent assay (ELISA), immunocytochemistry, immunofluorescence and flow cytometry.

88. The method of any one of embodiments 58 to 83, wherein the expression levels are assessed by a nucleic acid amplification assay, a hybridization assay, sequencing, or a combination thereof.

89. The method of embodiment 88, wherein the nucleic acid amplification assay, the hybridization assay, or the sequencing is performed using a nucleic acid sample from the subject.

90. The method of embodiment 89, wherein the nucleic acid sample comprises a nucleic acid selected from the group consisting of genomic DNA, cDNA, ctDNA, cell-free DNA, RNA and mRNA.

91. The method of embodiment 89 or 90, wherein the nucleic acid is from a head and neck squamous cell carcinoma cell.

92. The method of any one of embodiments 58 to 83, wherein the expression levels are assessed using an nCounter® analysis system.

93. The method of any one of embodiments 58 to 91, wherein the fourth reference level and/or the fifth reference level is obtained by assessing expression of (1) AREG, CDH3, COL17A1, EGFR, HIF1A, ITGB1, KRT1 , KRT9, NRG1 , SLC16A1 , SLC22A _I and VEGFA; and/or (2) DCUN1D1, PIK3CA, PRKCI, SOX2 and TP63, respectively, in a biological sample from a subject having a squamous cell carcinoma exhibiting low sensitivity to treatment with the ERK inhibitor.

94. The method of any one of embodiments 58 to 93, wherein the fourth reference level represents the average total expression level of AREG, CDH3, COL17A1 , EGFR, HIF1A, ITGB1, KRT1, KRT9, NRG1, SLC16A1, SLC22A1 and VEGFA in a plurality of squamous cell carcinoma samples.

95. The method of any one of embodiments 58 to 94, wherein the fifth reference level represents the average total expression level of DCUN1D1, PIK3CA, PRKCI, SOX2 and TP63 in a plurality of squamous cell carcinoma samples.

96. A method of treating squamous cell carcinoma in a subject in need thereof, comprising administering an effective dose of an inhibitor of an extracellular signal-regulated kinase (ERK) to the subject, said subject comprising a genome having a copy number profile that comprises copy number amplification of at least one mitogen-activated protein kinase (MAPK) pathway gene.

97. A method of downregulating MAPK signaling output in a plurality of squamous cell carcinoma cells with an ERK inhibitor, comprising:
(a) assessing, in a biological sample comprising a nucleic acid from the subject, a copy number profile of at least one MAPK pathway gene; and
(b) administering an effective dose of the ERK inhibitor to the plurality of cells if the copy number profile comprises an average copy number of the at least one MAPK pathway gene of greater than 2.

98. A method of categorizing a squamous cell carcinoma status of a subject, comprising:
(a) obtaining a biological sample from the subject, the sample comprising genomic and/or transcriptomic material from a squamous cell carcinoma cell of the subject;
(b) assessing a copy number profile of at least one MAPK pathway gene in the sample; and
(c) categorizing the squamous cell carcinoma status of the subject based on the copy number profile.

99. The method of embodiment 98, wherein the squamous cell carcinoma status is categorized as likely sensitive to treatment with an ERK inhibitor if the copy number profile comprises an average copy number of the at least one MAPK pathway gene of greater than 2.

100. The method of embodiment 98 or 99, wherein the categorizing step includes calculating, using a computer system, a likelihood of response of the subject to treatment with an ERK inhibitor based on the copy number profile, wherein the likelihood is adjusted upward for each additional copy number of the at least one MAPK pathway gene in excess of 2.

101. The method of embodiment 100, further comprising preparing a report comprising a prediction of the likelihood of response of the subject to treatment with the ERK inhibitor.

102. A method of assessing a likelihood of a subject having squamous cell carcinoma exhibiting a clinically beneficial response to treatment with an ERK inhibitor, the method comprising:
(a) assessing a copy number profile of at least one MAPK pathway gene in a biological sample comprising genomic and/or transcriptomic material from a squamous cell carcinoma cell; and
(b) calculating, using a computer system, a weighted probability of ERK inhibitor responsiveness based on the copy number profile.

103. The method of embodiment 102, further comprising designating the subject as having a high probability of exhibiting a clinically beneficial response to treatment with the ERK inhibitor if the weighted probability corresponds to at least 1.5 times a baseline probability, wherein the baseline probability represents a likelihood that the subject will exhibit a clinically beneficial response to treatment with the ERK inhibitor before obtaining the weighted probability of (b).

104. The method of embodiment 103, further comprising transmitting information concerning the likelihood to a receiver.

105. The method of any one of embodiments 102 to 104, further comprising providing a recommendation based on the weighted probability.

106. The method of embodiment 105, wherein the recommendation comprises treating the subject with the ERK inhibitor.

107. The method of embodiment 105, wherein the recommendation comprises discontinuing therapy, chemotherapy, immunotherapy, radiotherapy or surgery.

108. The method of any one of embodiments 102 to 107, further comprising selecting a treatment based on the weighted probability.

109. The method of any one of embodiments 102 to 108, further comprising administering the ERK inhibitor based on the weighted probability.

110. The method of any one of embodiments 96 to 109, wherein the copy number profile of the at least one MAPK pathway gene is assessed by a method selected from the group consisting of in situ hybridization, Southern blot, immunohistochemistry (IHC), polymerase chain reaction (PCR), quantitative PCR (qPCR), quantitative real-time PCR (qRT-PCR), comparative genomic hybridization, microarray-based comparative genomic hybridization, and ligase chain reaction (LCR).

111. The method of embodiment 110, wherein the copy number profile of the at least one MAPK pathway gene is assessed by a method selected from the group consisting of fluorescent in situ hybridization, chromogenic in situ hybridization, and silver in situ hybridization.

112. The method of embodiment 110 or 111, wherein the copy number profile is assessed using a nucleic acid sample from the subject.

113. The method of embodiment 112, wherein the nucleic acid sample comprises a nucleic acid selected from the group consisting of genomic DNA, cDNA, ctDNA, cell-free DNA, RNA and mRNA.

114. The method of embodiment 112 or 113, wherein the nucleic acid is from a squamous cell carcinoma cell.

115. The method of any one of embodiments 96 to 114, wherein the at least one MAPK pathway gene is selected from CDK4, CDK6, EGFR, ERK1, CCND1 , KRAS, ERK2, and HRAS

116. The method of embodiment 115, wherein the at least one MAPK pathway gene is EGFR.

117. The method of embodiment 116, wherein the squamous cell carcinoma is esophageal squamous cell carcinoma.

118. The method of any one of the preceding embodiments, wherein the biological sample is a tissue sample.

119. The method of embodiment 118, wherein the tissue sample is fixed, paraffin-embedded, fresh or frozen.

120. The method of embodiment 118 or 119, wherein the tissue sample is derived from fine needle, core or other types of biopsy.

121. The method of any one of embodiments 1 to 117, wherein the biological sample is a whole blood or plasma sample.

122. The method of any one of the preceding embodiments, wherein the squamous cell carcinoma is selected from lung, esophagus, cervical and head and neck squamous cell carcinomas.

123. A method of treating cancer in a subject in need thereof, comprising administering an effective dose of an inhibitor of an extracellular signal-regulated kinase (ERK) to the subject, wherein the subject exhibits resistance to a treatment with a Ras, Raf or MEK inhibitor.

124. A method of treating a subject having cancer, comprising:
(a) screening the subject for resistance to a treatment with a Ras, Raf or MEK inhibitor; and
(b) administering an ERK inhibitor to the subject if the subject is determined to be resistant to the treatment with the Ras, Raf or MEK inhibitor.

125. The method of embodiment 123 or 124, wherein the subject exhibits resistance to a treatment with a B-Raf inhibitor.

126. The method of embodiment 125, wherein the B-Raf inhibitor is selected from vemurafenib, GDC-0879, PLX-4720, PLX-3603, PLX-4032, RAF265, XL281, AZ628, sorafenib, dabrafenib and LGX818.

127. The method of embodiment 126, wherein the B-Raf inhibitor is vemurafenib.

128. The method of embodiment 123 or 124, wherein the subject exhibits resistance to a treatment with an MEK inhibitor.

129. The method of embodiment 128, wherein the MEK inhibitor is selected from trametinib, cobimetinib, binimetinib, selumetinib, PD-325901, CI-1040, PD-035901, TAK-733, PD98059, PD184352, U0126, RDEA119, AZD8330, RO4987655, RO4927350, R05068760, AS703026 and E6201.

130. The method of embodiment 129, wherein the MEK inhibitor is trametinib.

131. The method of any one of embodiments 123 to 130, wherein the cancer comprises a B-Raf or N-Ras mutation.

132. The method of any one of embodiments 123 to 131, wherein the cancer is selected from breast cancer, pancreatic cancer, lung cancer, thyroid cancer, seminomas, melanoma, bladder cancer, liver cancer, kidney cancer, myelodysplastic syndrome, acute myelogenous leukemia and colorectal cancer.

133. The method of embodiment 132, wherein the cancer is selected from pancreatic cancer, lung cancer, melanoma and colorectal cancer.

134. The method of embodiment 133, wherein the cancer is melanoma.

135. A method of inhibiting growth of a cancer cell, the method comprising administering to the cell an ERK inhibitor, wherein the cell exhibits resistance to a treatment with a Ras, Raf or MEK inhibitor.

136. The method of embodiment 135, wherein the cell exhibits resistance to a treatment with a B-Raf inhibitor.

137. The method of embodiment 136, wherein the B-Raf inhibitor is selected from vemurafenib, GDC-0879, PLX-4720, PLX-3603, PLX-4032, RAF265, XL281, AZ628, sorafenib, dabrafenib and LGX818.

138. The method of embodiment 137, wherein the B-Raf inhibitor is vemurafenib.

139. The method of embodiment 135, wherein the cell exhibits resistance to a treatment with an MEK inhibitor.

140. The method of embodiment 139, wherein the MEK inhibitor is selected from trametinib, cobimetinib, binimetinib, selumetinib, PD-325901, CI-1040, PD-035901, TAK-733, PD98059, PD184352, U0126, RDEA119, AZD8330, RO4987655, RO4927350, R05068760, AS703026 and E6201.

141. The method of embodiment 140, wherein the MEK inhibitor is trametinib.

142. The method of any one of embodiments 135 to 141, wherein the cell comprises a B-Raf or N-Ras mutation.

143. The method of any one of embodiments 135 to 142, wherein the cell is selected from a pancreatic cancer cell, a lung cancer cell, a melanoma cell and a colorectal cancer cell.

144. The method of embodiment 143, wherein the cell is a melanoma cell.

145. The method of any one of the preceding embodiments, wherein the ERK inhibitor is administered as a monotherapy.

146. The method of any one of embodiments 1 to 144, wherein the ERK inhibitor is administered with at least one other anti-cancer therapy.

147. The method of any one of the preceding embodiments, wherein the ERK inhibitor is a compound of Formula I:

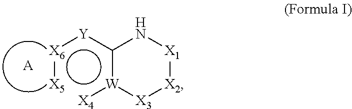

wherein:

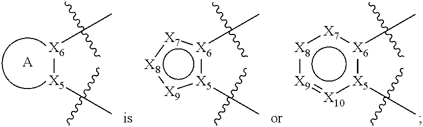

X₁ is C═O, C═S, SO, SO₂, or PO₂⁻; Y is CR₅; W is N or C;

X₂ is NR₁ or CR₁R₁′ and X₃ is null, CR₃R₃′ or C═O; or X₂-X₃ is R₁C═CR₃ or R₁C═N or N═CR₃ or NR₁₂—CR₁₁═CR₃;

X₄ is N or CR₄; X₅ is N or C; X₆ is N or C; X₇ is O, N, NR₇₂ or CR₇₁; X₈ is O, N, NR₈₂ or CR₈₁; X₉ is O, N, NR₂₂ or CR₂₁; X₁₀ is O, N, NR₉₂ or CR₉₁;

R₁ is —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —C₁₋₁₀alkyl-C₃₋₁₀aryl, —C₁₋₁₀arkyl-C₁₋₁₀hetaryl, —C₁₋₁₀alkyl-C₁₋₁₀cycloalkyl, —C₁₋₁₀alkyl-C₁₋₁₀heterocyclyl, —C₂₋₁₀alkenyl-C₃₋₁₀aryl, —C₂₋₁₀alkenyl-C₁₋₁₀hetaryl, —C₂₋₁₀alkenyl-C₃₋₁₀cycloalkyl, —C₂₋₁₀alkenyl-C₁₋₁₀heterocyclyl, —C₂₋₁₀alkynyl-C₃₋₁₀aryl, —C₂₋₁₀alkynyl-C₁₋₁₀hetaryl, —C₂₋₁₀alkynyl-C₃₋₁₀cycloalkyl, —C₂₋₁₀alkynyl-C₁₋₁₀heterocyclyl, —C₁₋₁₀heteroalkyl-C₃₋₁₀aryl, —C₁₋₁₀heteroalkyl-C₁₋₁₀hetaryl, —C₁₋₁₀heteroalkyl-C₃₋₁₀cycloalkyl, —C₁₋₁₀heteroalkyl-C₁₋₁₀heterocyclyl, —C₁₋₁₀alkoxy-C₃₋₁₀aryl, —C₁₋₁₀alkoxy-C₁₋₁₀hetaryl, —C₁₋₁₀alkoxy-C₃₋₁₀cycloalkyl, —C₁₋₁₀alkoxy-C₁₋₁₀heterocyclyl, —C₃₋₁₀aryl-C₁₋₁₀alkyl, —C₃₋₁₀aryl,-C₂₋₁₀alkenyl, —C₃₋₁₀aryl-C₂₋₁₀alkynyl, —C₃₋₁₀aryl-C₃₋₁₀hetaryl, —C₃₋₁₀aryl-C₃₋₁₀cycloalkyl, —C₃₋₁₀aryl-C₁₋₁₀heterocyclyl, —C₁₋₁₀hetaryl-C₁₋₁₀alkyl, —C₁₋₁₀hetaryl-C₂₋₁₀alkenyl, —C₁₋₁₀hetaryl-C₂₋₁₀alkynyl, —C₃₋₁₀hetaryl-C₃₋₁₀aryl, —C₁₋₁₀hetaryl-C₃₋₁₀cycloalkyl, —C₁₋₁₀hetaryl-C₁₋₁₀heterocyclyl, —C₃₋₁₀cycloalkyl-C₁₋₁₀alkyl, —C₃₋₁₀cycloalkyl-C₂₋₁₀alkenyl, —C₃₋₁₀cycloalkyl-C₂₋₁₀alkynyl, —C₃₋₁₀cycloalkyl-C₃₋₁₀aryl, —C₃₋₁₀cycloalkyl-C₁₋₁₀hetaryl, —C₃₋₁₀cycloalkyl-C₁₋₁₀heterocyclyl, —C₁₋₁₀heterocyclyl-C₁₋₁₀alkyl, —C₁₋₁₀heterocyclyl-C₂₋₁₀alkenyl, —C₁₋₁₀heterocyclyl-C₂₋₁₀alkynyl, —C₁₋₁₀heterocyclyl-C₃₋₁₀aryl, —C₁₋₁₀heterocyclyl-C₁₋₁₀hetaryl, or —C₁₋₁₀heterocyclyl-C₃₋₁₀cycloalkyl, each of which is unsubstituted or substituted by one or more independent R₁₀ or R₁₁ substituents;

R₁′ is hydrogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —C₁₋₁₀alkyl-C₁₋₁₀aryl, —C₁₋₁₀alkyl-C₁₋₁₀hetaryl, —C₁₋₁₀alkyl-C₃₋₁₀cycloalkyl, —C₁₋₁₀alkyl-C₁₋₁₀heterocyclyl, —C₂₋₁₀alkenyl-C₃₋₁₀aryl, —C₂₋₁₀alkenyl-C₁₋₁₀hetaryl, —C₂₋₁₀alkenyl-C₃₋₁₀cycloalkyl, —C₂₋₁₀alkenyl-C₁₋₁₀heterocyclyl, —C₂₋₁₀alkynyl-C₃₋₁₀aryl, —C₂₋₁₀alkynyl-C₁₋₁₀hetaryl, —C₂₋₁₀alkynyl-C₃₋₁₀cycloalkyl, —C₂₋₁₀alkynyl-C₁₋₁₀heterocyclyl, —C₁₋₁₀heteroalkyl-C₃₋₁₀aryl, —C₁₋₁₀heteroalkyl-C₁₋₁₀hetaryl, —C₁₋₁₀heteroalkyl-C₃₋₁₀cycloalkyl, —C₁₋₁₀heteroalkyl-C₁₋₁₀heterocyclyl, —C₁₋₁₀alkoxy-C₃₋₁₀aryl, —C₁₋₁₀alkoxy-C₁₋₁₀hetaryl, —C₁₋₁₀alkoxy-C₃₋₁₀cycloalkyl, —C₁₋₁₀alkoxy-C₁₋₁₀heterocyclyl, —C₃₋₁₀aryl-C₁₋₁₀alkyl, —C₃₋₁₀aryl-C₂₋₁₀alkenyl, —C₃₋₁₀aryl-C₂₋₁₀alkynyl, —C₃₋₁₀aryl-C₃₋₁₀hetaryl, —C₃₋₁₀aryl-C₃₋₁₀cycloalkyl, —C₃₋₁₀aryl-C₁₋₁₀heterocyclyl, —C₁₋₁₀hetaryl-C₁₋₁₀alkyl, —C₁₋₁₀hetaryl-C₂₋₁₀alkenyl, —C₁₋₁₀hetaryl-C₂₋₁₀alkynyl, —C₃₋₁₀hetaryl-C₃₋₁₀aryl, —C₁₋₁₀hetaryl-C₃₋₁₀cycloalkyl, —C₁₋₁₀hetaryl-C₁₋₁₀heterocyclyl, —C₃₋₁₀cycloalkyl-C₁₋₁₀alkyl, —C₃₋₁₀cycloalkyl-C₂₋₁₀alkenyl, —C₃₋₁₀cycloalkyl-C₂₋₁₀alkynyl, —C₃₋₁₀cycloalkyl-C₃₋₁₀aryl, —C₃₋₁₀cycloalkyl-C₁₋₁₀hetaryl, —C₃₋₁₀cycloalkyl-C₁₋₁₀heterocyclyl, —C₁₋₁₀heterocyclyl-C₁₋₁₀alkyl, —C₁₋₁₀heterocyclyl-C₂₋₁₀alkenyl, —C₁₋₁₀heterocyclyl-C₂₋₁₀alkynyl, —C₁₋₁₀heterocyclyl-C₃₋₁₀aryl, —C₁₋₁₀heterocyclyl-C₁₋₁₀hetaryl, or —C₁₋₁₀heterocyclyl-C₃₋₁₀cycloalkyl, each of which is unsubstituted or substituted by one or more independent R₁₀ or R₁₁ substituents;

R₂₁ is hydrogen, halogen, —OH, —CF₃, —OCF₃, —OR³¹, —NR³¹R³², —C(O)R³¹, —CO₂R³¹, —C(═O)NR³¹, —NO₂, —CN, —S(O)₀₋₂R₃₁, —SO₂NR³¹R³², —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³²R³³, —NR³¹C(═NR³²)OR³³, —NR³¹C(═NR³²)SR³³, —OC(═O)OR³³, —OC(═O)NR³¹R³², —OC(═O)SR³¹, —SC(═O)SR³¹, —P(O)OR⁻OR³², —SC(═O)NR³¹R³², -L-C₁₋₁₀alkyl , -L-C₂₋₁₀alkenyl, -L-C₂₋₁₀alkynyl, -L-C₁₋₁₀heteroalkyl, -L-C₃₋₁₀aryl, -L-C₁₋₁₀hetaryl, -L-C₃₋₁₀cycloalkyl, -L-C₁₋₁₀heterocyclyl, -L-C₁₋₁₀alkyl-C₃₋₁₀aryl, -L-C₁₋₁₀alkyl-C₁₋₁₀hetaryl, -L-C₁₋₁₀alkyl-C₃₋₁₀cycloalkyl, -L-C₁₋₁₀alkyl-C₁₋₁₀heterocyclyl, -L-C₂₋₁₀alkenyl-C₃₋₁₀aryl, -L-C₂₋₁₀alkenyl-C₁₋₁₀hetaryl, -L-C₂₋₁₀alkenyl-C₃₋₁₀cycloalkyl, -L-C₂₋₁₀alkenyl-C₁₋₁₀heterocyclyl, -L-C₂₋₁₀alkynyl-C₃₋₁₀aryl, -L-C₂₋₁₀alkynyl-C₁₋₁₀hetaryl, -L-C₂₋₁₀alkynyl-C₃₋₁₀cycloalkyl, -L-C₂₋₁₀alkynyl-C₁₋₁₀heterocyclyl, -L-C₁₋₁₀heteroalkyl-C₃₋₁₀aryl, -L-C₁₋₁₀heteroalkyl-C₁₋₁₀hetaryl, -L-C₁₋₁₀heteroalkyl-C₃₋₁₀cycloalkyl, -L-C₁₋₁₀heteroalkyl-C₁₋₁₀heterocyclyl, -L-C₁₋₁₀alkoxy-C₃₋₁₀aryl, -L-C₁₋₁₀alkoxy-C₁₋₁₀hetaryl, -L-C₁₋₁₀alkoxy-C₃₋₁₀cycloalkyl, -L-C₁₋₁₀alkoxy-C₁₋₁₀heterocyclyl, -L-C₃₋₁₀aryl-C₁₋₁₀alkyl, -L-C₃₋₁₀aryl-C₂₋₁₀alkenyl, -L-C₃₋₁₀aryl-C₂₋₁₀alkynyl, -L-C₃₋₁₀aryl-C₁₋₁₀hetaryl, -L-C₃₋₁₀aryl-C₃₋₁₀cycloalkyl, -L-C₃₋₁₀aryl-C₁₋₁₀heterocyclyl, -L-C₁₋₁₀hetaryl-C₁₋₁₀alkyl, -L-C₁₋₁₀hetaryl-C₂₋₁₀alkenyl, -L-C₁₋₁₀hetaryl-C₂₋₁₀alkynyl, -L-C₁₋₁₀hetaryl-C₃₋₁₀aryl, -L-C₁₋₁₀hetaryl-C₃₋₁₀cycloalkyl, -L-C₁₋₁₀hetaryl-C₁₋₁₀heterocyclyl, -L-C₃₋₁₀cycloalkyl-C₁₋₁₀alkyl, -L-C₃₋₁₀cycloalkyl-C₂₋₁₀alkenyl, -L-C₃₋₁₀cycloalkyl-C₂₋₁₀alkynyl, -L-C₃₋₁₀cycloalkyl-C₃₋₁₀aryl, -L-C₃₋₁₀cycloalkyl-C₁₋₁₀hetaryl, -L-C₃₋₁₀cycloalkyl-C₁₋₁₀heterocyclyl, -L-C₁₋₁₀heterocyclyl-C₁₋₁₀alkyl, -L-C₁₋₁₀heterocyclyl-C₂₋₁₀alkenyl, -L-C₁₋₁₀heterocyclyl-C₂₋₁₀alkynyl, -L-C₁₋₁₀heterocyclyl-C₃₋₁₀aryl, -L-C₁₋₁₀heterocyclyl-C₁₋₁₀hetaryl, or -L-C₁₋₁₀heterocyclyl-C₃₋₁₀cycloalkyl, each of which is unsubstituted or substituted by one or more independent R₁₂ substituents;

R₂₂ is hydrogen, —OH, —CF₃, —C(O)R³¹, —CO₂R³¹, —C(═O)NR³¹, —S(O)₀₋₂R³¹, —C(═S)OR³¹, —C(═O)SR³¹, -L-C₁₋₁₀alkyl, -L-C₂₋₁₀alkenyl, -L-C₂₋₁₀alkynyl, -L-C₁₋₁₀heteroalkyl, -L-C₃₋₁₀aryl, -L-C₁₋₁₀hetaryl, -L-C₃₋₁₀cycloalkyl, -L-C₁₋₁₀heterocyclyl, -L-C₁₋₁₀alkyl-C₃₋₁₀aryl, -L-C₁₋₁₀alkyl-C₁₋₁₀hetaryl, -L-C₁₋₁₀alkyl-C₃₋₁₀cycloalkyl, -L-C₁₋₁₀alkyl-C₁₋₁₀heterocyclyl, -L-C₂₋₁₀alkenyl-C₃₋₁₀aryl, -L-C₂₋₁₀alkenyl-C₁₋₁₀hetaryl, -L-C₂₋₁₀alkenyl-C₃₋₁₀cycloalkyl, -L-C₂₋₁₀alkenyl-C₁₋₁₀heterocyclyl, -L-C₂₋₁₀alkynyl-C₃₋₁₀aryl, -L-C₂₋₁₀alkynyl-C₁₋₁₀hetaryl, -L-C₂₋₁₀alkynyl-C₃₋₁₀cycloalkyl, -L-C₂₋₁₀alkynyl-C₁₋₁₀heterocyclyl, -L-C₁₋₁₀heteroalkyl-C₃₋₁₀aryl, -L-C₁₋₁₀heteroalkyl-C₁₋₁₀hetaryl, -L-C₁₋₁₀heteroalkyl-C₃₋₁₀cycloalkyl, -L-C₁₋₁₀heteroalkyl-C₁₋₁₀heterocyclyl, -L-C₁₋₁₀alkoxy-C₃₋₁₀aryl, -L-C_{alkoxy}-C₁₋₁₀hetaryl, -L-C₁₋₁₀alkoxy-C₃₋₁₀cycloalkyl, -L-C₁₋₁₀alkoxy-C₁₋₁₀heterocyclyl, -L-C₃₋₁₀aryl-C₁₋₁₀alkyl, -L-C₃₋₁₀aryl-C₂₋₁₀alkenyl, -L-C₃₋₁₀aryl-C₂₋₁₀alkynyl, -L-C₃₋₁₀aryl-C₁₋₁₀hetaryl, -L-C₃₋₁₀aryl-C₃₋₁₀cycloalkyl, -L-C₃₋₁₀aryl-C₁₋₁₀heterocyclyl, -L-C₁₋₁₀hetaryl-C₁₋₁₀alkyl, -L-C₁₋₁₀hetaryl-C₂₋₁₀alkenyl, -L-C₁₋₁₀hetaryl-C₂₋₁₀alkynyl, -L-C₁₋₁₀hetaryl-C₃₋₁₀aryl, -L-C₁₋₁₀hetaryl-C₃₋₁₀cycloalkyl, -L-C₁₋₁₀hetaryl-C₁₋₁₀heterocyclyl, -L-C₃₋₁₀cycloalkyl-C₁₋₁₀alkyl, -L-C₃₋₁₀cycloalkyl-C₂₋₁₀alkenyl, -L-C₃₋₁₀cycloalkyl-C₂₋₁₀alkynyl, -L-C₃₋₁₀cycloalkyl-C₃₋₁₀aryl, -L-C₃₋₁₀cycloalkyl-C₁₋₁₀hetaryl, -L-C₃₋₁₀cycloalkyl-C₁₋₁₀heterocyclyl, -L-C₁₋₁₀heterocyclyl-C₁₋₁₀alkyl, -L-C₁₋₁₀heterocyclyl-C₂₋₁₀alkenyl, -L-C₁₋₁₀heterocyclyl-C₂₋₁₀alkynyl, -L-C₁₋₁₀heterocyclyl-C₃₋₁₀aryl, -L-C₁₋₁₀heterocyclyl-C₁₋₁₀hetaryl, or -L-C₁₋₁₀heterocyclyl-C₃₋₁₀cycloalkyl, each of which is unsubstituted or substituted by one or more independent R₁₂ substituents;

L is a bond, —O—, —N(R³¹)—, —S(O)₀₋₂—, —C(═O)—, —C(═O)O—, —OC(═O)—, —C(═O)N(R³¹)—, —N(R³¹)C(═O)—, —NR³¹C(═O)O—, —NR³¹C(═O)NR³²—, —NR³¹S(O)₀₋₂—, —S(O)₀₋₂N(R³¹)—, —C(═S)O—, —C(═O)S—, —NR³¹C(═NR³²)NR³²—, —NR³¹C(═NR³²)O—, —NR³¹C(═NR³²)S—, —OC(═O)O—, —OC(═O)NR³¹—, —OC(═O)S—, —SC(═O)S—, —P(O)OR⁻O—, —SC(═O)NR³¹—;

each of R₃, R₃′ and R₄ is independently hydrogen, halogen, —OH, —CF₃, —OCF₃, —OR³¹, —NR³¹R³², —C(O)R³¹, —CO₂R⁻, —C(═O)NR³¹, —NO₂, —CN, —S(O)₀₋₂R³¹, —SO₂NR³¹R³², —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³²R³³, —NR³¹C(═NR³²)OR³³, —NR³¹C(═NR³²)SR³³, —OC(═O)OR³³, —OC(═O)NR³¹R³², —OC(═O)SR³¹, —SC(═O)SR³¹, —P(O)OR⁻OR³², —SC(═O)NR³¹R³², —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —C₁₋₁₀alkyl-C₃₋₁₀aryl, C₁₋₁₀alkyl-C₁₋₁₀hetaryl, —C₁₋₁₀alkyl-C₃₋₁₀cycloalkyl, —C₁₋₁₀alkyl-C₁₋₁₀heterocyclyl, —C₂₋₁₀alkenyl-C₃₋₁₀aryl, —C₂₋₁₀alkenyl-C₁₋₁₀hetaryl, —C₂₋₁₀alkenyl-C₃₋₁₀cycloalkyl, —C₂₋₁₀alkenyl-C₁₋₁₀heterocyclyl, —C₂₋₁₀alkynyl-C₃₋₁₀aryl, —C₂₋₁₀alkynyl-C₁₋₁₀hetaryl, —C₂₋₁₀alkynyl-C₃₋₁₀cycloalkyl, —C₂₋₁₀alkynyl-C₁₋₁₀heterocyclyl, —C₁₋₁₀heteroalkyl-C₃₋₁₀aryl, —C₁₋₁₀heteroalkyl-C₁₋₁₀hetaryl, —C₁₋₁₀heteroalkyl-C₃₋₁₀cycloalkyl, —C₁₋₁₀heteroalkyl-C₁₋₁₀heterocyclyl, —C₁₋₁₀alkoxy-C₃₋₁₀aryl, —C₁₋₁₀alkoxy-C₁₋₁₀hetaryl, —C₁₋₁₀alkoxy-C₃₋₁₀cycloalkyl, —C₁₋₁₀alkoxy-C₁₋₁₀heterocyclyl, —C₃₋₁₀aryl-C₁₋₁₀alkyl, —C₃₋₁₀aryl-C₂₋₁₀alkenyl, —C₃₋₁₀aryl-C₂₋₁₀alkynyl, —C₃₋₁₀aryl-C₃₋₁₀hetaryl, —C₃₋₁₀aryl-C₃₋₁₀cycloalkyl, —C₃₋₁₀aryl-C₁₋₁₀heterocyclyl, —C₁₋₁₀hetaryl-C₁₋₁₀alkyl, —C₁₋₁₀hetaryl-C₂₋₁₀alkenyl, —C₁₋₁₀hetaryl-C₂₋₁₀alkynyl, —C₃₋₁₀hetaryl-C₃₋₁₀aryl, —C₁₋₁₀hetaryl-C₃₋₁₀cycloalkyl, —C₁₋₁₀hetaryl-C₁₋₁₀heterocyclyl, —C₃₋₁₀cycloalkyl-C₁₋₁₀alkyl, —C₃₋₁₀cycloalkyl-C₂₋₁₀alkenyl, —C₃₋₁₀cycloalkyl-C₂₋₁₀alkynyl, —C₃₋₁₀cycloalkyl-C₃₋₁₀aryl, —C₃₋₁₀cycloalkyl-C₁₋₁₀hetaryl, —C₃₋₁₀cycloalkyl-C₁₋₁₀heterocyclyl, —C₁₋₁₀heterocyclyl-C₁₋₁₀alkyl, —C₁₋₁₀heterocyclyl-C₂₋₁₀alkenyl, —C₁₋₁₀heterocyclyl-C₂₋₁₀alkynyl, —C₁₋₁₀heterocyclyl-C₃₋₁₀aryl, —C₁₋₁₀heterocyclyl-C₁₋₁₀hetaryl, or —C₁₋₁₀heterocyclyl-C₃₋₁₀cycloalkyl, each of which is unsubstituted or substituted by one or more independent R₁₃ substituents; or R₃′ is —OR⁶, —NR⁶R³⁴, —S(O)₀₋₂R⁶, —C(═O)R⁶, —C(═O)OR⁶, —OC(═O)R⁶, —C(═O)N(R³⁴)R⁶, or —N(R³⁴)C(═O)R⁶, wherein R⁶ together with R³⁴ can optionally form a heterocyclic ring; or R₃′ is —OR⁶, —NR⁶R³⁴, —S(O)₀₋₂R⁶, —C(═O)R⁶, —C(═O)OR⁶, —OC(═O)R⁶, —C(═O)N(R³⁴)R⁶, or —N(R³⁴)C(═O)R⁶, wherein R⁶ together with R³⁴ can optionally form a heterocyclic ring;

each of R₅, R₇₁, R₈₁ and R₉₁ is independently hydrogen, halogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —OH, —CF₃, —OCF₃, —OR³¹, —NR³¹R³², —C(O)R³¹, —CO₂R³¹, —C(═O)NR³¹, —NO₂, —CN, —S(O)₀₋₂R³¹, —SO₂NR³¹R³², —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³²R³³, —NR³¹C(═NR³²)OR³³, —NR³¹C(═NR³²)SR³³, —OC(═O)OR³³, —OC(═O)NR³¹R³², —OC(═O)SR³¹, —SC(═O)SR³¹, —P(O)OR⁻OR³², or —SC(═O)NR³¹NR³²;

R₆ is hydrogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —C₁₋₁₀alkyl-C₃₋₁₀aryl, —C₁₋₁₀alkyl-C₁₋₁₀hetaryl, —C₁₋₁₀alkyl-C₃₋₁₀cycloalkyl, —C₁₋₁₀alkyl-C₁₋₁₀heterocyclyl, —C₂₋₁₀alkenyl-C₃₋₁₀aryl, —C₂₋₁₀alkenyl-C₁₋₁₀hetaryl, —C₂₋₁₀alkenyl-C₃₋₁₀cycloalkyl, —C₂₋₁₀alkenyl-C₁₋₁₀heterocyclyl, —C₂₋₁₀alkynyl-C₃₋₁₀aryl, —C₂₋₁₀alkynyl -C₁₋₁₀hetaryl, —C₂₋₁₀alkynyl-C₃₋₁₀cycloalkyl, —C₂₋₁₀alkynyl-C₁₋₁₀heterocyolyl, —C₁₋₁₀heteroalkyl-C₃₋₁₀aryl, —C₁₋₁₀heteroalkyl-C₁₋₁₀hetaryl, —C₁₋₁₀heteroalkyl-C₃₋₁₀cycloalkyl, —C₁₋₁₀heteroalkyl-C₁₋₁₀heterocyclyl, —C₁₋₁₀alkoxy-C₃₋₁₀aryl, —C₁₋₁₀alkoxy-C₁₋₁₀hetaryl, —C₁₋₁₀alkoxy-C₃₋₁₀cycloalkyl, —C₁₋₁₀alkoxy-C₁₋₁₀heterocyclyl, —C₃₋₁₀aryl-C₁₋₁₀alkyl, —C₃₋₁₀aryl-C₂₋₁₀alkenyl, —C₃₋₁₀aryl-C₂₋₁₀alkynyl, —C₃₋₁₀aryl-C₃₋₁₀hetaryl, —C₃₋₁₀aryl-C₃₋₁₀cycloalkyl, —C₃₋₁₀aryl-C₁₋₁₀heterocyclyl, —C₁₋₁₀hetaryl-C₁₂₋₁₀alkyl, C₁₋₁₀hetaryl-C₂₋₁₀alkenyl, —C₁₋₁₀hetaryl-C₂₋₁₀alkynyl, —C₃₋₁₀hetaryl-C₃₋₁₀aryl, —C₁₋₁₀hetaryl-C₃₋₁₀cycloalkyl, —C₁₋₁₀hetaryl-C₁₋₁₀heterocyclyl, —C₃₋₁₀cycloalkyl-C₁₋₁₀alkyl, —C₃₋₁₀cycloalkyl-C₂₋₁₀alkenyl, —C₃₋₁₀cycloalkyl-C₂₋₁₀alkynyl, —C₃₋₁₀cycloalkyl-C₃₋₁₀aryl, —C₃₋₁₀cycloalkyl-C₁₋₁₀hetaryl, —C₃₋₁₀cycloalkyl-C₁₋₁₀heterocyclyl, —C₁₋₁₀heterocyclyl-C₁₋₁₀alkyl, —C₁₋₁₀heterocyclyl-C₂₋₁₀alkenyl, —C₁₋₁₀heterocyclyl-C₂₋₁₀alkynyl, —C₁₋₁₀heterocyclyl-C₃₋₁₀aryl, —C₁₋₁₀heterocyclyl-C₁₋₁₀hetaryl, or —C₁₋₁₀heterocyclyl-C₃₋₁₀cycloalkyl, each of which is unsubstituted or substituted by one or more independent R₁₄ or R₁₅ substituents;

each of R₇₂, R₈₂ and R₉₂ is independently hydrogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —OH, —CF₃, —C(O)R³¹, —CO₂R³¹, —C(═O)NR³¹, —S(O)₀₋₂R³¹, —C(═S)OR³¹, —C(═O)SR³¹;

each of R₁₀and R₁₄ is independently —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, optionally substituted by one or more independent R₁₁ substituents;

each of R₁₁, R₁₂, R₁₃ and R₁₅ is independently hydrogen, halogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —OH, —CF₃, —OCF₃, —OR³¹, —NR³¹R³², —C(O)R³¹, —CO₂R³¹, —C(═O)NR³¹, —NO₂, —CN, —S(O)₀₋₂R³¹, —SO₂NR³¹R³², —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³²R³³, —NR³¹C(═NR³²)OR³³, —NR³¹C(═NR³²)SR³³, —OC(═O)OR³³, —OC(═O)NR³¹R³², —OC(═O)SR³¹, —SC(═O)SR³¹, —P(O)OR⁻OR³², or —SC(═O)NR³¹NR³²;

each of R³¹, R³², R³³ and R³⁴ is independently hydrogen, halogen, —C₁₋₁₀alkyl, —C₂₋₁₀alkenyl, —C₂₋₁₀alkynyl, —C₁₋₁₀heteroalkyl, —C₃₋₁₀aryl, —C₃₋₁₀cycloalkyl, —C₁₋₁₀heterocyclyl, or wherein R³¹ together with R³² form a heterocyclic ring;

wherein ring A comprises one or more heteroatoms selected from N, O, or S; and

wherein if X₇ is O or X₂-X₃ is R₁C═CR₃, ring A comprises at least two heteroatoms selected from N, O, or S; and

wherein if X₂-X₃ is R₁C═N, at least one of X₇ or X₉ is not N.

148. The method of embodiment 147, wherein the ERK inhibitor is a compound of Formula I-A:

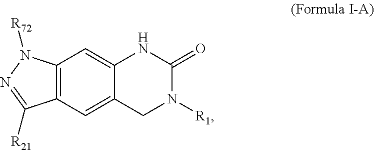

or a pharmaceutically acceptable salt thereof.

149. The method of embodiment 147 or 148, wherein:

R₁ is —C₁₋₁₀alkyl, —C₁₋₁₀alkyl-C₃₋₁₀aryl, or —C₁₋₁₀heterocyclyl-C₁₋₁₀alkyl, each of which is unsubstituted or substituted by one or more independent R₁₀ or R₁₁ substituents;

R₂₁ is -L-C₃₋₁₀aryl or -L-C₁₋₁₀hetaryl, each of which is unsubstituted or substituted by one or more independent R₁₂ substituents;

L is a bond or —N(R³¹)—;

R₇₂ is hydrogen;

each of R₁₀ is independently —C₃₋₁₀aryl, —C₁₋₁₀hetaryl, or —C₁₋₁₀heterocyclyl, optionally substituted by one or more independent R₁₁ substituents;

each of R₁₁ and R₁₂ is independently halogen, —C₁₋₁₀alkyl, —OH, —CF₃ or —OR³¹; and each of R³¹ is independently hydrogen or —C₁₋₁₀alkyl.

150. The method of any one of the preceding embodiments, wherein the ERK inhibitor is selected from the group consisting of:

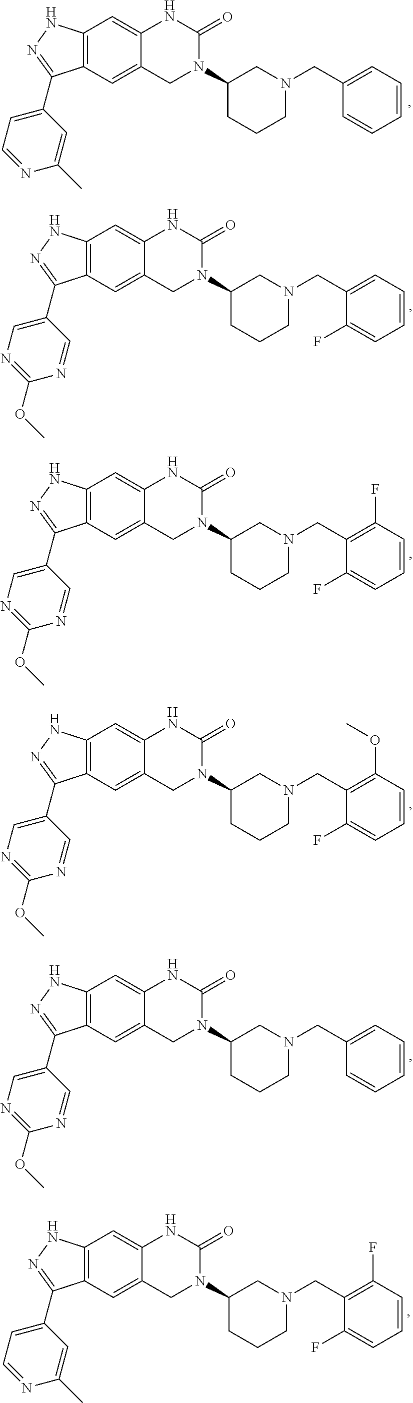

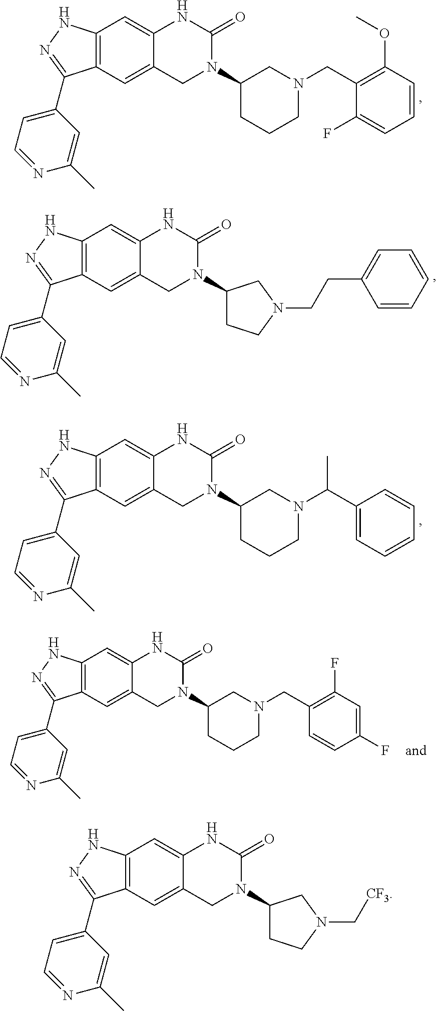

151. The method of any one of embodiments 1 to 146, wherein the ERK inhibitor is selected from the group consisting of ulixertinib, BVD-523, RG7842, GDC-0094, GDC-0994, CC-90003, LTT-462, ASN-007, AMO-01, KO-947, AEZS-134, AEZS-131, AEZS-140, AEZS-136, AEZS-132, D-87503, KIN-2118, RB-1, RB-3, SCH-722984, SCH-772984, MK-8353, SCH-900353, FR-180204, IDN-5491, hyperforin trimethoxybenzoate, ERK1-2067, ERK1-23211, and ERK1-624.

152. The method of any one of embodiments 1 to 146, wherein the ERK inhibitor is selected from the group consisting of:

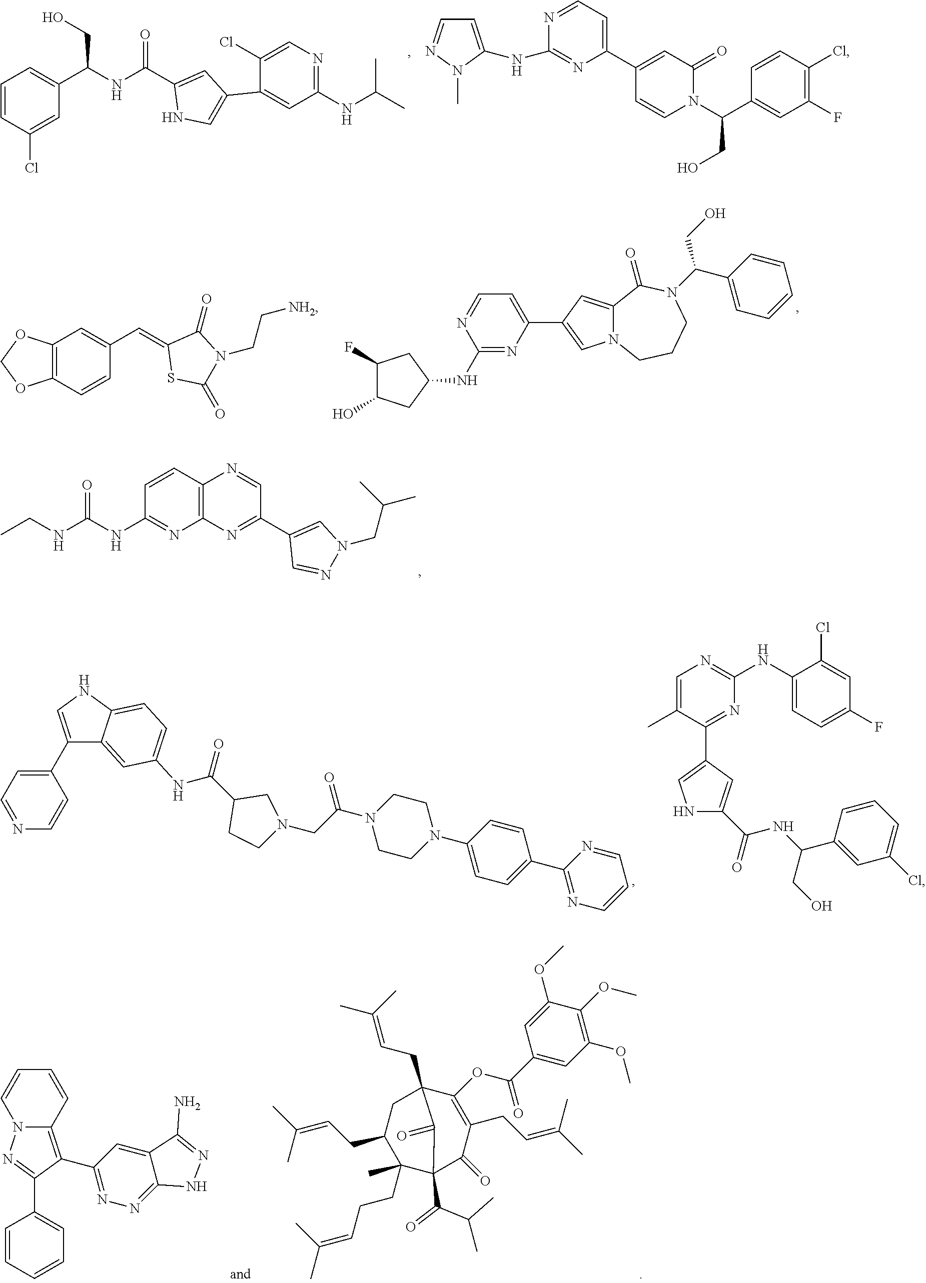

153. The method of any one of the preceding embodiments, further comprising administering a second therapeutic agent to the subject.

154. A method of treating squamous cell carcinoma in a subject in need thereof, comprising administering to said subject an ERK inhibitor and a second therapeutic agent.

155. The method of embodiment 153 or 154, wherein the second therapeutic agent is a chemotherapeutic agent.

156. The method of embodiment 153 or 154, wherein the second therapeutic agent is selected from gemcitabine, cisplatin, an EGFR inhibitor and a CDK inhibitor.

157. The method of embodiment 156, wherein the second therapeutic agent is selected from gemcitabine, cisplatin, palbociclib, osimertinib, olmutinib, icotinib hydrochloride, afatinib, necitumumab, lapatinib, pertuzumab, vandetanib, nimotuzumab, panitumumab, erlotinib, gefitinib and cetuximab.

158. The method of embodiment 156, wherein the second therapeutic agent is selected from gemcitabine, cisplatin, cetuximab, erlotinib and palbociclib.

159. The method of any one of the preceding embodiments, further comprising administering chemotherapy, immunotherapy or radiotherapy to the subject.

160. A system for assessing a likelihood of a subject having squamous cell carcinoma exhibiting a clinically beneficial response to treatment with an ERK inhibitor, the system comprising:

(a) a memory unit configured to store information concerning:
i. a first total expression level of at least two genes selected from the group consisting of EGFR, ERK 1 , CCND1, KRAS, ERK2, and HRAS;
ii. a second total expression level of at least two genes selected from the group consisting of DUSP5 , DUSP6, DUSP2 , DUSP4 , SPRY2 , SPRY4, and SPRED1;
iii. a third total expression level of at least two genes selected from the group consisting of CCND1, CRAF , DUSP5 , EGFR, ERK 1, and KRAS;
iv. a copy number profile of at least one MAPK pathway gene;
v. a fourth total expression level of AREG, CDH3, COL17A1 , EGFR, HIF1A, ITGB1, KRT1, KRT9 , NRG1, SLC16A1, SLC22A1 and VEGFA;
vi. a fifth total expression level of DCUN1D1, PIK3CA, PRKCI, SOX2 and TP63; and/or
vii. expression levels of HIF1A and TP63;
   in a biological sample comprising genomic and/or transcriptomic material from a squamous cell carcinoma cell;

(b) one or more processors alone or in combination programmed to:
(1) determine a weighted probability of ERK inhibitor responsiveness based on the first total expression level, the second total expression level, the copy number profile, the third total expression level, the fourth total expression level, the fifth total expression level, and/or the expression levels of HIF1A and TP63; and
(2) designate the subject as having a high probability of exhibiting a clinically beneficial response to treatment with the ERK inhibitor if the weighted probability corresponds to at least 1.5 times a baseline probability, wherein the baseline probability represents a likelihood that the subject will exhibit a clinically beneficial response to treatment with the ERK inhibitor before obtaining the weighted probability of (b)(1).

161. The system of embodiment 160, wherein the first total expression level, the second total expression level, the third total expression level, the fourth total expression level, the fifth total expression level, and/or the expression levels of HIF1A and TP 63 are assessed by:
(a) detecting a level of mRNA;
(b) detecting a level of cDNA produced from reverse transcription of mRNA;
(c) detecting a level of polypeptide;
(d) detecting a level of cell-free DNA; or
(e) a nucleic acid amplification assay, a hybridization assay, sequencing, or a combination thereof

162. The system of embodiment 160, wherein the copy number profile of the at least one MAPK pathway gene is assessed by a method selected from the group consisting of in situ hybridization, Southern blot, immunohistochemistry (IHC), polymerase chain reaction (PCR), quantitative PCR (qPCR), quantitative real-time PCR (qRT-PCR), comparative genomic hybridization, microarray-based comparative genomic hybridization, and ligase chain reaction (LCR).

163. The system of any one of embodiments 160 to 162, wherein the at least one MAPK pathway gene is selected from EGFR, ERK1 , CCND1 , KRAS, ERK2 and HRAS . 164. The system of embodiment 163, wherein the at least one MAPK pathway gene is EGFR.

165. The system of any one of embodiments 160 to 164, wherein the squamous cell carcinoma is selected from lung, esophagus, cervical and head and neck squamous cell carcinomas.

166. The system of embodiment 165, wherein the squamous cell carcinoma is head and neck squamous cell carcinoma.

167. A method of treating cancer in a subject in need thereof, comprising administering an effective dose of an inhibitor of an extracellular signal-regulated kinase (ERK) to the subject, said subject comprising a genome that exhibits amplification and/or overexpression of at least one gene located at chromosome 11q13.3-13.4.

168. The method of embodiment 167, comprising:
(a) screening the subject for amplification and/or overexpression of the at least one gene located at chromosome 11q13.3-13.4; and
(b) administering the ERK inhibitor to the subject if the amplification and/or overexpression is determined to be present.

169. A method of treating a subject having cancer, comprising:
(a) screening the subject for amplification and/or overexpression of at least one gene located at chromosome 11q13.3-13.4 or a gene that co-amplifies with a gene located at chromosome 11q13.3-13.4; and
(b) administering an ERK inhibitor to the subject if the amplification and/or overexpression is determined to be present.

170. The method of embodiment 168 or 169, further comprising applying an alternative therapy to the subject if the amplification and/or overexpression is absent.

171. The method of any one of embodiments 167 to 170, wherein the screening comprises performing nucleic acid analysis of a nucleic acid isolated from the subject.

172. The method of embodiment 171, wherein the nucleic acid is from a cancer cell.

173. The method of embodiment 168 or 169, comprising administering the ERK inhibitor to the subject if both amplification and overexpression of the at least one gene are determined to be present.

174. The method of any one of embodiments 167 to 172, comprising administering the ERK inhibitor to the subject if the subject exhibits amplification and/or overexpression of CCND1 or ANO1.

175. The method of any one of embodiments 167 to 172, comprising administering the ERK inhibitor to the subject if the subject exhibits amplification or overexpression of CCND1 and ANO1.

176. The method of any one of embodiments 167 to 172, comprising administering the ERK inhibitor to the subject if the subject exhibits amplification and overexpression of CCND1 and ANO1.

177. A method of downregulating MAPK signaling output in a plurality of cancer cells with an ERK inhibitor, comprising:
(a) assessing, in a biological sample comprising a nucleic acid from the plurality of cells, a copy number profile and/or expression profile of at least one gene located at chromosome 11q13.3-13.4; and
(b) administering an effective dose of the ERK inhibitor to the plurality of cells if the copy number profile comprises an average copy number of the at least one gene of >2 and/or if the expression profile is greater than a reference level, wherein the reference level is indicative of low sensitivity to the ERK inhibitor.

178. A method of categorizing a cancer status of a subject, comprising:
(a) obtaining a biological sample from the subject, the sample comprising genomic and/or transcriptomic material from a cancer cell of the subject;
(b) assessing a copy number profile and/or expression profile of at least one gene located at chromosome 11q13.3-13.4 in the sample; and
(c) categorizing the cancer status of the subject of (a) based on the copy number profile and/or the expression profile.

179. The method of embodiment 178, wherein the cancer status is categorized as likely sensitive to treatment with an ERK inhibitor if the copy number profile comprises an average copy number of the at least one gene of >2.

180. The method of embodiment 178 or 179, wherein the cancer status is categorized as likely sensitive to treatment with an ERK inhibitor if the expression profile is greater than a reference level, wherein the reference level is indicative of low sensitivity to the ERK inhibitor.

181. The method of any one of embodiments 178 to 180, wherein the categorizing step includes calculating, using a computer system, a likelihood of response of the subject to treatment with an ERK inhibitor based on the copy number profile and/or the expression profile, wherein the likelihood is adjusted upward for each additional copy number of the at least one gene in excess of 2 and for each fold increase in the expression profile relative to a reference level, wherein the reference level is indicative of low sensitivity to the ERK inhibitor.

182. The method of embodiment 181, further comprising preparing a report comprising a prediction of the likelihood of response of the subject to treatment with the ERK inhibitor.

183. A method of assessing a likelihood of a subject having cancer exhibiting a clinically beneficial response to treatment with an ERK inhibitor, the method comprising:
(a) assessing a copy number profile and/or expression profile of at least one gene located at chromosome 11q13.3-13.4 in a biological sample comprising genomic and/or transcriptomic material from a cancer cell; and
(b) calculating, using a computer system, a weighted probability of ERK inhibitor responsiveness based on the copy number profile and/or the expression profile.

184. The method of embodiment 183, further comprising designating the subject as having a high probability of exhibiting a clinically beneficial response to treatment with the ERK inhibitor if the weighted probability corresponds to at least 1.5 times a baseline probability, wherein the baseline probability represents a likelihood that the subject will exhibit a clinically beneficial response to treatment with the ERK inhibitor before obtaining the weighted probability of (b).

185. The method of embodiment 184, further comprising transmitting information concerning the likelihood to a receiver.

186. The method of any one of embodiments 183 to 185, further comprising providing a recommendation based on the weighted probability.

187. The method of embodiment 186, wherein the recommendation comprises treating the subject with the ERK inhibitor.

188. The method of embodiment 186, wherein the recommendation comprises discontinuing therapy, chemotherapy, immunotherapy, radiotherapy or surgery.

189. The method of any one of embodiments 183 to 188, further comprising selecting a treatment based on the weighted probability.

190. The method of any one of embodiments 183 to 189, further comprising administering the ERK inhibitor based on the weighted probability.

191. The method of any one of embodiments 167 to 190, wherein the expression is assessed by detecting a level of mRNA transcribed from the at least one gene.

192. The method of any one of embodiments 167 to 190, wherein the expression is assessed by detecting a level of cDNA produced from reverse transcription of mRNA transcribed from the at least one gene.

193. The method of any one of embodiments 167 to 190, wherein the expression is assessed by detecting a level of polypeptide encoded by the at least one gene.

194. The method of embodiment 193, wherein the detecting a level of polypeptide comprises at least one technique selected from the group consisting of immunohistochemistry mass spectrometry, Western blotting, enzyme-linked immunosorbent assay (ELISA), immunocytochemistry, immunofluorescence and flow cytometry.

195. The method of any one of embodiments 167 to 190, wherein the expression is assessed by a nucleic acid amplification assay, a hybridization assay, sequencing, or a combination thereof.

196. The method of embodiment 195, wherein the nucleic acid amplification assay, the hybridization assay, or the sequencing is performed using a nucleic acid sample from the subject.

197. The method of embodiment 196, wherein the nucleic acid sample comprises a nucleic acid selected from the group consisting of genomic DNA, cDNA, ctDNA, cell-free DNA, RNA and mRNA.

198. The method of embodiment 196 or 197, wherein the nucleic acid is from a cancer cell.

199. The method of any one of embodiments 167 to 190, wherein the expression is assessed using an nCounter® analysis system.

200. The method of any one of embodiments 167 to 199, wherein the reference level is obtained by assessing, in a biological sample from a subject having a cancer exhibiting low sensitivity to treatment with the ERK inhibitor, expression of the at least one gene.

201. The method of any one of embodiments 167 to 199, wherein the reference level represents the average total expression level of the at least one gene in a plurality of cancer samples.

202. The method of any one of embodiments 167 to 201, wherein the copy number profile of the at least one gene is assessed by a method selected from the group consisting of in situ hybridization, Southern blot, immunohistochemistry (IHC), polymerase chain reaction (PCR), quantitative PCR (qPCR), quantitative real-time PCR (qRT-PCR), comparative genomic hybridization, microarray-based comparative genomic hybridization, and ligase chain reaction (LCR).

203. The method of embodiment 202, wherein the copy number profile of the at least one gene is assessed by a method selected from the group consisting of fluorescent in situ hybridization, chromogenic in situ hybridization, and silver in situ hybridization.

204. The method of embodiment 202 or 203, wherein the copy number profile is assessed using a nucleic acid sample from the subject.

205. The method of embodiment 204, wherein the nucleic acid sample comprises a nucleic acid selected from the group consisting of genomic DNA, cDNA, ctDNA, cell-free DNA, RNA and mRNA.

206. The method of embodiment 204 or 205, wherein the nucleic acid is from a cancer cell.

207. The method of any one of embodiments 167 to 206, wherein the at least one gene is selected from CCND1, CTTN, FADD, ORAOV1, ANO1, PPFIA1 and SHANK2 .

208. The method of embodiment 207, wherein the at least one gene is CCND1 or ANO1 .

209. The method of embodiment 207, wherein the at least one gene is CCND1 and ANO1 .

210. The method of any one of embodiments 167 to 209, wherein the biological sample is a tissue sample.

211. The method of embodiment 210, wherein the tissue sample is fixed, paraffin-embedded, fresh or frozen.

212. The method of embodiment 210 or 211, wherein the tissue sample is derived from fine needle, core or other types of biopsy.

213. The method of any one of embodiments 167 to 209, wherein the biological sample is a whole blood or plasma sample.

214. The method of any one of embodiments 167 to 213, wherein the cancer is selected from the group consisting of squamous cell carcinoma and adenocarcinoma.

215. The method of any one of embodiments 167 to 213, wherein the cancer is a squamous cell carcinoma selected from the group consisting of lung, esophageal, cervical, head and neck, bladder and gastric squamous cell carcinomas.

216. The method of embodiment 215, wherein the squamous cell carcinoma is esophageal squamous cell carcinoma.

217. The method of any one of embodiments 167 to 213, wherein the cancer is an adenocarcinoma selected from the group consisting of esophageal and pancreatic adenocarcinomas.

218. The method of any one of embodiments 167 to 213, wherein the cancer is selected from the group consisting of lung, esophageal, cervical, head and neck, bladder, gastric and pancreatic cancer.

219. The method of any one of embodiments 167 to 213, wherein the cancer is selected from breast cancer, pancreatic cancer, lung cancer, thyroid cancer, seminomas, melanoma, bladder cancer, liver cancer, kidney cancer, myelodysplastic syndrome, acute myelogenous leukemia and colorectal cancer.

220. The method of any one of embodiments 167 to 219, wherein the ERK inhibitor is administered as a monotherapy.

221. The method of any one of embodiments 167 to 219, wherein the ERK inhibitor is administered with at least one other anti-cancer therapy.

222. The method of any one of embodiments 167 to 221, wherein the ERK inhibitor is a compound of Formula I:

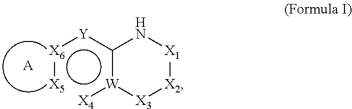

wherein:

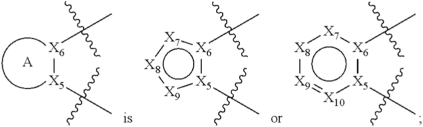

X₁ is C═O, C═S, SO, SO₂, or PO₂⁻; Y is CR₅; W is N or C;

X₂ is NR₁ or CR₁R₁′ and X₃ is null, CR₃R₃′ or C═O; or X₂-X₃ is R₁C═CR₃ or R₁C═N or N═CR₃ or NR₁₂-CR₁₁═CR₃;

X₄ is N or CR₄; X₅ is N or C; X₆ is N or C; X₇ is O, N, NR₇₂ or CR₇₁; X₈ is O, N, NR₈₂ or CR₈₁; X₉ is O, N, NR₂₂ or CR₂₁; X₁₀ is O, N, NR₉₂ or CR₉₁;

R₁ is —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —C₁₋₁₀alkyl-C₃₋₁₀aryl, —C₁₋₁₀alkyl-C₁₋₁₀hetary;, —C₁₋₁₀alkyl-C₃₋₁₀cycloalkyl, —C₁₋₁₀alkyl-C₁₋₁₀heterocyclyl, —C₂₋₁₀alkenyl-C₃₋₁₀aryl, —C₂₋₁₀alkenyl-C₁₋₁₀hetaryl, —C₂₋₁₀alkenyl-C₃₋₁₀cycloalkyl, —C₂₋₁₀alkenyl-C₁₋₁₀heterocyclyl, —C₂₋₁₀alkynyl-C₃₋₁₀aryl, —C₂₋₁₀alkynyl-C₁₋₁₀hetaryl, —C₂₋₁₀alkynyl-C₃₋₁₀cycloalkyl, —C₂₋₁₀alkynyl-C₁₋₁₀heterocyclyl, —C₁₋₁₀heteroalkyl-C₃₋₁₀aryl, —C₁₋₁₀heteroalkyl-C₁₋₁₀hetaryl, —C₁₋₁₀heteroalkyl-C₃₋₁₀cycloalkyl, —C₁₋₁₀heteroalkyl-C₁₋₁₀heterocyclyl, —C₁₋₁₀alkoxy-C₃₋₁₀aryl, —C₁₋₁₀alkoxy-C₁₋₁₀hetaryl, —C₁₋₁₀alkoxy-C₃₋₁₀cycloalkyl, —C₁₋₁₀alkoxy-C₁₋₁₀heterocyclyl, —C₃₋₁₀aryl-C₁₋₁₀alkyl, —C₃₋₁₀aryl-C₂₋₁₀alkenyl, —C₃₋₁₀aryl-C₂₋₁₀alkynyl, —C₃₋₁₀aryl-C₃₋₁₀hetaryl, —C₃₋₁₀aryl-C₃₋₁₀cycloalkyl, —C₃₋₁₀aryl-C₁₋₁₀heterocyclyl, —C₁₋₁₀hetaryl-C₁₋₁₀alkyl, —C₁₋₁₀hetaryl-C₂₋₁₀alkenyl, —C₁₋₁₀hetaryl-C₂₋₁₀alkynyl, —C₃₋₁₀hetaryl-C₃₋₁₀aryl, —C₁₋₁₀hetaryl-C₃₋₁₀cycloalkyl, —C₁₋₁₀hetaryl-C₁₋₁₀heterocyclyl, —C₃₋₁₀cycloalkyl-C₁₋₁₀alkyl, —C₃₋₁₀cycloalkyl-C₂₋₁₀alkenyl, —C₃₋₁₀cycloalkyl-C₂₋₁₀alkynyl, —C₃₋₁₀cycloalkyl-C₃₋₁₀aryl, —C₃₋₁₀cycloalkyl-C₁₋₁₀hetaryl, —C₃₋₁₀cycloalkyl-C₁₋₁₀heterocyclyl, —C₁₋₁₀heterocyclyl-C₁₋₁₀alkyl, —C₁₋₁₀heterocyclyl-C₂₋₁₀alkenyl, —C₁₋₁₀heterocyclyl-C₂₋₁₀alkynyl, —C₁₋₁₀heterocyclyl-C₃₋₁₀aryl, —C₁₋₁₀heterocyclyl-C₁₋₁₀hetaryl, or —C₁₋₁₀heterocyclyl-C₃₋₁₀cycloalkyl, each of which is unsubstituted or substituted by one or more independent R₁₀ or R₁₁ sub stituents;

R₁′ is hydrogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —C₁₋₁₀alkyl-C₃₋₁₀aryl, —C₁₋₁₀alkyl-C₁₋₁₀hetaryl, —C₁₋₁₀alkyl-C₃₋₁₀cycloalkyl, —C₁₋₁₀alkyl-C₁₋₁₀heterocyclyl, —C₂₋₁₀alkenyl-C₃₋ₘaryl, —C₂₋₁₀alkenyl-C₁₋₁₀hetaryl, —C₂₋₁₀alkenyl-C₃₋₁₀cycloalkyl, —C₂₋₁₀alkenyl-C₁₋₁₀heterocyclyl, —C₂₋₁₀alkynyl-C₃₋₁₀aryl, —C₂₋₁₀alkynyl-C₁₋₁₀hetaryl, —C₂₋₁₀alkynyl-C₃₋₁₀cycloalkyl, —C₂₋₁₀alkynyl-C₁₋₁₀heterocyclyl, —C₁₋₁₀heteroalkyl-C₃₋₁₀aryl, —C₁₋₁₀heteroalkyl-C₁₋₁₀hetaryl, —C₁₋₁₀heteroalkyl-C₃₋₁₀cycloalkyl, —C₁₋₁₀heteroalkyl-C₁₋₁₀heterocyclyl, —C₁₋₁₀alkoxy-C₃₋₁₀aryl, —C₁₋₁₀alkoxy-C₁₋₁₀hetaryl, —C₁₋₁₀alkoxy-C₃₋₁₀cycloalkyl, —C₁₋₁₀alkoxy-C₁₋₁₀heterocyclyl, —C₃₋₁₀aryl-C₁₋₁₀alkyl, —C₃₋₁₀aryl-C₂₋₁₀alkenyl, —C₃₋₁₀aryl-C₂₋₁₀alkynyl, —C₃₋₁₀aryl-C₃₋₁₀hetaryl, —C₃₋₁₀aryl-C₃₋₁₀cycloalkyl, —C₃₋₁₀aryl-C₁₋₁₀heterocyclyl, —C₁₋₁₀hetaryl-C₁₋₁₀alkyl, —C₁₋₁₀hetaryl-C₂₋₁₀alkenyl, —C₁₋₁₀hetaryl-C₂₋₁₀alkynyl, —C₃₋₁₀hetaryl-C₃₋₁₀aryl, —C₁₋₁₀hetaryl-C₃₋₁₀cycloalkyl, —C₁₋₁₀hetaryl-C₁₋₁₀heterocyclyl, —C₃₋₁₀cycloalkyl-C₁₋₁₀alkyl, —C₃₋₁₀cycloalkyl-C₂₋₁₀alkenyl, —C₃₋₁₀cycloalkyl-C₂₋₁₀alkynyl, —C₃₋₁₀cycloalkyl-C₃₋₁₀aryl, —C₃₋₁₀cycloalkyl-C₁₋₁₀hetaryl, —C₃₋₁₀cycloalkyl-C₁₋₁₀heterocyclyl, —C₁₋₁₀heterocyclyl-C₁₋₁₀alkyl, —C₁₋₁₀heterocyclyl-C₂₋₁₀alkenyl, —C₁₋₁₀heterocyclyl-C₂₋₁₀alkynyl, —C₁₋₁₀heterocyclyl-C₃₋₁₀aryl, —C₁₋₁₀heterocyclyl-C₁₋₁₀hetaryl, or —C₁₋₁₀heterocyclyl-C₃₋₁₀cycloalkyl, each of which is unsubstituted or substituted by one or more independent R₁₀ or R₁₁ sub stituents;

R₂₁ is hydrogen, halogen, —OH, —CF₃, —OCF₃, —OR³¹, —NR³¹R³², —C(O)R³¹, —CO₂R⁻, —C(═O)NR³¹, —NO₂, —CN, —S(O)₀₋₂R³¹, —SO₂NR³¹R³², —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³²R³³, —NR³¹C(═NR³²)OR³³, —NR³¹C(═NR³²)SR³³, —OC(═O)OR³³, —OC(═O)NR³¹R³², —OC(═O)SR³¹, —SC(═O)SR³¹, —P(O)OR⁻OR³², —SC(═O)NR³¹R³², -L-C₁₋₁₀alkyl, -L-C₂₋₁₀alkenyl, -L-C₂₋₁₀alkynyl, -L-C₁₋₁₀heteroalkyl, -L-C₃₋₁₀aryl, -L-C₁₋₁₀hetaryl, -L-C₃₋₁₀cycloalkyl, C₁₋₁₀heterocyclyl, -L-C₁₋₁₀alkyl-C₁₋₁₀aryl, -L-C₁₋₁₀alkyl,-C₁₋₁₀hetaryl, -L-C₁₋₁₀alkyl-C₃₋₁₀cycloalkyl, -L-C₁₋₁₀alkyl-C₁₋₁₀heterocyclyl, -L-C₂₋₁₀alkenyl-C₃₋₁₀aryl, -L-C₂₋₁₀alkenyl-C₁₋₁₀hetaryl, -L-C₂₋₁₀alkenyl-C₃₋₁₀cycloalkyl, -L-C₂₋₁₀alkenyl-C₁₋₁₀heterocyclyl, -L-C₂₋₁₀alkynyl-C₃₋₁₀aryl, -L-C₂₋₁₀alkynyl-C₁₋₁₀hetaryl, -L-C₂₋₁₀alkynyl-C₃₋₁₀cycloalkyl, -L-C₂₋₁₀alkynyl-C₁₋₁₀heterocyclyl, -L-C₁₋₁₀heteroalkyl-C₃₋₁₀aryl, -L-C₁₋₁₀heteroalkyl-C₁₋₁₀hetaryl, -L-C₁₋₁₀heteroalkyl-C₃₋₁₀cycloalkyl, -L-C₁₋₁₀heteroalkyl-C₁₋₁₀heterocyclyl, -L-C₁₋₁₀alkoxy-C₃₋₁₀aryl, -L-C₁₋₁₀alkoxy-C₁₋₁₀hetaryl, -L-C₁₋₁₀alkoxy-C₃₋₁₀cycloalkyl, -L-C₁₋₁₀alkoxy-C₁₋₁₀heterocyclyl, -L-C₃₋₁₀aryl-C₁₋₁₀alkyl, -L-C₃₋₁₀aryl-C₂₋₁₀alkenyl, -L-C₃₋₁₀aryl-C₂₋₁₀alkynyl, -L-C₃₋₁₀aryl-C₁₋₁₀hetaryl, -L-C₃₋₁₀aryl-C₃₋₁₀cycloalkyl, -L-C₃₋₁₀aryl-C₁₋₁₀heterocyclyl, -L-C₁₋₁₀hetaryl-C₁₋₁₀alkyl, -L-C₁₋₁₀hetaryl-C₂₋₁₀alkenyl, -L-C₁₋₁₀hetaryl-C₂₋₁₀alkynyl, -L-C₁₋₁₀hetaryl-C₃₋₁₀aryl, -L-C₁₋₁₀hetaryl-C₃₋₁₀cycloalkyl, -L-C₁₋₁₀hetaryl-C₁₋₁₀heterocyclyl, -L-C₃₋₁₀cycloalkyl-C₁₋₁₀alkyl, -L-C₃₋₁₀cycloalkyl-C₂₋₁₀alkenyl, -L-C₃₋₁₀cycloalkyl-C₂₋₁₀alkynyl, -L-C₃₋₁₀cycloalkyl-C₃₋₁₀aryl, -L-C₃₋₁₀cycloalkyl-C₁₋₁₀hetaryl, -L-C₃₋₁₀cycloalkyl-C₁₋₁₀heterocyclyl, -L-C₁₋₁₀heterocyclyl-C₁₋₁₀alkyl, -L-C₁₋₁₀heterocyclyl-C₂₋₁₀alkenyl, -L-C₁₋₁₀heterocyclyl-C₂₋₁₀alkynyl, -L-C₁₋₁₀heterocyclyl-C₃₋₁₀aryl, -L-C₁₋₁₀heterocyclyl-C₁₋₁₀hetaryl, or -L-C₁₋₁₀heterocyclyl-C₃₋₁₀cycloalkyl, each of which is unsubstituted or substituted by one or more independent R₁₂ substituents;

R₂₂ is hydrogen, —OH, —CF₃, —C(O)R³¹, —CO₂R³¹, —C(═O)NR³¹, —S(O)₀₋₂R³¹, —C(═S)OR³¹, —C(═O)SR³¹, -L-C₁₋₁₀alkyl, -L-C₂₋₁₀alkenyl, -L-C₂₋₁₀alkynyl, -L-C₁₋₁₀heteroalkyl, -L-C₃₋₁₀aryl, -L-C₁₋₁₀hetaryl, -L-C₃₋₁₀cycloalkyl, -L-C₁₋₁₀heterocyclyl, -L-C₁₋₁₀alkyl-C₃₋₁₀aryl, -L-C₁₋₁₀alkyl-C₁₋₁₀hetaryl, -L-C₁₋₁₀alkyl-C₃₋₁₀cycloalkyl, -L-C₁₋₁₀alkyl-C₁₋₁₀heterocyclyl, -L-C₂₋₁₀alkenyl-C₃₋₁₀aryl, -L-C₂₋₁₀alkenyl-C₁₋₁₀hetaryl, -L-C₂₋₁₀alkenyl-C₃₋₁₀cycloalkyl, -L-C₂₋₁₀alkenyl-C₁₋₁₀heterocyclyl, -L-C₂₋₁₀alkynyl-C₃₋₁₀aryl, -L-C₂₋₁₀alkynyl-C₁₋₁₀hetaryl, -L-C₂₋₁₀alkynyl-C₃₋₁₀cycloalkyl, -L-C₂₋₁₀alkynyl-C₁₋₁₀heterocyclyl, -L-C₁₋₁₀heteroalkyl-C₃₋₁₀aryl, -L-C₁₋₁₀heteroalkyl-C₁₋₁₀hetaryl, -L-C₁₋₁₀heteroalkyl-C₃₋₁₀cycloalkyl, -L-C₁₋₁₀heteroalkyl-C₁₋₁₀heterocyclyl, -L-C₁₋₁₀alkoxy-C₃₋₁₀aryl, -L-C₁₋₁₀alkoxy-C₁₋₁₀hetaryl, -L-C₁₋₁₀alkoxy-C₃₋₁₀cycloalkyl, -L-C₁₋₁₀alkoxy-C₁₋₁₀heterocyclyl, -L-C₃₋₁₀aryl-C₁₋₁₀alkyl, -L-C₃₋₁₀aryl-C₂₋₁₀alkenyl, -L-C₃₋₁₀aryl-C₂₋₁₀alkynyl, -L-C₃₋₁₀aryl-C₁₋₁₀hetaryl, -L-C₃₋₁₀aryl-C₃₋₁₀cycloalkyl, -L-C₃₋₁₀aryl-C₁₋₁₀heterocyclyl, -L-C₁₋₁₀hetaryl-C₁₋₁₀alkyl, -L-C₁₋₁₀hetaryl-C₂₋₁₀alkenyl, -L-C₁₋₁₀hetaryl-C₂₋₁₀alkynyl, -L-C₁₋₁₀hetaryl-C₃₋₁₀aryl, -L-C₁₋₁₀hetaryl-C₃₋₁₀cycloalkyl, -L-C₁₋₁₀hetaryl-C₁₋₁₀heterocyclyl, -L-C₃₋₁₀cycloalkyl-C₁₋₁₀alkyl, -L-C₃₋₁₀cycloalkyl-C₂₋₁₀alkenyl, -L-C₃₋₁₀cycloalkyl-C₂₋₁₀alkynyl, -L-C₃₋₁₀cycloalkyl-C₃₋₁₀aryl, -L-C₃₋₁₀cycloalkyl-C₁₋₁₀hetaryl, -L-C₃₋₁₀cycloalkyl-C₁₋₁₀heterocyclyl, -L-C₁₋₁₀heterocyclyl-C₁₋₁₀alkyl, _l_C₁₋₁₀heterocyclyl-C₂₋₁₀alkenyl, -L-C₁₋₁₀heterocyclyl-C₂₋₁₀alkynyl, -L-C₁₋₁₀heterocyclyl-C₃₋₁₀aryl, -L-C₁₋₁₀heterocyclyl-C₁₋₁₀hetaryl, or -L-C₁₋₁₀heterocyclyl-C₃₋₁₀cycloalkyl, each of which is unsubstituted or substituted by one or more independent R₁₂ substituents;

L is a bond, —O—, —N(R³¹)—, —S(O)₀₋₂—, —C(═O)—, —C(═O)O—, —OC(═O)—, —C(═O)N(R³¹)—, —N(R³¹)C(═O)—, —NR³¹C(═O)O—, —NR³¹C(═O)NR³²—, —NR³¹S(O)₀₋₂—, —S(O)₀₋₂N(R³¹)—, —C(═S)O—, —C(═O)S—, —NR³¹C(═NR³²)NR³²—, —NR³¹C(═NR³²)O—, —NR³¹C(═NR³²)S—, —OC(═O)O—, —OC(═O)NR³¹—, —OC(═O)S—, —SC(═O)S—, —P(O)OR³¹ O—, —SC(═O)NR³¹—;

each of R₃, R₃′ and R₄ is independently hydrogen, halogen, —OH, —CF₃, —OCF₃, —OR³¹, —NR³¹R³², —C(O)R³¹, —CO₂R³¹, —C(═O)NR³¹, —NO₂, —CN, —S(O)₀₋₂R³¹, —SO₂NR³¹R³², —NR³¹C(═O)_{R}³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³²R³³, —NR³¹_{C(═NR}³²)OR³³_{, —NR}³¹C(═NR³²)SR³³, —OC(═O)OR³³, —OC(═O)NR³¹R³², —OC(═O)SR³¹, —SC(═O)SR³¹, —P(O)OR⁻OR³², —SC(═O)NR³¹R³², —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —C₁₋₁₀alkyl-C₃₋₁₀aryl, —C₁₋₁₀alkyl-C₁₋₁₀hetaryl, —C₁₋₁₀alkyl-C₃₋₁₀cycloalkyl, —C₁₋₁₀alkyl-C₁₋₁₀heterocyclyl, —C₂₋₁₀alkenyl-C₃₋₁₀aryl, —C₂₋₁₀alkenyl-C₁₋₁₀hetaryl, —C₂₋₁₀alkenyl-C₃₋₁₀cycloalkyl, —C₂₋₁₀alkenyl-C₁₋₁₀heterocyclyl, —C₂₋₁₀alkynyl-C₃₋₁₀aryl, —C₂₋₁₀alkynyl-C₁₋₁₀hetaryl, —C₂₋₁₀alkynyl-C₃₋₁₀cycloalkyl, —C₂₋₁₀alkynyl-C₁₋₁₀heterocyclyl, —C₁₋₁₀heteroalkyl-C₃₋₁₀aryl, —C₁₋₁₀heteroalkyl-C₁₋₁₀hetaryl, —C₁₋₁₀heteroalkyl-C₃₋₁₀cycloalkyl, —C₁₋₁₀heteroalkyl-C₁₋₁₀heterocyclyl, —C₁₋₁₀alkoxy-C₃₋₁₀aryl, —C₁₋₁₀alkoxy-C₁₋₁₀hetaryl, —C₁₋₁₀alkoxy-C₃₋₁₀cycloalkyl, —C₁₋₁₀alkoxy-C₁₋₁₀heterocyclyl, —C₃₋₁₀aryl-C₁₋₁₀alkyl, —C₃₋₁₀aryl-C₂₋₁₀alkenyl, —C₃₋₁₀aryl-C₂₋₁₀alkynyl, —C₃₋₁₀aryl-C₃₋₁₀hetaryl, —C₃₋₁₀aryl-C₃₋₁₀cycloalkyl, —C₃₋₁₀aryl-C₁₋₁₀heterocyclyl, —C₁₋₁₀hetaryl-C₁₋₁₀alkyl, —C₁₋₁₀hetaryl-C₂₋₁₀alkenyl, —C₁₋₁₀hetaryl-C₂₋₁₀alkynyl, —C₃₋₁₀hetaryl-C₃₋₁₀aryl, —C₁₋₁₀hetaryl-C₃₋₁₀cycloalkyl, —C₁₋₁₀hetaryl-C₁₋₁₀heterocyclyl, —C₃₋₁₀cycloalkyl-C₁₋₁₀alkyl, —C₃₋₁₀cycloalkyl-C₂₋₁₀alkenyl, —C₃₋₁₀cycloalkyl-C₂₋₁₀alkynyl, —C₃₋₁₀cycloalkyl-C₃₋₁₀aryl, —C₃₋₁₀cycloalkyl-C₁₋₁₀hetaryl, —C₃₋₁₀cycloalkyl-C₁₋₁₀heterocyclyl, —C₁₋₁₀heterocyclyl-C₁₋₁₀alkyl, —C ₁₋₁₀heterocyclyl-C₂₋₁₀alkenyl, —C₁₋₁₀heterocyclyl-C₂₋₁₀alkynyl, —C₁₋₁₀heterocyclyl-C₃₋₁₀aryl, —C₁₋₁₀heterocyclyl-C₁₋₁₀hetaryl, or —C₁₋₁₀heterocyclyl-C₃₋₁₀cycloalkyl, each of which is unsubstituted or substituted by one or more independent R₁₃ substituents; or R₃′ is —OR⁶, —NR⁶R³⁴, —S(O)₀₋₂R⁶, —C(═O)R⁶, —C(═O)OR⁶, —OC(═O)R⁶, —C(═O)N(R³⁴)R⁶, or —N(R³⁴)C(═O)R⁶, wherein R⁶ together with R³⁴ can optionally form a heterocyclic ring; or R₃′ is —OR⁶, —NR⁶R³⁴, —S(O)₀₋₂R⁶, —C(═O)OR⁶, —OC(═O)R⁶, —C(═O)N(R³⁴)R⁶, or —N(R³⁴)C(═O)R⁶, wherein R⁶ together with R³⁴ can optionally form a heterocyclic ring;

each of R₅, R₇₁, R₈₁ and R₉₁ is independently hydrogen, halogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —OH, —CF₃, —OCF₃, —OR³¹, —NR³¹R³², —C(O)R³¹, —CO₂R³¹, —C(═O)NR³¹, —NO₂, —CN, —S(O)₀₋₂R³¹, —SO₂NR³¹R³², —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³²R³³, —NR³¹C(═NR³²)OR³³, —NR³¹C(═NR³²)SR³³, —OC(═O)OR³³, —OC(═O)NR³¹R³², —OC(═O)SR³¹, —SC(═O)SR³¹, —P(O)OR⁻OR³², or —SC(═O)NR³¹NR³²;

R₆ is hydrogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —C₁₋₁₀alkyl-C₃₋₁₀aryl, —C₁₋₁₀alkyl-C₁₋₁₀hetaryl, —C₁₋₁₀alkyl-C₃₋₁₀cycloalkyl, —C₁₋₁₀alkyl-C₁₋₁₀heterocyclyl, —C₂₋₁₀alkenyl-C₃₋₁₀aryl, —C₂₋₁₀alkenyl-C₁₋₁₀hetaryl, —C₂₋₁₀alkenyl-C₃₋₁₀cycloalkyl, —C₂₋₁₀alkenyl-C₁₋₁₀heterocyclyl, —C₂₋₁₀alkynyl-C₃₋₁₀aryl, —C₂₋₁₀alkynyl-C₁₋₁₀hetaryl, —C₂₋₁₀alkynyl-C₃₋₁₀cycloalkyl, —C₂₋₁₀alkynyl-C₁₋₁₀heterocyclyl, —C₁₋₁₀heteroalkyl-C₃₋₁₀aryl, —C₁₋₁₀heteroalkyl-C₁₋₁₀hetaryl, —C₁₋₁₀heteroalkyl-C₃₋₁₀cycloalkyl, —C₁₋₁₀heteroalkyl-C₁₋₁₀heterocyclyl, —C₁₋₁₀alkoxy-C₃₋₁₀aryl, —C₁₋₁₀alkoxy-C₁₋₁₀hetaryl, —C₁₋₁₀alkoxy-C₃₋₁₀cycloalkyl, —C₁₋₁₀alkoxy-C₁₋₁₀heterocyclyl, —C₃₋₁₀aryl-C₁₋₁₀alkyl, —C₃₋₁₀aryl-C₂₋₁₀alkenyl, —C₃₋₁₀aryl-C₂₋₁₀alkynyl, —C₃₋₁₀aryl-C₃₋₁₀hetaryl, —C₃₋₁₀aryl-C₃₋₁₀cycloalkyl, —C₃₋₁₀aryl-C₁₋₁₀heterocyclyl, —C₁₋₁₀hetaryl-C₁₋₁₀alkyl, —C₁₋₁₀hetaryl-C₂₋₁₀alkenyl, —C₁₋₁₀hetaryl-C₂₋₁₀alkynyl, —C₃₋₁₀hetaryl-C₃₋₁₀aryl, —C₁₋₁₀hetaryl-C₃₋₁₀cycloalkyl, —C₁₋₁₀hetaryl-C₁₋₁₀heterocyclyl, —C₃₋₁₀cycloalkyl-C₁₋₁₀alkyl, —C₃₋₁₀cycloalkyl-C₂₋₁₀alkenyl, —C₃₋₁₀cycloalkyl-C₂₋₁₀alkynyl, —C₃₋₁₀cycloalkyl-C₃₋₁₀aryl, —C₃₋₁₀cycloalkyl-C₁₋₁₀hetaryl, —C₃₋₁₀cycloalkyl-C₁₋₁₀heterocyclyl, —C₁₋₁₀heterocyclyl-C₁₋₁₀alkyl, —C₁₋₁₀heterocyclyl-C₂₋₁₀alkenyl, —C₁₋₁₀heterocyclyl-C₂₋₁₀alkynyl, —C₁₋₁₀heterocyclyl-C₃₋₁₀aryl, —C₁₋₁₀heterocyclyl-C₁₋₁₀hetaryl, or —C₁₋₁₀heterocyclyl-C₃₋₁₀cycloalkyl, each of which is unsubstituted or substituted by one or more independent R₁₄ or R₁₅ substituents;

each of R₇₂, R₈₂ and R₉₂ is independently hydrogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —OH, —CF₃, —C(O)R³¹, —CO₂R³¹, —C(═O)NR³¹, —S(O)₀₋₂R³¹, —C(═S)OR³¹, —C(═O)SR³¹;

each of R₁₀and R₁₄ is independently —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyolyl, optionally substituted by one or more independent R₁₁ substituents;

each of R₁₁, R_{12,} R₁₃ and R₁₅ is independently hydrogen, halogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —OH, —CF₃, —OCF₃, —OR³¹, —NR³¹R³², —C(O)R³¹, CO₂R³¹, —C(═O)NR³¹, —NO₂, —CN, —S(O)₀₋₂R³¹, —SO₂NR³¹R³², —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³²R³³, —NR³¹C(═NR³²)OR³³, —NR³¹C(═NR³²)SR³³, —OC(═O)OR³³, —OC(═O)NR³¹R³², —OC(═O)SR³¹, —SC(═O)SR³¹, —P(O)OR⁻OR³², or —SC(═O)NR³¹NR³²;

each of R³¹, R³², R³³ and R³⁴ is independently hydrogen, halogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, or wherein R³¹ together with R³² form a heterocyclic ring;

wherein ring A comprises one or more heteroatoms selected from N, O, or S; and

wherein if X₇ is O or X₂-X₃ is R₁C═CR₃, ring A comprises at least two heteroatoms selected from N, O, or S; and

wherein if X₂-X₃ is R₁C═N, at least one of X₇ or X₉ is not N.

223. The method of embodiment 222, wherein the ERK inhibitor is a compound of Formula I-A:

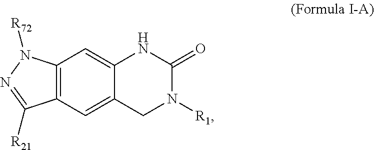

or a pharmaceutically acceptable salt thereof.

224. The method of embodiment 222 or 223, wherein:

R₁ is —C₁₋₁₀alkyl, —C₁₋₁₀alkyl-C₃₋₁₀aryl, or —C₁₋₁₀heterocyclyl-C₁₋₁₀alkyl, each of which is unsubstituted or substituted by one or more independent R₁₀ or R₁₁ substituents;

R₂₁ is -L-C₃₋₁₀aryl or LC₁₋₁₀hetaryl, each of which is unsubstituted or substituted by one or more independent R₁₂ substituents;

L is a bond or —N(R³¹)—;

R₇₂ is hydrogen;

each of R₁₀ is independently —C₃₋₁₀aryl, —C₁₋₁₀hetaryl, or —C₁₋₁₀heterocyclyl, optionally substituted by one or more independent R₁₁ substituents;

each of R₁₁ and R₁₂ is independently halogen, —C₁₋₁₀alkyl, —OH, —CF₃ or —OR³¹; and each of R³¹ is independently hydrogen or —C₁₋₁₀alkyl.

225. The method of any one of embodiments 167 to 224, wherein the ERK inhibitor is selected from the group consisting of:

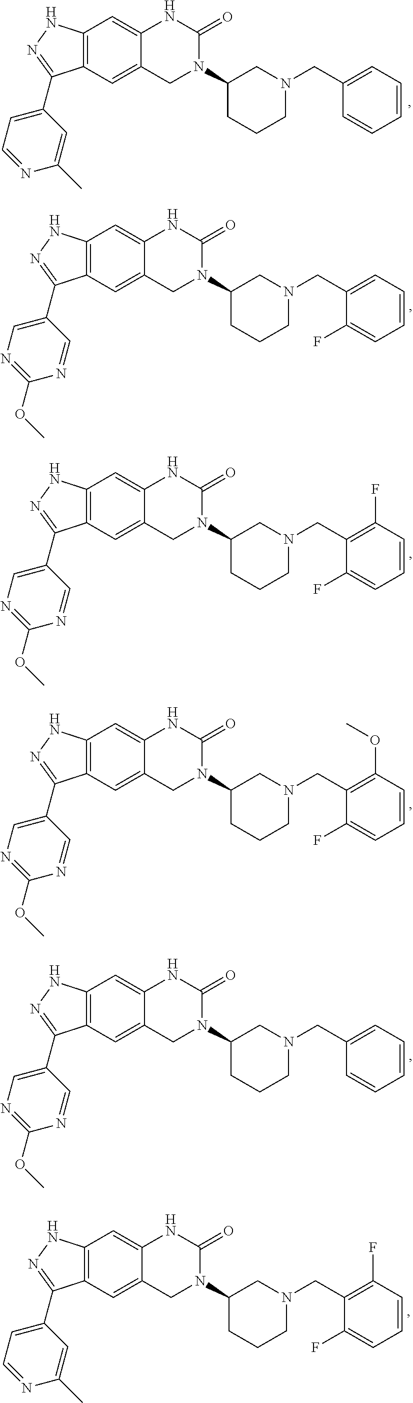

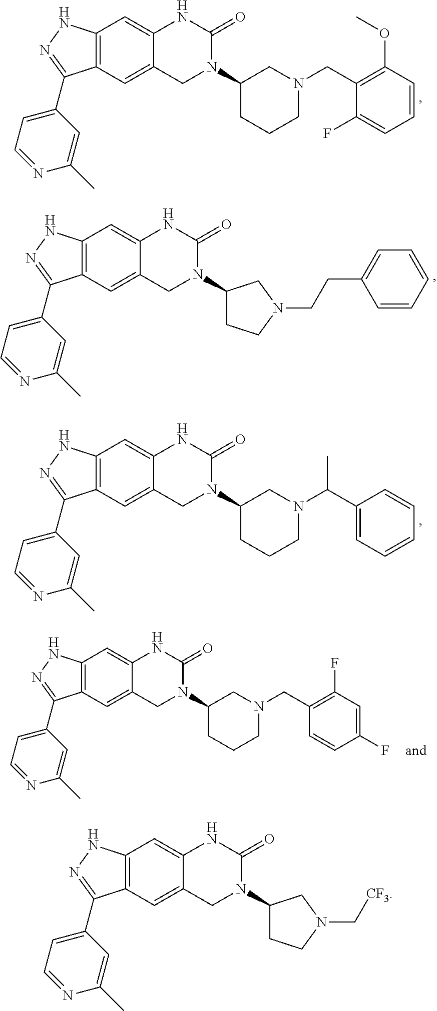

226. The method of any one of embodiments 167 to 221, wherein the ERK inhibitor is selected from the group consisting of ulixertinib, BVD-523, RG7842, GDC-0094, GDC-0994, CC-90003, LTT-462, ASN-007, AMO-01, KO-947, AEZS-134, AEZS-131, AEZS-140, AEZS-136, AEZS-132, D-87503, KIN-2118, RB-1, RB-3, SCH-722984, SCH-772984, MK-8353, SCH-900353, FR-180204, IDN-5491, hyperforin trimethoxybenzoate, ERK1-2067, ERK1-23211, and ERK1-624.

227. The method of any one of embodiments 167 to 221, wherein the ERK inhibitor is selected from the group consisting of:

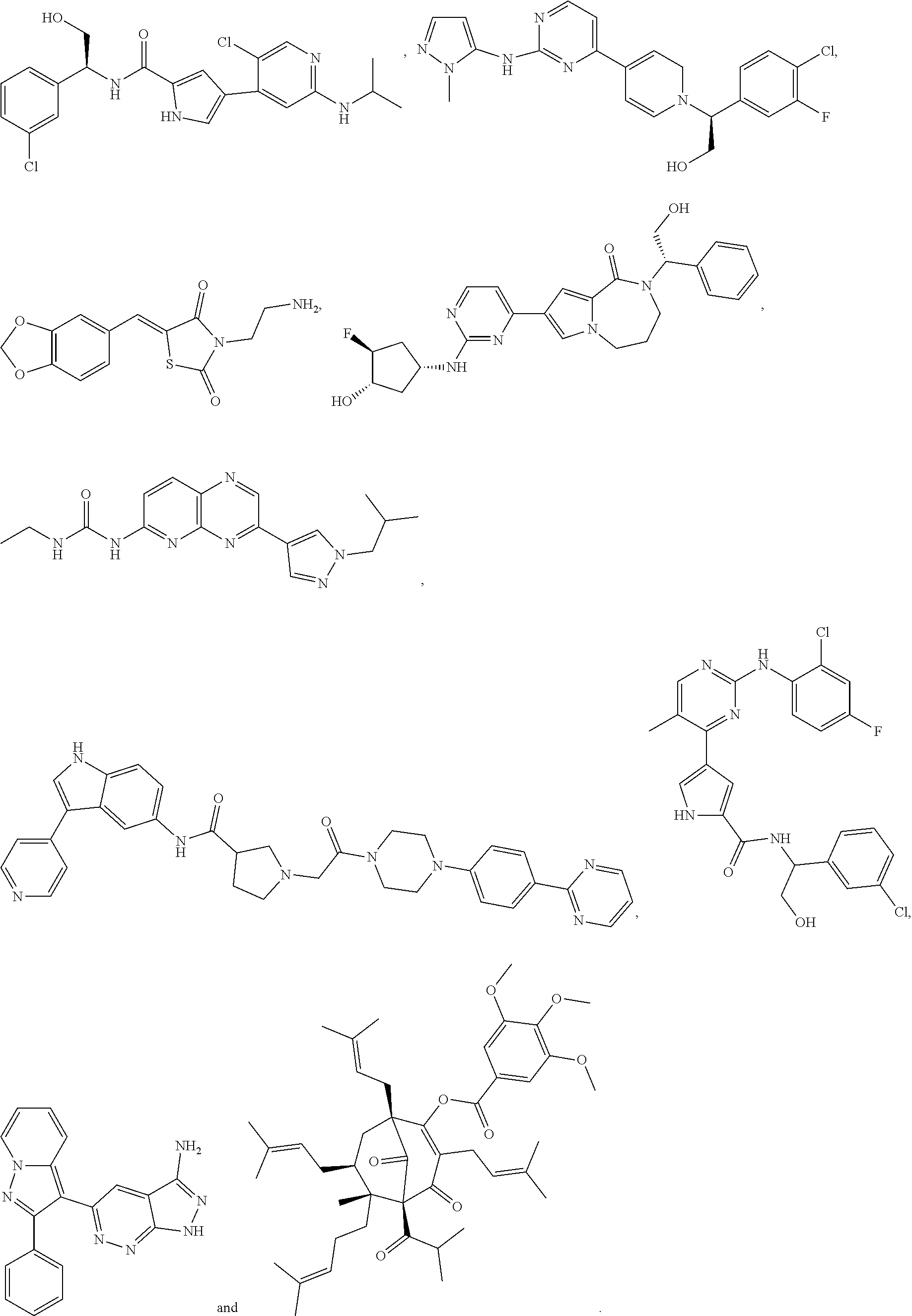

228. The method of any one of embodiments 167 to 227, further comprising administering a second therapeutic agent to the subject.

229. A system for assessing a likelihood of a subject having cancer exhibiting a clinically beneficial response to treatment with an ERK inhibitor, the system comprising:
(a) a memory unit configured to store information concerning a copy number profile and/or expression level of at least one gene located at chromosome 11q13.3-13.4 in a biological sample comprising genomic and/or transcriptomic material from a cancer cell; and
(b) one or more processors alone or in combination programmed to:
   (1) determine a weighted probability of ERK inhibitor responsiveness based on the copy number profile and/or the expression level; and
   (2) designate the subject as having a high probability of exhibiting a clinically beneficial response to treatment with the ERK inhibitor if the weighted probability corresponds to at least 1.5 times a baseline probability, wherein the baseline probability represents a likelihood that the subject will exhibit a clinically beneficial response to treatment with the ERK inhibitor before obtaining the weighted probability of (b)(1).

230. The system of embodiment 229, wherein the expression level is assessed by:
(a) detecting a level of mRNA;
(b) detecting a level of cDNA produced from reverse transcription of mRNA;
(c) detecting a level of polypeptide;
(d) detecting a level of cell-free DNA; or
(e) a nucleic acid amplification assay, a hybridization assay, sequencing, or a combination thereof.

231. The system of embodiment 229, wherein the copy number profile of the at least one gene is assessed by a method selected from the group consisting of in situ hybridization, Southern blot, immunohistochemistry (IHC), polymerase chain reaction (PCR), quantitative PCR (qPCR), quantitative real-time PCR (qRT-PCR), comparative genomic hybridization, microarray-based comparative genomic hybridization, and ligase chain reaction (LCR).

232. The system of any one of embodiments 229 to 231, wherein the at least one gene is selected from CCND1, CTTN, FADD, ORAOV1, ANO1 , PPFIA1 and SHANK2 .

233. The system of embodiment 232, wherein the at least one gene is CCND1 or ANO1 .

234. The system of embodiment 232, wherein the at least one gene is CCND1 and ANO1 .

235. The system of any one of embodiments 229 to 234, wherein the cancer is selected from the group consisting of squamous cell carcinoma and adenocarcinoma.

236. The system of any one of embodiments 229 to 234, wherein the cancer is a squamous cell carcinoma selected from the group consisting of lung, esophageal, cervical, head and neck, bladder and gastric squamous cell carcinomas.

237. The system of embodiment 236, wherein the squamous cell carcinoma is esophageal squamous cell carcinoma.

238. The system of any one of embodiments 229 to 234, wherein the cancer is an adenocarcinoma selected from the group consisting of esophageal and pancreatic adenocarcinomas.

239. The system of any one of embodiments 229 to 234, wherein the cancer is selected from the group consisting of lung, esophageal, cervical, head and neck, bladder, gastric and pancreatic cancer.

240. The system of any one of embodiments 229 to 234, wherein the cancer is selected from breast cancer, pancreatic cancer, lung cancer, thyroid cancer, seminomas, melanoma, bladder cancer, liver cancer, kidney cancer, myelodysplastic syndrome, acute myelogenous leukemia and colorectal cancer.

1. A method of treating cancer in a subject in need thereof, comprising administering an effective dose of an inhibitor of an extracellular signal-regulated kinase (ERK) to the subject, said subject comprising a genome that exhibits amplification and/or overexpression of at least one gene located at chromosome 11q13.3-13.4.

2. The method of claim 1, comprising:
(a) screening the subject for amplification and/or overexpression of the at least one gene located at chromosome 11q13.3-13.4; and
(b) administering the ERK inhibitor to the subject if the amplification and/or overexpression is determined to be present.

3. The method of claim 1, comprising administering the ERK inhibitor to the subject if the subject exhibits amplification and/or overexpression of CCND1 or ANO1.

4. The method of claim 1, comprising administering the ERK inhibitor to the subject if the subject exhibits amplification or overexpression of CCND1 and ANO1.

5. The method of claim 1, wherein the amplification is assessed by a method selected from the group consisting of in situ hybridization, Southern blot, immunohistochemistry (IHC), polymerase chain reaction (PCR), quantitative PCR (qPCR), quantitative real-time PCR (qRT-PCR), comparative genomic hybridization, microarray-based comparative genomic hybridization, and ligase chain reaction (LCR).

6. The method of claim 1, wherein the amplification is assessed using a nucleic acid sample from the subject.

7. The method of claim 6, wherein the nucleic acid sample comprises a nucleic acid selected from the group consisting of genomic DNA, cDNA, ctDNA, cell-free DNA, RNA and mRNA.

8. The method of claim 6, wherein the nucleic acid is from a cancer cell.

9. (canceled)

10. A method of assessing a likelihood of a subject having cancer exhibiting a clinically beneficial response to treatment with an ERK inhibitor, the method comprising:
(a) assessing a copy number profile and/or expression profile of at least one gene located at chromosome 11q13.3-13.4 in a biological sample comprising genomic and/or transcriptomic material from a cancer cell; and
(b) calculating, using a computer system, a weighted probability of ERK inhibitor responsiveness based on the copy number profile and/or the expression profile.

11. The method of claim 10, further comprising designating the subject as having a high probability of exhibiting a clinically beneficial response to treatment with the ERK inhibitor if the weighted probability corresponds to at least 1.5 times a baseline probability, wherein the baseline probability represents a likelihood that the subject will exhibit a clinically beneficial response to treatment with the ERK inhibitor before obtaining the weighted probability of (b).

12. The method of claim 1, wherein the at least one gene is selected from CCNDJ, CTTN, FADD, ORAOVJ, ANO1 , PPFIA1 and SHANK2.

13. The method of claim 12, wherein the at least one gene is CCND1 or ANO1 .

14. The method of claim 1, wherein the cancer is selected from the group consisting of squamous cell carcinoma and adenocarcinoma.

15. The method of claim 14, wherein the cancer is a squamous cell carcinoma selected from the group consisting of lung, esophageal, cervical, head and neck, bladder and gastric squamous cell carcinomas.

16. The method of 10 claim 1, wherein the ERK inhibitor is a compound of Formula I: 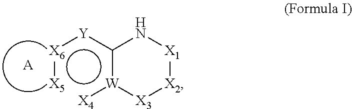
wherein: 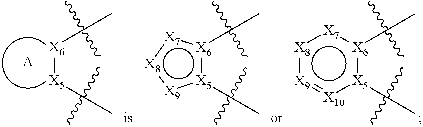
X₁ is C═O, C═S, SO, SO₂, or PO₂⁻; Y is CR₅; W is N or C;
X₂ is NR₁ or CR₁R₁′ and X₃ is null, CR₃R₃′ or C═O; or X₂-X₃ is R₁C═CR₃ or R₁C═N or N═CR₃ or NR₁₂—CR₁₁═CR₃;
X₄ is N or CR₄; X₅ is N or C; X₆ is N or C; X₇ is O, N, NR₇₂ or CR₇₁; X₈ is O, N, NR₈₂ or CR₈₁; X₉ is O, N, NR₂₂ or CR₂₁; X₁₀ is O, N, NR₉₂ or CR₉₁;
R₁ is —C₁₋₁₀alkyl, —C₂₋₁₀alkenyl, —C₂₋₁₀alkynyl, C₁₋₁₀heteroalkyl, —C₃₋₁₀aryl, —C₁₋₁₀hetaryl, —C₃₋₁₀cycloalkyl, —C₁₋₁₀heterocyclyl, —C₁₋₁₀alkyl-C₃₋₁₀aryl, —C₁₋₁₀alkyl-C₁₋₁₀hetaryl, C₁₋₁₀alkyl-C₃₋₁₀cycloalkyl, —C₁₋₁₀alkyl-C₁₋₁₀heterocyclyl, —C₂₋₁₀alkenyl-C₃₋₁₀aryl, —C₂₋₁₀alkenyl-C₁₋₁₀hetaryl, —C₂₋₁₀alkenyl-C₃₋₁₀cycloalkyl, —C₂₋₁₀alkenyl-C₁₋₁₀heterocyclyl, —C₂₋₁₀alkynyl-C₃₋₁₀aryl, —C₂₋₁₀alkynyl-C₁₋₁₀hetaryl, —C₂₋₁₀alkynyl-C₃₋₁₀cycloalkyl, —C₂₋₁₀alkynyl-C₁₋₁₀heterocyclyl, —C₁₋₁₀heteroalkyl-C₃₋₁₀aryl, —C₁₋₁₀heteroalkyl-C₁₋₁₀hetaryl, —C₁₋₁₀heteroalkyl-C₃₋₁₀cycloalkyl, —C₁₋₁₀heteroalkyl-C₁₋₁₀heterocyclyl, —C₁₋₁₀alkoxy-C₃₋₁₀aryl, —C₁₋₁₀alkoxy-C₁₋₁₀hetaryl, —C₁₋₁₀alkoxy-C₃₋₁₀cycloalkyl, —C₁₋₁₀alkoxy-C₁₋₁₀heterocyclyl, —C₃₋₁₀aryl-C₁₋₁₀alkyl, —C₃₋₁₀aryl-C₂₋₁₀alkenyl, —C₃₋₁₀aryl-C₂₋₁₀alkynyl, —C₃₋₁₀aryl-C₃₋₁₀hetaryl, —C₃₋₁₀aryl-C₃₋₁₀cycloalkyl, —C₃₋₁₀aryl-C₁₋₁₀heterocyclyl, —C₁₋₁₀hetaryl-C₁₋₁₀alkyl, —C₁₋₁₀hetaryl-C₂₋₁₀alkenyl, —C₁₋₁₀hetaryl-C₂₋₁₀alkynyl, —C₃₋₁₀hetaryl-C₃₋₁₀aryl, —C₁₋₁₀hetaryl-C₃₋₁₀cycloalkyl, —C₁₋₁₀hetaryl-C₁₋₁₀heterocyclyl, —C₃₋₁₀cycloalkyl-C₁₋₁₀alkyl, —C₃₋₁₀cycloalkyl-C₂₋₁₀alkenyl, —C₃₋₁₀cycloalkyl-C₂₋₁₀alkynyl, —C₃₋₁₀cycloalkyl-C₃₋₁₀aryl, —C₃₋₁₀cycloalkyl-C₁₋₁₀hetaryl, —C₃₋₁₀cycloalkyl-C₁₋₁₀heterocyclyl, —C₁₋₁₀heterocyclyl-C₁₋₁₀alkyl, —C₁₋₁₀heterocyclyl-C₂₋₁₀alkenyl, —C₁₋₁₀heterocyclyl-C₂₋₁₀alkynyl, —C₁₋₁₀heterocyclyl-C₃₋₁₀aryl, —C₁₋₁₀heterocyclyl-C₁₋₁₀hetaryl, or —C₁₋₁₀heterocyclyl-C₃₋₁₀cycloalkyl, each of which is unsubstituted or substituted by one or more independent R₁₀ or R₁₁ sub stituents;
R₁′ is hydrogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —C₁₋₁₀alkyl-C₃₋₁₀aryl, —C₁₋₁₀alkyl-C₁₋₁₀hetaryl, —C₁₋₁₀alkyl-C₃₋₁₀cycloalkyl, —C₁₋₁₀alkyl-C₁₋₁₀heterocyclyl, —C₂₋₁₀alkenyl-C₃₋₁₀aryl, —C₂₋₁₀alkenyl-C₁₋₁₀hetaryl, —C₂₋₁₀alkenyl-C₃₋₁₀cycloalkyl, —C₂₋₁₀alkenyl-C₁₋₁₀heterocyclyl, —C₂₋₁₀alkynyl-C₃₋₁₀aryl, —C₂₋₁₀alkynyl-C₁₋₁₀hetaryl, —C₂₋₁₀alkynyl-C₃₋₁₀cycloalkyl, —C₂₋₁₀alkynyl-C₁₋₁₀heterocyclyl, —C₁₋₁₀heteroalkyl-C₃₋₁₀aryl, —C₁₋₁₀heteroalkyl-C₁₋₁₀hetaryl, —C₁₋₁₀heteroalkyl-C₃₋₁₀cycloalkyl, —C₁₋₁₀heteroalkyl-C₁₋₁₀heterocyclyl, —C₁₋₁₀alkoxy-C₃₋₁₀aryl, —C₁₋₁₀alkoxy-C₁₋₁₀hetaryl, —C₁₋₁₀alkoxy-C₃₋₁₀cycloalkyl, —C₁₋₁₀alkoxy-C₁₋₁₀heterocyclyl, —C₃₋₁₀aryl-C₁₋₁₀alkyl, —C₃₋₁₀aryl-C₂₋₁₀alkenyl, —C₃₋₁₀aryl-C₂₋₁₀alkynyl, —C₃₋₁₀aryl-C₃₋₁₀hetaryl, —C₃₋₁₀aryl-C₃₋₁₀cycloalkyl, —C₃₋₁₀aryl-C₁₋₁₀heterocyclyl, —C₁₋₁₀hetaryl-C₁₋₁₀alkyl, —C₁₋₁₀hetaryl-C₂₋₁₀alkenyl, —C₁₋₁₀hetaryl-C₂₋₁₀alkynyl, —C₃₋₁₀hetaryl-C₃₋₁₀aryl, —C₁₋₁₀hetaryl-C₃₋₁₀cycloalkyl, —C₁₋₁₀hetaryl-C₁₋₁₀heterocyclyl, —C₃₋₁₀cycloalkyl-C₁₋₁₀alkyl, —C₃₋₁₀cycloalkyl-C₂₋₁₀alkenyl, —C₃₋₁₀cycloalkyl-C₂₋₁₀alkynyl, —C₃₋₁₀cycloalkyl-C₃₋₁₀aryl, —C₃₋₁₀cycloalkyl-C₁₋₁₀hetaryl, —C₃₋₁₀cycloalkyl-C₁₋₁₀heterocyclyl, —C₁₋₁₀heterocyclyl-C₁₋₁₀alkyl, C₁₋₁₀heterocyclyl-C₂₋₁₀alkenyl, —C₁₋₁₀heterocyclyl-C₂₋₁₀alkynyl, —C₁₋₁₀heterocyclyl-C₃₋₁₀aryl, —C₁₋₁₀heterocyclyl—C₁₋₁₀hetaryl, or —C₁₋₁₀heterocyclyl-C₃₋₁₀cycloalkyl, each of which is unsubstituted or substituted by one or more independent R₁₀ or R₁₁ substituents;
R₂₁ is hydrogen, halogen, —OH, —CF₃, —OCF₃, —OR³¹, —NR³¹R³², —C(O)R³¹, —CO₂R³¹, —C(═O)NR³¹, —NO₂, —CN, —S(O)₀₋₂R³¹, —SO₂NR³¹R³², —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³²R³³, —NR³¹C(═NR³²)OR³³, —NR³¹C(═NR³²)SR³³, —OC(═O)OR³³, —OC(═O)NR³¹R³², —OC(═O)SR³¹, —SC(═O)SR³¹, —P(O)OR⁻OR³², —SC(═O)NR³¹R³², -L-C₁₋₁₀alkyl, -L-C₂₋₁₀alkenyl, -L-C₂₋₁₀alkynyl, -L-C₁₋₁₀heteroalkyl, -L-C₃₋₁₀aryl, -L-C₁₋₁₀hetaryl, -L-C₃₋₁₀cycloalkyl, -L-C₁₋₁₀heterocyclyl, -L-C₁₋₁₀alkyl-C₃₋₁₀aryl, -L-C₁₋₁₀alkyl-C₁₋₁₀hetaryl, -L-C₁₋₁₀alkyl-C₃₋₁₀cycloalkyl, -L-C₁₋₁₀alkyl-C₁₋₁₀heterocyclyl, -L-C₂₋₁₀alkenyl-C₃₋₁₀aryl, -L-C₂₋₁₀alkenyl-C₁₋₁₀hetaryl, -L-C₂₋₁₀alkenyl-C₃₋₁₀cycloalkyl, -L-C₂₋₁₀alkenyl-C₁₋₁₀heterocyclyl, -L-C₂₋₁₀alkynyl-C₃₋₁₀aryl, -L-C₂₋₁₀alkynyl-C₁₋₁₀hetaryl, -L-C₂₋₁₀alkynyl-C₃₋₁₀cycloalkyl, -L-C₂₋₁₀alkynyl-C₁₋₁₀heterocyclyl, -L-C₁₋₁₀heteroalkyl-C₃₋₁₀aryl, -L-C₁₋₁₀heteroalkyl-C₁₋₁₀hetaryl, -L-C₁₋₁₀heteroalkyl-C₃₋₁₀cycloalkyl, -L-C₁₋₁₀heteroalkyl-C₁₋₁₀heterocyclyl, -L-C₁₋₁₀alkoxy-C₃₋₁₀aryl, -L-C₁₋₁₀alkoxy-C₁₋₁₀hetaryl, -L-C₁₋₁₀alkoxy-C₃₋₁₀cycloalkyl, -L-C₁₋₁₀alkoxy-C₁₋₁₀heterocyclyl, -L-C₃₋₁₀aryl-C₁₋₁₀alkyl, -L-C₃₋₁₀aryl-C₂₋₁₀alkenyl, -L-C₃₋₁₀aryl-C₂₋₁₀alkynyl, -L-C₃₋₁₀aryl-C₁₋₁₀hetaryl, -L-C₃₋₁₀aryl-C₃₋₁₀cycloalkyl, -L-C₃₋₁₀aryl-C₁₋₁₀heterocyclyl, -L-C₁₋₁₀hetaryl-C₁₋₁₀alkyl, -L-C₁₋₁₀hetaryl-C₂₋₁₀alkenyl, -L-C₁₋₁₀hetaryl-C₂₋₁₀alkynyl, -L-C₁₋₁₀hetaryl-C₃₋₁₀aryl, -L-C₁₋₁₀hetaryl-C₃₋₁₀cycloalkyl, -L-C₁₋₁₀hetaryl-C₁₋₁₀heterocyclyl, -L-C₃₋₁₀cycloalkyl-C₁₋₁₀alkyl, -L-C₃₋₁₀cycloalkyl-C₂₋₁₀alkenyl, -L-C₃₋₁₀cycloalkyl-C₂₋₁₀alkynyl, -L-C₃₋₁₀cycloalkyl-C₃₋₁₀aryl, -L-C₃₋₁₀cycloalkyl-C₁₋₁₀hetaryl, -L-C₃₋₁₀cycloalkyl-C₁₋₁₀heterocyclyl, -L-C₁₋₁₀heterocyclyl-C₁₋₁₀alkyl, -L-C₁₋₁₀heterocyclyl-C₂₋₁₀alkenyl, -L-C₁₋₁₀heterocyclyl-C₂₋₁₀alkynyl, -L-C₁₋₁₀heterocyclyl-C₃₋₁₀aryl, -L-C₁₋₁₀heterocyclyl-C₁₋₁₀hetaryl, or -L-C₁₋₁₀heterocyclyl-C₃₋₁₀cycloalkyl, each of which is unsubstituted or substituted by one or more independent R₁₂ substituents;
R₂₂ is hydrogen, —OH, —CF₃, —C(O)R³¹, —CO₂R³¹, —C(═O)NR³¹, —S(O)₀₋₂R³¹, —C(═S)OR³¹, —C(═O)SR³¹, -L-C₁₋₁₀alkyl, -L-C₂₋₁₀alkenyl, -L-C₂₋₁₀alkynyl, -L-C₁₋₁₀heteroalkyl, -L-C₃₋₁₀aryl, -L-C₁₋₁₀hetaryl, -L-C₃₋₁₀cycloalkyl, -L-C₁₋₁₀heterocyclyl, -L-C₁₋₁₀alkyl-C₃₋₁₀aryl, -L-C₁₋₁₀alkyl-C₁₋₁₀hetaryl, -L-C₁₋₁₀alkyl-C₃₋₁₀cycloalkyl, -L-C₁₋₁₀alkyl-C₁₋₁₀heterocyclyl, -L-C₂₋₁₀alkenyl-C₃₋₁₀aryl, -L-C₂₋₁₀alkenyl-C₁₋₁₀hetaryl, -L-C₂₋₁₀alkenyl-C₃₋₁₀cycloalkyl, -L-C₂₋₁₀alkenyl-C₁₋₁₀heterocyclyl, -L-C₂₋₁₀alkynyl-C₃₋₁₀aryl, -L-C₂₋₁₀alkynyl-C₁₋₁₀hetaryl, -L-C₂₋₁₀alkynyl-C₃₋₁₀cycloalkyl, -L-C₂₋₁₀alkynyl-C₁₋₁₀heterocyclyl, -L-C₁₋₁₀heteroalkyl-C₃₋₁₀aryl, -L-C₁₋₁₀heteroalkyl-C₁₋₁₀hetaryl, -L-C₁₋₁₀heteroalkyl-C₃₋₁₀cycloalkyl, -L-C₁₋₁₀heteroalkyl-C₁₋₁₀heterocyclyl, -L-C₁₋₁₀alkoxy-C₃₋₁₀aryl, -L-C₁₋₁₀alkoxy-C₁₋₁₀hetaryl, -L-C₁₋₁₀alkoxy-C₃₋₁₀cycloalkyl, -L-C₁₋₁₀alkoxy-C₁₋₁₀heterocyclyl, -L-C₃₋₁₀aryl-C₁₋₁₀alkyl, -L-C₃₋₁₀aryl-C₂₋₁₀alkenyl, -L-C₃₋₁₀aryl-C₂₋₁₀alkynyl, -L-C₃₋₁₀aryl-C₁₋₁₀hetaryl, -L-C₃₋₁₀aryl-C₃₋₁₀cycloalkyl, -L-C₃₋₁₀aryl-C₁₋₁₀heterocyclyl, -L-C₁₋₁₀hetaryl-C₁₋₁₀alkyl, -L-C₁₋₁₀hetaryl-C₂₋₁₀alkenyl, -L-C₁₋₁₀hetaryl-C₂₋₁₀alkynyl, -L-C₁₋₁₀hetaryl-C₃₋₁₀aryl, -L-C₁₋₁₀hetaryl-C₃₋₁₀cycloalkyl, -L-C₁₋₁₀hetaryl-C₁₋₁₀heterocyclyl, -L-C₃₋₁₀cycloalkyl-C₁₋₁₀alkyl, -L-C₃₋₁₀cycloalkyl-C₂₋₁₀alkenyl, -L-C₃₋₁₀cycloalkyl-C₂₋₁₀alkynyl, -L-C₃₋₁₀cycloalkyl-C₃₋₁₀aryl, -L-C₃₋₁₀cycloalkyl-C₁₋₁₀hetaryl, -L-C₃₋₁₀cycloalkyl-C₁₋₁₀heterocyclyl, -L-C₁₋₁₀heterocyclyl-C₁₋₁₀alkyl, -L-C₁₋₁₀heterocyclyl-C₂₋₁₀alkenyl, -L-C₁₋₁₀heterocyclyl-C₂₋₁₀alkynyl, -L-C₁₋₁₀heterocyclyl-C₃₋₁₀aryl, -L-C₁₋₁₀heterocyclyl-C₁₋₁₀hetaryl, or -L-C₁₋₁₀heterocyclyl-C₃₋₁₀cycloalkyl, each of which is unsubstituted or substituted by one or more independent R₁₂ substituents;
L is a bond, —O—, —N(R³¹)—, —S(O)₀₋₂—, —C(═O)—, —C(═O)O—, —OC(═O)—, —C(═O)N(R³¹)—, —N(R³¹)C(═O)—, —NR³¹C(═O)O—, —NR³¹C(═O)NR³²—, —NR³¹S(O)₀₋₂—, —S(O)₀₋₂N(R³¹)—, —C(═S)O—, —C(═O)S—, —NR³¹C(═NR³²)NR³²—, —NR³¹C(═NR³²)O—, —NR³¹C(═NR³²)S—, —OC(═O)O—, —OC(═O)NR³¹—, —OC(═O)S—, —SC(═O)S—, —P(O)OR⁻O—, —SC(═O)NR³¹—;
each of R₃, R₃′ and R₄ is independently hydrogen, halogen, —OH, —CF₃, —OCF₃, —OR³¹, —NR³¹R³², —C(O)R³¹, —CO₂R³¹, —C(O)NR³¹, —NO₂, —CN, —S(O)₀₋₂R³¹, —SO₂NR³¹R³², —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³²R³³, —NR³¹C(═NR³²)OR³³, —NR³¹C(═NR³²)SR³³, —OC(═O)OR³³, —OC(═O)NR³¹R³², —OC(═O)SR³¹, —SC(═O)SR³¹, —P(O)OR⁻OR³², —SC(═O)NR³¹R³², —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —C₁₋₁₀alkyl-C₃₋₁₀aryl, —C₁₋₁₀alkyl-C₁₋₁₀hetaryl, —C₁₋₁₀alkyl-C₃₋₁₀cycloalkyl, —C₁₋₁₀alkyl-C₁₋₁₀heterocyclyl, —C₂₋₁₀alkenyl-C₃₋₁₀aryl, —C₂₋₁₀alkenyl-C₁₋₁₀hetaryl, —C₂₋₁₀alkenyl-C₃₋₁₀cycloalkyl, —C₂₋₁₀alkenyl-C₁₋₁₀heterocyclyl, —C₂₋₁₀alkynyl-C₃₋₁₀aryl, —C₂₋₁₀alkynyl-C₁₋₁₀hetaryl, —C₂₋₁₀alkynyl-C₃₋₁₀cycloalkyl, —C₂₋₁₀alkynyl-C₁₋₁₀heterocyclyl, —C₁₋₁₀heteroalkyl-C₃₋₁₀aryl, —C₁₋₁₀heteroalkyl-C₁₋₁₀hetaryl, —C₁₋₁₀heteroalkyl-C₃₋₁₀cycloalkyl, —C₁₋₁₀heteroalkyl-C₁₋₁₀heterocyclyl, —C₁₋₁₀alkoxy-C₃₋₁₀aryl, —C₁₋₁₀alkoxy-C₁₋₁₀hetaryl, —C₁₋₁₀alkoxy-C₃₋₁₀cycloalkyl, —C₁₋₁₀alkoxy-C₁₋₁₀heterocyclyl, —C₃₋₁₀aryl-C₂₋₁₀alkenyl, —C₃₋₁₀aryl-C₂₋₁₀alkenyl, —C₃₋₁₀-C₂₋₁₀alkynyl, —C₃₋₁₀aryl-C₃₋₁₀hetaryl, —C₃₋₁₀aryl-C₃₋₁₀cycloalkyl, —C₃₋₁₀aryl-C₁₋₁₀heterocyclyl, —C₁₋₁₀hetaryl-C₁₋₁₀alkyl, —C₁₋₁₀hetaryl-C₂₋₁₀alkenyl, —C₁₋₁₀hetaryl-C₂₋₁₀alkynyl, —C₃₋₁₀hetaryl-C₃₋₁₀aryl, —C₁₋₁₀hetaryl-C₃₋₁₀cycloalkyl, —C₁₋₁₀hetaryl-C₁₋₁₀heterocyclyl, —C₃₋₁₀cycloalkyl-C₁₋₁₀alkyl, —C₃₋₁₀cycloalkyl-C₂₋₁₀alkenyl, —C₃₋₁₀cycloalkyl-C₂₋₁₀alkynyl, —C₃₋₁₀cycloalkyl-C₃₋₁₀aryl, —C₃₋₁₀cycloalkyl-C₁₋₁₀hetaryl, —C₃₋₁₀cycloalkyl-C₁₋₁₀heterocyclyl, —C₁₋₁₀heterocyclyl-C₁₋₁₀alkyl, —C₁₋₁₀heterocyclyl-C₂₋₁₀alkenyl, —C₁₋₁₀heterocyclyl-C₂₋₁₀alkynyl, —C₁₋₁₀heterocyclyl-C₃₋₁₀aryl, —C₁₋₁₀heterocyclyl-C₁₋₁₀hetaryl, or —C₁₋₁₀heterocyclyl-C₃₋₁₀cycloalkyl, each of which is unsubstituted or substituted by one or more independent R₁₃ substituents; or R₃′ is —OR⁶, —NR⁶R³⁴, —S(O)₀₋₂R⁶, —C(═O)R⁶, —C(═O)OR⁶, —OC(═O)R⁶, —C(═O)N(R³⁴)R⁶, or —N(R³⁴)C(═O)R⁶, wherein R⁶ together with R³⁴ can optionally form a heterocyclic ring; or R₃′ is —OR⁶, —NR⁶R³⁴, —S(O)₀₋₂R⁶, —C(═O)R⁶, —C(═O)OR⁶, —OC(═O)R⁶, —C(═O)N(R³⁴)R⁶, or —N(R³⁴)C(═O)R⁶, wherein R⁶ together with R³⁴ can optionally form a heterocyclic ring;
each of R₅, R₇₁, R₈₁ and R₉₁ is independently hydrogen, halogen, —C₁₋₁₀alkyl, —C₂₋₁₀alkenyl, —C₂₋₁₀alkynyl, —C₁₋₁₀heteroalkyl, —C₃₋₁₀aryl, —C₃₋₁₀hetaryl, —C₃₋₁₀cycloalkyl, —C₁₋₁₀heterocyclyl, —OH, —CF₃, —OCF₃, —OR³¹, —NR³¹R³², —C(O)R³¹, —CO₂R³¹, —C(═O)NR³¹, —NO₂, —CN, —S(O)₀₋₂R³¹, —SO₂NR³¹R³², —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³²R³³, —NR³¹C(═NR³²)OR³³, —NR³¹C(═NR³²)SR³³, —OC(═O)OR³³, —OC(═O)NR³¹R³², —OC(═O)SR³¹, —SC(═O)SR³¹, —P(O)OR⁻OR³², or —SC(═O)NR³¹NR³²;
R₆ is hydrogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —C₁₋₁₀alkyl-C₃₋₁₀aryl, —C₁₋₁₀alkyl-C₁₋₁₀hetaryl, —C₁₋₁₀alkyl-C₃₋₁₀cycloalkyl, —C₁₋₁₀alkyl-C₁₋₁₀heterocyclyl, —C₂₋₁₀alkenyl-C₃₋₁₀aryl, —C₂₋₁₀alkenyl-C₁₋₁₀hetaryl, —C₂₋₁₀alkenyl-C₃₋₁₀cycloalkyl, —C₂₋₁₀alkenyl-C₁₋₁₀heterocyclyl, —C₂₋₁₀alkynyl-C₃₋₁₀aryl, —C₂₋₁₀alkynyl-C₁₋₁₀hetaryl, —C₂₋₁₀alkynyl-C₃₋₁₀cycloalkyl, —C₂₋₁₀alkynyl-C₁₋₁₀heterocyclyl, —C₁₋₁₀heteroalkyl-C₃₋₁₀aryl, —C₁₋₁₀heteroalkyl-C₁₋₁₀hetaryl, —C₁₋₁₀heteroalkyl-C₃₋₁₀cycloalkyl, —C₁₋₁₀heteroalkyl-C₁₋₁₀heterocyclyl, —C₁₋₁₀alkoxy-C₃₋₁₀aryl, —C₁₋₁₀alkoxy-C₁₋₁₀hetaryl, —C₁₋₁₀alkoxy-C₃₋₁₀cycloalkyl, —C₁₋₁₀alkoxy-C₁₋₁₀heterocyclyl, —C₃₋₁₀aryl-C₁₋₁₀alkyl, —C₃₋₁₀aryl-C₂₋₁₀alkenyl, —C₃₋₁₀aryl-C₂₋₁₀alkynyl, —C₃₋₁₀aryl-C₃₋₁₀hetaryl, —C₃₋₁₀aryl-C₃₋₁₀cycloalkyl, —C₃₋₁₀aryl-C₁₋₁₀heterocyclyl, —C₁₋₁₀hetaryl-C₁₋₁₀alkyl, —C₁₋₁₀hetaryl-C₂₋₁₀alkenyl, —C₁₋₁₀hetaryl-C₂₋₁₀alkynyl, —C₃₋₁₀hetaryl-C₃₋₁₀aryl, —C₁₋₁₀hetaryl-C₃₋₁₀cycloalkyl, —C₁₋₁₀hetaryl-C₁₋₁₀heterocyclyl, —C₃₋₁₀cycloalkyl-C₁₋₁₀alkyl, —C₃₋₁₀cycloalkyl-C₂₋₁₀alkenyl, —C₃₋₁₀cycloalkyl-C₂₋₁₀alkynyl, —C₃₋₁₀cycloalkyl-C₃₋₁₀aryl, —C₃₋₁₀cycloalkyl-C₁₋₁₀hetaryl, —C₃₋₁₀cycloalkyl-C₁₋₁₀heterocyclyl, —C₁₋₁₀heterocyclyl-C₁₋₁₀alkyl, —C₁₋₁₀heterocyclyl-C₂₋₁₀alkenyl, —C₁₋₁₀heterocyclyl-C₂₋₁₀alkynyl, —C₁₋₁₀heterocyclyl-C₃₋₁₀aryl, —C₁₋₁₀heterocyclyl—C₁₋₁₀hetaryl, or —C₁₋₁₀heterocyclyl-C₃₋₁₀cycloalkyl, each of which is unsubstituted or substituted by one or more independent R₁₄ or R₁₅ substituents;
each of R₇₂, R₈₂ and R₉₂ is independently hydrogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, —OH, —CF₃, —C(O)R³¹, —CO₂R³¹, —C(═O)NR³¹, —S(O)₀₋₂R³¹, —C(═S)OR³¹, —C(═O)SR³¹;
each of R₁₀and R₁₄ is independently —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, optionally substituted by one or more independent R₁₁ substituents;
each of R₁₁, R₁₂, R₁₃ and R₁₅ is independently hydrogen, halogen, —C₁₋₁₀alkyl, —C₂₋₁₀alkenyl, —C₂₋₁₀alkynyl, —C₁₋₁₀heteroalkyl, —C₃₋₁₀aryl, —₁₋₁₀hetaryl, —C₃₋₁₀cycloalkyl, —C₁₋₁₀heterocyclyl, —OH, —CF₃, —OCF₃, —OR³¹, —NR³¹R³², —C(O)R³¹, —CO₂R³¹, —C(═O)NR³¹, —NO₂, —CN, —S(O)₀₋₂R³¹, —SO₂NR³¹R³², —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³²R³³, —NR³¹C(═NR³²)OR³³, —NR³¹C(═NR³²)SR³³, —OC(═O)OR³³, —OC(═O)NR³¹R³², —OC(═O)SR³¹, —SC(═O)SR³¹, —P(O)OR⁻OR³², or —SC(═O)NR³¹NR³²;
each of R³¹, R³², R³³ and R³⁴ is independently hydrogen, halogen, —C₁₋₁₀alkyl, —C₂₋₁₀alkenyl, —C₂₋₁₀alkynyl, —C₁₋₁₀heteroalkyl, —C₃₋₁₀aryl, —C₁₋₁₀hetaryl, —C₃₋₁₀cycloalkyl, —C₁₋₁₀heterocyclyl, or wherein R³¹ together with R³² form a heterocyclic ring;
wherein ring A comprises one or more heteroatoms selected from N, O, or S; and
wherein if X₇ is O or X₂-X₃ is R₁C═CR₃, ring A comprises at least two heteroatoms selected from N, O, or S; and
wherein if X₂-X₃ is R₁C═N, at least one of X₇ or X₉ is not N.

17. The method of claim 16, wherein the ERK inhibitor is a compound of Formula I-A: 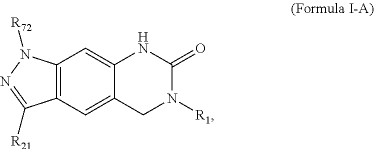
or a pharmaceutically acceptable salt thereof.

18. The method of claim 16, wherein:
R₁ is —C₁₋₁₀alkyl, —C₁₋₁₀alkyl-C₃₋₁₀aryl, or —C₁₋₁₀heterocyclyl-C₁₋₁₀alkyl, each of which is unsubstituted or substituted by one or more independent R₁₀ or R₁₁ substituents;
R₂₁ is -L-C₃₋₁₀aryl or -L-C₁₋₁₀hetaryl, each of which is unsubstituted or substituted by one or more independent R₁₂ substituents;
L is a bond or —N(R³¹)—;
R₇₂ is hydrogen;
each of R₁₀ is independently —C₃₋₁₀aryl, —C₁₋₁₀hetaryl, or —C₁₋₁₀heterocyclyl, optionally substituted by one or more independent R₁₁ substituents;
each of R₁₁ and R₁₂ is independently halogen, —C₁₋₁₀alkyl, —OH, —CF₃ or —OR³¹; and
each of R³¹ is independently hydrogen or —C₁₋₁₀alkyl.

19. The method of claim 16, wherein the ERK inhibitor is selected from the group consisting of

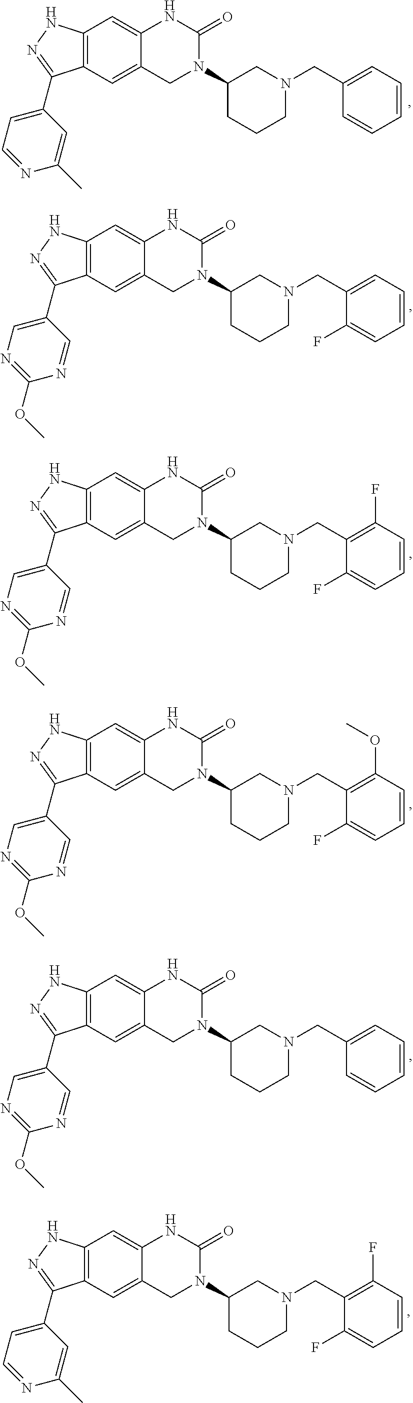

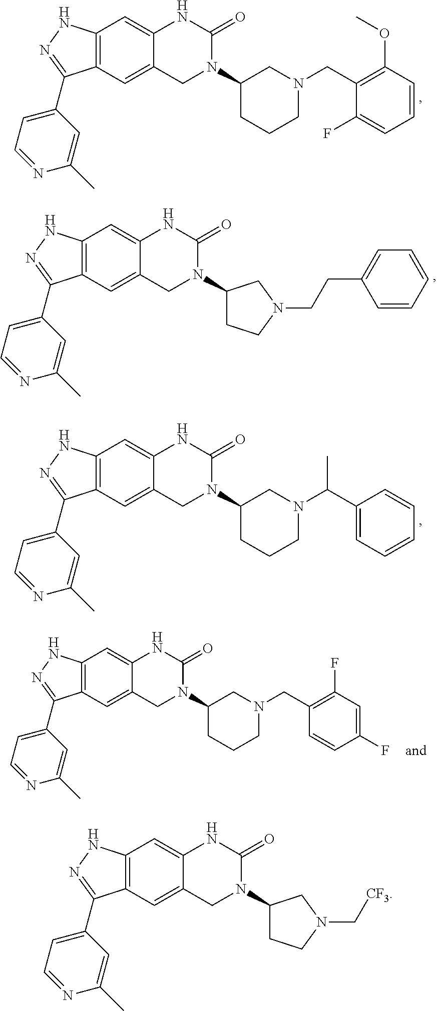

20. The method of 10 claim 1, wherein the ERK inhibitor is selected from the group consisting of ulixertinib, BVD-523, RG7842, GDC-0094, GDC-0994, CC-90003, LTT-462, ASN-007, AMO-01, KO-947, AEZS-134, AEZS-131, AEZS-140, AEZS-136, AEZS-132, D-87503, KIN-2118, RB-1, RB-3, SCH-722984, SCH-772984, MK-8353, SCH-900353, FR-180204, IDN-5491, hyperforin trimethoxybenzoate, ERK1-2067, ERK1-23211, and ERK1-624.

21. The method of any one of claims 1, 2 and 10 claim 1, wherein the ERK inhibitor is selected from the group consisting of: [chemical structure]
